SEQUENCE LISTING (88 sequences)

SEQ ID NO: 1 - protein, 369 aa, Mus musculus
MLKLLLSPRSFLVLQLLLLRAGWSSKVLMSSANEDIKADLILTSTAPEHLSAPTLPLPEVQCFVFNIEYMNCTWNSSSEPQATNLTLHYRYKVSDNNTFQECSHYLFSKEITSGCQIQKEDIQLYQTFVVQLQDPQKPQRRAVQKLNLQNLVIPRAPENLTLSNLSESQLELRWKSRHIKERCLQYLVQYRSNRDRSWTELIVNHEPRFSLPSVDELKRYTFRVRSRYNPICGSSQQWSKWSQPVHWGSHTVEENPSLFALEAVLIPVGTMGLIITLIFVYCWLERMPPIPPIKNLEDLVTEYQGNFSAWSGVSKGLTESLQPDYSERFCHVSEIPPKGGALGEGPGGSPCSLHSPYWPPPCYSLKPEA

SEQ ID NO: 2 - protein, 369 aa, Homo sapiens
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 3 - DNA, 4070 bp, synthetic construct
  source 1..4070: mol_type = other DNA
attaggagcactgcctgttcttccagaggaccaagggtcatttccaagcactcacataatggctaacaatagtatgtaagtcccctgcctgaggttctgatgccttcttctgagttcctctggcacagggcatgacatgtactgtgtagatttacatggaggcaaaatgctcaaacaaataaaataaaaattagctgggtgatggtcgcacacacgtttagtcgcatcatccaggagacggaagcaatcagatctctgtgagttcaaggccagcctggtctacagaatgaatttcaggacagccacaattacacggagaaaccctgtccccaaaaactaaataaataaattaaataaataaataaataaataaaagccacaaattcataaatctgggtctggagagatggttcagtgcttaagaatatttgtggcttctccagaagacccgagttagcctctcatcacaaacatggtagctcaaaaccatctgtagcttcaactccaaagggtctaatgccctcttctcacccccacaggtaccaggcatacatgtagtactcataagaacatgcaaacatgccgggcagtggtggcacatgcctttaatcccagcacttgggaggcagaggcaggtggatttctgagtttgaagccagtctggtctacagagtgagttccaggaaagccagggctacacagagaaaccctgtctcgaaaaaataaataaataaataaataatgaagaaaaagaaaaagaacatgcaaacaaaaactcgtgtgctttaaaataaataaatctaaaaaactttagtctttttaaaattaaaaataaatttaagaagtccctttgctatatagtaagaagcactcactgtgtcacatgctttaggtgttacaatttagagactattagtgtttgcattttctgaatgaggaagctaattttgtttgctttttagttagtttgtttgtttgagaaaggttctctccatgtagtcctacttctcctagagctcccagagtcacagtgatcctcctgacactacctctcatgtgcatgtgctgggattaaaggcctacactaccatccctaagggttttttttttttcttcgagacagggtttctctgtataaccctgctggctgtcctggaactcactctgtagaccaggctgacctcgaactcagaaatccgcctacctctgcctcccaagtgctgggattaaaggcgtgtgtcaccaccgcccggcccacccctaaatttaaggaagctgattcattcattcattcattcattcattctctccctctccctctccctctcctctccctctcctctccctctccctctcctctccctctcctctcccctcccctcctctcctccccctctccccacctctcctccccctttcccccctctccctcctcccccttcctccccctctctcccctcccctcccccctctctccccttccttccttcctttctttctttctttctttctttctttctttctttctttctttctttctttcttgttttgcaacagggtttctttgtatatccttggctgtcctggaacttactcacagcctcctaagtgctgggattaaaggcacttgttaccactgccaggccaggaagctgatttgattttcaaagagtgataatttgtttgcctaatattatatagcctatattctttctattatggtgggtttttgtttttgttttttttgagaaaagacagctaaagtgccataataaaattaccaaaatcagggccagctaaatggctccacaagtaaaggtgcttgctgccaagcctgacagcctgagttcaaccctcagaacccatatggttgaaggaacttacttgcccactgcatatacacatgcatataaaatctatatctgggtatggtggtgcaacaactgtaatcctagcctctgagtgtgaagcccatagagagttagggggcaaccactagatagactctgtctttaaaaaaaaaacaaaaacaaatacataaataagtaatagcaaaaacaaaatgctaaaacccactgtggagatcaagcttgccatgccagtgtttaggaggccaaggaaggctgtttaggctacaaagtgcattgaagtaaaaagtgaaaaggctggtaatggtccacaagtctgaacccttagcagtgaggccacaggaggaggagagttgcaaaggccagccttaaaaatatgatctgattgggtagaagaaaatatactggatgtggtaactttgatttaactctgaagggcttcaaaaacataaatagcagctgagtatagtgatacatgcctgtattcccaacactggagattggaggtgaaagatcaggaattttagatcatcttcagctacacagtaaatttaagactttgaaaaaaattgtagcaattcctggttcacagcaaggtagagttagttccttctgtttaatattgctgtgagcccgaaactgctccaattcactccctctcctcctcgtctcacctaagagtggtgcattttattacaattgatgtatttatattctacactgatatatcattatcacccaaaatacagagggcttttaaaaagccaagcatgctggcactgtattcccagcactttaggagatggagacagagggaccaggagttcaaggccagccccattgcttaccaagtttgaggccaggctgagttacatgagaccttgcttcaacatcaccactaacactctctcccccagaaaaaaggaagcaactaaattgaccaagatggaaagaggatagttcaaggacactttactgaagagatgcttttaaagcagaatctgaaaaaattagtgtaattttaccagagagaaaatagagtagcaaatcaggcagaggcaaagagcagctggctggttcctacctttgtttaactgtgttttggagaatctccacatctatgctgtagaactcattacagaacattgtattatttatatgttccccacttatctctgagcttctaaaatgatgatgtcttatttgtcttatgttctcagaacataagcactgtaccgagcacatattaaagactcaataaatgttggctggataaacaatttcagtaaatggcttctccaatcaaccctgtgctctgagggaaggtaatctggccacagaatgaaggatggactggagagcagaggcccttgagaaaggggaccagtttgtgggttacgggaataatcatgactggaggtaatgaaaggctgatttagcacagtggctgtggttaacagaaaggaggaaactgctgggagaaataccgcagaaacagggctgattggattctcagtgtgagagagaagggggggacatgaaaaggatcctgaggtttcaagtcgggcaggtgatgatgctatttattaagcaggacaaggaagacagaaagacaagcagatttgcagggagctaggaagtctgaagttagtactagttagtactgctctcccatcttagaagccggctctcactagggtcaacagggaccaaactcaggcagcagttaggggtggctattctagtttggattaggtcagaggaaagacagctatgttaagtacccacatgaatcatgtcagtactttccatccatcctccctagactggagaactttgacagatgtttaagatagcctagagggaaaaggtggctgggaatgatggtgtgtggtgggggtgggtgttcagcagagccttcctggacctagggtcctgaagggtcttgattctgtgacactgttgttgatcaagtattaatcttgacagaacatcaccttagagcagaacccaaatctccctggggacttagcttatgtcactgaacacatttaccaaccccccctctctctacagcgtggtttctaaggttctttccaccggaagctacgacaaaaggaaatgtatgggtggggagggcttgtgggagagtggttcagggttctgacacagactacacccagagaaagaagagcaagcacc

SEQ ID NO: 4 - DNA, 3939 bp, synthetic construct
  source 1..3939: mol_type = other DNA
attcaactgtttccaaatcaacaagaaataaagtttttaaccaatgatcatcaagaatgtctgttacaggggttggggagcaggggaaatatggcgaacaatgaagttgggaggaatggggtgagggtaggccaaaggggtgggatagtcatactgaggaaggaagaggaccacagattagctgaaaggagtactgttttggctacttactattctctggtaaatttctgggctcactccaggggtcagtttagagttatctttgccatgttggcccaattcctcgaagctcctttacagatcccatccagtgtcttctctccctggccttactctgccttctattgcacataacacttctgacctcacaattctttaactgcagtcccaggccccacccctatagatgctgacaacccccaacccccaaaagctctctcttgtctgcctagtctgggcctcctccagtctctaaaataatctgctccttcatgcccaacccctatgcttaggaccctagggtcctagagcaccttccatctgcatgatccatcaaggggtgaaggatgaaatgacataagtagcatgtttatcatcatcaaacatggaggtgaggggcaacccagggcaacagtctgggggtggacatggggagggatgaggacagctgagatcactgagacggtagaattcttccatcctatctcttctcagacaatcaagagtttctggtcaattccaattgctctgtcctcctgttgctacattacatccgaaagaaaatgaaattacggaaaacaggtaagtggtttagggaggagtagtgacgaaggtcagaagcaataagctgcctctagactctaaactttcctcccaccaagaaggcttgtcctcctatccttgaaccttcctcaaatccattctgtagaggacctctggccctttttactcatagtctctgttcttccctatcccaattaccccatggccttcctctagacaccattgatttgtgtgatgaatcgggaaccatgaagttactcttcctgtcaaagacacctggagactctgccagcaaattccttacagctcgaaatacctactatgtttgtaaagtggaacgtggggcaccaggtagaggcttgggagtgttctccatagatcaaatccctcaagatggactatgaaacttgtccttaaaataaggcaggatttctcttgtaatcctagcactcaaggaactaagacaggaggactgtgagttaaagggcagtctgggctttatagcaagaactcatctcaaacacaaatgccacacacacaaacaccacagacaaacaatataaagcagggccaggatatagctcacttggcagagtgcttgcctagcatacacaagattctggatgtcatttccaacatggcataaaatttggtgtggtggaacttgcctataatcctagtattgggaggtggaggccggaggatctgaagtttgggatcatttttaactatacagaaactctgaggatagcctgggctacataaaatcctgtctcaaaatgaataaattctttagacatggaggtacggaagcttgagagttggaggcccggtagctcaaaagaaaacacccacaaggtagagccaggaatattaggagttcaacgggaacttgggctaatatgagatcctatcccaagatcccccaaacaaaataagtaaaacagggcattcttagccagatctaaacagaccataactagtcagctcaaagaacactactttgatgacagtaggttaggtacgtatattccaagccacctacttcatcctaggatctgtgtaggtctatgacaaaggggaagaaaaatgtgtaccagctcaagaaagtgtttattgccagaatcctgaaatcttgggcttcaaaagggcaaagactttgtatgtatctggggctggagatggcctaatgttatgtttacaggtaccagaattgagaattcctacaaagctattgtgcccatgctgaagaatccagaacctgaactagttggtaaggcactgaactagatgtgggaggtaggacatagaaagtgctgtacacagctgaggattttgagttaaaggcaccctgggcttaaatgtggaggactctgtcagagaagggggaggggtggtatgaatgtaggggtggggctagcatagtgtgataggtgagatcatgtgggcctaggaaaagggggggcaagggtctaggtaaactttagagggggtgtctggtttaaaccatatgaagcacgtgtatcaccctacttccttcccatcatttctatcttgggccaagctactctgaccgatccatagaatgacagaagatcacgatggattccatctgtttctcagaggggctgctgctttttcttgttttctagactcgctgcgcacacaatgtgacttcctggagaggagcagaataaagatgcttagaactctagaagccaagagactggcagccatggaatcctctgtaaatctcccagcaaggtctcccaagggtggtggagcccagcagactactagcacaagcagtcaactgaaggttcgaatttcctttctaaattccatttcttttttttttaagacttatttatttattatatgtaagtacatcatagctgtcttcagacattccagaagagggtgtcagatctcattacagatggttgtgagccaccatgtggttgcttgggatttgaactcaggaccttcggaagagtagtcggtgctcttaaccgctgagccatctctccagaccctaaattccatttctatgttcactatcggaccccaacctttctccttcctttggccctagacagtaggaatctttcccttcagagcctctccattaatagatttattttccttttccagtctaaaggtgggcgatcagatgaagacggaccaccttccacccgcagaccattctacaagactagagcagactttctcaagagacatcgttaactcaatacaaagaccaagtgagagcagcagctaccaggagagagcatgaaattactgggatttaacgtgaggtttctatttctagaagggctcctactcccagcatttgcacaaaggcttcagtaggctgtaggcaccagggaggccgagggtggaccagagcaacaacagagggctggagtgaacacttgagggggggggcggtgaggaaagactgtgaagtctacttgctagacttttagtacaataagcctttgagggccagagaagggggtggggtcttctcaaaccagactacctcccctcgggggaggcctatcttgcagccgcctactcctgctggggttaggtgtccctcccccaacacactccattacctcccaggctgaaattcccccacctcccagcctgcgggcagtccctcccaccagcccttcgcctaactgcccttcgcctttccgcctccctccctccccccatattggacggggagggggggggcagcaaaggcagcagaaaccagaaaccagatctgattatagacatttattgaactcatttattgatttgacacacttccccactccaatctagcacatgatacaatctgggtaggccaggcgctgacacaggaaatgatgggaacccacagcaggagtgggggaggggacaatgcaaaggcgctgggggaggggctgcttgggggctggggtggaggagccccagatggcctctctggcaggtgctagtaaacctgatccacattccccccaaccctgctcatttcccagaccctcagccaaactcgtcaggctctcccttccctgttcctctccacagccctgcttgctgctacacagccttccacataaccctggcagaccacaacagggaagaagggcagggaca

SEQ ID NO: 5 - DNA, 4200 bp, synthetic construct
  source 1..4200: mol_type = other DNA
atgttgaagccatcattaccattcacatccctcttattcctgcagctgcccctgctgggagtggggctgaacacgacaattctgacgcccaatgggaatgaagacaccacagctggtgggaaatctgggactggagggggctggtgagaagggtggctgtgggaaggggccgtacagagatctggtgcctgccactggccattacaatcatgtgggcagaattgaaaagtggagtgggaagggcaagggggagggttccctgcctcacgctacttcttctttctttcttgtttgtttgtttctttctttcttttgaggcagggtctcactatgttgcctaggctggtctcaaactcctggcctctagtgatcctcctgcctcagcctttcaaagcaccaggattacagacatgagccaccgtgcttggcctcctccttctgaccatcatttctctttccctccctgccttcattttctccccaatctagatttcttcctgaccactatgcccactgactccctcagtgtttccactctgcccctcccagaggttcagtgttttgtgttcaatgtcgagtacatgaattgcacttggaacagcagctctgagccccagcctaccaacctcactctgcattattggtatgagaagggacgagggggaggggatgaagaagaggtgggttggatcagagaccaagagagagggtagcaagtctcccaggtaccccactgttttctcctggggtaagtcataagtcggttgaggggagatgaggctaggctctggatatctgcagtacccagattggccccactgttcctcttccttccaacctttctcctctaggtacaagaactcggataatgataaagtccagaagtgcagccactatctattctctgaagaaatcacttctggctgtcagttgcaaaaaaaggagatccacctctaccaaacatttgttgttcagctccaggacccacgggaacccaggagacaggccacacagatgctaaaactgcagaatctgggtaatttggaaagaaagggtcaagagaccagggatactgtgggacattggagtctacagagtagtgttcttttatcataagggtacatgggcagaaaagaggaggtaggggatcatgatgggaagggaggaggtattaggggcactaccttcaggatcctgacttgtctaggccaggggaatgaccacatatgcacacatatctccagtgatcccctgggctccagagaacctaacacttcacaaactgagtgaatcccagctagaactgaactggaacaacagattcttgaaccactgtttggagcacttggtgcagtaccggactgactgggaccacagctggactgtgagtgactagggacgtgaatgtagcagctaaggccaagaaagtagggctaaaggattcaaccagacagatagaaggacctaatatcaagctcctgttctctgcctcccagcttctctgctcaccccctaccctccctcctccaactcctttcccccctattttctccagtgagttttctttttttcttttcttttctttctttctttcttttttttttttttttgagacagagcctcactctgttgcccaggcttgagtgcagtggggcgatcttgggctcactgcgacctctgtctccctggttcaagtgattctcctgcttcagcctcccaagtagctgggagcatgcacaaccatgcctggctaatttttgtatttttagtaaagacagggttttgccatgttggtcaggctggtcttgaactcctgacctcaggtgatctgcccacctcggcctcccaaagtgctgggattacaggcgtgagccaccattcctgacaccagtgagttttcattagggattccctacccatactcttcctgataccagatagacaagtaaacaaaaggaagccattaaggggatccagaggggaggcattagattcaagtcagtgaagggagcagtgtggcttgagtagtcaagagatgagagagaaactgggcagtagcagagatgacactggtgggtgttcaggagtatgttttaattctcccttctctcatagacacccactttccctcatcctctttctcctcaaggaacaatcagtggattatagacataagttctccttgcctagtgtggatgggcagaaacgctacacgtttcgtgttcggagccgctttaacccactctgtggaagtgctcagcattggagtgaatggagccacccaatccactgggggagcaatacttcaaaaggtaaaatgggcccacatgacccaatccatgagcccaacaccccagcctttctaacaccactgtcttttgctccacttccctgtcactaaagcccctaaacttggtgccccatctctccacactgtctaaccccaacctctagaaatcaaggtttttctgtgtagggttgggttagcgtgttgttagagtaggggagtggattgagaaggaggctgaggggtactcaagggggctatagaatgtataggatttccctgaagcattcctagagagcctgcaaggtgaagatggctttggaaccagctggatctaggctgtgccacatactacctctttggccttggccacatccctaaactcttggattctgtttcctaagatgtaagatggaggtaattgttcctgcctcacaggagctgttgtgaggattaaacagagagtatgtctttagcgcggtgcctggcaccagtgcctggcatgtagtaggggcacaacaaatataaggtccactttgcttttcttttttctatagagaatcctttcctgtttgcattggaagccgtggttatctctgttggctccatgggattgattatcagccttctctgtgtgtatttctggctggaacggtgagatttggagaagcccagaaaaatgaggggaacggtagctgacaatagcagaggagggttttgcagggtctttaggagtaaaggatgagacagtaagtaatgagagattacccaagagggtttggtgatggaaggaagccacaggcacagagaacacagaatcactttatttcatatgggacaactgggagaagggtgataaaaaagctttaacctatgtgctcctgctccctctttctcccctgtcaggacgatgccccgaattcccaccctgaagaacctagaggatcttgttactgaataccacgggaacttttcggtgagaacgctgtcataagcatgctgcagtctatcaactgccaactgcctgccagcaagacagacagagtgtgggggtgggggcagagaggagagggaaggaggccctgcactaactgtcaggatgtggccgaccaaatggggcatggactatacagagagagacacacacagaagtgcagattatagattgaatgaggcagatggcaactggtattgggggcccaggagcctgtgtatcccttctgtaatcaattacagtggttgcagacatcatgagtactcctttggcacagagctcggtcttttacttcctgcccctaattgacccctgacctggacatatctgtctttaggcctggagtggtgtgtctaagggactggctgagagtctgcagccagactacagtgaacgactctgcctcgtcagtgagattcccccaaaaggaggggcccttggggaggggcctggggcctccccatgcaaccagcatagcccctactgggcccccccatgttacaccctaaagcctgaaacctgaaccccaatcctctgacagaagaaccccagggtcctgtagccctaagtggtactaactttccttcattcaacccacctgcgtctcatactcacctcaccccactgtggctgatttggaattttgtgcccccatgtaagcaccccttcatttggcattccccacttgagaattacccttttgccccgaacatgtttttcttctccctcagtctggcccttccttttcgcaggattcttcctccctccctctttccctcccttcctctttccatctaccctccgattgttcctgaaccgatgagaaataaagtttctgttgataatcatcaaaaatgtctgtcatgggggtggggagcaggggaagcctgggatgcaatgagggtgggaggggaagaaa

SEQ ID NO: 6 - DNA, 73 bp, synthetic construct
  source 1..73: mol_type = other DNA
gaagcctgggatgcaatgagggtgggaggggaagaaaaagcttgatatcgaattccgaagttcctattctcta

SEQ ID NO: 7 - DNA, 64 bp, synthetic construct
  source 1..64: mol_type = other DNA
ttcatcagtcaggtacataatggtggatccgatatcattcaactgtttccaaatcaacaagaaa

SEQ ID NO: 8 - DNA, 1716 bp, synthetic construct
  source 1..1716: mol_type = other DNA
cccaacactggagattggaggtgaaagatcaggaattttagatcatcttcagctacacagtaaatttaagactttgaaaaaaattgtagcaattcctggttcacagcaaggtagagttagttccttctgtttaatattgctgtgagcccgaaactgctccaattcactccctctcctcctcgtctcacctaagagtggtgcattttattacaattgatgtatttatattctacactgatatatcattatcacccaaaatacagagggcttttaaaaagccaagcatgctggcactgtattcccagcactttaggagatggagacagagggaccaggagttcaaggccagccccattgcttaccaagtttgaggccaggctgagttacatgagaccttgcttcaacatcaccactaacactctctcccccagaaaaaaggaagcaactaaattgaccaagatggaaagaggatagttcaaggacactttactgaagagatgcttttaaagcagaatctgaaaaaattagtgtaattttaccagagagaaaatagagtagcaaatcaggcagaggcaaagagcagctggctggttcctacctttgtttaactgtgttttggagaatctccacatctatgctgtagaactcattacagaacattgtattatttatatgttccccacttatctctgagcttctaaaatgatgatgtcttatttgtcttatgttctcagaacataagcactgtaccgagcacatattaaagactcaataaatgttggctggataaacaatttcagtaaatggcttctccaatcaaccctgtgctctgagggaaggtaatctggccacagaatgaaggatggactggagagcagaggcccttgagaaaggggaccagtttgtgggttacgggaataatcatgactggaggtaatgaaaggctgatttagcacagtggctgtggttaacagaaaggaggaaactgctgggagaaataccgcagaaacagggctgattggattctcagtgtgagagagaagggggggacatgaaaaggatcctgaggtttcaagtcgggcaggtgatgatgctatttattaagcaggacaaggaagacagaaagacaagcagatttgcagggagctaggaagtctgaagttagtactagttagtactgctctcccatcttagaagccggctctcactagggtcaacagggaccaaactcaggcagcagttaggggtggctattctagtttggattaggtcagaggaaagacagctatgttaagtacccacatgaatcatgtcagtactttccatccatcctccctagactggagaactttgacagatgtttaagatagcctagagggaaaaggtggctgggaatgatggtgtgtggtgggggtgggtgttcagcagagccttcctggacctagggtcctgaagggtcttgattctgtgacactgttgttgatcaagtattaatcttgacagaacatcaccttagagcagaacccaaatctccctggggacttagcttatgtcactgaacacatttaccaaccccccctctctctacagcgtggtttctaaggttctttccaccggaagctacgacaaaaggaaatgtatgggtggggagggcttgtgggagagtggttcagggttctgacacagactacacccagagaaagaagagcaagcacc

SEQ ID NO: 9 - DNA, 1596 bp, synthetic construct
  source 1..1596: mol_type = other DNA
attcaactgtttccaaatcaacaagaaataaagtttttaaccaatgatcatcaagaatgtctgttacaggggttggggagcaggggaaatatggcgaacaatgaagttgggaggaatggggtgagggtaggccaaaggggtgggatagtcatactgaggaaggaagaggaccacagattagctgaaaggagtactgttttggctacttactattctctggtaaatttctgggctcactccaggggtcagtttagagttatctttgccatgttggcccaattcctcgaagctcctttacagatcccatccagtgtcttctctccctggccttactctgccttctattgcacataacacttctgacctcacaattctttaactgcagtcccaggccccacccctatagatgctgacaacccccaacccccaaaagctctctcttgtctgcctagtctgggcctcctccagtctctaaaataatctgctccttcatgcccaacccctatgcttaggaccctagggtcctagagcaccttccatctgcatgatccatcaaggggtgaaggatgaaatgacataagtagcatgtttatcatcatcaaacatggaggtgaggggcaacccagggcaacagtctgggggtggacatggggagggatgaggacagctgagatcactgagacggtagaattcttccatcctatctcttctcagacaatcaagagtttctggtcaattccaattgctctgtcctcctgttgctacattacatccgaaagaaaatgaaattacggaaaacaggtaagtggtttagggaggagtagtgacgaaggtcagaagcaataagctgcctctagactctaaactttcctcccaccaagaaggcttgtcctcctatccttgaaccttcctcaaatccattctgtagaggacctctggccctttttactcatagtctctgttcttccctatcccaattaccccatggccttcctctagacaccattgatttgtgtgatgaatcgggaaccatgaagttactcttcctgtcaaagacacctggagactctgccagcaaattccttacagctcgaaatacctactatgtttgtaaagtggaacgtggggcaccaggtagaggcttgggagtgttctccatagatcaaatccctcaagatggactatgaaacttgtccttaaaataaggcaggatttctcttgtaatcctagcactcaaggaactaagacaggaggactgtgagttaaagggcagtctgggctttatagcaagaactcatctcaaacacaaatgccacacacacaaacaccacagacaaacaatataaagcagggccaggatatagctcacttggcagagtgcttgcctagcatacacaagattctggatgtcatttccaacatggcataaaatttggtgtggtggaacttgcctataatcctagtattgggaggtggaggccggaggatctgaagtttgggatcatttttaactatacagaaactctgaggatagcctgggctacataaaatcctgtctcaaaatgaataaattctttagacatggaggtacggaag

SEQ ID NO: 10 - DNA, 23 bp, synthetic construct
  source 1..23: mol_type = other DNA
gttgttgagaggaaggctatggg

SEQ ID NO: 11 - DNA, 23 bp, synthetic construct
  source 1..23: mol_type = other DNA
tttggaaacagttgaatcatagg

SEQ ID NO: 12 - DNA, 132 bp, synthetic construct
  source 1..132: mol_type = other DNA
gaattctaatacgactcactatagggggtcttcgagaagacctgttttagagctagaaatagcaagttaaaataaggctagtccgttatcaacttgaaaaagtggcaccgagtcggtgcttttaaaggatcc

SEQ ID NO: 13 - DNA, 25 bp, synthetic construct
  source 1..25: mol_type = other DNA
caagcttgccatgccagtgtttagg

SEQ ID NO: 14 - DNA, 26 bp, synthetic construct
  source 1..26: mol_type = other DNA
cattcactccaatgctgagcacttcc

SEQ ID NO: 15 - DNA, 25 bp, synthetic construct
  source 1..25: mol_type = other DNA
gaagccattaaggggatccagaggg

SEQ ID NO: 16 - DNA, 25 bp, synthetic construct
  source 1..25: mol_type = other DNA
gagctggtacacatttttcttcccc

SEQ ID NO: 17 - DNA, 25 bp, synthetic construct
  source 1..25: mol_type = other DNA
ggacctagggtcctgaagggtcttg

SEQ ID NO: 18 - DNA, 25 bp, synthetic construct
  source 1..25: mol_type = other DNA
ccccctctgcacacttcgttttagt

SEQ ID NO: 19 - DNA, 25 bp, synthetic construct
  source 1..25: mol_type = other DNA
cccactccacttttcaattctgccc

SEQ ID NO: 20 - DNA, 25 bp, synthetic construct
  source 1..25: mol_type = other DNA
gaatgtaggggtggggctagcatag

SEQ ID NO: 21 - DNA, 25 bp, synthetic construct
  source 1..25: mol_type = other DNA
ctagtagtctgctgggctccaccac

SEQ ID NO: 22 - DNA, 27 bp, synthetic construct
  source 1..27: mol_type = other DNA
gttggctggataaacaatttcagtaaa

SEQ ID NO: 23 - DNA, 26 bp, synthetic construct
  source 1..26: mol_type = other DNA
ctaactagtactaacttcagacttcc

SEQ ID NO: 24 - DNA, 6125 bp, synthetic construct
  source 1..6125: mol_type = other DNA
ggtgttcatagctcaagatcccgaaagccgccatcttgcccagcccgtaccgcctgaggcgcctgcagggccaggcgaggtggggagggaaggggcggagggccgaggacgggagggagcacggggacagcgagccgggcggcggtagagaggaccgaggccgagagaaggggggtggcggaggcggggcacgttagaaactctcgattactgccggaaactgccgagggacgctgggtaccgtcggacaatactgactgcagagtgggtggggcctcgccggcgcggggcggagccccacagcccgggagctttgggccctaggagggaaagtcaatttcacacactagaaatccaccggcgtggccggggctggaggcgcagacctggaatagggaaggcagccgcggcgctgagctctctcgctggaactggcttcccgtggcctcgctcagttcttggcctaaaataagtgctcgggaatttttagtttagttaataactgcgtcttttactgatcacttcgtttcttggttctcacatctggataatgggcatgctaaacttccttcaaaggacagttgtaaagattagaaactactattgttttaagaaagcacctctgatctgatacaaaagatccggtctagccaggcgcaggcctttaacccctgcagaggcagagacagacggatctctgtgaagtgcttgccttacacaaaaagaaagcaactgtaaaactgaagtgcttataatctctccaggtggttacaggccctctgcaccacaaccccctaaccctttgttgttgttgttaacccttaacctggcggtgcacacctttagtcccagcagaagaaagcagatctctgtgaattcaaggccagcctggtttacacaatgacttccagaacagccaagtccatgtatgaactgtgtaaccctgtctcaacaacaacaacaacaaaaccaaagaaacaaacaaaaagtagccagattgatggcaaacacctttagtcctagcaactagagaggcaggagcaggagtatctgagttcaaggccagcctggtctacacactgagctccagaacagccagaattacatagcgcccttgtctcaaaagaaaaaaaaccacagggtttggggatttacctcaatggtagagcacttgcctagcaaatacaagaccctgggtacccgtcctcagctctggaaggaaaaaaacaaaccaaaaaaaaaaaaaaatgccaacgtgtcatcagagtcagatgtggtggggcaagcaggtcatccagagcaacagtgactgacacataccctgtctcaaaaagaaatccattgacacttacattatgtgtgtcacacacctgtaattcccacagctcgggagattggcaaaaggataacaagttacttccagcccaaattacatagacacacacacacacatacacacacctcttttctgagacaaggtcatctcatcatgtaatcgcaggtggcctaaaactttccatatagcccacgctggcctagaactcacagaaagctcagtgagccatctgcctcctaaagcttccaggccggatcaaaaggaaaagggaggaggagcaggaggaggaagaaacaggaatcttcacagaatagtagttatacaaattgtcagatattttctatcatgaagctttgtatatctgaactccaacttatgaaccttcacaatagggccaggcagaaggcagaagatgcttgcttgccattgtacctgattaccaggatagaatcttccggtttcacatgatgaaagcagagaactgacttttgcaaactctcctctgagcaccgtgtacatgcatggaacaaacaaacaaataaataaaatgtacaaacgactctttctcagacttagtggcaaagggcactaacccggcactcctgtgacagaagtcggcagtctctttgacttccagaacagccaggactacacagtgagatcttgtctcaataaataaataatctaaaaaaaagaggagctgcagagatggctcagggattaggagcactgcctgttcttccagaggaccaagggtcatttccaagcactcacataatggctaacaatagtatgtaagtcccctgcctgaggttctgatgccttcttctgagttcctctggcacagggcatgacatgtactgtgtagatttacatggaggcaaaatgctcaaacaaataaaataaaaattagctgggtgatggtcgcacacacgtttagtcgcatcatccaggagacggaagcaatcagatctctgtgagttcaaggccagcctggtctacagaatgaatttcaggacagccacaattacacggagaaaccctgtccccaaaaactaaataaataaattaaataaataaataaataaataaaagccacaaattcataaatctgggtctggagagatggttcagtgcttaagaatatttgtggcttctccagaagacccgagttagcctctcatcacaaacatggtagctcaaaaccatctgtagcttcaactccaaagggtctaatgccctcttctcacccccacaggtaccaggcatacatgtagtactcataagaacatgcaaacatgccgggcagtggtggcacatgcctttaatcccagcacttgggaggcagaggcaggtggatttctgagtttgaagccagtctggtctacagagtgagttccaggaaagccagggctacacagagaaaccctgtctcgaaaaaataaataaataaataaataatgaagaaaaagaaaaagaacatgcaaacaaaaactcgtgtgctttaaaataaataaatctaaaaaactttagtctttttaaaattaaaaataaatttaagaagtccctttgctatatagtaagaagcactcactgtgtcacatgctttaggtgttacaatttagagactattagtgtttgcattttctgaatgaggaagctaattttgtttgctttttagttagtttgtttgtttgagaaaggttctctccatgtagtcctacttctcctagagctcccagagtcacagtgatcctcctgacactacctctcatgtgcatgtgctgggattaaaggcctacactaccatccctaagggttttttttttttcttcgagacagggtttctctgtataaccctgctggctgtcctggaactcactctgtagaccaggctgacctcgaactcagaaatccgcctacctctgcctcccaagtgctgggattaaaggcgtgtgtcaccaccgcccggcccacccctaaatttaaggaagctgattcattcattcattcattcattcattctctccctctccctctccctctcctctccctctcctctccctctccctctcctctccctctcctctcccctcccctcctctcctccccctctccccacctctcctccccctttcccccctctccctcctcccccttcctccccctctctcccctcccctcccccctctctccccttccttccttcctttctttctttctttctttctttctttctttctttctttctttctttctttcttgttttgcaacagggtttctttgtatatccttggctgtcctggaacttactcacagcctcctaagtgctgggattaaaggcacttgttaccactgccaggccaggaagctgatttgattttcaaagagtgataatttgtttgcctaatattatatagcctatattctttctattatggtgggtttttgtttttgttttttttgagaaaagacagctaaagtgccataataaaattaccaaaatcagggccagctaaatggctccacaagtaaaggtgcttgctgccaagcctgacagcctgagttcaaccctcagaacccatatggttgaaggaacttacttgcccactgcatatacacatgcatataaaatctatatctgggtatggtggtgcaacaactgtaatcctagcctctgagtgtgaagcccatagagagttagggggcaaccactagatagactctgtctttaaaaaaaaaacaaaaacaaatacataaataagtaatagcaaaaacaaaatgctaaaacccactgtggagatcaagcttgccatgccagtgtttaggaggccaaggaaggctgtttaggctacaaagtgcattgaagtaaaaagtgaaaaggctggtaatggtccacaagtctgaacccttagcagtgaggccacaggaggaggagagttgcaaaggccagccttaaaaatatgatctgattgggtagaagaaaatatactggatgtggtaactttgatttaactctgaagggcttcaaaaacataaatagcagctgagtatagtgatacatgcctgtattcccaacactggagattggaggtgaaagatcaggaattttagatcatcttcagctacacagtaaatttaagactttgaaaaaaattgtagcaattcctggttcacagcaaggtagagttagttccttctgtttaatattgctgtgagcccgaaactgctccaattcactccctctcctcctcgtctcacctaagagtggtgcattttattacaattgatgtatttatattctacactgatatatcattatcacccaaaatacagagggcttttaaaaagccaagcatgctggcactgtattcccagcactttaggagatggagacagagggaccaggagttcaaggccagccccattgcttaccaagtttgaggccaggctgagttacatgagaccttgcttcaacatcaccactaacactctctcccccagaaaaaaggaagcaactaaattgaccaagatggaaagaggatagttcaaggacactttactgaagagatgcttttaaagcagaatctgaaaaaattagtgtaattttaccagagagaaaatagagtagcaaatcaggcagaggcaaagagcagctggctggttcctacctttgtttaactgtgttttggagaatctccacatctatgctgtagaactcattacagaacattgtattatttatatgttccccacttatctctgagcttctaaaatgatgatgtcttatttgtcttatgttctcagaacataagcactgtaccgagcacatattaaagactcaataaatgttggctggataaacaatttcagtaaatggcttctccaatcaaccctgtgctctgagggaaggtaatctggccacagaatgaaggatggactggagagcagaggcccttgagaaaggggaccagtttgtgggttacgggaataatcatgactggaggtaatgaaaggctgatttagcacagtggctgtggttaacagaaaggaggaaactgctgggagaaataccgcagaaacagggctgattggattctcagtgtgagagagaagggggggacatgaaaaggatcctgaggtttcaagtcgggcaggtgatgatgctatttattaagcaggacaaggaagacagaaagacaagcagatttgcagggagctaggaagtctgaagttagtactagttagtactgctctcccatcttagaagccggctctcactagggtcaacagggaccaaactcaggcagcagttaggggtggctattctagtttggattaggtcagaggaaagacagctatgttaagtacccacatgaatcatgtcagtactttccatccatcctccctagactggagaactttgacagatgtttaagatagcctagagggaaaaggtggctgggaatgatggtgtgtggtgggggtgggtgttcagcagagccttcctggacctagggtcctgaagggtcttgattctgtgacactgttgttgatcaagtattaatcttgacagaacatcaccttagagcagaacccaaatctccctggggacttagcttatgtcactgaacacatttaccaaccccccctctctctacagcgtggtttctaaggttctttccaccggaagctacgacaaaaggaaatgtatgggtggggagggcttgtgggagagtggttcagggttctgacacagactacacccagagaaagaagagcaagcacc

SEQ ID NO: 25 - DNA, 3648 bp, synthetic construct
  source 1..3648: mol_type = other DNA
ttgtttgcactggaagctgtgcttatccctgttggcaccatggggttgattattaccctgatctttgtgtactgttggttggaacggtgagatttcaggaagcccttaaaatgaggtgggggtagtggggttatttcaagatctccagggtagatcatttaagagctatgataagagtgttgagaggaagctagaggttccatgctggccagttggataaagggtaataaaggatctttcatttaatgctgctccttctttctctctgctcagaatgcctccaattccccccatcaagaatctagaggatctggttactgaataccaagggaacttttcggtgagaacactaccatacacatactacagtttatcaactgccaactgccagtcagcaagacagacttgggggggggggcagtgcacagcggagggaggaggggccctgtaccatcaggatgtggctgaccaaatggagggtgggctaggcagagagacggagccaaagagatttgtgtgatacagacggaaactacagggcattaggagccctggagcccagatagcccttctgtaatcacaatggttacagatttgtgagagatccgttggcccagagcctgggtcttttgcttcctgcccctaattgacctctgacctggagctatctgtctttaggcctggagtggtgtgtctaaagggctgactgagagtctgcagccagactacagtgaacggttctgccacgtcagcgagattccccccaaaggaggggccctaggagaggggcctggaggttctccttgcagcctgcatagcccttactggcctcccccatgttattctctgaagccggaagcctgaacatcaatcctttgatggaacctcaaagtcctatagtcctaagtgacgctaacctcccctactcaccttggcaatctggatccaatgctcactgccttcccttggggctaagtttcgatttcctgtcccatgtaactgcttttctgttccatatgccctacttgagagtgtcccttgccctctttccctgcacaagccctcccatgcccagcctaacacctttccactttctttgaagagagtcttaccctgtagcccagggtggctgggagctcactatgtaggccaggttggcctccaactcacaggctatcctcccacctctgcctcataagagttggggttactggcatgcaccaccacacccagcatggtccttctcttttataggattctccctccctttttctacctatgattcaactgtttccaaatcaacaagaaataaagtttttaaccaatgatcatcaagaatgtctgttacaggggttggggagcaggggaaatatggcgaacaatgaagttgggaggaatggggtgagggtaggccaaaggggtgggatagtcatactgaggaaggaagaggaccacagattagctgaaaggagtactgttttggctacttactattctctggtaaatttctgggctcactccaggggtcagtttagagttatctttgccatgttggcccaattcctcgaagctcctttacagatcccatccagtgtcttctctccctggccttactctgccttctattgcacataacacttctgacctcacaattctttaactgcagtcccaggccccacccctatagatgctgacaacccccaacccccaaaagctctctcttgtctgcctagtctgggcctcctccagtctctaaaataatctgctccttcatgcccaacccctatgcttaggaccctagggtcctagagcaccttccatctgcatgatccatcaaggggtgaaggatgaaatgacataagtagcatgtttatcatcatcaaacatggaggtgaggggcaacccagggcaacagtctgggggtggacatggggagggatgaggacagctgagatcactgagacggtagaattcttccatcctatctcttctcagacaatcaagagtttctggtcaattccaattgctctgtcctcctgttgctacattacatccgaaagaaaatgaaattacggaaaacaggtaagtggtttagggaggagtagtgacgaaggtcagaagcaataagctgcctctagactctaaactttcctcccaccaagaaggcttgtcctcctatccttgaaccttcctcaaatccattctgtagaggacctctggccctttttactcatagtctctgttcttccctatcccaattaccccatggccttcctctagacaccattgatttgtgtgatgaatcgggaaccatgaagttactcttcctgtcaaagacacctggagactctgccagcaaattccttacagctcgaaatacctactatgtttgtaaagtggaacgtggggcaccaggtagaggcttgggagtgttctccatagatcaaatccctcaagatggactatgaaacttgtccttaaaataaggcaggatttctcttgtaatcctagcactcaaggaactaagacaggaggactgtgagttaaagggcagtctgggctttatagcaagaactcatctcaaacacaaatgccacacacacaaacaccacagacaaacaatataaagcagggccaggatatagctcacttggcagagtgcttgcctagcatacacaagattctggatgtcatttccaacatggcataaaatttggtgtggtggaacttgcctataatcctagtattgggaggtggaggccggaggatctgaagtttgggatcatttttaactatacagaaactctgaggatagcctgggctacataaaatcctgtctcaaaatgaataaattctttagacatggaggtacggaagcttgagagttggaggcccggtagctcaaaagaaaacacccacaaggtagagccaggaatattaggagttcaacgggaacttgggctaatatgagatcctatcccaagatcccccaaacaaaataagtaaaacagggcattcttagccagatctaaacagaccataactagtcagctcaaagaacactactttgatgacagtaggttaggtacgtatattccaagccacctacttcatcctaggatctgtgtaggtctatgacaaaggggaagaaaaatgtgtaccagctcaagaaagtgtttattgccagaatcctgaaatcttgggcttcaaaagggcaaagactttgtatgtatctggggctggagatggcctaatgttatgtttacaggtaccagaattgagaattcctacaaagctattgtgcccatgctgaagaatccagaacctgaactagttggtaaggcactgaactagatgtgggaggtaggacatagaaagtgctgtacacagctgaggattttgagttaaaggcaccctgggcttaaatgtggaggactctgtcagagaagggggaggggtggtatgaatgtaggggtggggctagcatagtgtgataggtgagatcatgtgggcctaggaaaagggggggcaagggtctaggtaaactttagagggggtgtctggtttaaaccatatgaagcacgtgtatcaccctacttccttcccatcatttctatcttgggccaagctactctgaccgatccatag

SEQ ID NO: 26 - DNA, 2855 bp, synthetic construct
  source 1..2855: mol_type = other DNA
atgttgaagccatcattaccattcacatccctcttattcctgcagctgcccctgctgggagtggggctgaacacgacaattctgacgcccaatgggaatgaagacaccacagctggtgggaaatctgggactggagggggctggtgagaagggtggctgtgggaaggggccgtacagagatctggtgcctgccactggccattacaatcatgtgggcagaattgaaaagtggagtgggaagggcaagggggagggttccctgcctcacgctacttcttctttctttcttgtttgtttgtttctttctttcttttgaggcagggtctcactatgttgcctaggctggtctcaaactcctggcctctagtgatcctcctgcctcagcctttcaaagcaccaggattacagacatgagccaccgtgcttggcctcctccttctgaccatcatttctctttccctccctgccttcattttctccccaatctagatttcttcctgaccactatgcccactgactccctcagtgtttccactctgcccctcccagaggttcagtgttttgtgttcaatgtcgagtacatgaattgcacttggaacagcagctctgagccccagcctaccaacctcactctgcattattggtatgagaagggacgagggggaggggatgaagaagaggtgggttggatcagagaccaagagagagggtagcaagtctcccaggtaccccactgttttctcctggggtaagtcataagtcggttgaggggagatgaggctaggctctggatatctgcagtacccagattggccccactgttcctcttccttccaacctttctcctctaggtacaagaactcggataatgataaagtccagaagtgcagccactatctattctctgaagaaatcacttctggctgtcagttgcaaaaaaaggagatccacctctaccaaacatttgttgttcagctccaggacccacgggaacccaggagacaggccacacagatgctaaaactgcagaatctgggtaatttggaaagaaagggtcaagagaccagggatactgtgggacattggagtctacagagtagtgttcttttatcataagggtacatgggcagaaaagaggaggtaggggatcatgatgggaagggaggaggtattaggggcactaccttcaggatcctgacttgtctaggccaggggaatgaccacatatgcacacatatctccagtgatcccctgggctccagagaacctaacacttcacaaactgagtgaatcccagctagaactgaactggaacaacagattcttgaaccactgtttggagcacttggtgcagtaccggactgactgggaccacagctggactgtgagtgactagggacgtgaatgtagcagctaaggccaagaaagtagggctaaaggattcaaccagacagatagaaggacctaatatcaagctcctgttctctgcctcccagcttctctgctcaccccctaccctccctcctccaactcctttcccccctattttctccagtgagttttctttttttcttttcttttctttctttctttcttttttttttttttttgagacagagcctcactctgttgcccaggcttgagtgcagtggggcgatcttgggctcactgcgacctctgtctccctggttcaagtgattctcctgcttcagcctcccaagtagctgggagcatgcacaaccatgcctggctaatttttgtatttttagtaaagacagggttttgccatgttggtcaggctggtcttgaactcctgacctcaggtgatctgcccacctcggcctcccaaagtgctgggattacaggcgtgagccaccattcctgacaccagtgagttttcattagggattccctacccatactcttcctgataccagatagacaagtaaacaaaaggaagccattaaggggatccagaggggaggcattagattcaagtcagtgaagggagcagtgtggcttgagtagtcaagagatgagagagaaactgggcagtagcagagatgacactggtgggtgttcaggagtatgttttaattctcccttctctcatagacacccactttccctcatcctctttctcctcaaggaacaatcagtggattatagacataagttctccttgcctagtgtggatgggcagaaacgctacacgtttcgtgttcggagccgctttaacccactctgtggaagtgctcagcattggagtgaatggagccacccaatccactgggggagcaatacttcaaaaggtaaaatgggcccacatgacccaatccatgagcccaacaccccagcctttctaacaccactgtcttttgctccacttccctgtcactaaagcccctaaacttggtgccccatctctccacactgtctaaccccaacctctagaaatcaaggtttttctgtgtagggttgggttagcgtgttgttagagtaggggagtggattgagaaggaggctgaggggtactcaagggggctatagaatgtataggatttccctgaagcattcctagagagcctgcaaggtgaagatggctttggaaccagctggatctaggctgtgccacatactacctctttggccttggccacatccctaaactcttggattctgtttcctaagatgtaagatggaggtaattgttcctgcctcacaggagctgttgtgaggattaaacagagagtatgtctttagcgcggtgcctggcaccagtgcctggcatgtagtaggggcacaacaaatataaggtccactttgcttttcttttttctatagagaatcctttc

SEQ ID NO: 27 - DNA, 71 bp, synthetic construct
  source 1..71: mol_type = other DNA
aatgtataggatttccctgaagcattcctagagagcaagcttgatatcgaattccgaagttcctattctct

SEQ ID NO: 28 - DNA, 75 bp, synthetic construct
  source 1..75: mol_type = other DNA
taggaacttcatcagtcaggtacataatggtggatccgatatcctgcaaggtgaagatggctttggaaccagctg

SEQ ID NO: 29 - RNA, 1660 nt, synthetic construct
  source 1..1660: mol_type = other RNA
aggaaatgtatgggtggggagggcttgtgggagagtggttcagggttctgacacagactacacccagagaaagaagagcaagcaccatgttgaagccatcattaccattcacatccctcttattcctgcagctgcccctgctgggagtggggctgaacacgacaattctgacgcccaatgggaatgaagacaccacagctgatttcttcctgaccactatgcccactgactccctcagtgtttccactctgcccctcccagaggttcagtgttttgtgttcaatgtcgagtacatgaattgcacttggaacagcagctctgagccccagcctaccaacctcactctgcattattggtacaagaactcggataatgataaagtccagaagtgcagccactatctattctctgaagaaatcacttctggctgtcagttgcaaaaaaaggagatccacctctaccaaacatttgttgttcagctccaggacccacgggaacccaggagacaggccacacagatgctaaaactgcagaatctggtgatcccctgggctccagagaacctaacacttcacaaactgagtgaatcccagctagaactgaactggaacaacagattcttgaaccactgtttggagcacttggtgcagtaccggactgactgggaccacagctggactgaacaatcagtggattatagacataagttctccttgcctagtgtggatgggcagaaacgctacacgtttcgtgttcggagccgctttaacccactctgtggaagtgctcagcattggagtgaatggagccacccaatccactgggggagcaatacttcaaaagagaatcctttcttgtttgcactggaagctgtgcttatccctgttggcaccatggggttgattattaccctgatctttgtgtactgttggttggaacgaatgcctccaattccccccatcaagaatctagaggatctggttactgaataccaagggaacttttcggcctggagtggtgtgtctaaagggctgactgagagtctgcagccagactacagtgaacggttctgccacgtcagcgagattccccccaaaggaggggccctaggagaggggcctggaggttctccttgcagcctgcatagcccttactggcctcccccatgttattctctgaagccggaagcctgaacatcaatcctttgatggaacctcaaagtcctatagtcctaagtgacgctaacctcccctactcaccttggcaatctggatccaatgctcactgccttcccttggggctaagtttcgatttcctgtcccatgtaactgcttttctgttccatatgccctacttgagagtgtcccttgccctctttccctgcacaagccctcccatgcccagcctaacacctttccactttctttgaagagagtcttaccctgtagcccagggtggctgggagctcactatgtaggccaggttggcctccaactcacaggctatcctcccacctctgcctcataagagttggggttactggcatgcaccaccacacccagcatggtccttctcttttataggattctccctccctttttctacctatgattcaactgtttccaaatcaacaagaaataaagtttttaaccaatgatca

SEQ ID NO: 30 - protein, 368 aa, synthetic construct
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVLIPVGTMGLIITLIFVYCWLERMPPIPPIKNLEDLVTEYQGNFSAWSGVSKGLTESLQPDYSERFCHVSEIPPKGGALGEGPGGSPCSLHSPYWPPPCYSLKPEA

SEQ ID NO: 31 - DNA, 1400 bp, synthetic construct
  source 1..1400: mol_type = other DNA
atggagacagagggaccaggagttcaaggccagccccattgcttaccaagtttgaggccaggctgagttacatgagaccttgcttcaacatcaccactaacactctctcccccagaaaaaaggaagcaactaaattgaccaagatggaaagaggatagttcaaggacactttactgaagagatgcttttaaagcagaatctgaaaaaattagtgtaattttaccagagagaaaatagagtagcaaatcaggcagaggcaaagagcagctggctggttcctacctttgtttaactgtgttttggagaatctccacatctatgctgtagaactcattacagaacattgtattatttatatgttccccacttatctctgagcttctaaaatgatgatgtcttatttgtcttatgttctcagaacataagcactgtaccgagcacatattaaagactcaataaatgttggctggataaacaatttcagtaaatggcttctccaatcaaccctgtgctctgagggaaggtaatctggccacagaatgaaggatggactggagagcagaggcccttgagaaaggggaccagtttgtgggttacgggaataatcatgactggaggtaatgaaaggctgatttagcacagtggctgtggttaacagaaaggaggaaactgctgggagaaataccgcagaaacagggctgattggattctcagtgtgagagagaagggggggacatgaaaaggatcctgaggtttcaagtcgggcaggtgatgatgctatttattaagcaggacaaggaagacagaaagacaagcagatttgcagggagctaggaagtctgaagttagtactagttagtactgctctcccatcttagaagccggctctcactagggtcaacagggaccaaactcaggcagcagttaggggtggctattctagtttggattaggtcagaggaaagacagctatgttaagtacccacatgaatcatgtcagtactttccatccatcctccctagactggagaactttgacagatgtttaagatagcctagagggaaaaggtggctgggaatgatggtgtgtggtgggggtgggtgttcagcagagccttcctggacctagggtcctgaagggtcttgattctgtgacactgttgttgatcaagtattaatcttgacagaacatcaccttagagcagaacccaaatctccctggggacttagcttatgtcactgaacacatttaccaaccccccctctctctacagcgtggtttctaaggttctttccaccggaagctacgacaaaaggaaatgtatgggtggggagggcttgtgggagagtggttcagggttctgacacagactacacccagagaaagaagagcaagcacc

SEQ ID NO: 32 - DNA, 1400 bp, synthetic construct
  source 1..1400: mol_type = other DNA
ttgtttgcactggaagctgtgcttatccctgttggcaccatggggttgattattaccctgatctttgtgtactgttggttggaacggtgagatttcaggaagcccttaaaatgaggtgggggtagtggggttatttcaagatctccagggtagatcatttaagagctatgataagagtgttgagaggaagctagaggttccatgctggccagttggataaagggtaataaaggatctttcatttaatgctgctccttctttctctctgctcagaatgcctccaattccccccatcaagaatctagaggatctggttactgaataccaagggaacttttcggtgagaacactaccatacacatactacagtttatcaactgccaactgccagtcagcaagacagacttgggggggggggcagtgcacagcggagggaggaggggccctgtaccatcaggatgtggctgaccaaatggagggtgggctaggcagagagacggagccaaagagatttgtgtgatacagacggaaactacagggcattaggagccctggagcccagatagcccttctgtaatcacaatggttacagatttgtgagagatccgttggcccagagcctgggtcttttgcttcctgcccctaattgacctctgacctggagctatctgtctttaggcctggagtggtgtgtctaaagggctgactgagagtctgcagccagactacagtgaacggttctgccacgtcagcgagattccccccaaaggaggggccctaggagaggggcctggaggttctccttgcagcctgcatagcccttactggcctcccccatgttattctctgaagccggaagcctgaacatcaatcctttgatggaacctcaaagtcctatagtcctaagtgacgctaacctcccctactcaccttggcaatctggatccaatgctcactgccttcccttggggctaagtttcgatttcctgtcccatgtaactgcttttctgttccatatgccctacttgagagtgtcccttgccctctttccctgcacaagccctcccatgcccagcctaacacctttccactttctttgaagagagtcttaccctgtagcccagggtggctgggagctcactatgtaggccaggttggcctccaactcacaggctatcctcccacctctgcctcataagagttggggttactggcatgcaccaccacacccagcatggtccttctcttttataggattctccctccctttttctacctatgattcaactgtttccaaatcaacaagaaataaagtttttaaccaatgatcatcaagaatgtctgttacaggggttggggagcaggggaaatatggcgaacaatgaagttgggaggaatgg

SEQ ID NO: 33 - protein, 539 aa, Mus musculus
MATIALPWSLSLYVFLLLLATPWASAAVKNCSHLECFYNSRANVSCMWSHEEALNVTTCHVHAKSNLRHWNKTCELTLVRQASWACNLILGSFPESQSLTSVDLLDINVVCWEEKGWRRVKTCDFHPFDNLRLVAPHSLQVLHIDTQRCNISWKVSQVSHYIEPYLEFEARRRLLGHSWEDASVLSLKQRQQWLFLEMLIPSTSYEVQVRVKAQRNNTGTWSPWSQPLTFRTRPADPMKEILPMSWLRYLLLVLGCFSGFFSCVYILVKCRYLGPWLKTVLKCHIPDPSEFFSQLSSQHGGDLQKWLSSPVPLSFFSPSGPAPEISPLEVLDGDSKAVQLLLLQKDSAPLPSPSGHSQASCFTNQGYFFFHLPNALEIESCQVYFTYDPCVEEEVEEDGSRLPEGSPHPPLLPLAGEQDDYCAFPPRDDLLLFSPSLSTPNTAYGGSRAPEERSPLSLHEGLPSLASRDLMGLQRPLERMPEGDGEGLSANSSGEQASVPEGNLHGQDQDRGQGPILTLNTDAYLSLQELQAQDSVHLI

SEQ ID NO: 34 - protein, 551 aa, Homo sapiens
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 35 - DNA, 3426 bp, synthetic construct
  source 1..3426: mol_type = other DNA
aggagatcttatcagtgctaggtgtgtgtggagtttcaggctacctcatcaccccccatctctccacctcccattgggccttgcccctcatcctctatacaggaagtgggctcgccttgggtttcagtctgttgctggcctagctgttgtctgctacctaagctgcggagcaggagatggccgtggacccaacctccagcaggactgcagggaacatctcgacacaactccatgttgcagccaggtgatgcccctgtgtggtcccctgggagccctgggcaccccactacctcttgctggtcctcccaagggacagggagctgggagggggcgctcatctctaacttggttccctttgtagtttgtatctgggctttgatgattaaccttgtcttaggattccctggggaagggggtagcctggctgtcatggtttctcctttaagaataaaccccttccaatgtcagggcacagtggcttcttcccagtctactctgtgggaagctgtgtgccattttcgctaggagggacttccctggatcctgtgtggggatgtcttagcccagaagggtggaggaaaagatttgtagctcgtcagtattgggaggtggcatggcgtgtatgggacaggtgggattcaacgctgacgcctgtggagttcatctcagcctggagaattctccttagtgagaaagagagaaggaatctccccctgggacctggctgtccagtcagctatgttgattgacaggtttctgagggccctaggctggaggtcagaggtagagttctagcttagatttgtctctgacattaccgagtggctagagaggttaaggacatcgccaggcatggggctccgtctccgaggctgcctgaggtaggagcttcccaccgttagctgtgtttacagtacttcagggccaaggtgggtggctgaggaaggcagaactacctttgaaggtcccctttctttcacacactcctcagctgaactgaacagcaagaggtgaacagtggtgtgtgtgtgtgtgtgtgtgtgtgtgtgtgtgtctttgtgaatgtgtgtgtctctggagaggtggccagagaaaggatctctgaggtcaaaggccaccagatctgacagacaagctcttgcttgccgtccgtcctccctcagtcgaccctgtacctcacagacctgtgaggccttggactgccccagcactggaacctttatcctgggaaacaggctgctttaggccctgatcttcccatagggaaggagcagtcagtcacagaacctgctcttgtgctgtgagacctctctgtgacatgtgtgggtcagtctgacgcagggagtgtgacacgactgtcactcatggctgaccctcctcctccgtcagcatcaccactgtactggaaatcaggtgacccagttagagcagcctgggtctgtgggatgacagaccttttcagggcctcagcggagacatatatgagtcagggagatgaacgtctggtggttgcgaagggcatacgtgggtgagcctcagggggacagtgcctgggactctgagattcttgggctgtactcactctgcccgaggctggccttgtgctgtgtggttttggagtggttttcttagtctcccagagattcagtttccctcttgccgaaggaagctgcaaccaccatgactgcttgaagctaccctgccccccagcctgacccttccagaaccttccatatgctcttccgtctaaccctgtccatgatgggtggatgcttttgtgcctcttcagccaggggtggggagcaggtttgagtgccttttcttaggcattctctgtttttggctgtggcttgtggcccggctataatcttgcctctttcattcctcttcctctcccccagggttatgccctccctcaccccaaccccacgccatcatgaggctgctcagatgtctgggtccgacctggcttgctgccttattgaatgaacacgtagagtgcttcagatcctgttgggctcacaaggggctgtggaaggccccttgcttgggctgaacctgttagggctccctttctacccaagcacctgcggggagttgatcttccagggaagggaggtctggggtggaaccccagttccgtagacttggtcctgggatccttggtctccttgtctgcctagcaaagacaccaaatagctgctgagctgccaggcagcatagagtagctggaagtatgagggcctgggcacaaggcctgggcagtgtttgatgaacatacccggcaaacactgtcaccgggccgccacccatcacttgctgtcccacaaacccttcagtgcatgacatttattgagggcctcctgtgttccgaggtcagtggtttggagtgggcacactgccttggcagtgtggtaggtgacaagccagagggatggaaggcaacccaaggtagattgtccttccttcatgggggagaccagggcgagtgccacagaaccagcagaaaagagaatgagaaatgggataccaggagccttgggtgcaatgtgggaaggacaagtccagggaggcttttagaggtccagaccaaagccaccagcttatgtggactgcaagaaccacctaaggcaggccacggtcttgccttccatgctcctcagggctgaagtgggtgctcttggtagccctgtgataggtattcatttggagatcagagacccaggaatgaatggaatccaaatgtccttgtcctgatagactgtctatgactcatatacataggtacgtgaatgcatgcgtgtatgtgtgttttcaaactgaacactgttttaagtcatgtgcaaaagtcttgagctggtctctatcctgcttggggcctgagcgattccttttcccagaacagctatgatgtggatgcttctcgcctattagtcacccagggacttggctgtccaatcacagagctgagtccctgtcacctcacctcacctgggtacatggggtgggcatctgccaaagatgctttgggtgggagacacctcattcacaccgtgctccggagagccggtggctgcccctaggaactccttgcatgaccacctactgccaccagtttcttgtgatgggttggtcatttatagtgaaaacagagtccgtgtaaggactggtttggtctgacattttaggcacctctggaggaaggaagggagctcagagggagagagagcctgggcctgctttgctctggctgggcggagagagggtagagtggggtggggtttctgacttgttctttccttctccacttagggtttgcatcctcagctcctctcagctgtgatggctaccatagctcttccctggagcctgtccctctacgtcttcctcctgctcctggctacaccttgggcatctgcagcagtg

SEQ ID NO: 36 - DNA, 4846 bp, synthetic construct
  source 1..4846: mol_type = other DNA
accacaccagtcatacatcctgacccttgagggcttccactggaaactgtgtgccaatcagaaagtgcctcccctttctccggttagatatgtccacgttctgggtagcttagtggtagaccaagtagatcagtagatcgagtagattgtgatgtagggattcaacttcaaactgccattgtgaaatgcacagcgtcgccattccccaggggagtgttcaccaggaggtctctctcctgcctccctctctagcctgtgtagtccctcccactctacagccatcttttgactagcacacaggtctgtgtccacagactgggcaatcctattacatgtttggttcaggtctgtcttcatgtcagcatgtcagctccgggggagcagaggccttctttgctgtccactcatgtgtccccagcaccctgtgcagtgttctctgttggtgactgctcagtcaacatcattgaaggagcgaatgcaggatgggcccatcccctactctaggggctgtggggctgctggtgggacctcatgacacccactacctggtctcactcttcacccctccccctcttccttggccaggctgaagacagttctcaagtgccacatcccagatccttctgagttcttctcccagctgagctcccagcatgggggagaccttcaggtaggaaccaggggtagaaaacagagggatggttgatggacagcccaagagcaggctgcaggggagagatgtgatagggtgacaatggctttgccttctgtgaggaggctacaggatgtgaggcaagcagtttccccatggacaacagctctgtctccgaactgcctgggagccttctgtctaaggcgtgtggccttggctctaatcctggctctgccactgcctgcctctgtaaacttggaggagttacagatcttcccagtgcctcagtttccacccttatacggccctgggagtgttgctgggtgcagtacgggattcagggagctgcagagatccctgcctctgtgaacagagcaattccaggcagatccagtgagcagagtgattcctggcaggcctagtgattcatcacaggaatgcaagacctcaggccatctggactcacaaaacctgggcttgttttcaaactgtggcccacacagtcctatcttctgtccccctagtctacctgcctcaggctagatgcttcttgaggcaagaagtggatagagggtagaggaaatctactgtttctacccccgaccccactgggtttccatgatgggaagcgtgtggggggcagagtgacctgtggcactatgctgagaatgggaggcccacaggcctgctggactggcctgtgaccattagggctgagcagggcccttctagaatatctgagaggctcccagaattgaggatgtcttattatgatacttgatgtgtgtgtgtgtgtgtgtgtgtgtgtgttgtgtgcacacattcctgcatgcttgagtgtgtgcatgtaatggggtgagcagctccataaaaagtttgtccagactgtgttatgggggtgcacacctttgatcccagcactcgggaggcagaggcaagtggatctctgtgagttcgaggacagcctggtctacagaactagttctaggatagtcaaggctatacagagaaaccctgtcgcagaaaaccaaaccaaaccaaacaaaaaagctcaaaccaaatcaaaagaaacaaacaatcaaaagaccaaaataactctgtttttcctgaaatcccttatgcctttgatcgtggtaagatcctgaccaacagacaaatgcaaacaaaccttgaccttgactggctatgggtcagcaaggattcatgtcttagcctctttgggttgtggccagggctctgggtcatgaatctcactcacacacacgaaggttagcctgaggggtatatgcaggccatgacagggaggggcgagctggggatccaccagttcttgaccactgtgtcccttgtccttgctacaccagctgcctgtgacttgagcaggctagaccatcaaggacattggtcagctcattgactctggtcaagacagttgcgattgtccttctgtgttcgcacacctagttcagtccaggtacacagcaggtactcagtagccagccacattcacccctagccaacatcccccagacacaggtgagccagcactgagcctgtgttcatctaggatctgacctagggtgacaagttgagagagtccaccaagacagcaaggctacaagatgatatcaggttgggggcagggaggaggcggtcccgggtggtcctgaaggatggagaacaggtgggagggaagggagaggaggagttgagttcagtggtggggaggacgttgaacgacccacttactctcctcactttctctggcagaaatggctctcctcgcctgtccccttgtccttcttcagccccagtggccctgcccctgagatctctccgctggaagtgctcgacggagattccaaggccgtgcagctgctcctgttacagaaggactctgcccctttaccctcgcccagcggccactcacaggccagctgcttcaccaaccagggctacttcttcttccatctgcccaatgccttggagatcgaatcctgccaggtgtacttcacctatgacccctgtgtggaagaggaggtggaggaggatgggtcaaggctgcccgagggatctccccacccacctctgctgcctctggctggagaacaggatgactactgtgccttcccgcccagggatgacctgctgctcttctccccgagcctcagcacccccaacactgcctatgggggcagcagagcccctgaagaaagatctccactctccctgcatgagggacttccctccctagcatcccgtgacctgatgggcttacagcgccctctggagcggatgccggaaggtgatggagaggggctgtctgccaatagctctggggagcaggccagtgtcccagaaggcaaccttcatgggcaagatcaggacagaggccagggccccatcctgaccctgaacaccgatgcctatctgtctcttcaagaactacaggcccaagattcagtccacctaatatagcaggtggccaggactgggatccagctgcctggatcaggtcaggcttgaggaagactgcttaagaggtctcttgaggacagtccactgctgaggactgtcctggctatccctgccccccccctccaaacttaatcatccacttctgaactccatttgctacttcctggtctaaccagggtttggtggagggtggggagtgggggagcggtggtcagctccactgccctatttagtcatgaggtcactagcttcctgtacctgcctccttccctcctgcttgccttcacagggcagctagaacttgcttcccccaatacaaacataaccttgctcctcatttatccctgagcttgctggggctccttacttccctctgccccatttcctgtctcaactcttgccctgccagcacggctgtggaacagctgtcctcaggttgtgatgtggctgtacactgtcttaaaatcagagcagctttgacaacccaaacgaagaggtcctgataagactgaatgcagcctgttgactatggagggtccctcgtagaaggaggggttgggaagggtcatcagtcatcacctgagatcgtagccactctcctgcaccccatcctcagctattctgctcctggagcatgagttattttatgtgttcttgggcgatgctggtctatgtaaggggtgggggcgcggggttgattgaagtgagggctccttctatgttgctttggggaccttgtctattctggggtgggtttttctgtgaagcagcttctttggggtaacctagcccccttgtcagtaagcctaagagagtcaaaggaccaccgagctggacttgagtgtacgcctacttttgatgcaacatctgggaaaagtactttttttgttaatgtatccttgaagctaagaaaagcttacaccacaaacattgtgtcagcactgtcctttgtggagatctcacgaggatcctctggggtctcctggagtcatgatgtgtcattccctatcccctaccccaccccaccccactcccaaccccctcctttgcaggcactgttcattgtgctccgcctaggtatcaagcttctccatcatctgtaagtcccgcctacctagagggaggggcttctgtaaatcccgcctacctagagggaggggcttatcctataaatcccgcctacctagagggaggggcttctcctgtaagtcccgcctacctagagggagggacttctataagtcccgcctacctagagggaagggctttttttcttaaggcactcagaaggagatgaaggggatagcacttgacccatcttccttatcccgggctgtctcctgacctccctgtgatagactcagtttggacaccaatacctacccagcagtggctgttggatctttccacggctgcctcaccaactcatgaaggagtccactggaacctgagccccgagtcccaacagcccaggcatgctaagtgtaccacctcaccctcagaggagagcactgctctctacagaccactccattagcacagagccttaggtgtgggtcactgttctagggtgatcagatcgtatctcctcagggaagcagagatccagacctaactaatggctgccctacccacagtgcctgccttcgtggtcctgcacactgctcactggctgtcctgccagactacaggaccactggctgactagcaacttag

SEQ ID NO: 37 - DNA, 8654 bp, synthetic construct
  source 1..8654: mol_type = other DNA
aatggtgaggaccctgacatctgaacagctctgctttccctatggagagatggctaaggccctgctaaagcctcttcccagaaggggatggggccagagaccaggtgtccacctcctgcctcaccctctgtcctggacccctgccctggcatgcatgcatgaatgcgcgcacacacacacacacacacacacacacacacacacgcctctccaagccacaggcttgtgctactttctgtccctgccttgccttagtttgcctgtaggaacttgcaggctctggagctccatttcctcctctctgaagtgggtccatgaatcccttctccgcttgccagagttacccacctcctcctctggcaggtgtgggtgccaggtgcaggggcagtgggcgtgtcttgggggggctgtgggcacctacagtcagcactcacacctcatcttgcccggcaggcacttcccagttcacatgcttctacaactcgagagccaacatctcctgtgtctggagccaagatggggctctgcaggacacttcctgccaagtccatgcctggccggacagacggtgagtccactggccacactgcagaatcttaggatggtgggatctcatattctggatcctatgcactggactccaaggagtcaacgtttacagaatcttggactaatgggacccacagactttttgaatcacaggagttggggcccaggcctggaatcaagggatcgtttaagagaatttgggagtcagctatttagacttctagaacctgggtggagtccaggctgcagtggaggggctggggatgaactggacagggtagtttggggacactgagctgcaagcctgctatgccaaggctggactccactctagaggcaagcagagaggaagtggggagaaaggagggtccccaagctttttggataagctcggattttgagagaccctgtaggcagctgtggggacagggaacctgggagtggtttgggtgcctggtggatgggagagagatgtcactgtgatgtaaacatccaggcggccagtgagtgagggactgaccccaggtaggcttgatggggtcagggagaagaggaagaccacagagaggtggctgggcccaggtggattccatagggtgtggggtgggggaaaatggggagggagatggaggggtaaggcaggttcgggccctggcttctgggtgggtggtgattccacacggctctgaaggcctaattaaggatctatcttcatcctaagcctcatggagctttggagggctctaggtggggaggactgcctgtcttgaagtggtttgtgtttctagatcactccagctgccgtgggaccaggctgggatgagggagcaggacaaggtgcccatgagggaggaatgccatggagccagtgggggaggcagtgcccctcacagtggttggcacaggggccccagcctggccttgctgcctggtgccatcctggcagcagagagatctgagaagagtcccttgggccagctgtgtgggtccttcctgttcttctaaagagggtcttttgccatgaaaggcggtggaaccaaacctgtgagctgctccccgtgagtcaagcatcctgggcctgcaacctgatcctcggagccccagatgtgagtagcccacccagggagagagtgcagggagagggtggtctcccagctccgggctgcgatctcagggtgggccggatgccaggggaagagctgaggcccaatggctggggctgtggctaagcaggatgcccagcgctgagcaagaggcagagaaaaacaggtgacaccccagggagtggactcaggcctgccccagggccgacggaccctctgtatgtgaggcatctgccattctgcctctgctcctgctgtctcccctgcgctttgtgggtcctgtgacacttgtgttccttatcaagtaagacagcactccagtaactgtaatgaccacacccagctcttcccgggggcttggcagtgcagtgcagtgtggggggtgaagggtgtagactctggagctggatgctccagccctggctcaccagctgggtgacattgggcaacccctgtgccccagtctccccttctgtaaaatgggcataataatggcacctaccttgttgggtcaaggcccaacaagggccttgtttttcaggccctccccagcctgaaggtgggacctcaccagggacccgcctccttctgcccaggctgctcgggccaaggggcacctgcaggccagtgccaagctgtcccctgcacccccgtggcttcccatccccctgtgctcattggtgcctaaagtccagagggggccgaggcagctggggccagtgagtcagtgctgcctcgagcgtgccaggctgtggtagtgcctgggcttggtcccgctctgagattggagtgggcactggagcaggaggaggccagacagtgggagcagacacccctgagcttgtgggggcagtagagatgcctgggtcctggcttgggcagctgcagctgccccaggagagctcctggctacctcggaaggggcaggggtcccgcttgtccccgactcacactggatccacagagtgcgtagccccgactgtgcctccaggatcagatcgggtgccagaagcaggggagaggccaagcagtgggaacagacagctctgagcctgcggaggcaaggggggatcttcccagaccctgagagtgcagggatgcctgggtccacagccacaacttgggtagctgcagctgtgcccgggagggtgggtctcctgcgtgctccctgcttgcaagatcacctggaggcttgggtttggaccagggcccctgagagtgcagggacacctggctctgtggccacgacttgggtggctgcagctgcgcccaggagagcggggctcttgcctgatcccagcccccaacagcaccaggaggcctgggtccacagccaagacttaggcagctgcagccctgcctgggagggcagggctcctacctgctccgtggagcaggagaccccagctacgcctcccggctgcagccagagttttagcagcggccactccggatgggcagctgcggccttcacctacagccccatgggtcaacatcagcactgtccccttcttaccaatgaagacactgaggctcagagaggtatggtctctgcccaaggtcacacagccaggaagtacacagctgggattgaacccaggcctgcgggctcccagttcaggcttctcaccgttgcattaggctgcctcttgcagggcatatggccatgtccatatgagtgtacatcaaattattgtctgtcaaagcctcctgggcggcagcgggctccaaggacacccggaccacatcttccatcaggttatatgtcttcttgaggggcaggagagtgtgaagatggagtcacacagtgtagctgataaaatcacagcctctggaaccggatgctgttccccttgcagggagctctgtaggtggcctggtttctgatcctggctccaccatttcttagctgtgtgacctcaggcaagtccctaaggctctctgtgcttcagtctcctcagctgtagaatggaaatagtgacagtgcctgtgcccaagttgagagaattaaatgagacaatatatgtaaagtgctctcaagagtgcccggcacagtgtgagtgatcacgcgaatgtatgattggcaatatttttataacaataatagtaataataataataataataataataataataataataataatgagactactatcatcggggaaaatctagcccccatagcagctataagaaggctaggacagggtctatagggtggagaggaactggccagtttggggaattccaacgatcaggtttaaggtttgcccatagtgggtcttatgcagaccagagggacggtgcccaaggtcccggccctgaaatcagcatcctggaccccagatcctaagaggcctgcactagagcttcctcccgcagggctgccgggctgtagggttggcagctgcacagtggcaggatgccttaagctgctcactcggcagccctctgtcaggcatgacaagccaggactcggagatgcagtcaccctgtagggggacacagagggcctgggtttaaacttgggctcccctgctgttcatctgcatgacctggcgcaggtcccttgaccagcccaagcctcaggcttcccacccataccatgggatggatgctgtgtgcattgaggggctaaggcatctgagcagtgactgctcataggaaaatgctcaatacctggccattttcttccagaaaattcaaggcgtagggatggggagagataggggaggggagggcctttcacactaaaaaatgttaaacaggtatagttccagaactgtacctttgtgggggcttaccagcttgcatgcctcctgtaacagggagctcactccctctcaaggtagctcacttcattttagatcaagtgttggtaaactaccttgacccataggccatatccagcccactgcctgtgtttctaaataaagttttactggaacatggccctaatattcatttacatgttgcctacgtctgcttcatactacagtggcggagttgagtcatcgtgacagagaccatgtggccccataaagccaaaaatagttactatgtggccttttacagaaaacatttgctgcccctgccttgggtggcggggcatgtggagaggctgccatccttcccaggtcatcccggtggcctcccccttcccctgcctgaagctgctagaagtttcacccctgccctccttcccttgcagtctcagaaactgaccacagttgacatcgtcaccctgagggtgctgtgccgtgagggggtgcgatggagggtgatggccatccaggacttcaagccctttgagaaccgtgagtgaggaagccagggtggggctggggcagggctgggcacctccttccttccacctcctctgggatcttccctgctccagcctttctccccggcagcttccacacccacatctgatcacctcaccgcccgctgttctcgggatgacgtcctggctccttcctctggcattcaaggccgctcaggatccagccctgggcctcctccccggcactcctcctcttgaggcctatatttctgctacagccaaaggccaactctccaaacccaccttgcctatccctgcctctgagcccttgcacatgccgatcccactgcctggaaggccctttacacctggaaaattcccactcacgtttcgaggcttagttgaaagtcacttcctccaggaggccttcctagactcccctagccctttgtacctcctctttctgggaatgtatgtcctgggttgttactgcctgattgtaggtctgtctctttcactgggctgtgagcaccctgaaggcagagtctctgccacccctgtgtctccttgccacgcacccagcactaggtctcatcaggacagtggtctgggaaggtttgttgaatgcctgagcgactgcctgctgaccttgggggccatctccagggaagcagtggggtggaaagtgagagtcaggcctgaactggatgtgatctgactggcagaccatcgaaactttctgagcctgagttttggggagaatcggagtcacctacctaccaggcttgtaaggcatctttgaactgagacatagaaactgcttaccatgtagtcagcactcagcatctgcttatcacttatgtggttctatagagtcttagaagtatagaaactgagattcatttaacttcagaatcacagtgttataatcatcacacttaaaataaatgagatgatccatggaaagctctcaggtcaattgtaagtgcagagaaaagctgccgtgagtcattcatccagcagctactgaatcaagtgccaggtaatcttccaggtgccaaggacacagcagacaaccaagtccctgccctcaaggagctcacagtctgctgggggagacagaaaatgagtaaagaaaccaaaccatatatttcgtaggtcaggtgatgataagcactgggaataaaagtgaaacaggcaagggggatgcagagtggtgagtggtcagggaaggcttctctcaagaggtgatcattgagagaggcctggaggcagtgagggagcgggtcctggggctttgggtcggggggtaggggaggtggtagcatgtgccaggagggaggagtggcaggtacaaagtgggaggtggaggtcatggctgactgctctcccctaccccctgttgtctccagttcgcctgatggcccccatctccctccaagttgtccacgtggagacccacagatgcaacataagctgggaaatctcccaagcctcccactactttgaaagacacctggagttcgaggcccggacgctgtccccaggccacacctgggaggtgagaatgtggcccagtccgtacctgccttggttcctccttcctgtcctggtccatgtcctgtccctttttcattgtgtcgaggtcccctcagccccaggtggatggtgggtggctgagtccagtgctctttctgcactgcctggggctgcctccccgacttccacgtccttatctagcccccttaatcctggggatccagctggcagcaggttccaggctgacaccagggaaagattcagaaagccaggagataggcagctgcccagccccatgggctttcagaggccagggaagccaaccctccacgtgtcaaagcccatctgctccacatccgtgcgggcctgttgcttattcattcacttacactcatttcttcattcactcaactgctcactcaataaatatttgctgagcacctactatgcgctgaatgatggggagagagcggagggtgagctcagcaaacttccactctcaggaatctcagactggggcagggcaggcaggcagtcagtcagcagatgtagcgacagggtctgggatagtgaggcacccggatagtgataagttccatcaagaaaacccaacggggcaaggaatttgacagtgactggcacaggctacttcagactcaggtgaccagggaaggccccttggacacctgggtgtccagaaggagctgacctgcaaagatctggggagaagttttgcaaaattagaaagacctgccacggcaaaggcaggatgctgctatgtgtctggggagcaggaggaggcaaaaagagtgacccagggtgggagaggtgtgcggagacgtcccactttctggtcctgtggagactagactgcagcggggcacgggtgggaaccagagaccagctgcaggcttgggagagggcggcagctcaggcctggggtgagtgcggcggggagagagggggatggttctggaatggctatggaggtggaaccaggttctggaggtggaaccagctgattagctgatgacttgggctttaggggatgaccctggggtttgaggccccagcaactgggtagtgagggagctttgggtggcaaggaagtgggggtgcagctggcaggggttgggggggggttctgtgatgctcagcctcctggccctgccatgggaggtgaggtgggcgctgacagtcctgtctccgacccaggaggcccccctgctgactctcaagcagaagcaggaatggatctgcctggagacgctcaccccagacacccagtatgagtttcaggtgcgggtcaagcctctgcaaggcgagttcacgacctggagcccctggagccagcccctggccttcaggacaaagcctgcaggtactggtgggcggcaagggtgagagggggctcaggcaggagggggcggctggaggaggagctgctccattctctccctgcctggggtgagggtatagcagtcagctccatccctcacttgctgtaaccctgcagggggctcgcttttcagacaggaacaggggttcagtgaggggacggggcttgccatgggccttgtgagttggggaggggtcgaaaggctgttttcactcttctccaccctgaaatctccaactcctggtttcttctggggctgggggtggctgctttcacctctcggcctgatctgtgaaatgggtacagtgaccgtgaggcagagctgctgtgaggaaggcgtgtgatgtcccggcagcctggccctcagcaggtctgctgggcgcctgtccctcatcgcaccacatttcgagttcaagcccaagcaagacctggggtcctctaagtggtgcttggagggaggtggagagaggggtgcagccagctggagcccaggctgctgtgggggtggtggggggctgtaggctcaccaggggggcccatgggatctgtctggagctggaggagctcttagaatgccaccgtgtcatttctttttagcatgaaatctggcttcccagaggatgggcttagcagaaccctgtccccagccccccagacgcaggcctgcgtggcagggccctggatgggggtgttggggtgaccgacccagggtgagtgagggcacttctctaacccctctccttgctgtttctcccacccctgggcagcccttggg

SEQ ID NO: 38 - RNA, 2709 nt, synthetic construct
  source 1..2709: mol_type = other RNA
agtctgttgctggcctagctgttgtctgctacctaagctgcggagcaggagatggccgtggacccaacctccagcaggactgcagggaacatctcgacacaactccatgttgcagccagggtttgcatcctcagctcctctcagctgtgatggctaccatagctcttccctggagcctgtccctctacgtcttcctcctgctcctggctacaccttgggcatctgcagcagtgaatggcacttcccagttcacatgcttctacaactcgagagccaacatctcctgtgtctggagccaagatggggctctgcaggacacttcctgccaagtccatgcctggccggacagacggcggtggaaccaaacctgtgagctgctccccgtgagtcaagcatcctgggcctgcaacctgatcctcggagccccagattctcagaaactgaccacagttgacatcgtcaccctgagggtgctgtgccgtgagggggtgcgatggagggtgatggccatccaggacttcaagccctttgagaaccttcgcctgatggcccccatctccctccaagttgtccacgtggagacccacagatgcaacataagctgggaaatctcccaagcctcccactactttgaaagacacctggagttcgaggcccggacgctgtccccaggccacacctgggaggaggcccccctgctgactctcaagcagaagcaggaatggatctgcctggagacgctcaccccagacacccagtatgagtttcaggtgcgggtcaagcctctgcaaggcgagttcacgacctggagcccctggagccagcccctggccttcaggacaaagcctgcagcccttgggaaggagatcctccccatgtcatggctcagataccttctgctggtccttggttgtttttctggcttcttctcctgcgtctacattttggtcaagtgccggtaccttgggccatggctgaagacagttctcaagtgccacatcccagatccttctgagttcttctcccagctgagctcccagcatgggggagaccttcagaaatggctctcctcgcctgtccccttgtccttcttcagccccagtggccctgcccctgagatctctccgctggaagtgctcgacggagattccaaggccgtgcagctgctcctgttacagaaggactctgcccctttaccctcgcccagcggccactcacaggccagctgcttcaccaaccagggctacttcttcttccatctgcccaatgccttggagatcgaatcctgccaggtgtacttcacctatgacccctgtgtggaagaggaggtggaggaggatgggtcaaggctgcccgagggatctccccacccacctctgctgcctctggctggagaacaggatgactactgtgccttcccgcccagggatgacctgctgctcttctccccgagcctcagcacccccaacactgcctatgggggcagcagagcccctgaagaaagatctccactctccctgcatgagggacttccctccctagcatcccgtgacctgatgggcttacagcgccctctggagcggatgccggaaggtgatggagaggggctgtctgccaatagctctggggagcaggccagtgtcccagaaggcaaccttcatgggcaagatcaggacagaggccagggccccatcctgaccctgaacaccgatgcctatctgtctcttcaagaactacaggcccaagattcagtccacctaatatagcaggtggccaggactgggatccagctgcctggatcaggtcaggcttgaggaagactgcttaagaggtctcttgaggacagtccactgctgaggactgtcctggctatccctgccccccccctccaaacttaatcatccacttctgaactccatttgctacttcctggtctaaccagggtttggtggagggtggggagtgggggagcggtggtcagctccactgccctatttagtcatgaggtcactagcttcctgtacctgcctccttccctcctgcttgccttcacagggcagctagaacttgcttcccccaatacaaacataaccttgctcctcatttatccctgagcttgctggggctccttacttccctctgccccatttcctgtctcaactcttgccctgccagcacggctgtggaacagctgtcctcaggttgtgatgtggctgtacactgtcttaaaatcagagcagctttgacaacccaaacgaagaggtcctgataagactgaatgcagcctgttgactatggagggtccctcgtagaaggaggggttgggaagggtcatcagtcatcacctgagatcgtagccactctcctgcaccccatcctcagctattctgctcctggagcatgagttattttatgtgttcttgggcgatgctggtctatgtaaggggtgggggcgcggggttgattgaagtgagggctccttctatgttgctttggggaccttgtctattctggggtgggtttttctgtgaagcagcttctttggggtaacctagcccccttgtcagtaagcctaagagagtcaaaggaccaccgagctggacttgagtgtacgcctacttttgatgcaacatctgggaaaagtactttttttgttaatgtatccttgaagctaagaaaagcttacaccacaaaaaaaaaaaaaaaaaa

SEQ ID NO: 39 - protein, 538 aa, synthetic construct
MATIALPWSLSLYVFLLLLATPWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKEILPMSWLRYLLLVLGCFSGFFSCVYILVKCRYLGPWLKTVLKCHIPDPSEFFSQLSSQHGGDLQKWLSSPVPLSFFSPSGPAPEISPLEVLDGDSKAVQLLLLQKDSAPLPSPSGHSQASCFTNQGYFFFHLPNALEIESCQVYFTYDPCVEEEVEEDGSRLPEGSPHPPLLPLAGEQDDYCAFPPRDDLLLFSPSLSTPNTAYGGSRAPEERSPLSLHEGLPSLASRDLMGLQRPLERMPEGDGEGLSANSSGEQASVPEGNLHGQDQDRGQGPILTLNTDAYLSLQELQAQDSVHLI

SEQ ID NO: 40 - DNA, 73 bp, synthetic construct
  source 1..73: mol_type = other DNA
actcttgtctaaccctccttagagattcccccatatgccgaagttcctattctctagaaagtataggaacttc

SEQ ID NO: 41 - DNA, 94 bp, synthetic construct
  source 1..94: mol_type = other DNA
gaagttcctattctctagaaagtataggaacttcatcagtcaggtacataatggtggatccaccacaccagtcatacatcctgacccttgaggg

SEQ ID NO: 42 - protein, 263 aa, Mus musculus
MASPQLRGYGVQAIPVLLLLLLLLLLPLRVTPGTTCPPPVSIEHADIRVKNYSVNSRERYVCNSGFKRKAGTSTLIECVINKNTNVAHWTTPSLKCIRDPSLAHYSPVPTVVTPKVTSQPESPSPSAKEPEAFSPKSDTAMTTETAIMPGSRLTPSQTTSAGTTGTGSHKSSRAPSLAATMTLEPTASTSLRITEISPHSSKMTKVAISTSVLLVGAGVVMAFLAWYIKSRQPSQPCRVEVETMETVPMTVRASSKEDEDTGA

SEQ ID NO: 43 - protein, 267 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 44 - DNA, 5262 bp, synthetic construct
  source 1..5262: mol_type = other DNA
gctggcccaagaaaagacaataacaataacgttaagaagcaggcttgggacaaacaagagaaagggtagttcttttagatgtcttcctctagccttttccctgaagagctcacaagtctctggagatactgatcgatagctggaaaatgtccagccacaaataatgtaatagctcactcacaggtccaaaggtccaggctttctcagacagagggaacattttctaaaaggtcaggcctagtatgtttcactgaattctccattctggtctactgccaagtgtccttaaggtcatttgaattggtttttatgacagttccttcccaccccacaccattaatccatccccaagtatttcccaagcaatgtaacatacggctaagtaccccaaatgaggatttaaagatttggaaacacagagctaaacacatgggactggcaagccacacacacacacacacacacacacacacacacacacacacacacacacaaataaagaaataaataaatgtattttttaaagttaaatgaggcagaaccttcccctaatgaataaagatttcaagggaccaatcactgggcaagtaggtgggaatttccagtcagtgagaggaatagggaagtcaggagaaagatacttccttttggaccagaaagagcatggagacacaatgtgggtagcgagaggcccacgatttaggtagggatcctctgggatgcgccactggaggatttataacttaaatagctaataaaattaggatgtgtcttagttagagttttactgctgtgaacagacaccatggccaaggcaagtcttataaaaacaacatttaattgatgctggcttgcaggttcagaggttcagtccattaccatcaaggtgggagcatggcagcattcaggcaggcatggtgcaggaggagctgagagttctatgtcttcatccaaaggctgctagtgaaagactgactttcaggcaactaggatgaggatctcaagcccacacccacagtgacacacctactccaaccaggtcacacctattccaacaaggccacacctccaaatggtgccactccctggtccaagaatatacaaaccatcacaggatgctagtttccgcacccagcaattgtgttacctgtagattctaaactaagattgtgtggtgttttcctttactcggtgactcaactgagttccagagaaaagtacggtggcagggcaccccagccagccatgggaatttggaagtgtagggcatgtgtagcaacaccccgccatgggaacttagcgagccgggtggagagattttgaagctctgaatcggagagtctgctgggcagagaacaggctgggctgtccactggtgccttgctggtggtagtgtggattgcttttttatatatttcacgcaacagttaaagccctagagtagaagtttaggtgatcttataaagacaaatgtgtaaaagaaatctccatttatggagtcaatgccatgatctggaagatggtgtggacaggctgctgttcacacttttgtgtcataaacaaagcaccatttgctctgctgcacttgtagacagcagcagtccccaaaccacagttcctggacctgcagagcctgtaccatctggaacttggaaatacaggttcccgggctctgcctcagatcctccagctcagtagttctgggttggagccagctagttttaacaagctctacagatggatccacacataactcctgagcattactgaaaacatggacctgaagtgacaggaggcagcagaggaaggatgccactgcagcagtgataagctcgccctgggagacaaagctacggctaagactccaaacacagaccaagtggaattaacagcacgggaccctggtcttgaaggaaggagctctaaatgttgtaaataggtggttaataagaagagggagtggtgccaagtcaggtatcaaaagccacagctagaggagtaactaaaggaaactgtcttctgcaactcacaatcttggctgaactgtcagtgcctcttagaggttccttcaagaaggatcctggcagagcacttgtatagcatatgcaaggccctgagtttgatccatagcactgaagaggaggaggggggaagagaaagaagaagaggaggaggaagaagaggaagaggaggagaaggaggtctaagaatagttaattttctattgcaataatagaatacctgagactctgtggttcatagagagtcaaaatttatttagctcatgttgctacaacctgggaaatcctggcatgggaccatctccacatattctaaattatataatgcatttacagttatacgtgggatagctatataatgttaagtggaattatgacctggctattgtttccatttggaataataataagggaataataataagggaatgtgaactgtgtgtcaagttgacaagattcggacttgtgataagatactctaggttgtcaacttgatgacatctgagattaactaaaacccatgctgctgggcacacctgtgagggagtatcttggttggctcttctgagctgcacctctgggtggcagcccacttgaaagaacatgaaggaaggaaggaaggaaggaaggaagtgtttgctttttgtctgcttgccctcactctggctggcaagttcatccattctgtttctcagttcgtttgctggtatcttcagggttccgttgtagactgaggaccagcacctctctaggacttccctgggactctagttccacatgagtatgctgataacgtttggtctcgtggactgaacaactacctttctctcaggaggtagccattgttgactatgttataattactgctaacacattctccagtttacagcctctccaatgataaattctgaatgcttttatctggacgtatgtgatgagtgctcagagttgaaagaattctggagaaaacttcaatacttagaagatgtgaaacaacttgttgataaaaaaaaaaaaatccctgagtctgaaggcaaaagagaaagcagtggagatgaaaaggtggtcctgaaatcatgggtggattcacactcaaactatttcaaattcaaacctgttgaacctaagtaaaaatgttctaaaaggaagtcaggcttgttctgtgcgattggagacaaagagaaaagaaagcccaagatgggctggagagatagcttagcaggtaacagtacttgcctctcttacagagaacccaggctcagttccttccatccacatggtggctcacagtcatttctgactctagattccaagttctagagcatccaacaccctcttctgacttactgggcacagtgcgcacatatagcacacatacaaacattcaggtaaaacactcaaacatataaggtaaaatttaaaaatccaaaaggaatgcctgggtagagaaatggcttggtggttaagaacactggccgcttgtccataggacctggatttgattcccagcatccacagagcagttttaaaaaatcatatgtagctccagctctaggagatctgacaacctcttctggactccctgggcaccaaggatacgtgtggtccacacgtattcaggcaaggcactaatgcacataaaggttttggttttgtttgttttgttgttgttttgagacagggtctttctgtgtagtcctggctgtcctagagcttgctgtgtagaccaggctagcttctaactcacagaaatcctcctgcctctgcctctggagttctggggttaaaaagtcaaacacaactctggcatagaaatagccaacattttggacccctcatctatttctagaaccctcagacatcatgttctaaaactcctcacctctaccacacagaattctcccattgaagccagttttagctggagttttgcagtttgagagctgacctatctaggcagttcatctttgggacagtttgtctcgttcctagtttcttctgaaaaagtgaaataatcagagaaactgactacgtataggaaataaaacaaaccagactaaatcacagcagggagtcaaactccagcttgttactgaagaaaagtgtctatccatcatcctttccattgtttgttttaaacaaggcctcactatgtagccctagctggtctggaactcactaagtagaccaggctggcctcaaactcacagagttctgcctgcctctctgcctctgcctctgcctctgcctcttcctctgcctctgcctctgcctctgcctctgcctctgcctctgcctctgcctctgcctctgcctctgcctctgcctctgcctctgccgagtgctgagattaaagcttgagcctccacaccaagcctaactataattctttccatacatatctgcacatctagtatttccaggtattgtactaggatcaaaaatacaatgttaagcaaattaggactgggccaaatatagatgggaaagatcatagaaccttaggactcaggttaccagtaaacaaatacaggttttcttagaaggtaaacatggcagtcagcaaagaaagttggtgggaggtagtcagcggaaagtgtggtgggttgtgaaagaggatcggggagaaacaaaggtcttcgattatggggaaccaaagggtaaagacatctggaagggcagattctgacaaaaaccttgtaggcttcttcatgaatttgtggttgtatttttttttttcctgtgagagtagcaggctccgaaggaagagtaaagtcaagtacacctggctaacaattatgctggtggctgggtggggggtggcacacaaggacacaggtctatagactcttgctattgatcaaaaacaacaatgtgacatccctcaaaaaagcttgatccaaatggggaaagatggatagcttcgagagctatcttgagcttggaggtgggtgggcagtggagggtaaagggaatgtttgctgatggctcttgggtatccagccgcactgaatggatggaatttaaaggaaactcctacaagatttttgtatagtggtgtcctttctcaccatacccatgtgtttttgcctggcttctacactcaggggcccctgggatatgttcaaccctagctacagagctgatacttgggctcccttgaggacacccatgagtaaacccagtcttctcgtctctctcccctctcatgcactttgcaggcaccacgtgtccacctccc

SEQ ID NO: 45 - DNA, 3757 bp, synthetic construct
  source 1..3757: mol_type = other DNA
atgacacagcgcagctcactcaccagttccctccacttcctgacatttcagtttatatctttcatttttctctgaagtacgtttatcattccttcctttaaaaaaatattcctcacgtgacatattggctttgaccttgcacttttatcatttagcaatgaactgacatagttgttaactactgacatagttttttaactctgtaccgtatacgtctctagatcctgcttccgggttcttgcaggagtgactattagcattcttgaaatgactattagcatgcagtcactaggactaaagtgttaactaactatccatgtaaaaggataggacttcatctggagatgcagtcagttgacaaaacacaacctggccctggattttaccaccagaacttatatatatataactgtttgagcctggttttgtctctgtaactctctgaaacatagtgatgaattctgaaagaaagctattaatcaaaattcaccataagaattgacagctgttaatgtaagatcatttgatacaaatactgtaagccccagaagtcattaataaatcactaaactaagattagtatatagtcaagaattttctttctttcttttttttttttttttttttggtttttcaagacagagtttctctgtgtagtcctggctgtcctggaactcactctgaccaggctggtcttaaactcagaaatcctcctgcctcttcctcgcaagtgctgggattaaaggcatgcgccaccaccgcccagctaagaattttcttaaaaggcataaaaaaaaaatggtgctggagagatggctcaatggttgagagcactgactggggttcaattcccagcacccatgcggcagctcacaactgtctgtaactccattcccagtaattccagttccagaggatctgacaccctcacacagacatacatgcaggctaaacaccaatgcacatgaaattaaagttaaaaataaatcttttttttaaaaatcaaaaaaatgctacatgctaacgaacatagaaagtgattttaaagcattttcaaaatatctttataacagagtggtttttaatctaatatcctgaatttagagttatagccttgtgagataataattacttaatatatccagaaatgtatgtgtcatctttttaataaataatatattatattcttaaattaattggtaaatttggtatatacagccaataaacatcacaatagaataatattataacaatattttaaagtacatctcggtgcattttaaagcagtaattgtaacatctttgtcatctgctgaatcaattctttccattgactgatttttttttttttaacaaattggttgatgggtagatttgccattctacagacaggctttggtgtactcatccatatttctgatttctggtttatccatatttctgatttctggtttctgtctcacacaaaactctttacttaatcatggcttgaaaatttatttatccataagaaaacattatatgagccatggtagatatcactgaccaagatacttagggcctaaaaaccaagggaactcccagacaggtagacacacagacacagccagacacacagacacagccagacacacagacacagaatacagagcctccccctgctcttgctatccgggagcttttcactgtatttaatgctaaatacaagaatgtttttttaaaaagccttgtgagtatctagcaaccttcatccataccatcaagcctggtccaccatggtccccgtctcctctctccatcccttgctacccatgctggtttctagaggatgatcagcggagttggagggagggtggggtggcatggtctgttccagcaaccttgctaaagccctggtggatggatggcaaggttgaagatctctaaataggattcaaactcttgctatgtgcacattcgggaagcctaggttttcctaatctctaaacagcacagacctctgagagtgggggtggagggtgacatttgctaatgtaacccactcagtaccgggcatacatgttcagtaagagatatgttgactgaagggtggtcctgtcctctttttaaaacctgtgaatgaactgctttataggcagccttctcagccgtgccgtgttgaggtggaaaccatggaaacagtaccaatgactgtgagggccagcagcaaggaggatgaagacacaggagcctaaccacacgatttcagaaactcagggagacctgcccagctgatcctgaagagaagatacaagggcagttagtccagctgcgcctggacacaggagaaagcccaatgtgataatagaggtctctggtatgatctgttttactgagcctgggtgcctcccatattcaggcctggatccccggagtccggaaggacaccacctttcttcctggttcatcacagaggccaacttcccagagtacaagcagcctgagctctcctgggagtctccattaaaagcctcgagttccactcagagattaactcaaagcagctgatctgctagactctttttctattccctactctgacttgcagtttacagagattgacaaggctcccattgtcttccaaggctcctctggcacaggagatgtctgtaaagaagacagcaacatttgagctcctgaagacttggccctttgctgctttgcacctattggaggagagcagagaacagaagaagagatactgagccaatgaaccctttcgtataggattcatgacaaaaccaaactcagtgactatatatgtatgtgtgtgtgtgtgtgtatgtaaaagtgtatatttacatatacatttatatttatactttcttttctattatatctacatattgtatatgatttatatttgaaagtgctttgtgtagacaaaataaaatatctattttcagtacaaaaagcattaaaattatcaacaaaaaaaaatgttcctcaaaggaaaaaaggagaaagaggggtgaaatggttctaatgtgaaagagatctcaggttctctttcaaaaacagagtcacaccaccactgccctgcttttggtgggatttttaaccaaatttcctagtgagtagaagcaacgtgaactaaacaatggcaagtctgagcccagctagcattagacacatacctttctctctctctctccccactccctccctttctcctccctccctccttcccaccttcttcatttctccccttcctacccctttcctgttatcacaaccatttataaaatcataagacaaattccacttctttgttttatttatttatttattattattatttgttggagagccgtggataaaaagggatttgcccctagtaggcaagtatttcaccactaaatcatatccacatatctacctttacagacaaaataatagtagcctggagaagttgatctgcccaaagtcacatagctcaactaatgctaccacttggacaaaactggggtgggaagttgggtaggagaggagctctggtcatgagtcagctgccaatgggcctcctgcatccctgggtaaaggggaaccaccataagtatttgggagatgagttaaacacctggtgcacaagctaccattcctaactggcttctgccagacatcagtgtatgtggtttctttcatcaaaactaaaggaagtctttccgcataggtcatgggcttcctcagaacatggtagactagttaggttgtttaa

SEQ ID NO: 46 - DNA, 9882 bp, synthetic construct
  source 1..9882: mol_type = other DNA
atgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagtgagtagcccttgccaccccaccttcctccctcccccaccctgccaaggctgcacagagagaagaccccatctcaggggtcaagggatctgtgctgaggtctgaggctaaacacaccagtgggggccgggcacggtggctcacacctgtaatcccagcactttgggaggccgaggcgggaggatcacttgaagtcaggagttccagatcagcccggccaacatggtgaaaccccgtttctactaaaaatacaaaaagtagccgggcgtggtggcaggcgcctgtaatcccagctacttgggaggttgaggcaggagaattgcttgaacccgggaggtagaggtcacagtgagccgcgatcacaccactgcactccagcctgggtgacacagcaagactccatctcaaaaaagaaagaaaaaaagaaagacaccagtgtgatcctcagcaacagctcctggcgttggaaacacacaggtgctggggtcacaggattctgggtttaaatcctggctttgccgcctaccacccaggaggccttgggcagctcttgtgatattctggcactctggctggtcatctggaaaacggacttgatatccacactgcgggctgtgggagggtggcgtgaaccctggtaaatattaactcttattacatgtagtgctttgatttgagacgatgctggcaggagaatttggctgcagagctgggcgggcactcatgagcacgctagtgtgagaaatgtttggatttcccatgagaaggtggatgttggaagccatcactgctcctctctcagtacccagccccggggatgggcacaggccctggagcagggctgccctttgctcagtcacctgtgcatggtgtccagcaggggtgagggaaggtggcaagaatcaacttttttttttaactgtctgcacctggataaacgtcccagaagcagccaaaccatctcgggtgggaaccacttggagctgtttctggaacactcaggcccacctctgcaggcttctctccttccccagttcatctctggggcagggatgagcgcgcccctgcatggagcggccccatagctcacgccctctgcagcaggggacagaggggagctccttgaggcgctggacattgccactggcttggtagtggtagctcagcctcctcccccagaacagagcgagtcacacaggcagcagagggaaggcagggaggaagcctccgacttgttccaagaaatcactcgctggggacacagcaccccgtggcatcagtgagtgaatgggaaagctgagcgtggcggggtgagcaggatctgtgggaaccacggtgggtcttactctgccagtcctgggactccctggaacctgtctgcccgaggtaaaatgagggctttctcactgcagggcaagtcaggtctcctcgacactccatgtcctttgggcaaatgaatccgggatggagaagagctatgggctccagggtcaggatgggagaggaaagaagagaacccggaatcaggattttagagtccaaagactggaggcctctttaggtttcacctagcccaaaggatccttccacgttctgatcactcacaactccctctgttatgcttcccaaactcttgtgctttgaatgacttttttcctgctcaacaagtgaaatgtaagccaggtatggtggcttacacctgtaatcccagcactttgggaggccgagatgggagggatttcttgagaccaggagttcgagaccagcctcagcaacatagcaagaccctgtctctaccaaaaaaattaaaagataaaaattacctgggcattgaggcacgtacatgtagtcccagctactcaggagactgaggcaggaggatctcttgagcccgggaggtcaaggctgcagtgagttgtgatcacactactgcactccaacctgggcaacggagcaagaccctgtctcaaacaaacaaaatagaatctattgagtgatcatttattctcccaagaataaccagcattataaaattgaattgatatcattaatataatctcagccaaaataaatttgatatttaacttgtgcagtctttttggaaatgcaagaaaaatagtttgcagtctatctttgcattcgggtggggaaacattgtttcttatgcttttaacctgtctgaggaagccagaggcctccactgtaagttcatgtgttgatatattttatttgtatcccgttctgaagccagcccaggttccaggctcaggatcataagtgccatgtggttatcctctctctaggagaccctgccctggttcaccaaaggccagcgccaccctccacagtaacgacggcaggggtgaccccacagccagagagcctctccccttctggaaaaggtaggaaggtcagaaacttctcgaggacattcattccctcccaacacccagaggcccgctcaccaatcctgcagactcacggacctctgtggtgtgccactgcagacttaggcaatagcgcaaacgtcttattcaatcaagaataagaacgtccaacttaattttctttttcaaatcaaagttgcttgatatccaggtgttatatgtgcagaattaatagtggtttaaaactcactaatatgtcatatcaaatttagttgtagacacttatctttgtcaagaactgaaagcagactgaggtttgaggttgcatgtgtgttagccctcaacattttcatgggtgctggcagaaaagagggcactccaggatgggaggccagcaggcagcgggagcttcagcatgtttacatcaattccctcgtcccctagccagcaggggcagcacagttgggcctgaacagatgcctgtccactcagcgggctgcactgtgggagaggaagcctgggcttccgagacccaagcgttttgtcatgggcagtaatcacacctgctctctgctccaggggagaccatgtccccgtcttccaaggcgcttgctgtagaagcacccttgagaggatggtccagagaaagggccatcagctcctctgtctgcaagatgtgcaaaaatgtgagagacctgtggagagctgcccccaccagacctaaacaccagcacatcccagtgactgctctattatagaggcgctgcctgaagctgccagtgtcctgtgtttcaacagagtctttggtgcttgtcaggcccaatttcctcagcgctttctcagtgttcagaaaatttgaaatcttggatgctttcttgggatcctccgccctgcatgctatgatacctttgtgctacagttcactcttggctttagtgttgcatcgtccagcctgtggctttccctggtcctgtctactcctttgcccccggggggatgggcctgtctgttgtcattcatactacaggactcagccatcctttgcggagtgtcagttgtcagccgtgggtacacactgacatcacctgggcactccagttggcctgggtgcaatctgggactcacgcctctccagccccaggtggatcttttttttttttttttttgagatggggtcttgctctattgcccagtctggagggcagaggtgaaatcttggctcactgcaacctctgcctcccaggttcaagtgattttcctgcctcagcctcccaagtagctgggatcacaggcgtgcaccaccacgcctggctaatttctgtatttttagtagagacagggtttcaccatgttggtcagtctggtctcaaactcctgacctcaggtgatccgcaagccttagtctcccaaagtgctgggattacaggtgtgagccactgcacccagcccacaggtggttcttaagtggccaaggctgaggcctgcatcttaagggaggaagaagcagctaaacaaggttccctcctagtgagtcacctgcacagggaggaaggggttgaggggctggctccatttaaacttgaagtaattctacaacccctttgatctgagtcacacctggttcacccaaggaggagaatgatcaagtaggctctcccagccccaccttcgcatccccaatacctggtccgatggccattgtcgagacacaatggtgcagtcagaagccccgatgtgacagcagcctttgacccgagctggcacgaccgtgggcacttggctgaggatgccttggtgcgtcctgtgcagggagccctggagcattggcagtgagcgtagggcaagttatatgaaaggtggtgacacaggattaaggggcgtgaggccttctcatgacctgtgtggctgggagaccatgggaacgcggcaacaacagtgcgtccagagagccacagcgcttggcctcacgtttcctagattccgtggctgtcacaatggaagacacatttttcatggagagggaacagcacagttacgccacacccttacagtgcaggggcagccaccttccagggaaggacaaggaagacagggaagacgctgaacacaaggcagcctctgttcctgagagcaagctcatcaggacgctttcctcccacacagcccggagagttcaggccagagcccaggcttcccgtgtttcacacgcagccgctccgagcgtcctcggccagcgcagcttcacctgctctcagctgagcctccagcgttgggcctgccttctctagtaaacaagctgaacgactcagatctttgacaccttttcttttcttccccccacctttttttttaagagacaggggtctcactctgtcccccaggctagagtgcaatggctcaattataggtcactgcagcctcaaactcctaggctaaggtgattctcttgcctcagcctctcagatagctggaactataggcacatgcccaccacacctggataatttatcgttattcttatttttgtagaaacagggtcttgcggcagggcacagtggctctcgcccgtaatcccagcactttgggaggccaaggtgggtggatcacctaaggtcaggagtttgagaccggcctggccaacgtggtgaaaccccatctctattaaaaatacaaaaattagctgggcgtggtggtggacacctgtaattccagctacttgggaggctgaggcagggagaattgcctgaacccggaaggcagaggttgcagtgagctgagatcgcgccactgcactccaaccttggcaacagagcaagattccatctcaaaaaaagaaggaaaagaaacagggtcttgctgtgttgcccaggctggtcttaaactcctgggctcaagtgatcctcacacctcagcctcccaaagtgttgggattataggcatgacccactgtgtctggctgatcttttctttatacgtgccgtgatagtagccatgttctttagtgttttcacaggtcctatggcccttccagaggaacggctatgtcaccctctcaaggagcagttggctgtgagagagggagggctggtcagggtgactcactatggcagctcccacatcaccccgtgctgcagctgagaggaggggagtctgcacactgatgttgtccctgccttggactctcctacagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggttagcactcgctttgccccaggagaggtcagttccccattgctgcttctgagatcggggctgagatccggcacaaggcagctttataccatcaatccaaagtcaggattcactgggcagcctgctggccccggccaccagccacggcgtcctggcctgacctctttctccgtggctttctctgtggctggggcagctgcaaaagcaagcccacacccccccaagatgtgacagggccagccttatcacccgcatggcagagcctgtgcagctgggagtggcatctgtgccctcctgcagtaggaggtggtgaagctgcctacagtccggggccaggatctgtggaatggaggagctgagggtaatggggaccttctgtaaggcttatctgattcttaagtgtggccagcacctcagtgagctaccctgcttgttctttgctaaaatgagtctggttacgccagccacgagtctcccagggtgctaagatgggagccaagccatgcagattttctcctgcttcatgaggaactaggacctctaggactcgagccgtgatgctgtcctctcttttccgcaggtgtgtatccacagggccacagcgacaccactggtaagtgtgtccctttgtccggtatgtttatgatcagggtgaccgcagtcccagggtgctgggtggtcctggcccaggcccgctgccccagcacagccctgacagcagccccttccacactcagaaggcctccatcagataataagttaagtaatagctgatttatggaattctctgtttgatgtgcccatttttctaaatattggtagcttctagctgtcaggctactggggttgaacccagagagctggcaccccaaataagggctcaaggtcagctcttcacagcagggtgcacctcttttgcatggagcatccaaaactacaggtgctgatgccagcactgacgttacagcatctctgccagaagctgccaattcctctccagggcgtggcaccgtgcacttaacagagaagctgtccacattttcggtataaaatgctactcttggccaggcatggtggctcacacctgtaatcccagcagtttgggaggccgaaatgggaggattgcttgaggccagaatttgaggccagcctggtcaacatagcaagaccccatctctattaaaaaaaaaaaaaaagaaaaagaaaaagaaaaaaagttaacaaaacaaaataaaaccaaaaatctgctcctctttacagaagtcacacgcaatgctaaggctaggagaatagaaaagagaacgtgcaagtgatgataaggaggaaaaggggaaagcgtttacaagactaaaagaaaaagctgaagggaggggaggagagctgagagaggaggggagaacaggaggaagaagggaagcaggcagggaggaagaacgcaggtaatgagaagaatgcaagtcatgctccaggctgacaagcttccaaagtaacaaaaaacccaactgtaatttgatatggacaaaaattattttaatcttctgttcataaatttttctgctagagaatgactcatatttttgtggtgtcatttttacctgcatcaaacgtcatctgggaaaacatcaagtgactttggacattttagcagttaattgattagaatcgtttaaaaaacaatcaggctgggcgaggtagctcacacctataatcccagcagtttgggaggccgaggggggctgatcactggaggccaggagtcccagaccagcctggacaacatgacaaaaccccatctttactaaaaatacaaaaaattagccagacatggtggcacatgcctgtagtcccacctactcgggatgctgaggtgggagaattgctggaacccaggaggcagaggttgcagtgagctgtgatcacaccactgcactctggcctgggcaacaggacaggaccctgtctcaaaaaaaagacgaaaagcaaatcccatttctaactcaaaaatccccttgcaggaaggaagcaacacttgctaccagatttccaggcatccttccagagagactgtataaataaacatatagaggataaggaatgctggacagttgcttcttttctccttttctgtaccatgtttgtttccacttgctggtataccttgtagatcctcccatatcaggacacaaaaaccatctcatcgtttttaacagctacagaatattccactgtatggaattcctaccattaatttagccaaccccctatctggacatgtgattgctacattctgttgttatcttacaaacagccattaattgaaaatccataggtgcaagaacatctgtaggataaaatcctacatataaagttcaggggtcattttttctatatacattttccaacaactatttgatgatgggctataatctataatctgcttttgctagttcatgacttttattagattaaaaaatcagccgggcgctgtggctcatgcctgtaattccaacactttgggaggccaaggtgggcagatcacttgaggccaggagttcgagaccagcctggccaacatagcaaaacaccatctctaccaaaaatacaaaaattagccaggtgtggtggcaggcgcctgaaatcccagctactcaggaggctgaggtaggagaatcgtttgaacctgggagggggaggttgcagtgagcaaactcgagccgctgcactccagcctgggcaacacagtgagattccgtcgcagaaaaaaaaaaaaaaagaagaagaaaagaaaaagttggccaggcgtggtggttcacgcctgtaatcccagcacttcgggaggctaaggcaggcggatcacgaggtcaggagttcaagaccagcctgcctaacatagtgaaaccctgtctctactaaaaatacaaaaattagccgggcatggtggcacctgcctgtagtcccagctactcgggaggctgaggcaggagaattgcttgaacactggagctggaggttgtagtgagctgagatcacgccactgcactccagtttgggcaacaaaatgacacttcatctcaaaaagaaagaaagaaagaaaaagttgtaggtagactggacgcagtggctcatgcctgtaaacccagcactttgggaggctgaggcaggtggatcacttgaggtcaggagtttgagactagcctggccaacatggtgaaaccccgtctctactgaaaatacaaaaaattagccaggcgtggtggtgtgtgcctgtaatcccagctactcagaaggctgaggcaggagaatcacttgagttcaggaggcagaggttgcaatgagccgagatagtgcctctgcactccagtctaggtgacagagggagaccctgtctcaaaaaaaaaaaaaaaaaaaattgcaagtgaaaaaaaaatgaaattggaaggaaaatggcactgtgtgctattgtttctaaatctgtgaagttgaagcaaagtgggcgttgtactggggacagcagaggaggggtgtgtccctgtctgctttttcaaccctggctttagcagcaggaatgctctctcctcatcgcactcttggctttgcccttgaggctcttactgctccctcctgacttgctccgtccctgccccgtcaccccaggcactctgcctgagaaggacaaggatgaggagcagaggtgtgggacgactgggagctggcaccctcagcaggaaagagaatgtgtttttgcctagacatagaatcgggggaattgcatggaccttaagaaatgtccttagcaggaggctggttgggaggcacttggatccctctgtaagaacatccatggtaaagaatttgtggttcgtagcaatgaatgtgcttctgggtatctcagcgtggtctcctccctttcagtggctatctccacgtcc

SEQ ID NO: 47 - DNA, 87 bp, synthetic construct
  source 1..87: mol_type = other DNA
catgtcagccttgattctgtatttctaatagcagaacatatggaattccgaagttcctattctctagaaagtataggaacttcaggt

SEQ ID NO: 48 - DNA, 102 bp, synthetic construct
  source 1..102: mol_type = other DNA
tctctagaaagtataggaacttcatcagtcaggtacataatggtggatccatgacacagcgcagctcactcaccagttccctccacttcctgacatttcagt

SEQ ID NO: 49 - RNA, 1673 nt, synthetic construct
  source 1..1673: mol_type = other RNA
tctctgcaatccgggggctaaagcaaaagcgaaagcgaatactccttagcagcggtctggtcctggagctgagctcgccacctcccagggaccccagccttgcgtcccgttgggtcactgctggggacaattggccatggcctcgccgcagctccggggctatggagtccaggccattcctgtgttgctgctgctgctgttgctactgttgctcccgctgagggtgacgccgggcaccacgtgtccacctcccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgtggctatctccacgtccgtcctcttggttggtgcaggggttgtgatggctttcctggcctggtacatcaaatcaaggcagccttctcagccgtgccgtgttgaggtggaaaccatggaaacagtaccaatgactgtgagggccagcagcaaggaggatgaagacacaggagcctaaccacacgatttcagaaactcagggagacctgcccagctgatcctgaagagaagatacaagggcagttagtccagctgcgcctggacacaggagaaagcccaatgtgataatagaggtctctggtatgatctgttttactgagcctgggtgcctcccatattcaggcctggatccccggagtccggaaggacaccacctttcttcctggttcatcacagaggccaacttcccagagtacaagcagcctgagctctcctgggagtctccattaaaagcctcgagttccactcagagattaactcaaagcagctgatctgctagactctttttctattccctactctgacttgcagtttacagagattgacaaggctcccattgtcttccaaggctcctctggcacaggagatgtctgtaaagaagacagcaacatttgagctcctgaagacttggccctttgctgctttgcacctattggaggagagcagagaacagaagaagagatactgagccaatgaaccctttcgtataggattcatgacaaaaccaaactcagtgactatatatgtatgtgtgtgtgtgtgtgtatgtaaaagtgtatatttacatatacatttatatttatactttcttttctattatatctacatattgtatatgatttatatttgaaagtgctttgtgtagacaaaataaaatatctattttcagtacaaaaagcattaaaattatcaac

SEQ ID NO: 50 - protein, 266 aa, synthetic construct
MASPQLRGYGVQAIPVLLLLLLLLLLPLRVTPGTTCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSVLLVGAGVVMAFLAWYIKSRQPSQPCRVEVETMETVPMTVRASSKEDEDTGA

SEQ ID NO: 51 - protein, 162 aa, Mus musculus
MKILKPYMRNTSISCYLCFLLNSHFLTEAGIHVFILGCVSVGLPKTEANWIDVRYDLEKIESLIQSIHIDTTLYTDSDFHPSCKVTAMNCFLLELQVILHEYSNMTLNETVRNVLYLANSTLSSNKNVAESGCKECEELEEKTFTEFLQSFIRIVQMFINTS

SEQ ID NO: 52 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 53 - DNA, 3845 bp, synthetic construct
  source 1..3845: mol_type = other DNA
agaactgacaggttctccttccctgaagagtgagcatggatagccaggagttgtaaaacaattcattcaagtacaagggctgttcgttgctttagagccacagtttgtctgagaagtaaacttctggtctgtgtttatagttgaactattaaaataccgcattgaattcctatcatgtgcaactgttgctcagcatcactgtttaaggatcccgtggtttttcctgcctactatactcagtcagagtctgcaggttgtttgtgaaggtcacaggcaacctggcaccacacttctacaactgtatctcttgacttccaccagtgactcttcagagagtggcatcattcaaggagtctcacggtgcagtggtttcccgcaataacctctttgcttctgtgcaaagtttattccctcatatggccacttgactccaaagcgtcctctattttggaaacatgtattgatattgcttattattcttgtgccatgataaagttgcttctccctctgcctatgaaattttattgcatgcttattcagacgtaatttgtatattgtatgcagtttattgttttcttcataacaaatttaatccactgtgagagtgattatacctgagaagagggaggaaaatggtagaagaccagatgaggcagagggctgggagacaggtctttcctctgagatgcttgggaacatgcctaggtagtagcacacagggaatacggtccatgggcctaaatttctgcagtgtttgggatctagcgtcatctgatttctgtacagaacagaactgatggaatgactcactattacaaaggagagttaccagcttggcttacacagttaggagtagggagttcaatggaccctgcacactgagaggtaactgctcagaccgacatgcctctgaagtgctgatctaataatcaaggcctggagtgttccttttgagtccattttggaaaactgaagaagccgcatccaatgtcattgaaggatgacagcagcaatggcaacagtagagtagatgtgttcaccagcagggggagaagggaagcaggcaaaaagcagctttccctcaggccaccccaaggatatatgctggtctaccttccccacttcttaaaggaaacaaccacccagatccttccataggcatttttcttaactgattgcaggttaatcaagttgataaccaagagtgaactgcacagatctctaagaaagaggattccatgttaatattgaaattatgtgtgcaggcctgcatatctttatagagatgacatataaaaaatattaaacatcttaactgatcatttgttgttatcttgtgaaaaactgtaagatttaacatccctgaagctggacacctctaaaggaaagaggcatatttaatgcacagtgttggacaacaccaagtgacctcattaatgtgtcacttggtcgcagtgccgttggggtatatgtggcttccccatggtgaaggtacacacaagaaggagagagcacatggtgagagagtaatgtgggttggcattcttataacagctcgttctgatgggaactggatgagattcctgtagagttccactaacgccttccaagggcagctctaagactgccaatgactgaaaaactgccactatgagttacaagacaagtaaagtttaaatgaagctggagtaagggacacatagaagccagatgtcatcagcaaagcaaatatttgcttgccagaagatctgttttctcagttggtaagtaggaggcaccagccacctattttgatgacaaagctgaactgagatgcagccccattcatcatctgccgccattttcaagctatcgtggacattgatgtaattacaagaaaaaccataagactttccaagtatgtgctacttattacctggcccttggtagaaaattactttaatttgtacagtaatgataactgttatctcctacttttctgccataaatgaacgcttgggtggacatagtcttgctcaggtgctttctccccaacttcacactatttcagttctcattcatggttaacatctgaccatatctcaaggtagggctaaaataacactttgctataaagacctgcaatgtagggaacgaatagggcatgcttcacattccactggccttaatacttccaagctccctgctcccttctggtctgtgctcacttgtcatacgcagattttaatccatttctctgccttttccaggtaatctgtcacttgtgggttgccatgcctggagcatacacagacataacacagagataacataataggctcttgttaccagtgaactggaaagccatggtccttagcaaaaagactacagtcagagcacttttccagacaaagaacacacgttgtcctgaggagaagttaatcaagctttactgaaatgaaagcaaggcagttaagaacagacctcacaaaagtcattgcgggctgagtgggaaaccctggctgactctcaagtctttgctgcgtttactttccattgtgggctcaggggacttaggctttaggggcaaacgtcagccctaggtgtaaaccagcctctctgtttaataaatgaaccactgctggagggggaagtctaatgccagtgtcacagggaacgggaacgtggggtgaagacttcatggaagcattgtactagccacacagtctagtgtactgtttcataccctggtaaatgttaggtttattttctcttaagaatgatgtaatatacactgtagagtgtaaacaagatagcaagatgactcagtaggtaacagtccttgctgcccagtttcatggcctaagtttgttcctagatccccaagtggtgaaaggagagcagcaactctcacaatttgttctttgacctccacacatgtaccatggcacgtgtatgagcatacacacacacacacacacacacacacacacacacacacacacacacacagagacacacacacatagtaaatgtaaaaaaataagagttatacattgcacataatttatagcaaatattttaatattaggaagcatctgaaatgtgtaaaatattgagataattgattgtgataaattatatcagtgaagcaacagaccttgatattttcattggtaaggtttactaatgcgtaacatttttgacagcaagactatccatatttgagatattttgagcctaaatattttaatgaaatggagtttgatgggagtgtttaaacttcacatgttcccctgttttggtttagaaaaaaatataattttgttatatagaaaattcacaaatggtgttctcattaaactttattttaaagaacatacctaagacatctatgtgaagtctgtagatgaggctgttgttgtaaattcgactatcagctggtggttatgtgaatctttgtatttgattgctcttattcaaattgagatggccttgaaacctgtcagatctgggacactgtgtgaaataatgtctttgttcttttattcagacaaactaaaatggttttagtccgggcagtcatgggatttctgtgacgccagatcagattttccaaatgatgctctcaggagggctaaatctgatgcgtgtgttaaggaacacaaagcctaccctatggaaagcagatgtggcataagcaccaggcgtttctctatctgcttctggcttactcgcttgtgttttgatagtcatccttcatcctggttctgttgcaggaagagttctggatggatggcagctggaagcccatcgccatagccagctcatcttcaacattgaagctcttacctgggcattaagta

SEQ ID NO: 54 - DNA, 4036 bp, synthetic construct
  source 1..4036: mol_type = other DNA
aactgctgtttacattagtctttcacgtttttattttattctcacaaattttaatgcaatacacacttgttgaattttttaaaggcctgatttattggactccatttggtttgacaaaaattcagttgttcataggtgctgtcaaaatggctcaggggtgggaatatttcctgcacaaccatggggatccacatttgcatccccagcatctttggaaagctggccatggctatgtctataccgcaacttgtggagaaacataaggatcattatggcttactggccactggcttggatacaggttcaacgagttacactgtcttcagaagatgtagaggagagtggtagaccaggacactccatacacatgtatacatacatgcattcaacaccgaaatgcaacttaaaataattattaataagcttttataatccttattaatatcattcattgtcctgtctaaaattagtaagctttgtagcattcccattgctaggaaatggggaaaatgtaccattttaaactttaattttccagtctaattgttaaagaaaagttggtccccatccagtgtttgtttatatgaggaaagggagaccaaaatgctagtcatatttctgatgttatttgatagtgactaataatccactcacattgtataatcaaatagaggatgaatttaaaatttggaatgttccagtgctcagagttactgcctctcaaatgtcacataccatagctgattttagaaaactatagttgttaaaatcctttaacttgatataaagtatgtgtgtgtgaagcggtcatatacaaccatcttttagttattataagagataaagtgagcatgtaccaccatttgaatttaacttcatcttcctgctcttaaagtcaaacagtgatgtctatttcaaagtccatgctttccacacccttccattggccagcctagggctatctccctggggaatactgtgtttacatttcacctactgccctaattgatgaaaatatttgtcatgcatttcctataattaaataagcacaaaatcatatctaatggcaaatggacaaggttagacaaataaaaattaaactgataatccaacatgcagttgatggatagattcttacattttctttatttcaagcaaagaaacaatgtttgaagggatattatctgcatgctttctctggtaatacagtaaaaggactctgaggtgggaccaggaaatacaagcataactaacaagtaatttgtgagccaagagaattttataacccaattacaaatctagtttcatataatctcattctttatctgagacgagcgaatcaatagatttaccttctgctgttgttactgttgctgcaaagagggaattatatgttctgatgtagtttatggctgacaaattcaataacttataaaatcaaatcgacatacacaaaataaaaatcaagtaatgtgattgttccctctttgctgtccccaagcaacaagaaaccccagagaaagtattcctgttactcctcctgtgtgagtacgtatctcaacaaaagctatctgcactcccaggcaagtcatgcttaacatatgcatgcaatggcccagaggaactcccattcccagggagatttgatttagtatatctgaacatgtctagtgactgaattgtgttctcaataagaatgtgttatgtaaaatatttagctgtatgaagagtcccttggttctaattgtgtactctacctaacacaacatagaagaaaattaacgaaacatagacaggcttttatcctgtaatttatttcttcaaccctgaatttggaaatagggattaaaaacaccgaggagatcgttgtaacatagaagcagatttaaaaactgctggaaatagggtcagactttttggactattttaatggaaggcagcatccacttccaagtcatttacgttcactgtcatgatcacattttttttttcattaagcattcactatgtcagtcatactggtaccttagttattggaaatctactctgtgtgaacattgtaacatgctgtaagaaggccatagtgtcagccttcctgggtttcacaatcttgcagaaacattgtattatcaagctctttttccgaggacttattttgtgtccatcaattctatgaaggaaatcaaggagaatcaggttgattcaaggtgtccgttcaaagagcaccccttggagggagtgacaggtgagctgagctgtcagggcatatcaaataagctagaggaggagtgaaaagactgacaacactccagtcagtgaagacaggagtgtcctagagcaaaagaaaatccagaagagccaggagggggaaatatagaaaaggaactgtgataatgaaagagttaactgcctccttgagctacactaatgacttttctatctgacttatagaaagaagactccttgtaaccttacccccccccaaaaaaaaaccctaacaggaaaaactaccttgaacagaaggctttaagtcaaagtctccttataaataccagataaataccaggctcataaaatttgtcactgtgtgggagtgccatctctatcatctttgatgatttcttggaacaccaaatggacagcaagatcagcaagtaattactcactgtgacagccctacatccagcacgtggtgcttgcaagactgaagtcagggccttatgttcaatgaagcattgtgatttacaggggcagaaaggtgtgtggggaaaaaaatcctgggacaaagaaggctttttgaatggaggattttctaaatcctacatcactgaaacctgtttgagataagggaagctacttcttctcatcaagatcaaccaagtagaggaaggcctgtggcgtgggtggcagcagggaagagttgaaaggaggaggtgggatgtaactgggctgaagtaaaattagccttacataacactggggacagatttaacaaggctcagtagtcatggaagctgatgggctcaaggggaggcagaggaaggaaaagcatccctcagtacatgacagtaaagagcctatagggaaatgaggatgtcagagcatggatggttcttaatggtgggcaccatcctttctaaaggacttgaagattattggtcttcaaccaagctcagtttccagagatgaattaacaaggtcaaggccactaatgtatctgtaagtcctcttctcaggatgcaccagggtgatggtagtaataatagttcttttatacaggtgtgagtctttacatttcacaaagcactttcatctatattattttattttgtgagaatgtttgcctctcctgggcactgattcaatgagaaacagccttggaaatatttgacttttgtcagtgatgtgtaaaaagagggaaaaaaatacagcaagaaatgacctgaaaattttgtctgaactcagctgtgtgtatatttatgccctattgccatttgtatttctcttaaaataaacatgagtttaatacttggggtggaaaattatgtcttattcaagagttcaagataatgtttaagttcatatttttgattcatattttgatactatatctacatacaattctattctgcttttctttttcgtggctcttcccacatgacaactgcaccagtaagcttatccaacctgaattaattacctttatatagcaaaagaaaaaagtctctcctttttacaaggaagatgtttgagctctatttatggataaataaagggagctgctagacacagcatttttaatagcatggcttcaaacattccatgtgaaaacattcccactctttttatttttcacaagaacatcagccttttgtgtccctgggtttgttaaggtgtgaaaaaccacagagtagcaatacttctacaaagcacccagttcagaagagcaagcaaaaggcagtatacttaatggtcactcttttgggggggccacttagcctggttctttaatgtccaccctgtgaagtgtaggcca

SEQ ID NO: 55 - DNA, 13384 bp, synthetic construct
  source 1..13384: mol_type = other DNA
atgagaatttcggtaagaaaaaaaatagatgaaaatatcctatggaatttcccttaaaggtcgtctgaatctcagagtctttgcaataagttacatggttattctccaaagatcttgagatatcacagatgtctgttcacatttggattgttcttattttgaaaataaataggtttttaaaaactctattgaccatcttgataggctcttcttgtcattataaatgtgttatttcacttatcccactcttttgtgttctctaaatgtctttgtactcactaaattgtgtagtcttagagggcagggtgtgggtatcattcatctttttgaaaggaagcatgaacaattagtggttaaggtggcagttaacactgctttctgaaactttaaaagcttcgacaacacaaaagaggcaggtgaaagtaccaaaactctggtaagttgtaggataaatgattttcctaaatcaccaagaaagattattagtcactctcaagatggaatacagtatctgtaattggttccattttatccaatttttcaccaaataatattaatataattcattttcataatttacatgaagttaatataactattgcatctctgttagtaatgcatgttattaacctgtgctgtataaaatattttgacctattaaaaaagaaagaaggatgaggaggaggtcaaagataatataaaaactttaagtggtgacattggtatgtatgtaaattaacaaatatgtcaactttgttaaattatctttgacatgcatctgcagaaatatctgttaaggaactttgtgcataagagacagagttggaggtgggagagaaagtgaaaaaagataaattatagttttattatttttgataaggggcattaattatgaaattatgacaaacttacatctttagacctaaaaatatgttgacatgttattttaaacctcactttttaactaaaaaatttaaccatttgtctatgattatatttttcagaaaccacatttgagaagtatttccatccagtgctacttgtgtttacttctaaacagtcattttctaactgaagctggcattcatgtcttcattttggggtaattttatctttaggcataaataacattatgttcatggtcatgatgattgtccttggatatattttccattaaattagtttacgttcagtttgcttatatctctaggtactcagtatctgggggatagaaggcagactacagagatttagagaggtttggtaaacacctaactgagcatttttctacattgtggtaatagtccaaacaagcattatagccctcaaataaattgggccttttgatatttaactgaacttggaagtataccacttattgatagactacatatttttcatggcaattaaaagtagatatttagaatttgcttatgttacttttttatctgtccctgacactgactcctcctatcccttgatctttatcctagttcagtttaaaacatttctttctttatttaagccttttgttgtttccttctaataaaattcatatgtttccatgactattaacttttttcttctctcttagatgcagctaatatacccagttggcccaaagcacctaacctatagttatataatctgactctcagttcagttttactctactaatgccttcatggtattgggaaccatagatttgtgcaggtaattcttcatcataagacagattagtgttgactatcatgcttctgtgtaagatccatcaggaagtgagtgattatttttcctcaagctctaagcatcattccaatgttacttggtcagtataagtgtattttgtctggatattaaattttaatttttatgaaatttgtgaaagtgagtaagtatatgatcattcagcctacactggaatgaagtctgtactgtttatggagaaagctgctgcaagcatagtaagagtgtgagcaacatgaaaggggcatgtttaatgtagtgtaaaaaaaatttactatcaaaatcattacatgaaaatgattattactcattttaagaattttacctatttattagacttcaaagtctctgtaaatctctacttaaaggaaactagtaattcattcaaaatcatttattgagcattgccacacatctggcactgcttctatttcattgtgataaattgagccagtttatttataaagtatgcctacagttaggcttttctctaatccgggttggtgaagcctcaagttatcataaaatgccaaaattgtactatatgctcaatgtttatgttcagttacaaggctgttgaatgcacagaagcaaggataacactgattttttcactggtcagaataaaaattattgattgctcttttgcttatagtattcatcaagatgaataggctccttcaaaatgctttgctctatggtttttctttcagctgtttcagtgcagggcttcctaaaacagaagccaactgggtgaatgtaataagtgatttgaaaaaaattgaagatcttattcaagtgagtactcatttttccataaaatgcctggtataactgctatggagtttgtctctctctgtgtttttctgtctgtcttaaggaaaccacacttgaaacaataatatttttgtagaaatttatgatcttataaagctcaaaaagaagtcttttccttactgttttagaatctaattatctttaaaacgtaatttataagcggcccagacggatgagttctagtctgcacaggtgaggtattctcttcctctatctggagaagccagatatcttggtggcacctgcagggagaatggcttgacccagggaacagctgttgttgcttgtggactaaaggccctcttctcccacctcaagaaaacaggtggcctgacctaaggaagtgatttttgccatagcaggtaataccagcttcagtggaagtctcgatggcaccaagcataccaaaatagcaccacaaagttctgaaaactaaattgtcattggaatcacagcattaaaaaataggttagaacttgcatgctaaatccaaacaacctgactgcatgttaaaatagaagatttaaataggaccagagtgtcctaacataatagacatgaagccaagatacaattggaaatcacccattgtaacaagaaataggaaagttacaacttaaatgagaaaatatagtcaacagattacaaaattgaagtggatcagatgttggaattctctgacatagatttgaaagcagacattataaaaagtgcttgaaaaagcaattacaaatgcccttgaagcaaatgaaaaactaaatgccttaacaaagaaatagaagatatgaaaggaaccatatagaaattatataactgaaaatataactgaaatataaagcaaactgtaaaggctcattagcagaatgaatgacagaggaatgatagttaatttgacaacagaatactagagagacagttgacttaagaaaaaaaaagcatcactgatcttgacgacagtaacaaaagattcagcattcctatggttagtatcacaggagagtatgagaatacaccaagtaaaagagagagattggttgaattcataacaaaacaccacccatctatactatctataagcagttcacttctagtacaatgacatacataggttgaaatttaaaaagacagaaaaagatatagcatactataatgggttgaatagtgtccccccaaatttcacatcctcttggaaccttaaaatgtgatcttattttgaaataatatctttgcaaatataattaatgaaattaagatgagatcatactagagtagggggggccctaaatccaatgacaagtgtctttattagaaaaggaaaggacacagagtaactcactgggaagaaagccatgtgaagacagaggaagagatcagagtgatgcggctataagccaaggaaggccagagattgccaggatccaccagaaccttggggaagtcaagaaagcattcttctctagacccctcagaaggaacatggccctaccaacaccttgactttggacttccagtctccagaactgtaaatgaatagatttctgttgttttaaaccacccaccttgtggtagtttattacagcagctctagactgatacacatgcatacattaattataaaaagcagaagtggctatattaacatcaaagttaatgttattaacataaagtagacttcacagaaaataaagttgctataatccagagggatattacaaaatgataaaaaataaatctgtgaggaaaacataatgttcttaaatgtatacacatcaaacaacgaagactcaaaatttgtgaaacaaaaaataatagggataaaagagaaatagacaaatccacaattattcttgaggacttcactgcctcctcctcagtaactaatggaagtactagacagaaaatcagaaagggcataaaaatacaatataaccaaccaacaggatctaatttaatacatagagcactacacccaacaacagcagaatatactttcttttatcaagactaaatatatactgagccataaaagaaactttaaaaattttttcagaacttaaatcatgcagaatttgttctcaaaccacaatggaatcaaaccagaaatcaagaaattaaatacaacgggcaaatctccaaatgcttggaaattaaatactatacctctatataatccatgggtcaaagagtaaatctcaaaggatattcataatacatagaatgaaaaaaaatgaaatacagcatatcaagatatatgtgatgtggctaaagcaatactgacaggacattttttaaaatcaaatgaatacattagaaaaaaaggagaattatcaaatcaaaaatctaagttactgcttcaagaaactagaagaagtagagcaaaacaaattcaatacaagcagaacaaagaaaatatttaaatataagagccaaaattaatgaagttgaaatcaagaaaacaatagaaaaaattaatgaaacataaaactggttatttggaaaatcaatacaattgatctttaattgacagaaataagagagaagacacaaatctcaggaatgaaatttggggctgtcactacagaatctgcagtcattagaaggataataagaaaaactatgaacaattttatgctcacaaatctgataacttagaggaaatggaccaattcttcagaaaccacaaactaccaaaacataatgaagatgaaatagacaatccaaacactcatataatcactaaatacattgaattcatcatacacaagctcccaaaaagggaccttcaagcctgctggttacactgaatcattctatcaaatatttacagaagcattaacaacaattgtacagtctcttctagaaaatagaagaggaaggaacattttccagttgattttaggaggccagtattactgtgatgctaaaaccaaataaagatattactaaaaaagacaactacaaaaatatctcccattaacttagatattaaaatgctctataaaatattaacaaatcaaattcagcaatgtataaaaagaattatacaccatgaccgagtgagatttatttcaggtacacaagtcttgctcaattatttgaaagtcaagcaatgtaatctaccctatcaacaggctaaagaagaaaatcatatgtagaaaaggtatctgataaaatgcaacacctctcatggcctgtgtccctccctatgcctcctggatttatatgttgaagtactaattgccagtatatcaaaatgtgactgtattttgagatagggtcagttaaaatgaggtcatgagggcaggtcctactgcaatatgactggtatctttagaaaaacagataagggtatagatacacagagatagaaaaccatgtgaagacactgggagaaaatggccacctataagccaaggagggaggactccaaagaaaccaacactgctggtaccctgatttcagacctctagcctcctgaattgtgagaaaataaagttctattatttaagtcaccaagtctgtggtactttttatagcagccctagcagactgatataaacacctattgaagataaaatttcttagaaagtaggtgtggagaactgcctcaacttggtacaaaacatgttcaaaaagtctagagctaccatacttaatggtgagtacaagactgaatgtttttctgctaggactggaaactaagcagggatatcttctctcaccacttttactcaacataattgtctgagttttctgttgcttataacagagtacttgaaatgcataatttataaagaaaggaatttatgtcttatggttatgagactgaaaagttcaaggttgaggagccacatctggtgagggccttcctgctggtgggggctctctggagtccagaggtgacaaaaagcatcacatggcgaggggctgaatgtactcatgtgctagctcaggtctctcttcctcttcttataaaactaccggttccctttccgtaataacacattgatactctaatccattcatgagggcagagccattgtgatccaattacctcttaaagccccacctctcaatactgccacattggggattaagtttcaatctgaattttggaggggacattcaaaccacagcaataatattggaagtttagccaaattatgaagcaggagagaaaaaatgtatgagaaaaaaataaataaaaggcacacagattggaaaggaaaattatacatcaccccaagttttttggagagaaagtagtaaaaatgggtatattttgtttagaaagtaatgcaaattaaaaccacaatgacatattattacatgcagctataagcgtatacagaataaaaaatagtgatgatgctaagtaaatataaatgaacaagggtgataatgctggcaaggatgtggagaaactggatcattcatacattgctggtgatgatgtaaaatgatacaggcactgtataaaatagtttggcagcatcctatgaaacaaaacatgcatttgccttacaatccaacaattgtgctcttagacattcatccttgataaatgaaaactacgtgcacacaaaaatatgtacatgaatgttcacagagattttttcatgactgccaaaaactggaaacagatgtgcttcagttggtaaaatctgtgatatatcatgtcagggaatactacagagcaataaaaaggaacaaactagtgatacccacaagttaggtggatctccagggtattatgatgagtgaaaaaagccagtcttcataaagaaactgtggtacatttataaaatagaatattatccagtgataaaaagaaataagctatcaagccatgaaaagaaatggagaaaccttcaaagtcatattgttgagtgaaagatggggacgccaatctgaaaaggcttcacactgtatgatcccaactatgacattttgaaaaaggcaaagctatggagacaataaaaagatcagtggttgctaggggtttggagggaaggagaaatggctaggcagagcacaggctacttttaaagcagtgaaaatattaatatatgatgatgggtggcattatacatttgtcaaaacctgtaaaacatacaacacaaataatgagccctaatattctgtacacttaagatcataatagtgattcaccaactataacaaacacaccacactaatgcaagatattaacaatagggaaactggggtggagaacaaaggggtgtataagtctctgtactttctgctagtttttctgtaaacctaaatggctcataaaaatgtctatcaattcttttaaaagctaatttcagaaggttacatatgatatgagtccgtttacataatattcttgaaatgacaaaattataggcatggagagcagagaagtggttgccagaatttagagagattgggtgatgatgaggtatgtatggggagaaaggtgactttggttatgaaagaatagcagcagaatccttgtggtgatgaaactattttgtgtcttaactgtggtggtggtgatgtgaatctatgcaaatgataaaattgcataaaacttcacacatatgcaagcacacacactgtggcacataaaacttgtgaaatctgaacaaggtgagtgcattgtattaatgtcaattttctgattgtaatactgtactgtggttatggaagacattaccactgggagaagcttgttgaagggtacacaggatctctctgtatttactcttacagtttcatatgaattttaaattatctcaaaataaaaaaagaattaaaaacaatttaatgttttcagtaggttgttctcataatatttaaagcagggcatagtttttaatattgtctaattttgtttcctttcagtctatgcatattgatgctactttatatacggaaagtgatgttcacgtgagtatactttttttcaaaattgctatttgtcttgttattaaagtttttaaaagtatatttttgtaacaactaaaatatggtagttttaaaatctaacttttggtgtgtttctataatatatggtcaatgagttttattagtgattattgagatgggacttggggacagatagctacatcaatgctatttttgtaatggtgccaaatcaaaatattaaaagagtcttttccatcaatgtcaaacaagatacattatacgtttttgtgatgcagttgcatcatggtgcaattagatgcaaaaatcagattaactgtttcaatttaagagtagccaatatatcagttatttccaatgctgtaatgtatcaccatctgcaatcatattttccataaattgcttaaaatcattcatgttctgcattttacatgattctctacatttgtcagaaaaagattctaattattctctatgtgagaaaggaaacataacccatgaaacttattagaactgaaaaatggtcttgaacttgtttcattaataccctgacatttcctaaggaatgtaggatttgtcatgttggtggtggttcttcccttcagtattgttactaaaaccaagaaatgaatccccaacttcacgtcagggaaaacaaactctatgtcctctctccattattcctcaattagaacttctcttacatagatgactttctaagcattggaaagaaaccatagtcagaaactagaaaaaagagatattcactaaccaattgggtcatgctgctgcacaaagaaattgtctccctcctctggcttcagcatggattctgtttttctttgcattttttgtaaaatggaaagtagtcaattagccaccaaattcagcatcctgaaattgcttttttgcaagaagataactttgcaacccatgggttagagaaacaaccttagattttagattttagattttagaagtttagattttaaaactgactccaccactttttacttgtgttaccttggagattgctaaacatttctgaagctcatttttattattgctgatatggtgacagtatttgtctttcaatcctaagcagtgactgttgggtttaaggaatattaccagtgctgggcacatgggtgtattccttcattttttttatccattcagtaagcatttgtgaagtgtctgcaggctcccaggtactgtgcaaggtatcgaggacacaaagtcaaataatggtcaaatccctagatattccttgtttcctttctttaagttattgttaaagtaaatgttgtggaaaacatatttttgtggcacctaagagagccttttctgatttgatctgcctgtaagtggacactagtcctgctttgatttttctaataaatttcttggaattacttctctccctctggaactttggaacttgaaagaacagtataattagcagctaccactatttaaaatgttaacaacacaatttgattctaaaaattcttacaaataaactgtctatattgatgttttcagactctaaggccagtattattaccctttccacaggggaaatgtgctcggtgagaaaaactggccactatttctgcattttctagtttcatgttcttctgtacaatattgccatccttcttgattgtgttttgttggtttgttttggtttttattttaaagccaaaagaaaaaaaattaagctgcctcctatgtaaaatttcttaaggatattgtgtatctttataaaatatttggattgactttttgaaaatcaaaattacatcagtatgaaaattaataatcagaaaaaagtaattgttctttataacttttatttttagcccagttgcaaagtaacagcaatgaagtgctttctcttggagttacaagttatttcacttgagtccggagatgcaagtattcatgatacagtagaaaatctgatcatcctagcaaacaacagtttgtcttctaatggggtgagttttccaacagttgcttagagttgcatcttatgttttgggcctgatttggagaagtcattcatggacaagcagatcctcctaaggggcaatcaggagaaggagtcctgttccagtggagtggtgtgcaaaagtggcaccatctccagcatgcacacaatgctaaactgttcaatgattgattcagtagacaaacatttacagaataatgccaggtagtgtgcttcagtgctagaattatagttgcaaaccagacagacaggtcactgacctcaaggtgtttattgtcaggtgcagtggatagacattaatctaattacatactcaaatgtctaacatcaataatagtaactcacatttattgagttattactgtatgccagacactattctttgagctttaaattgataattcatttaatcctcatgataaatctgtgagatagttatctttatttctgtttgaatgttgaagaaattgtagcactgagagcctggatcactcgcttcagttcatacagctagtaaaatggggagccacaactcttacccgagcagtctgtctgcagagtctgttatctcatgagaagataaatgctttaaaaagataacaatgaaaatattaatgattcgagtataatctgggacatcccaggcaacttgagcagtagagggttccaggagagcaaattctaggtactcgtatgataggctggggaatcagctgtgcaatggccctgaagtgcaaagaattagggctttcatggcaaaacaccagtggccaaagcaaggtagagagaaagcagttttgcaacaaagaggcataatcaaattagttcatttcataaatactttttagccctcaggagacagtatgaaaaaatacccataatccctgctgttaaagagggaggaaacagaaataaaccaagcactcagcaaatgagtaataagttagttatttatgaagtccagtggtgaaaaaggacacagggtaggggcactggggctccagttagggtaggctgagctaataagctgccatttgacaaagatgtgagagaatgagccttaagggtatctagggacatatgtttaggcagagacaactgaaagtgcaaaggccctaagcttgtgtgggtggacaccttgacttttctctaaaaatacaagactgctttaggtctttgcttcagtgtcaccttctcagtgatttacctatttacagttgcaatcccttccctatctgagcactccctgtccccttctctgcgtgtcccatatctggaatatgatatattttatgtaatgggtattgtctgtttttccctactagaatgtcagcttaaaatggacaagtgtttttgttttttattagtactatatccaaattcccagaacagtgtctcatatatattaaatactttacatatgtatgaaatgaatttattaaggtttataatactcttatgatgttatctccatcttattggtggaaagagtgagccacgcaacaggtaactcatggcaaaataaatgatgagctctttaatgtgaatgttgatagtcatcctcatcatgacagagtgtgacatacgtgctggaatgcgtgggcaggactgcacagtcagaagacctggatgggaagaatttcagttgattatgtcagttgagatgagtcaatatctctgtcttcctatctgacatgattattttcaggatcaaataggacagcattatatgaaatatgacagtgcttggcatagaggatgtgcttgataaatgtcgctgattctgaattccaaatagttaatggatataaaatctaatttttatttttgctggcacagccttcatggttcagatgatctctaaaaagtttattcttaattgctacatttggaacattctagttatccatgagactgtgtttgaagtacaacaatttgtggtattcacctaaatggctcagttcaaccaatgggtgattacagtgtgtcaggccctgtgctaaatgctagggaaacaaacatactggctctgaccttctggcagagagcttagagaatagtattacagtataaaaagagtaaaacttcataatatgaatagaatacatgacatattgtgagagaggtctaaataaggggttctgagatgtaaagatgggacattgagtccagccctaggagcccaggggttcagagaagcctccctaaaagagtacctgaataatcttaagtgcgagtagcagcagctaaaataggaatgaatgtaacagagagaaaagcaaaggcagagaggcatctaatagtatgctgtgcatggcaaggaaagccagatgctcactcagtgtttctagggcataacattgaggcaagggggatatgtgaaataaataggtaaggaacagctcatgaagagcctgtgtgctatgcagggaggcatggtctttatgctagaagcagatgataggaaaccattgaagggctcaaagcaggaactccgtggtctgatagacacagcttatttttctcaatgtccttaacccattattggaaaaataaacatatatcaaatctccctgttctctttcctcgtagttcttcctaatatgctatttatttgctaaagctttcatatctcaagacctcaccatatggttcaaacgatatgatattcccattcccatgaatgtatatttaatttaattataaattgccaatttaatctctctctatattttgcagaatgtaacagaatctggatgcaaagaatgtgaggaactggaggaaaaaaatattaaagaatttttgcagagttttgtacatattgtccaaatgttcatcaacacttcttga

SEQ ID NO: 56 - DNA, 87 bp, synthetic construct
  source 1..87: mol_type = other DNA
gtgatagtccttcacggaaagtacaagaatacacagaagtcgacggtatcgataagcttgatatcgaattccgaagttcctattctc

SEQ ID NO: 57 - DNA, 76 bp, synthetic construct
  source 1..76: mol_type = other DNA
ctctagaaagtataggaacttcatcagtcaggtacataatggtggatccaactgctgtttacattagtctttcacg

SEQ ID NO: 58 - RNA, 1287 nt, synthetic construct
  source 1..1287: mol_type = other RNA
ttcttgaccaagacttcaatactcagtggcactgtattccccttctgtccagccactcttccccagagttctcttcttcatcctcccccttgcagagtagggcagcttgcaggtcctcctgcaagtctctcccaattctctgcgcccaaaagacttgcagtgcatctccttacgcgctgcagggaccttgccagggcaggactgcccccgcccagttgcagagttggacgaagacgggatcctgctgtgtttggaaggctgagttccacatctaacagctcagagagaatccaccttgacacatggccctctggctcttcaaagcactgcctcttcatggtccttgctggtgaggtccttaagaacacagaaacccatgtcagcagataaccagcctacaggaggccaagaagagttctggatggatggcagctggaagcccatcgccatagccagctcatcttcaacattgaagctcttacctgggcattaagtaatgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgactgcatgcgagcctcttccgtgtttctgttattaaggtacctccacctgctgctcagaggcagcacagctccatgcatttgaaatctgctgggcaaactaagcttcctaacaaggagataatgagccacttggatcacatgaaatcttggaaatgaagagaggaaaagagctcgtctcagacttatttttgcttgcttatttttaatttattgcttcatttgtacatatttgtaatataacagaagatgtggaataaagttgtatggatattttatcaattgaaatttaaaaaaaaaaaaaa

SEQ ID NO: 59 - DNA, 25 bp, synthetic construct
  source 1..25: mol_type = other DNA
gggcttggtatcaagaatgaggggt

SEQ ID NO: 60 - DNA, 23 bp, synthetic construct
  source 1..23: mol_type = other DNA
accccacagaacctctactggga

SEQ ID NO: 61 - DNA, 21 bp, synthetic construct
  source 1..21: mol_type = other DNA
ccccaagtcatgtttgcaccc

SEQ ID NO: 62 - DNA, 23 bp, synthetic construct
  source 1..23: mol_type = other DNA
gtcctccctgaatcctgcacctg

SEQ ID NO: 63 - DNA, 21 bp, synthetic construct
  source 1..21: mol_type = other DNA
ggatcggccattgaacaagat

SEQ ID NO: 64 - DNA, 22 bp, synthetic construct
  source 1..22: mol_type = other DNA
cagaagaactcgtcaagaaggc

SEQ ID NO: 65 - DNA, 20 bp, synthetic construct
  source 1..20: mol_type = other DNA
agtccgggcagtcatgggat

SEQ ID NO: 66 - DNA, 23 bp, synthetic construct
  source 1..23: mol_type = other DNA
tgtctgtcgcagccagagatgtt

SEQ ID NO: 67 - DNA, 25 bp, synthetic construct
  source 1..25: mol_type = other DNA
cctgccctctaagactacacaattt

SEQ ID NO: 68 - DNA, 23 bp, synthetic construct
  source 1..23: mol_type = other DNA
gattacaggcacttgccacatcc

SEQ ID NO: 69 - DNA, 23 bp, synthetic construct
  source 1..23: mol_type = other DNA
ggatccccatggttgtgcaggaa

SEQ ID NO: 70 - DNA, 25 bp, synthetic construct
  source 1..25: mol_type = other DNA
gacaagcgttagtaggcacatatac

SEQ ID NO: 71 - DNA, 24 bp, synthetic construct
  source 1..24: mol_type = other DNA
gctccaatttcccacaacattagt

SEQ ID NO: 72 - DNA, 20 bp, synthetic construct
  source 1..20: mol_type = other DNA
gctgacatccgggtcaagaa

SEQ ID NO: 73 - DNA, 20 bp, synthetic construct
  source 1..20: mol_type = other DNA
tgcctgtccctgtagttcct

SEQ ID NO: 74 - DNA, 21 bp, synthetic construct
  source 1..21: mol_type = other DNA
tactccagggagcggtacatt

SEQ ID NO: 75 - DNA, 21 bp, synthetic construct
  source 1..21: mol_type = other DNA
gagttcccagttcttggctgt

SEQ ID NO: 76 - DNA, 22 bp, synthetic construct
  source 1..22: mol_type = other DNA
tcaccatcttccaggagcgaga

SEQ ID NO: 77 - DNA, 21 bp, synthetic construct
  source 1..21: mol_type = other DNA
gaaggccatgccagtgagctt

SEQ ID NO: 78 - DNA, 20 bp, synthetic construct
  source 1..20: mol_type = other DNA
gggctgtgtcagtgtaggtc

SEQ ID NO: 79 - DNA, 23 bp, synthetic construct
  source 1..23: mol_type = other DNA
actctgtgaaggttttctcctcc

SEQ ID NO: 80 - DNA, 21 bp, synthetic construct
  source 1..21: mol_type = other DNA
ccatccagtgctacttgtgtt

SEQ ID NO: 81 - DNA, 20 bp, synthetic construct
  source 1..20: mol_type = other DNA
ggggtgaacatcactttccg

SEQ ID NO: 82 - protein, 448 aa, synthetic construct
EVQLVQSGAEVKKPGESLKISCKVSGYFFTTYWIGWVRQMPGKGLEYMGIIYPGDSDTRYSPSFQGQVTISADKSISTAYLQWSSLKASDTAMYYCARGGNWNCFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 214 aa, synthetic construct
EIVLTQSPGTLSLSPGERATLSCRASQSVSSSYLAWYQQKPGQAPRLLIYGASRRATGIPDRFSGSGSGTDFTLTISRLEPEDFAVYYCQRYGSSHTFGQGTKLEISRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 84 - RNA, 1528 nt, synthetic construct
  source 1..1528: mol_type = other RNA
aggaaatgtatgggtggggagggcttgtgggagagtggttcagggttctgacacagactacacccagagaaagaagagcaagcaccatgttgaagccatcattaccattcacatccctcttattcctgcagctgcccctgctgggagtggggctgaacacgacaattctgacgcccaatgggaatgaagacaccacagctgatttcttcctgaccactatgcccactgactccctcagtgtttccactctgcccctcccagaggttcagtgttttgtgttcaatgtcgagtacatgaattgcacttggaacagcagctctgagccccagcctaccaacctcactctgcattattggtacaagaactcggataatgataaagtccagaagtgcagccactatctattctctgaagaaatcacttctggctgtcagttgcaaaaaaaggagatccacctctaccaaacatttgttgttcagctccaggacccacgggaacccaggagacaggccacacagatgctaaaactgcagaatctggtgatcccctgggctccagagaacctaacacttcacaaactgagtgaatcccagctagaactgaactggaacaacagattcttgaaccactgtttggagcacttggtgcagtaccggactgactgggaccacagctggactgaacaatcagtggattatagacataagttctccttgcctagtgtggatgggcagaaacgctacacgtttcgtgttcggagccgctttaacccactctgtggaagtgctcagcattggagtgaatggagccacccaatccactgggggagcaatacttcaaaagagaatcctttcctgtttgcattggaagccgtggttatctctgttggctccatgggattgattatcagccttctctgtgtgtatttctggctggaacggacgatgccccgaattcccaccctgaagaacctagaggatcttgttactgaataccacgggaacttttcggcctggagtggtgtgtctaagggactggctgagagtctgcagccagactacagtgaacgactctgcctcgtcagtgagattcccccaaaaggaggggcccttggggaggggcctggggcctccccatgcaaccagcatagcccctactgggcccccccatgttacaccctaaagcctgaaacctgaaccccaatcctctgacagaagaaccccagggtcctgtagccctaagtggtactaactttccttcattcaacccacctgcgtctcatactcacctcaccccactgtggctgatttggaattttgtgcccccatgtaagcaccccttcatttggcattccccacttgagaattacccttttgccccgaacatgtttttcttctccctcagtctggcccttccttttcgcaggattcttcctccctccctctttccctcccttcctctttccatctaccctccgattgttcctgaaccgatgagaaataaagtttctgttgataatcatcaaaaa

SEQ ID NO: 85 - protein, 368 aa, Rattus norvegicus
MLKPLLPSRSFLLLQLLLLRVGWSSKVLMSSGNEDTKSDLLLTSMDLKHLSVPTLPLPEVQCFVFNVEYMNCTWNSSSEPQPTNLTMHYRYKGSDNNTFQECSHYLFSKEITSGCQIQKEDIQLYQTFVVQLQDPQKPQRRAEQKLNLQNLVIPWAPENLTLYNLSESQVELRWKSRYIERCLQYLVQYRSNRDRSWTEQIVDHEPRFSLPSVDEQKLYTFRVRSRFNPICGSTQQWSKWSQPIHWGSHTAEENPSLFALEAVLIPVGTMGLIITLIFVYCWLERMPRIPAIKNLEDLVTEYHGNFSAWSGVSKGLTESLQPDYSERFCHVSEIPPKGGALGEGPGGSPCSLHSPYWPPPCYSLKPEA

SEQ ID NO: 86 - protein, 538 aa, Rattus norvegicus
MATVDLSWRLPLYILLLLLATTWVSAAVNDCSHLKCFYNSRANVSCMWSPEEALNVTSCHIHAKSDMRHWNKTCELTPVRQASWACNLILGPLPDSQSLTSVDLLSLSVVCWEEKGWRRVKTCNFHPFDNLRLIAPHSLQVLHIETRRCNISWEVSQVSHYVNPYLEFEARRRLLDRSWEDASVFSLKQRQQWIFLETLTPDTSYELQVRVIAQRGKTRTWSPWSQPVAFRTRPADPKEIFPLPWLRCLLLVLGCFFGFLSCVCVLVKCRYLGPWLKTLLKCHIPDPSEFFSQLSSQHGGDLQKWLSSPVPQSFFSPTGSAPEISPLEVLDRDSKTMQMLLFQKEKASSPSPSGHSQASCFTNQGYFFFHLSNALEIESCQVYFTYDPCMEEDVEEDGPRLPEESPLPPLLPFTGEQDDYCAFPPRDDLLLFSPSLSTPNTAYGNSITPEERPPLSLQEGLPSLTSPDLMGLQHPLELVLGDDGEGMSTNSSGQQASVPEGSSHGHDQDRGQGPVLTLNTDAYLSLQELQAQDSAHLI

SEQ ID NO: 87 - protein, 277 aa, Rattus norvegicus
MASPQLRGCGVHALPMLLPLLLLLLLLLPLRVTPGITCPTPISIEHADIRVKNYSVNSRERYVCNSGFKRKAGTSTLTECVINKNTNVAHWTTPNLKCIRDPSLAHYSPVPTVVTPKVTSQPESLSPPEKEPEALSPKSDTTVATETAIVPGSRLTPSQAASAGTTGTGRHKSSPAPSLATTMTLEPTASTSLRITEISPHSSEMTKVAIPTSVLLVAAGIGLVFLTRYIKSRQASQPRRVEVETMETVPMTVRARSKEDEDHTTSETQGDQPTRGV

SEQ ID NO: 88 - protein, 162 aa, Rattus norvegicus
MKILKPYMRNTSILYYLCFLLNSHFLTEAGIHVFILGCVSVGLPKTEANWIDVRYDLEKIESLIQFIHIDTTLYTDSDFHPSCKVTAMNCFLLELQVILHEYSNMTLNETVRNVLYLANSTLSSNKNVIESGCKECEELEERNFTEFLQSFIHIVQMFINTS

(19) United States
(12) Patent Application Publication
(10) Pub. No.: US 2025/0302016 A1
(43) Pub. Date: Oct. 2, 2025

(54) GENETICALLY MODIFIED NON-HUMAN ANIMAL WITH HUMAN OR CHIMERIC GENES

(71) Applicant: Biocytogen Pharmaceuticals (Beijing) Co., Ltd., Beijing (CN)

(72) Inventors: Chengzhang Shang, Beijing (CN); Linlin Wang, Beijing (CN); Suman Zhao, Beijing (CN); Jiangfeng Yuan, Beijing (CN)

(21) Appl. No.: 18/865,460

(22) PCT Filed: May 23, 2023
(86) PCT No.: PCT/CN2023/095823

§ 371 Date: Nov. 13, 2024

(30) Foreign Application Priority Data
May 23, 2022 (CN) 202210565505.7
Sep. 30, 2022 (CN) 202211214520.3
Nov. 2, 2022 (CN) 202211364219.0

(51) Int. Cl. A01K 67/0278; A61K 49/00; C07K 14/54; C07K 14/715

(52) U.S. Cl. CPC A01K 67/0278; A61K 49/0008; C07K 14/5443; C07K 14/7155; A01K 2207/15; A01K 2217/072; A01K 2227/105

(57) ABSTRACT

The present disclosure relates to genetically modified non-human animals that express a human or chimeric (e.g., humanized) IL2RG, IL2RB, IL15RA, and/or IL15, and methods of use thereof.

### CLAIM OF PRIORITY

This application claims the benefit of Chinese Patent Application App. No. 202210565505.7, filed on May 23, 2022; Chinese Patent Application App. No. 202211214520.3, filed on Sep. 30, 2022; and Chinese Patent Application App. No. 202211364219.0, filed on Nov. 2, 2022. The entire contents of the foregoing applications are incorporated herein by reference.

### TECHNICAL FIELD

This disclosure relates to genetically modified animal expressing human or chimeric (e.g., humanized) IL2RG, IL2RB, IL15RA, and/or IL15, and methods of use thereof.

### BACKGROUND

The traditional drug research and development typically use in vitro screening approaches. However, these screening approaches cannot provide the body environment (such as tumor microenvironment, stromal cells, extracellular matrix components and immune cell interaction, etc.), resulting in a higher rate of failure in drug development. In addition, in view of the differences between humans and animals, the test results obtained from the use of conventional experimental animals for in vivo pharmacological test may not reflect the real disease state and the interaction at the targeting sites, resulting in that the results in many clinical trials are significantly different from the animal experimental results.

Therefore, the development of humanized animal models that are suitable for human antibody screening and evaluation will significantly improve the efficiency of new drug development and reduce the cost for drug research and development.

### SUMMARY

This disclosure is related to an animal model with human or chimeric IL2RG, IL2RB, IL15RA, and/or IL15. The animal model can express human or chimeric (e.g., humanized) IL2RG, IL2RB, IL15RA, and/or IL15 proteins in its body. It can be used in the studies on the function of IL2RG, IL2RB, IL15RA, and/or IL15 genes, and can be used in the screening and evaluation of antibodies or drugs targeting IL2RG, IL2RB, IL15RA, and/or IL15. In addition, the animal models prepared by the methods described herein can be used in drug screening, pharmacodynamics studies, treatments for immune-related diseases, and cancer therapy that targets human IL2 or IL15 signaling pathways; they can also be used to facilitate the development and design of new drugs, and save time and cost. In summary, this disclosure provides a powerful tool for studying the function of IL2RG, IL2RB, IL15RA, and/or IL15 proteins and a platform for screening drugs targeting immune disorders (e.g., psoriasis).

In one aspect, the disclosure is related to a genetically-modified, non-human animal whose genome comprises at least one chromosome comprising a sequence encoding a human or chimeric Interleukin 2 Receptor Subunit Gamma (IL2RG). In some embodiments, the sequence encoding the human or chimeric IL2RG is operably linked to an endogenous regulatory element at the endogenous IL2RG gene locus in the at least one chromosome. In some embodiments, the sequence encoding a human or chimeric IL2RG comprises a sequence encoding an amino acid sequence that is at least 70%, 75%, 80%, 85%, 90%, 95%, 99%, or 100% identical to human IL2RG (NP_000197.1 (SEQ ID NO: 2)). In some embodiments, the sequence encoding a human or chimeric IL2RG comprises a sequence encoding an amino acid sequence that is at least 70%, 75%, 80%, 85%, 90%, 95%, 99%, or 100% identical to SEQ ID NO: 30. In some embodiments, the sequence encoding a human or chimeric IL2RG comprises a sequence encoding an amino acid sequence that is at least 70%, 75%, 80%, 85%, 90%, 95%, 99%, or 100% identical to amino acids 1-256 of SEQ ID NO: 2. In some embodiments, the animal is a mammal, e.g., a monkey, a rodent, a mouse, or a rat. In some embodiments, the animal does not express endogenous IL2RG or expresses a decreased level of endogenous IL2RG as compared to IL2RG expression level in a wild-type animal. In some embodiments, the animal has one or more cells expressing human or chimeric IL2RG. In some embodiments, the animal has one or more cells expressing human or chimeric IL2RG, and the expressed human or chimeric IL2RG can interact with human or endogenous Interleukin-2 Receptor Subunit Alpha (IL2RA) and Interleukin 2 Receptor Subunit Beta (IL2RB), forming a functional IL2 receptor complex. In some embodiments, the animal has one or more cells expressing human or chimeric IL2RG, and the expressed human or chimeric IL2RG can interact with human or endogenous Interleukin-15 Receptor Subunit Alpha (IL15RA) and Interleukin 2 Receptor Subunit Beta (IL2RB), forming a functional IL15 receptor complex.

In one aspect, the disclosure is related to a genetically-modified, non-human animal, in some embodiments, the genome of the animal comprises a replacement of a sequence encoding a region of endogenous IL2RG with a sequence encoding a corresponding region of human IL2RG at an endogenous IL2RG gene locus. In some embodiments, the sequence encoding the corresponding region of human IL2RG is operably linked to an endogenous regulatory element at the endogenous IL2RG locus, and one or more cells of the animal expresses a human or chimeric IL2RG. In some embodiments, the animal does not express endogenous IL2RG or expresses a decreased level of endogenous IL2RG as compared to IL2RG expression level in a wild-type animal. In some embodiments, the replaced sequence encodes the full-length IL2RG; or the signal peptide and all or a portion of the extracellular region of IL2RG. In some embodiments, the animal has one or more cells expressing human IL2RG. In some embodiments, the animal has one or more cells expressing a chimeric IL2RG having a signal peptide, an extracellular region, a transmembrane region, and a cytoplasmic region, in some embodiments, the extracellular region comprises a sequence that is at least 50%, 60%, 70%, 80%, 90%, 95%, or 99% identical to the extracellular region of human IL2RG (NP_000197.1 (SEQ ID NO: 2)). In some embodiments, the extracellular region of the chimeric IL2RG has a sequence that has at least 10, 20, 30, 40, 50, 60, 70, 80, 90, 100, 110, 120, 130, 140, 150, 160, 170, 180, 190, 200, 210, 220, 230, 231, 232, 233, 234, 235, 236, 237, 238, 239, or 240 contiguous amino acids that are identical to a contiguous sequence present in the extracellular region of human IL2RG (e.g., amino acids 23-256 or 23-262 of SEQ ID NO: 2). In some embodiments, the signal peptide of the chimeric IL2RG has a sequence that has at least 1, 2, 3, 4, 5, 6, 7, 8, 9, 10, 11, 12, 13, 14, 15, 16, 17, 18, 19, 20, 21, or 22 contiguous amino acids that are identical to a contiguous sequence present in the signal peptide of human IL2RG (e.g., amino acids 1-22 of SEQ ID NO: 2). In some embodiments, the sequence encoding a region of endogenous IL2RG (e.g., mouse IL2RG) comprises exon 1, exon 2, exon 3, exon 4, exon 5, exon 6, exon 7, and/or exon 8 (e.g., exon 1, exon 2, exon 3, exon 4, exon 5, and exon 6), or a part thereof, of the endogenous IL2RG gene. In some embodiments, the animal is heterozygous or homozygous with respect to the replacement at the endogenous IL2RG gene locus.

In one aspect, the disclosure is related to a non-human animal comprising at least one cell comprising a nucleotide sequence encoding a human or humanized IL2RG polypeptide, in some embodiments, the human or humanized IL2RG polypeptide comprises at least 50 contiguous amino acid residues that are identical to the corresponding contiguous amino acid sequence of a human IL2RG, in some embodiments, the animal expresses the human or humanized IL2RG polypeptide. In some embodiments, the human or humanized IL2RG polypeptide has at least 50, 60, 70, 80, 90, 100, 110, 120, 130, 140, 150, 160, 170, 180, 190, 200, 210, 220, 230, 231, 232, 233, 234, 235, 236, 237, 238, 239, or 240 contiguous amino acid residues that are identical to the corresponding contiguous amino acid sequence of human IL2RG extracellular region (e.g., amino acids 23-256 or 23-262 of SEQ ID NO: 2). In some embodiments, the human or humanized IL2RG polypeptide has at least 1, 2, 3, 4, 5, 6, 7, 8, 9, 10, 11, 12, 13, 14, 15, 16, 17, 18, 19, 20, 21, or 22 contiguous amino acid residues that are identical to the corresponding contiguous amino acid sequence of human IL2RG signal peptide (e.g., amino acids 1-22 of SEQ ID NO: 2). In some embodiments, the human or humanized IL2RG polypeptide comprises a sequence that is at least 90%, 95%, or 99% identical to amino acids 1-256 or 1-262 of SEQ ID NO: 2. In some embodiments, the nucleotide sequence is operably linked to an endogenous IL2RG regulatory element of the animal. In some embodiments, the nucleotide sequence is integrated to an endogenous IL2RG gene locus of the animal. In some embodiments, the nucleotide sequence encodes a humanized IL2RG polypeptide, in some embodiments, the humanized IL2RG polypeptide comprises an endogenous IL2RG transmembrane region and/or an endogenous IL2RG cytoplasmic region. In some embodiments, the humanized IL2RG polypeptide has at least one mouse IL2RG activity and/or at least one human IL2RG activity.

In one aspect, the disclosure is related to a method for making a genetically-modified, non-human animal, comprising: replacing in at least one cell of the animal, at an endogenous IL2RG gene locus, a sequence encoding a region of endogenous IL2RG with a sequence encoding a corresponding region of human IL2RG. In some embodiments, the sequence encoding the corresponding region of human IL2RG comprises exon 1, exon 2, exon 3, exon 4, exon 5, exon 6, exon 7, and/or exon 8, or a part thereof, of a human IL2RG gene. In some embodiments, the sequence encoding the corresponding region of human IL2RG comprises a portion of exon 1, exon 2, exon 3, exon 4, exon 5, exon 6, exon 7, and exon 8, optionally at least 1, 2, 3, 4, 5, 6, 7, 8, 9, 10, 20, 30, 40, 50, 60, 61, 62, 63, 64, 65, 70, 80, 90, or 100 nucleotides downstream of exon 8, of a human IL2RG gene. In some embodiments, the sequence encoding the corresponding region of human IL2RG comprises a portion of exon 1, exon 2, exon 3, exon 4, exon 5, and a portion of exon 6, of a human IL2RG gene. In some embodiments, the sequence encoding the corresponding region of human IL2RG encodes amino acids 1-256, 1-262, or 1-369 of SEQ ID NO: 2. In some embodiments, the region comprises the full-length IL2RG; or the signal peptide and all or a portion of the extracellular region of IL2RG. In some embodiments, the sequence encoding a region of endogenous IL2RG comprises exon 1, exon 2, exon 3, exon 4, exon 5, exon 6, exon 7, and/or exon 8, or a part thereof, of the endogenous IL2RG gene. In some embodiments, the animal is a mouse, and the sequence encoding a region of endogenous IL2RG comprises a portion of exon 1, exon 2, exon 3, exon 4, exon 5, exon 6, exon 7, and a portion of exon 8 of the endogenous IL2RG gene. In some embodiments, the animal is a mouse, and the sequence encoding a region of endogenous IL2RG comprises a portion of exon 1, exon 2, exon 3, exon 4, exon 5, and a portion of exon 6 of the endogenous IL2RG gene.

In one aspect, the disclosure is related to a method of making a genetically-modified animal cell that expresses a chimeric IL2RG, the method comprising: replacing at an endogenous IL2RG gene locus, a nucleotide sequence encoding a region of endogenous IL2RG with a nucleotide sequence encoding a corresponding region of human IL2RG, thereby generating a genetically-modified animal cell that includes a nucleotide sequence that encodes the chimeric IL2RG, in some embodiments, the animal cell expresses the chimeric IL2RG. In some embodiments, the chimeric IL2RG comprises a human or humanized IL2RG extracellular region; and a transmembrane and/or a cytoplasmic region of mouse IL2RG. In some embodiments, the chimeric IL2RG further comprises a human or humanized IL2RG signal peptide.

In one aspect, the disclosure is related to a method of making a genetically-modified animal cell that expresses a human IL2RG, the method comprising: replacing at an endogenous IL2RG gene locus, a nucleotide sequence encoding a region of endogenous IL2RG with a nucleotide sequence encoding a corresponding region of human IL2RG, thereby generating a genetically-modified animal cell that includes a nucleotide sequence that encodes the human IL2RG, in some embodiments, the animal cell expresses the human IL2RG.

In some embodiments, the animal is a mouse. In some embodiments, the nucleotide sequence encoding the chimeric IL2RG is operably linked to an endogenous IL2RG regulatory region, e.g., promoter.

In some embodiments, the animal described herein further comprises a sequence encoding an additional human or chimeric protein, e.g., Interleukin 2 Receptor Subunit Beta (IL2RB), Interleukin 15 (IL15), Interleukin-15 Receptor Subunit Alpha (IL15RA), Interleukin 2 (IL2), Interleukin 2 Receptor Subunit Alpha (IL2RA), programmed cell death protein 1 (PD-1), programmed death-ligand 1 (PD-L1), Interleukin 10 Receptor Subunit Alpha (IL10RA), and/or cytotoxic T-lymphocyte-associated protein 4 (CTLA4).

In one aspect, the disclosure is related to a genetically-modified, non-human animal whose genome comprises at least one chromosome comprising a sequence encoding a human or chimeric Interleukin-15 (IL15). In some embodiments, the sequence encoding the human or chimeric IL15 is operably linked to an endogenous regulatory element at the endogenous IL15 gene locus in the at least one chromosome. In some embodiments, the sequence encoding a human or chimeric IL15 comprises a sequence encoding an amino acid sequence that is at least 70%, 75%, 80%, 85%, 90%, 95%, 99%, or 100% identical to human IL15 (NP_000576.1 (SEQ ID NO: 52)). In some embodiments, the sequence encoding a human or chimeric IL15 comprises a sequence that is at least 70%, 75%, 80%, 85%, 90%, 95%, 99%, or 100% identical to SEQ ID NO: 55. In some embodiments, the animal is a mammal, e.g., a monkey, a rodent, a mouse, or a rat. In some embodiments, the animal does not express endogenous IL15 or expresses a decreased level of endogenous IL15 as compared to IL15 expression level in a wild-type animal. In some embodiments, the animal has one or more cells expressing human or chimeric IL15. In some embodiments, the animal has one or more cells expressing human or chimeric IL15, and the expressed human or chimeric IL15 is functional that can interact with a human, chimeric, or endogenous IL15 receptor complex.

In one aspect, the disclosure is related to a genetically-modified, non-human animal, in some embodiments, the genome of the animal comprises a replacement of a sequence encoding a region of endogenous IL15 with a sequence encoding a corresponding region of human IL15 at an endogenous IL15 gene locus. In some embodiments, the sequence encoding the corresponding region of human IL15 is operably linked to an endogenous regulatory element at the endogenous IL15 locus, and one or more cells of the animal expresses a human or chimeric IL15. In some embodiments, the animal does not express endogenous IL15 or expresses a decreased level of endogenous IL15 as compared to IL15 expression level in a wild-type animal. In some embodiments, the replaced sequence encodes the full-length IL15. In some embodiments, the sequence encoding a region of endogenous IL15 (e.g., mouse IL15) comprises exon 1, exon 2, exon 3, exon 4, exon 5, exon 6, exon 7, and/or exon 8 (e.g., exon 3, exon 4, exon 5, exon 6, exon 7, and exon 8), or a part thereof, of the endogenous IL15 gene. In some embodiments, the animal is heterozygous or homozygous with respect to the replacement at the endogenous IL15 gene locus.

In one aspect, the disclosure is related to a non-human animal comprising at least one cell comprising a nucleotide sequence encoding a human or humanized IL15 polypeptide, in some embodiments, the human or humanized IL15 polypeptide comprises at least 50, 60, 70, 80, 90, 100, 110, 120, 130, 140, 150, 160, 161, or 162 contiguous amino acid residues that are identical to the corresponding contiguous amino acid sequence of a human IL15, in some embodiments, the animal expresses the human or humanized IL15 polypeptide. In some embodiments, the human or humanized IL15 polypeptide comprises a sequence that is at least 90%, 95%, or 99% identical to SEQ ID NO: 52. In some embodiments, the nucleotide sequence is operably linked to an endogenous IL15 regulatory element of the animal. In some embodiments, the nucleotide sequence is integrated to an endogenous IL15 gene locus of the animal.

In one aspect, the disclosure is related to a method for making a genetically-modified, non-human animal, comprising: replacing in at least one cell of the animal, at an endogenous IL15 gene locus, a sequence encoding a region of endogenous IL15 with a sequence encoding a corresponding region of human IL15. In some embodiments, the sequence encoding the corresponding region of human IL15 comprises exon 1, exon 2, exon 3, exon 4, exon 5, exon 6, exon 7, and/or exon 8, or a part thereof, of a human IL15 gene. In some embodiments, the sequence encoding the corresponding region of human IL15 comprises a portion of exon 3, exon 4, exon 5, exon 6, exon 7, and a portion of exon 8, of a human IL15 gene. In some embodiments, the sequence encoding the corresponding region of human IL15 encodes SEQ ID NO: 52. In some embodiments, the sequence encoding a region of endogenous IL15 comprises exon 1, exon 2, exon 3, exon 4, exon 5, exon 6, exon 7, and/or exon 8, or a part thereof, of the endogenous IL15 gene. In some embodiments, the animal is a mouse, and the sequence encoding a region of endogenous IL15 comprises a portion of exon 3, exon 4, exon 5, exon 6, exon 7, and a portion of exon 8, of the endogenous IL15 gene.

In one aspect, the disclosure is related to a method of making a genetically-modified animal cell that expresses a human or humanized IL15, the method comprising: replacing at an endogenous IL15 gene locus, a nucleotide sequence encoding a region of endogenous IL15 with a nucleotide sequence encoding a corresponding region of human IL15, thereby generating a genetically-modified animal cell that includes a nucleotide sequence that encodes the human or humanized IL15, in some embodiments, the animal cell expresses the human or humanized IL15. In some embodiments, the animal is a mouse. In some embodiments, the nucleotide sequence encoding the human or humanized IL15 is operably linked to an endogenous IL15 regulatory region, e.g., promoter.

In some embodiments, the animal described herein further comprises a sequence encoding an additional human or chimeric protein, e.g., Interleukin 2 Receptor Subunit Beta (IL2RB), Interleukin 2 Receptor Subunit Gamma (IL2RG), Interleukin-15 Receptor Subunit Alpha (IL15RA), Interleukin 2 (IL2), Interleukin 2 Receptor Subunit Alpha (IL2RA), programmed cell death protein 1 (PD-1), programmed death-ligand 1 (PD-L1), Interleukin 10 Receptor Subunit Alpha (IL10RA), and/or cytotoxic T-lymphocyte-associated protein 4 (CTLA4).

In one aspect, the disclosure is related to a method of determining effectiveness of a therapeutic agent for treating an allergic disorder (e.g., allergy, asthma, and/or atopic dermatitis), comprising: a) administering the therapeutic agent to the animal described herein, in some embodiments, the animal has the allergic disorder; and b) determining effects of the therapeutic agent in treating the allergic disorder.

In one aspect, the disclosure is related to a method of determining effectiveness of a therapeutic agent for reducing an inflammation (e.g., skin inflammation or infection), comprising: a) administering the therapeutic agent to the animal described herein, in some embodiments, the animal has the inflammation; and b) determining effects of the therapeutic agent for reducing the inflammation.

In one aspect, the disclosure is related to a method of determining effectiveness of a therapeutic agent for treating an immune disorder, comprising: a) administering the agent to the animal described herein, in some embodiments, the animal has the immune disorder; and b) determining effects of the therapeutic agent for treating the immune disorder. In some embodiments, the immune disorder is psoriasis. In some embodiments, the immune disorder is an autoimmune disease, e.g., graft versus host disease (GVHD), psoriasis, allergy, asthma, myocarditis, nephritis, hepatitis, systemic lupus erythematosus, rheumatoid arthritis, scleroderma, hyperthyroidism, idiopathic thrombocytopenia purpura, autoimmune hemolytic anemia, ulcerative colitis, autoimmune liver disease, diabetes, pain or neurological disorders.

In some embodiments, the therapeutic agent described herein includes an anti-IL2RB antibody, an anti-IL2RG antibody, an anti-IL15RA antibody, and/or an anti-IL15 antibody, or a corticosteroid (e.g., dexamethasone).

In one aspect, the disclosure is related to a method of determining effectiveness of a therapeutic agent for treating a cancer, comprising: a) administering the therapeutic agent to the animal described herein, in some embodiments, the animal has the cancer; and b) determining inhibitory effects of the therapeutic agent for treating the cancer. In some embodiments, the therapeutic agent includes an anti-IL2RB antibody, an anti-IL2RG antibody, an anti-IL15RA antibody, and/or an anti-IL15 antibody. In some embodiments, the cancer is a tumor, and determining the inhibitory effects of the treatment involves measuring the tumor volume in the animal. In some embodiments, the cancer comprises one or more cancer cells that are injected into the animal. In some embodiments, the cancer is breast cancer, ovarian cancer, endometrial cancer, melanoma, kidney cancer, lung cancer, or liver cancer.

In one aspect, the disclosure is related to a method of determining toxicity of an anti-IL2RB antibody, an anti-IL2RG antibody, an anti-IL15RA antibody, and/or an anti-IL15 antibody, comprising: a) administering the anti-IL2RB antibody, the anti-IL2RG antibody, the anti-IL15RA antibody, and/or the anti-IL15 antibody to the animal described herein; and b) determining effects of the therapeutic agent to the animal. In some embodiments, determining effects of the therapeutic agent to the animal involves measuring the body weight, red blood cell count, hematocrit, and/or hemoglobin of the animal.

In one aspect, the disclosure is related to a protein comprising an amino acid sequence, in some embodiments, the amino acid sequence is one of the following: (a) an amino acid sequence set forth in SEQ ID NO: 1, 2, 30, 33, 34, 39, 42, 43, 50, 51, or 52; (b) an amino acid sequence that is at least 90% identical to SEQ ID NO: 1, 2, 30, 33, 34, 39, 42, 43, 50, 51, or 52; (c) an amino acid sequence that is at least 91%, 92%, 93%, 94%, 95%, 96%, 97%, 98%, or 99% identical to SEQ ID NO: 1, 2, 30, 33, 34, 39, 42, 43, 50, 51, or 52; (d) an amino acid sequence that is different from the amino acid sequence set forth in SEQ ID NO: 1, 2, 30, 33, 34, 39, 42, 43, 50, 51, or 52 by no more than 10, 9, 8, 7, 6, 5, 4, 3, 2 or 1 amino acid; and (c) an amino acid sequence that comprises a substitution, a deletion and/or insertion of one, two, three, four, five or more amino acids to the amino acid sequence set forth in SEQ ID NO: 1, 2, 30, 33, 34, 39, 42, 43, 50, 51, or 52.

In one aspect, the disclosure is related to a nucleic acid comprising a nucleotide sequence, in some embodiments, the nucleotide sequence is one of the following: (a) a sequence that encodes the protein described herein; (b) SEQ ID NO: 3, 4, 5, 6, 7, 8, 9, 24, 25, 26, 27, 28, 29, 31, 32, 35, 36, 37, 38, 40, 41, 44, 45, 46, 47, 48, 49, 53, 54, 55, 56, 57, or 58; (c) a sequence that is at least 90% identical to SEQ ID NO: 3, 4, 5, 6, 7, 8, 9, 24, 25, 26, 27, 28, 29, 31, 32, 35, 36, 37, 38, 40, 41, 44, 45, 46, 47, 48, 49, 53, 54, 55, 56, 57, or 58; and (d) a sequence that is at least 91%, 92%, 93%, 94%, 95%, 96%, 97%, 98%, or 99% identical to SEQ ID NO: 3, 4, 5, 6, 7, 8, 9, 24, 25, 26, 27, 28, 29, 31, 32, 35, 36, 37, 38, 40, 41, 44, 45, 46, 47, 48, 49, 53, 54, 55, 56, 57, or 58.

In one aspect, the disclosure is related to a cell comprising the protein and/or the nucleic acid described herein. In one aspect, the disclosure is related to an animal comprising the protein and/or the nucleic acid described herein.

The disclosure further relates to a IL2RG, IL2RB, IL15RA, or IL15 genomic DNA sequence of a humanized mouse, a DNA sequence obtained by a reverse transcription of the mRNA obtained by transcription thereof is consistent with or complementary to the DNA sequence; a construct expressing the amino acid sequence thereof; a cell comprising the construct thereof; a tissue comprising the cell thereof.

The disclosure further relates to the use of the non-human mammal or an offspring thereof, or the tumor bearing non-human mammal, the animal model generated through the method as described herein in the development of a product related to an immunization processes of human cells, the manufacture of a human antibody, or the model system for a research in pharmacology, immunology, microbiology and medicine.

The disclosure also relates to the use of the non-human mammal or an offspring thereof, or the tumor bearing non-human mammal, the animal model generated through the method as described herein in the production and utilization of an animal experimental disease model of an immunization processes involving human cells, the study on a pathogen, or the development of a new diagnostic strategy and/or a therapeutic strategy.

The disclosure further relates to the use of the non-human mammal or an offspring thereof, or the tumor bearing non-human mammal, the animal model generated through the methods as described herein, in the screening, verifying, evaluating or studying the IL2RG, IL2RB, IL15RA, and/or IL15 gene functions, human IL2RG, IL2RB, IL15RA, and/or IL15 antibodies, the drugs or efficacies for human IL2 or IL15 signaling pathways, and the drugs for immune-related diseases and antitumor drugs.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a schematic diagram showing mouse and human IL2RG gene loci.

FIG. 2 is a schematic diagram showing humanized IL2RG gene locus.

FIG. 3 is a schematic diagram showing a IL2RG gene targeting strategy.

FIG. 4 is a schematic diagram showing a IL2RG gene targeting strategy using the CRISPR/Cas system.

FIG. 5A shows mouse tail PCR identification results of F0 generation mice by primers R-GT-F and R-GT-R. M is a marker. WT is a wild-type control. PC is a positive control. H₂O is a water control.

FIG. 5B shows mouse tail PCR identification results of F0 generation mice by primers L-GT-F and L-GT-R. M is a marker. WT is a wild-type control. PC is a positive control. H₂O is a water control.

FIG. 6A shows mouse tail PCR identification results of F1 generation mice by primers WT-F and WT-R. M is a marker. WT is a wild-type control. PC is a positive control. H₂O is a water control.

FIG. 6B shows mouse tail PCR identification results of F1 generation mice by primers WT-F and Mut-R. M is a marker. WT is a wild-type control. PC is a positive control. H₂O is a water control.

FIG. 7 shows Southern Blot results of F1 generation mice using the 3′ Probe and LR Probe. WT is a wild-type control.

FIG. 8 is a schematic diagram showing humanized IL2RG gene locus.

FIG. 9 is a schematic diagram showing a IL2RG gene targeting strategy.

FIG. 10 is a schematic diagram showing a IL2RG gene targeting strategy using the CRISPR/Cas system.

FIG. 11 is a schematic diagram showing mouse and human IL2RB gene loci.

FIG. 12 is a schematic diagram showing humanized IL2RB gene locus.

FIGS. 13A-13B show the percentages of leukocyte subtypes (A) and T cell subtypes (B), respectively, in the spleen of C57BL/6 wild-type mice (+/+) and IL2RB/IL2RG double-gene humanized heterozygous mice (H/+; H/+).

FIGS. 14A-14B show the percentages of leukocyte subtypes (A) and T cell subtypes (B), respectively, in the lymph nodes of C57BL/6 wild-type mice (+/+) and IL2RB/IL2RG double-gene humanized heterozygous mice (H/+; H/+).

FIGS. 15A-15B show the percentages of leukocyte subtypes (A) and T cell subtypes (B), respectively, in the peripheral blood of C57BL/6 wild-type mice (+/+) and IL2RB/IL2RG double-gene humanized heterozygous mice (H/+; H/+).

FIG. 16 is a schematic diagram showing mouse and human IL15RA gene loci.

FIG. 17 is a schematic diagram showing humanized IL15RA gene locus.

FIG. 18 is a schematic diagram showing a IL15RA gene targeting strategy.

FIG. 19 is a schematic diagram showing mouse and human IL15 gene loci.

FIG. 20 is a schematic diagram showing humanized IL15 gene locus.

FIG. 21 is a schematic diagram showing a IL15 gene targeting strategy.

FIG. 22 shows Southern Blot results of cells after recombination using the 5′ Probe, 3′ Probe, and Neo Probe-5 (3′). WT is a wild-type control.

FIG. 23 a schematic diagram showing the FRT recombination process in IL15 gene humanized mice.

FIGS. 24A-24D show mouse tail PCR identification results of F1 generation mice by primer pairs WT-F/WT-R, WT-F/Mut-R, Frt-F1/Frt-R1, and Frt-F2/Frt-R2, respectively. M is a marker. WT is a wild-type control. PC is a positive control. H₂O is a water control.

FIG. 25A shows ELISA results of mouse IL15 in LPS-stimulated lung tissues of C57BL/6 wild-type mice (+/+) and IL15 gene humanized heterozygous mice (H/+).

FIG. 25B shows ELISA results of human IL15 in LPS-stimulated lung tissues of C57BL/6 wild-type mice (+/+) and IL15 gene humanized heterozygous mice (H/+).

FIG. 26 shows RT-PCR results of mouse IL15 (mIL15) and human IL15 (hIL15) in a C57BL/6 wild-type mouse (+/+) and a IL15/IL15RA double-gene humanized homozygous mouse (H/H). H₂O is a water control. GAPDH was detected as an internal control.

FIG. 27 shows RT-PCR results of mouse IL15RA (mIL15RA) and human IL15RA (hIL15RA) in a C57BL/6 wild-type mouse (+/+) and a IL15/IL15RA double-gene humanized homozygous mouse (H/H). H₂O is a water control. GAPDH was detected as an internal control.

FIG. 28A shows ELISA results of mouse IL15 in acetaminophen-stimulated lung tissues of C57BL/6 wild-type mice (+/+) and IL15/IL15RA double-gene humanized homozygous mice (H/H).

FIG. 28B shows ELISA results of human IL15 in acetaminophen-stimulated lung tissues of C57BL/6 wild-type mice (+/+) and IL15/IL15RA double-gene humanized homozygous mice (H/H).

FIGS. 29A-29B show the percentages of leukocyte subtypes (A) and T cell subtypes (B), respectively, in the spleen of C57BL/6 wild-type mice (+/+) and IL15/IL15RA double-gene humanized homozygous mice (H/H).

FIGS. 30A-30B show the percentages of leukocyte subtypes (A) and T cell subtypes (B), respectively, in the peripheral blood of C57BL/6 wild-type mice (+/+) and IL15/IL15RA double-gene humanized homozygous mice (H/H).

FIGS. 31A-31B show the percentages of leukocyte subtypes (A) and T cell subtypes (B), respectively, in the lymph nodes of C57BL/6 wild-type mice (+/+) and IL15/IL15RA double-gene humanized homozygous mice (H/H).

FIG. 32A shows ELISA results of mouse IL15/IL15RA in acetaminophen-stimulated lung tissues of C57BL/6 wild-type mice (+/+) and IL15/IL15RA double-gene humanized homozygous mice (H/H).

FIG. 32B shows ELISA results of human IL15 in acetaminophen-stimulated lung tissues of C57BL/6 wild-type mice (+/+) and IL15/IL15RA double-gene humanized homozygous mice (H/H).

FIGS. 33A-33B show the percentages of leukocyte subtypes in the spleen of C57BL/6 wild-type mice (+/+) and IL2RB/IL2RG/IL15/IL15RA four-gene humanized homozygous mice (H/H).

FIG. 34 shows the percentages of T cell subtypes in the spleen of C57BL/6 wild-type mice (+/+) and IL2RB/IL2RG/IL15/IL15RA four-gene humanized homozygous mice (H/H).

FIGS. 35A-35B show the percentages of leukocyte subtypes in the peripheral blood of C57BL/6 wild-type mice (+/+) and IL2RB/IL2RG/IL15/IL15RA four-gene humanized homozygous mice (H/H).

FIG. 36 shows the percentages of T cell subtypes in the peripheral blood of C57BL/6 wild-type mice (+/+) and IL2RB/IL2RG/IL15/IL15RA four-gene humanized homozygous mice (H/H).

FIG. 37 shows the percentages of leukocyte subtypes in the lymph nodes of C57BL/6 wild-type mice (+/+) and IL2RB/IL2RG/IL15/IL15RA four-gene humanized homozygous mice (H/H).

FIG. 38 shows the percentages of T cell subtypes in the lymph nodes of C57BL/6 wild-type mice (+/+) and IL2RB/IL2RG/IL15/IL15RA four-gene humanized homozygous mice (H/H).

FIG. 39 shows the body weight of control group mice (G1), model group mice (G2), and administration group mice (G3) using IL15/IL15RA double-gene humanized homozygous mice in an IMQ-induced psoriasis model.

FIG. 40 shows the body weight change of control group mice (G1), model group mice (G2), and administration group mice (G3) using IL15/IL15RA double-gene humanized homozygous mice in an IMQ-induced psoriasis model.

FIG. 41 shows the erythema scores of control group mice (G1), model group mice (G2), and administration group mice (G3) using IL15/IL15RA double-gene humanized homozygous mice in an IMQ-induced psoriasis model.

FIG. 42 shows the scaling scores of control group mice (G1), model group mice (G2), and administration group mice (G3) using IL15/IL15RA double-gene humanized homozygous mice in an IMQ-induced psoriasis model.

FIG. 43 shows the comprehensive PASI (Psoriasis Area Severity Index) scores of control group mice (G1), model group mice (G2), and administration group mice (G3) using IL15/IL15RA double-gene humanized homozygous mice in an IMQ-induced psoriasis model.

FIG. 44 shows the alignment between human IL2RG amino acid sequence (NP_000197.1; SEQ ID NO: 2) and mouse IL2RG amino acid sequence (NP_038591.1; SEQ ID NO: 1).

FIG. 45 shows the alignment between human IL2RG amino acid sequence (NP_000197.1; SEQ ID NO: 2) and rat IL2RG amino acid sequence (NP_543165.1; SEQ ID NO: 85).

FIG. 46 shows the alignment between human IL2RB amino acid sequence (NP_000869.1; SEQ ID NO: 34) and mouse IL2RB amino acid sequence (NP_032394.1; SEQ ID NO: 33).

FIG. 47 shows the alignment between human IL2RB amino acid sequence (NP_000869.1; SEQ ID NO: 34) and rat IL2RB amino acid sequence (NP_037327.2; SEQ ID NO: 86).

FIG. 48 shows the alignment between human IL15RA amino acid sequence (NP_002180.1; SEQ ID NO: 43) and mouse IL15RA amino acid sequence (NP_032384.1; SEQ ID NO: 42).

FIG. 49 shows the alignment between human IL15RA amino acid sequence (NP_002180.1; SEQ ID NO: 43) and rat IL15RA amino acid sequence (XP_002728555.2; SEQ ID NO: 87).

FIG. 50 shows the alignment between human IL15 amino acid sequence (NP_000576.1; SEQ ID NO: 52) and mouse IL15 amino acid sequence (NP_001241676.1; SEQ ID NO: 51).

FIG. 51 shows the alignment between human IL15 amino acid sequence (NP_000576.1; SEQ ID NO: 52) and rat IL15 amino acid sequence (NP_001388064.1; SEQ ID NO: 88).

### DETAILED DESCRIPTION

This disclosure relates to transgenic non-human animal with human or chimeric (e.g., humanized) IL2RG, IL2RB, IL15RA, and/or IL15, and methods of use thereof.

IL-2 exerts its biological activity by acting on IL-2R on the cell membrane. IL-2R is a complex composed of IL-2Rα (CD25), IL-2Rβ (CD122) and IL-2Rγ (CD132). IL-2Rα binds to IL-2 with low affinity and cannot conduct intracellular signal transduction. The γ subunit not only responds to IL-2, but also responds to IL-4, IL-7, IL-9, IL-15, and IL-21. When IL-2Rα, IL-2Rβ and IL-2Rγ form a trimer, the affinity is increased by 10-100 times. IL-2Rβ and IL-2Rγ belong to the type I cytokine receptor superfamily. The γ subunit does not bind to IL-2 alone but binds to the β subunit and forms a low-affinity dimer. IL-2 binds to the above three allosteric receptors collectively referred to as a component of the IL-2 and IL-2R signal. The β and γ subunits carry signal sequences in the tails of the cytoplasm, and their signal sequences are transduced through a variety of intracellular pathways such as JAK-STAT, PI3K and MAPK.

The JAK-STAT pathway accounts for 90% of IL-2 and IL-2R signal. IL-2 binding leads to heterodimerization of IL-2Rβ and IL-2Rγ, activating the tyrosine kinases JAK1 and JAK3, respectively, which phosphorylate tyrosine residues in IL-2Rβ. This promotes recruitment of signaling molecules such as PI3K, STAT5 or SHC1, which are phosphorylated by JAKs, resulting in specific pathway activation, nuclear translocation of transcription factors and finally targeted transcription regulation that induces cell activation, differentiation, and proliferation. PI3K phosphorylates phosphatidylinositol 4, 5-bisphosphate (PIP2), resulting in production of phosphatidylinositol-3, 4, 5-trisphosphate (PIP3), which promotes recruitment of phosphoinositide-dependent kinase 1 (PDK1) and AKT (also known as PKB) to the cell membrane. Phosphorylation of AKT by PDK1 and mTOR complex 2 (mTORC2) is necessary for full activation. AKT phosphorylation of tuberous sclerosis complex (TSC) proteins relieve TSC-mediated inhibition of RHEB to activate mTORC1, which phosphorylates p70 ribosomal S6 kinase (p70S6K), a kinase that is important for survival, proliferation, and protein translation. Tyrosine phosphorylation of STAT5 leads to its dimerization or tetramerization, nuclear translocation and transcription activation or repression. Phosphorylation of SHC1 promotes recruitment of GRB2 and SOS, forming a complex that catalysis GTP exchange on RAS and subsequent activation of the MAPK pathway. Depending on the concentration and duration of exposure, IL-2 induces different signals in conventional T cells compared with Treg cells, which influences the outcome of a localized immune response in a pro-inflammatory setting. Aside from natural IL-2, enhanced IL-2 formulations such as muteins or IL-2-anti-IL-2 antibody complexes can be targeted to Treg cells or conventional T cells in autoimmune or cancer settings, respectively, and, depending on modified binding properties, induce stronger IL-2 signal.

IL-15 is a member of the “four α-helix bundle” cytokine family that signals via the common γ chain (IL2Ry) and the IL2RB chain, and as a result the two cytokines share select biologic functions. IL-15 transcript is abundantly produced by a large variety of tissues and cell types: (i) tissues include the placenta, skeletal muscle, kidney, lung, and heart tissue; and (ii) cell types include epithelial cells, fibroblasts, keratinocytes, nerve cells, monocytes, macrophages, and dendritic cells. Transcriptional activation of IL-15 occurs via the binding of NF-κB and IRF-E to the 5′ regulatory region of IL-15, among other active motifs such as GC-binding factor (GCF), myb, and INF2. Despite the abundant expression of IL-15 transcript, IL-15 protein is stringently controlled and expressed primarily within monocytes, macrophages, and dendritic cells. This discrepancy between IL-15 transcript and protein expression is due to complex translation and intracellular protein trafficking culminating in barely detectable levels of the protein in vivo. IL-15 posttranscriptional checkpoints include a complex 5′-untranslated region (UTR) containing (i) multiple AUG sequences upstream of the initiation codon; (ii) a C-terminal negative regulatory element; and (iii) an inefficient signal peptide. Collectively, these mechanisms serve to limit IL-15 protein production and secretion from its vast stores of transcript.

Despite the lack of homology in the amino acid sequence between IL-15 and IL-2, the mature IL-15 protein binds to the IL-2Rβγ heterodimer, activating the intracellular signal leading to cell activation. The third component of the IL-15R complex is a unique α-chain (IL-15Rα). In contrast with the IL-2Rα chain that binds IL-2 with low affinity and confers high affinity for IL-2 only when noncovalently linked the IL-2Rβγ complex, IL-15Rα is by itself a high-affinity receptor for IL-15. Once IL-15 is secreted out of the cell, it binds to either the membrane bound or the soluble form of IL-15Rα and is presented in trans to and bound by the IL-2Rβγ complex expressed on nearby effector cells to initiate cellular activation. IL-15 utilizes select Janus-associated kinases (JAK) and signal transducer and activator of transcription (STAT) proteins as a means of initiating signal transduction for cellular activation. In lymphocytes, binding of IL-15 to the IL-2/15Rβγ heterodimer induces JAKI activation that subsequently phosphorylates STAT3 via the β-chain and JAK3/STAT5 activation via its γ-chain. Phosphorylated STAT3 and STAT5 proteins form heterodimers that then translocate to the nucleus, where they activate transcription of the antiapoptotic protein bcl-2 and proto-oncogenes c-myc, c-fos, and c-jun. Mice that have genetic disruption of IL-15, JAK3, or STAT5 show a profound lymphoid cell deficiency.

Thus, antibodies targeting the IL2 and/or IL15 signaling pathways can be potentially used as therapies for treating immune disorders or cancers.

Experimental animal models are an indispensable research tool for studying the effects of therapeutic agents (e.g., antibodies targeting IL2 or IL15 signaling pathways). Common experimental animals include mice, rats, guinea pigs, hamsters, rabbits, dogs, monkeys, pigs, fish and so on. However, there are many differences between human and animal genes and protein sequences, and many human proteins cannot bind to the animal's homologous proteins to produce biological activity, leading to that the results of many clinical trials do not match the results obtained from animal experiments. A large number of clinical studies are in urgent need of better animal models. With the continuous development and maturation of genetic engineering technologies, the use of human cells or genes to replace or substitute an animal's endogenous similar cells or genes to establish a biological system or disease model closer to human, and establish the humanized experimental animal models (humanized animal model) has provided an important tool for new clinical approaches or means. In this context, the genetically engineered animal model, that is, the use of genetic manipulation techniques, the use of human normal or mutant genes to replace animal homologous genes, can be used to establish the genetically modified animal models that are closer to human gene systems. The humanized animal models have various important applications. For example, due to the presence of human or humanized genes, the animals can express or express in part of the proteins with human functions, so as to greatly reduce the differences in clinical trials between humans and animals, and provide the possibility of drug screening at animal levels.

Unless otherwise specified, the practice of the methods described herein can take advantage of the techniques of cell biology, cell culture, molecular biology, transgenic biology, microbiology, recombinant DNA and immunology. These techniques are explained in detail in the following literature, for examples: Molecular Cloning A Laboratory Manual, 2nd Ed., cd. By Sambrook, Fritsch and Maniatis (Cold Spring Harbor Laboratory Press: 1989); DNA Cloning, Volumes I and II (D. N. Glovered., 1985); Oligonucleotide Synthesis (M. J. Gaited., 1984); Mullis et al U.S. Pat. No. 4,683,195; Nucleic Acid Hybridization (B. D. Hames & S. J. Higginseds. 1984); Transcription And Translation (B. D. Hames & S. J. Higginseds. 1984); Culture Of Animal Cell (R. I. Freshney, Alan R. Liss, Inc., 1987); Immobilized Cells And Enzymes (IRL Press, 1986); B. Perbal, A Practical Guide To Molecular Cloning (1984), the series, Methods In ENZYMOLOGY (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Caloseds., 1987, Cold Spring Harbor Laboratory); Immunochemical Methods In Cell And Molecular Biology (Mayer and Walker, eds., Academic Press, London, 1987); Hand book Of Experimental Immunology, Volumes V (D. M. Weir and C. C. Blackwell, eds., 1986); and Manipulating the Mouse Embryo, (Cold Spring Harbor Laboratory Press, Cold Spring Harbor, N.Y., 1986); each of which is incorporated herein by reference in its entirety.

### IL2RG

The common gamma chain (γ_{c}) (CD132), also known as interleukin-2 receptor subunit gamma, IL2RG, or IL2Rγ, is a cytokine receptor sub-unit that is common to the receptor complexes for at least six different interleukin receptors: IL-2, IL-4, IL-7, IL-9, IL-15, and IL-21 receptor. The γₑ glycoprotein is a member of the type I cytokine receptor family expressed on most lymphocyte (white blood cell) populations, and its gene is found on the X-chromosome of mammals. This protein is located on the surface of immature blood-forming cells in bone marrow. One end of the protein resides outside the cell where it binds to cytokines and the other end of the protein resides in the interior of the cell where it transmits signals to the cell's nucleus. The common gamma chain partners with other proteins to direct blood-forming cells to form lymphocytes (a type of white blood cell). The receptor also directs the growth and maturation of lymphocyte subtypes: T cells, B cells, and natural killer cells. These cells kill viruses, make antibodies, and help regulate the entire immune system.

Among γ_{c} family cytokines, IL-2 and IL-15 each are unusual in having three receptor chains rather than two, with each having a distinctive α chain (IL-2Rα and IL-15Rα, respectively), but both cytokines share IL-2Rβ and γ_{c}, IL-2Rα and IL-15Rα both have relatively short cytoplasmic domains and do not possess known signaling activity, but they participate in the formation of high-affinity receptor complexes and serve to increase the sensitivity of the cells to IL-2 and IL-15, respectively. Importantly, IL-15 can efficiently bind IL-15Rα to form an IL-15/IL-15Rα complex, allowing trans-presentation of IL-15 to neighboring cells bearing the IL-2Rβ/γ_{c} signaling complex. IL-2 signals mainly in cis, but can also signal in trans.

A detailed description of IL2RG and its function can be found, e.g., in Mitra, S., et al. “Biology of IL-2 and its therapeutic modulation: Mechanisms and strategies.” Journal of leukocyte biology 103.4 (2018): 643-655; and Lin, J. et al. “The common cytokine receptor γchain family of cytokines.” Cold Spring Harbor perspectives in biology 10.9 (2018): a028449; each of which is incorporated by reference in its entirety.

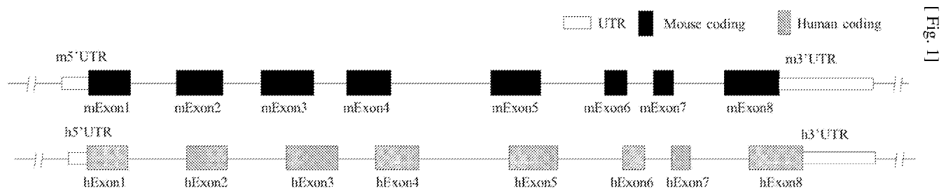

In human genomes, IL2RG gene (Gene ID: 3561) locus has eight exons, exon 1, exon 2, exon 3, exon 4, exon 5, exon 6, exon 7, and exon 8 (FIG. 1). The human IL2RG protein also has, from N-terminus to C-terminus, a signal peptide, an extracellular region, a transmembrane region, and a cytoplasmic region. The nucleotide sequence for human IL2RG mRNA is NM_000206.2, and the amino acid sequence for human IL2RG is NP_000197.1 (SEQ ID NO: 2). The location for each exon and each region in human IL2RG nucleotide sequence and amino acid sequence is listed below:

TABLE 1

| | | NP_000197.1 |
|---|---|---|
| Human IL2RG | NM_000206.2 | 369aa |
| (approximate location) | 1560 bp | SEQ ID NO: 2 |
| Exon 1 | 1-207 | 1-38 |
| Exon 2 | 208-361 | 39-90 |
| Exon 3 | 362-546 | 91-151 |
| Exon 4 | 547-686 | 152-198 |
| Exon 5 | 687-849 | 199-252 |
| Exon 6 | 850-946 | 253-285 |
| Exon 7 | 947-1016 | 286-308 |
| Exon 8 | 1017-1534 | 309-369 |
| Signal peptide | 93-158 | 1-22 |
| Extracellular | 159-878 | 23-262 |
| Transmembrane | 879-941 | 263-283 |
| Cytoplasmic | 942-1199 | 284-369 |
| Donor region (Example 1) | 93-1534 | 1-369 |
| Donor region (Example 2) | 93-860 | 1-256 |

The human IL2RG gene (Gene ID: 3561) is located in Chromosome X of the human genome, which is located from 71107404 to 71111631 of NC_000023.11. The 5′ UTR is from 71111631 to 71111540, Exon 1 is from 71111631 to 71111425, Exon 2 is from 71111050 to 71110897, Exon 3 is from 71110688 to 71110504, Exon 4 is from 71110295 to 71110156, Exon 5 is from 71109390 to 71109228, Exon 6 is from 71108695 to 71108599, Exon 7 is from 71108346 to 71108277, Exon 8 is from 71107921 to 71107404, and the 3′UTR is from 71107735 to 71107404, based on transcript NM_000206.2. All relevant information for human IL2RG locus can be found in the NCBI website with Gene ID: 3561, which is incorporated by reference herein in its entirety.

In mice, IL2RG gene locus has eight exons, exon 1, exon 2, exon 3, exon 4, exon 5, exon 6, exon 7, and exon 8 (FIG. 1). The mouse IL2RG protein also has, from N-terminus to C-terminus, a signal peptide, an extracellular region, a transmembrane region, and a cytoplasmic region. The nucleotide sequence for mouse IL2RG mRNA is NM_013563.4, the amino acid sequence for mouse IL2RG is NP_038591.1 (SEQ ID NO: 1). The location for each exon and each region in the mouse IL2RG nucleotide sequence and amino acid sequence is listed below:

TABLE 2

| | | NP_038591.1 |
|---|---|---|
| Mouse IL2RG | NM_013563.4 | 369 aa |
| (approximate location) | 1663 bp | SEQ ID NO: 1 |
| Exon 1 | 1-201 | 1-38 |
| Exon 2 | 202-355 | 39-90 |
| Exon 3 | 356-540 | 91-151 |
| Exon 4 | 541-683 | 152-199 |
| Exon 5 | 684-846 | 200-253 |
| Exon 6 | 847-943 | 254-286 |
| Exon 7 | 944-1010 | 287-308 |
| Exon 8 | 1011-1663 | 309-369 |
| Signal peptide | 87-152 | 1-22 |
| Extracellular | 153-875 | 23-263 |
| Transmembrane | 876-938 | 264-284 |
| Cytoplasmic | 939-1193 | 285-369 |
| Replaced region in Example 1 | 87-1613 | 1-369 |
| Replaced region in Example 2 | 87-857 | 1-257 |

The mouse IL2RG gene (Gene ID: 16186) is located in Chromosome X of the mouse genome, which is located from 100307991 to 100311861 of NC_000086.8 (GRCm39 (GCF_000001635.27)). The 5′ UTR is from 100311861 to 100311776, Exon 1 is from 100311861 to 100311661, Exon 2 is from 100311470 to 100311317, Exon 3 is from 100311101 to 100310917, Exon 4 is from 100310726 to 100310584, Exon 5 is from 100309949 to 100309787, Exon 6 is from 100309332 to 100309236, Exon 7 is from 100309048 to 100308982, Exon 8 is from 100308643 to 100307984, and the 3′UTR is from 100308457 to 100307984, based on transcript NM_013563.4. All relevant information for mouse Il2rg locus can be found in the NCBI website with Gene ID: 16186, which is incorporated by reference herein in its entirety.

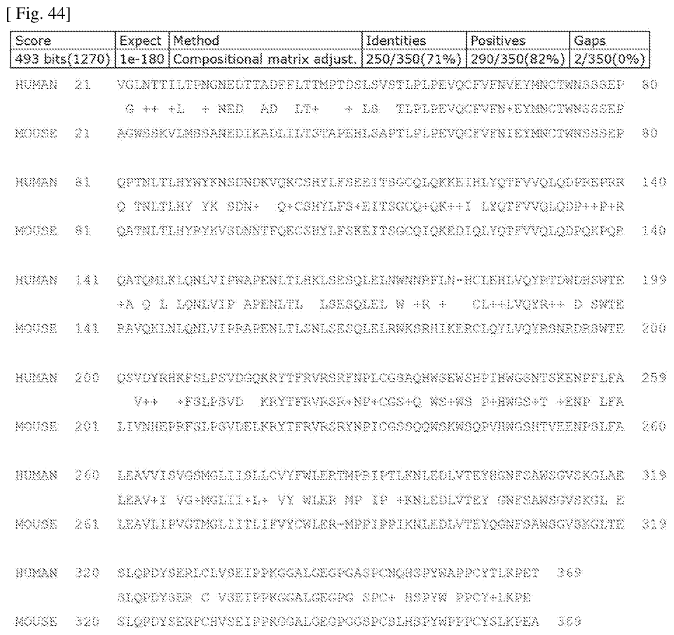

FIG. 44 shows the alignment between human IL2RG amino acid sequence (NP_000197.1; SEQ ID NO: 2) and mouse IL2RG amino acid sequence (NP_038591.1; SEQ ID NO: 1). Thus, the corresponding amino acid residue or region between human and mouse IL2RG can be found in FIG. 44.

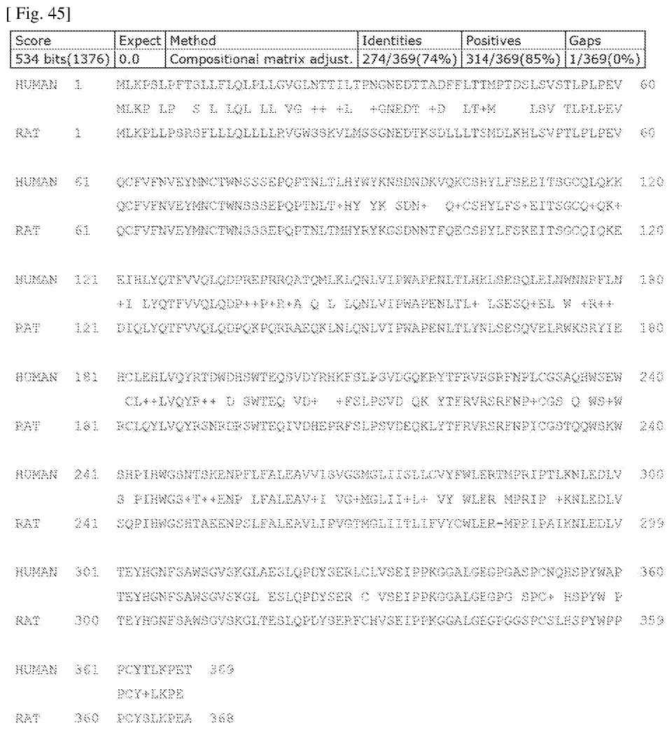

IL2RG genes, proteins, and locus of the other species are also known in the art. For example, the gene ID for IL2RG in Rattus norvegicus (rat) is 140924, the gene ID for IL2RG in Macaca mulatta (Rhesus monkey) is 641338, the gene ID for IL2RG in Canis lupus familiaris (dog) is 403851, and the gene ID for IL2RG in Sus scrofa (pig) is 397156. The relevant information for these genes (e.g., intron sequences, exon sequences, amino acid residues of these proteins) can be found, e.g., in NCBI database, which is incorporated by reference herein in its entirety. FIG. 45 shows the alignment between human IL2RG amino acid sequence (NP_000197.1; SEQ ID NO: 2) and rat IL2RG amino acid sequence (NP_543165.1; SEQ ID NO: 85). Thus, the corresponding amino acid residue or region between human and rodent IL2RG can be found in FIG. 45.

The present disclosure provides human or chimeric (e.g., humanized) IL2RG nucleotide sequence and/or amino acid sequences. In some embodiments, the entire sequence of mouse exon 1, exon 2, exon 3, exon 4, exon 5, exon 6, exon 7, exon 8, signal peptide, extracellular region, transmembrane region, and/or cytoplasmic region are replaced by the corresponding human sequence. In some embodiments, a “region” or “portion” of mouse exon 1, exon 2, exon 3, exon 4, exon 5, exon 6, exon 7, exon 8, signal peptide, extracellular region, transmembrane region, and/or cytoplasmic region are replaced by the corresponding human sequence. The term “region” or “portion” can refer to at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 250, 300, 350, 400, 450, 500, 550, 600, 650, 700, 750, 760, 765, 766, 767, 768, 769, 770, 771, 772, 773, 800, 850, 900, 950, 1000, 1100, 1200, 1300, 1400, 1440, 1441, 1442, 1443, 1450, 1500, 1520, 1525, 1526, or 1527 nucleotides, or at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, 250, 255, 256, 257, 258, 259, 260, 270, 280, 290, 300, 310, 320, 330, 340, 350, 360, 365, 366, 367, 368, or 369 amino acid residues. In some embodiments, the “region” or “portion” can be at least 50%, 55%, 60%, 65%, 70%, 75%, 80%, 85%, 90%, 95%, or 99% identical to exon 1, exon 2, exon 3, exon 4, exon 5, exon 6, exon 7, exon 8, signal peptide, extracellular region, transmembrane region, or cytoplasmic region. In some embodiments, a region, a portion, or the entire sequence of mouse exon 1, exon 2, exon 3, exon 4, exon 5, exon 6, exon 7, and/or exon 8 (e.g., a portion of exon 1, exons 2-5, and a portion of exon 6) are replaced by a region, a portion, or the entire sequence of the human exon 1, exon 2, exon 3, exon 4, exon 5, exon 6, exon 7, and/or exon 8 (e.g., a portion of exon 1, exons 2-5, and a portion of exon 6).

In some embodiments, a “region” or “portion” of the signal peptide, extracellular region, transmembrane region, cytoplasmic region, exon 1, exon 2, exon 3, exon 4, exon 5, exon 6, exon 7, and/or exon 8 is deleted.

In some embodiments, the present disclosure is related to a genetically-modified, non-human animal whose genome comprises a chimeric (e.g., humanized) IL2RG nucleotide sequence. In some embodiments, the chimeric (e.g., humanized) IL2RG nucleotide sequence encodes a IL2RG protein comprising a signal peptide, an extracellular region, a transmembrane region, and a cytoplasmic region. In some embodiments, the signal peptide comprises a sequence that is at least 80%, 85%, 90%, 95%, or 100% identical to amino acids 1-22 of SEQ ID NO: 2. In some embodiments, the signal peptide comprises all or part of human IL2RG signal peptide. In some embodiments, the extracellular region comprises a sequence that is at least 80%, 85%, 90%, 95%, or 100% identical to amino acids 23-256 or 23-262 of SEQ ID NO: 2. In some embodiments, the extracellular region comprises all or part of human IL2RG extracellular region. In some embodiments, the extracellular region comprises at least 1, 2, 3, 4, 5, or 6 amino acids at the C-terminus of endogenous IL2RG extracellular region. In some embodiments, the transmembrane region comprises a sequence that is at least 80%, 85%, 90%, 95%, or 100% identical to amino acids 264-284 of SEQ ID NO: 1. In some embodiments, the transmembrane region comprises all or part of endogenous IL2RG transmembrane region. In some embodiments, the cytoplasmic region comprises a sequence that is at least 80%, 85%, 90%, 95%, or 100% identical to amino acids 285-369 of SEQ ID NO: 1. In some embodiments, the cytoplasmic region comprises all or part of endogenous IL2RG cytoplasmic region. In some embodiments, the genome of the animal comprises a sequence that is at least 80%, 85%, 90%, 95%, or 100% identical to SEQ ID NO: 3, 4, 5, 6, 7, 8, 9, 24, 25, 26, 27, 28, 29, 31, or 32.

In some embodiments, the genetically-modified non-human animal described herein comprises a sequence encoding a human or humanized IL2RG protein. In some embodiments, the IL2RG protein comprises, from N-terminus to C-terminus, a signal peptide, an extracellular region, a transmembrane region, and a cytoplasmic region. In some embodiments, the humanized IL2RG protein comprises a human or humanized signal peptide. In some embodiments, the humanized IL2RG protein comprises an endogenous signal peptide. In some embodiments, the humanized IL2RG protein comprises a human or humanized extracellular region. In some embodiments, the humanized IL2RG protein comprises an endogenous extracellular region. In some embodiments, the humanized IL2RG protein comprises a human or humanized transmembrane region. In some embodiments, the humanized IL2RG protein comprises an endogenous transmembrane region. In some embodiments, the humanized IL2RG protein comprises a human or humanized cytoplasmic region. In some embodiments, the humanized IL2RG protein comprises an endogenous cytoplasmic region. In some embodiments, the humanized IL2RG protein comprises a human or humanized signal peptide, a human or humanized extracellular region, an endogenous transmembrane region, and an endogenous cytoplasmic region. In some embodiments, the humanized IL2RG protein comprises an endogenous sequence that corresponds to amino acids 258-369 of SEQ ID NO: 1.

In some embodiments, the genetically-modified non-human animal described herein comprises a human or humanized IL2RG gene. In some embodiments, the humanized IL2RG gene comprises 8 exons. In some embodiments, the humanized IL2RG gene comprises humanized exon 1, human exon 2, human exon 3, human exon 4, human exon 5, human exon 6, human exon 7, and/or human exon 8. In some embodiments, the humanized IL2RG gene comprises humanized exon 1, human exon 2, human exon 3, human exon 4, human exon 5, humanized exon 6, endogenous exon 7, and/or endogenous exon 8. In some embodiments, the humanized IL2RG gene comprises 7 introns. In some embodiments, the humanized IL2RG gene comprises human intron 1, human intron 2, human intron 3, human intron 4, human intron 5, human intron 6, and/or human intron 7. In some embodiments, the humanized IL2RG gene comprises human intron 1, human intron 2, human intron 3, human intron 4, human intron 5, endogenous intron 6, and/or endogenous intron 7. In some embodiments, the humanized IL2RG gene comprises human or humanized 5′ UTR. In some embodiments, the humanized IL2RG gene comprises human or humanized 3′ UTR. In some embodiments, the humanized IL2RG gene comprises endogenous 5′ UTR. In some embodiments, the humanized IL2RG gene comprises endogenous 3′ UTR.

Thus, in some embodiments, the present disclosure also provides a chimeric (e.g., humanized) IL2RG nucleotide sequence and/or amino acid sequences, wherein in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from mouse IL2RG mRNA sequence (e.g., NM_013563.4), mouse IL2RG amino acid sequence (e.g., SEQ ID NO: 1), or a portion thereof (e.g., a portion of exon 1, a portion of exon 6, and exons 7-8); and in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from human IL2RG mRNA sequence (e.g., NM_000206.2), human IL2RG amino acid sequence (e.g., SEQ ID NO: 2), or a portion thereof (e.g., a portion of exon 1, exons 2-5, and a portion of exon 6).

In some embodiments, the sequence encoding amino acids 1-257, or 23-257 of mouse IL2RG (SEQ ID NO: 1) is replaced. In some embodiments, the sequence is replaced by a sequence encoding a corresponding region of human IL2RG (e.g., amino acids 1-256, or 23-256 of human IL2RG (SEQ ID NO: 2)).

In some embodiments, the sequence encoding amino acids 1-369, or 23-369 of mouse IL2RG (SEQ ID NO: 1) is replaced. In some embodiments, the sequence is replaced by a sequence encoding a corresponding region of human IL2RG (e.g., amino acids 1-369, or 23-369 of human IL2RG (SEQ ID NO: 2)).

In some embodiments, the sequence encoding amino acids 1-263, or 23-263 of mouse IL2RG (SEQ ID NO: 1) is replaced. In some embodiments, the sequence is replaced by a sequence encoding a corresponding region of human IL2RG (e.g., amino acids 1-262, or 23-262 of human IL2RG (SEQ ID NO: 2)).

In some embodiments, the nucleic acids as described herein are operably linked to a promotor or regulatory element, e.g., an endogenous mouse IL2RG promotor, an inducible promoter, an enhancer, and/or mouse or human regulatory elements.

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, 100, 150, 200, 250, 300, 350, 400, 450, 500, 550, 600, 650, 700, 750, 760, 765, 766, 767, 768, 769, 770, 771, 772, 773, 800, 850, 900, 950, 1000, 1100, 1200, 1300, 1400, 1440, 1450, 1500, 1520, 1525, 1526, or 1527 nucleotides, e.g., contiguous or non-contiguous nucleotides) that are different from part of or the entire mouse IL2RG nucleotide sequence (e.g., a portion of exon 1, exons 2-5, and a portion of exon 6 of NM_013563.4).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 51, 52, 53, 55, 60, 70, 80, 85, 76, 87, 88, 89, 90, 91, 92, 93, 94, 95, 100, 150, 200, 250, 300, 350, 400, 450, 500, 550, 600, 650, 700, 750, 800, 805, 806, 807, 850, or 900 nucleotides, e.g., contiguous or non-contiguous nucleotides) that is the same as part of or the entire mouse IL2RG nucleotide sequence (e.g., a portion of exon 1, a portion of exon 6, and exons 7-8 of NM_013563.4).

In some embodiments, the nucleic acid sequence has at least a portion (e.g., at least 1, 2, 3, 4, 5, 6, 7, 8, 9, 10, 11, 12, 13, 14, 15, 20, 25, 26, 27, 30, 40, 50, 60, 70, 80, 90, 91, 92, 93, 95, 100, 150, 200, 250, 300, 350, 400, 450, 500, 550, 600, 660, 700, 750, or 800 nucleotides, e.g., contiguous or non-contiguous nucleotides) that is different from part of or the entire human IL2RG nucleotide sequence (e.g., a portion of exon 1, a portion of exon 6, and exons 7-8 of NM_000206.2).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, 100, 150, 200, 250, 300, 350, 400, 450, 500, 550, 600, 650, 700, 750, 760, 765, 766, 767, 768, 769, 770, 800, 850, 900, 950, 1000, 1100, 1200, 1300, 1400, 1441, 1442, 1443, or 1450 nucleotides, e.g., contiguous or non-contiguous nucleotides) that is the same as part of or the entire human IL2RG nucleotide sequence (e.g., a portion of exon 1, exons 2-5, and a portion of exon 6 of NM_000206.2).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, 100, 110, 120, 130, 140, 150, 160, 170, 180, 190, 200, 210, 220, 230, 240, 250, 255, 256, 257, 258, 259, 260, 270, 280, 290, 300, 310, 320, 330, 340, 350, 360, 365, 366, 367, 368, or 369 amino acid residues, e.g., contiguous or non-contiguous amino acid residues) that is different as part of or the entire mouse IL2RG amino acid sequence (e.g., amino acids 1-257 of NP_038591.1 (SEQ ID NO: 1)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, 100, 110, 111, or 112 amino acid residues, e.g., contiguous or non-contiguous amino acid residues) that is the same from part of or the entire mouse IL2RG amino acid sequence (e.g., amino acids 258-369 of NP_038591.1 (SEQ ID NO: 1)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, 100, 110, 111, 112, or 113 amino acid residues, e.g., contiguous or non-contiguous amino acid residues) that is different from part of or the entire human IL2RG amino acid sequence (e.g., amino acids 257-369 of NP_000197.1 (SEQ ID NO: 2)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, 100, 110, 120, 130, 140, 150, 160, 170, 180, 190, 200, 210, 220, 230, 240, 250, 251, 252, 253, 254, 255, 256, 257, 258, 259, 260, 270, 280, 290, 300, 310, 320, 330, 340, 350, 360, 365, 366, 367, 368, or 369 amino acid residues, e.g., contiguous or non-contiguous amino acid residues) that is the same as part of or the entire human IL2RG amino acid sequence (e.g., amino acids 1-256 of NP_000197.1 (SEQ ID NO: 2)).

The present disclosure also provides a humanized IL2RG mouse amino acid sequence, wherein the amino acid sequence is selected from the group consisting of:
a) an amino acid sequence shown in SEQ ID NO: 1, 2, or 30;
b) an amino acid sequence having a homology of at least 90% with or at least 90% identical to the amino acid sequence shown in SEQ ID NO: 1, 2, or 30;
c) an amino acid sequence encoded by a nucleic acid sequence, wherein the nucleic acid sequence is able to hybridize to a nucleotide sequence encoding the amino acid shown in SEQ ID NO: 1, 2, or 30 under a low stringency condition or a strict stringency condition;
d) an amino acid sequence having a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the amino acid sequence shown in SEQ ID NO: 1, 2, or 30;
e) an amino acid sequence that is different from the amino acid sequence shown in SEQ ID NO: 1, 2, or 30 by no more than 10, 9, 8, 7, 6, 5, 4, 3, 2 or no more than 1 amino acid; or
f) an amino acid sequence that comprises a substitution, a deletion and/or insertion of one or more amino acids to the amino acid sequence shown in SEQ ID NO: 1, 2, or 30.

The present disclosure also provides a humanized IL2RG amino acid sequence, wherein the amino acid sequence is selected from the group consisting of:
a) all or part of amino acids 23-256 or 23-262 of SEQ ID NO: 2;
b) an amino acid sequence have a homology of at least 90%, 91%, 92%, 93%, 94%, 95%, 96%, 97%, 98%, or 99% to amino acids 23-256 or 23-262 of SEQ ID NO: 2;
c) an amino acid sequence that is different from amino acids 23-256 or 23-262 of SEQ ID NO: 2 by no more than 10, 9, 8, 7, 6, 5, 4, 3, 2 or no more than 1 amino acid; and
d) an amino acid sequence that comprises a substitution, a deletion and/or insertion of one or more amino acids to amino acids 23-256 or 23-262 of SEQ ID NO: 2.

The present disclosure also provides a humanized IL2RG amino acid sequence, wherein the amino acid sequence is selected from the group consisting of:
a) all or part of amino acids 1-256 or 1-369 of SEQ ID NO: 2;
b) an amino acid sequence have a homology of at least 90%, 91%, 92%, 93%, 94%, 95%, 96%, 97%, 98%, or 99% to amino acids 1-256 or 1-369 of SEQ ID NO: 2;
c) an amino acid sequence that is different from amino acids 1-256 or 1-369 of SEQ ID NO: 2 by no more than 10, 9, 8, 7, 6, 5, 4, 3, 2 or no more than 1 amino acid; and
d) an amino acid sequence that comprises a substitution, a deletion and/or insertion of one or more amino acids to amino acids 1-256 or 1-369 of SEQ ID NO: 2.

The present disclosure also provides a humanized IL2RG amino acid sequence, wherein the amino acid sequence is selected from the group consisting of:
a) all or part of amino acids 258-369 of SEQ ID NO: 1;
b) an amino acid sequence have a homology of at least 90%, 91%, 92%, 93%, 94%, 95%, 96%, 97%, 98%, or 99% to amino acids 258-369 of SEQ ID NO: 1;
c) an amino acid sequence that is different from amino acids 258-369 of SEQ ID NO: 1 by no more than 10, 9, 8, 7, 6, 5, 4, 3, 2 or no more than 1 amino acid; and
d) an amino acid sequence that comprises a substitution, a deletion and/or insertion of one or more amino acids to amino acids 258-369 of SEQ ID NO: 1.

The present disclosure also relates to a IL2RG nucleic acid (e.g., DNA or RNA) sequence, wherein the nucleic acid sequence can be selected from the group consisting of:
a) a nucleic acid sequence as shown in SEQ ID NO: 3, 4, 5, 6, 7, 8, 9, 24, 25, 26, 27, 28, 29, 31, or 32, or a nucleic acid sequence encoding a homologous IL2RG amino acid sequence of a humanized mouse IL2RG;
b) a nucleic acid sequence that is able to hybridize to the nucleotide sequence as shown in SEQ ID NO: 3, 4, 5, 6, 7, 8, 9, 24, 25, 26, 27, 28, 29, 31, or 32 under a low stringency condition or a strict stringency condition;
c) a nucleic acid sequence that has a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the nucleotide sequence as shown in SEQ ID NO: 3, 4, 5, 6, 7, 8, 9, 24, 25, 26, 27, 28, 29, 31, or 32;
d) a nucleic acid sequence that encodes an amino acid sequence, wherein the amino acid sequence has a homology of at least 90% with or at least 90% identical to the amino acid sequence shown in SEQ ID NO: 1, 2, or 30;
e) a nucleic acid sequence that encodes an amino acid sequence, wherein the amino acid sequence has a homology of at least 90%, 91%, 92%, 93%, 94%, 95%, 96%, 97%, 98%, or 99% with, or at least 90%, 91%, 92%, 93%, 94%, 95%, 96%, 97%, 98%, or 99% identical to the amino acid sequence shown in SEQ ID NO: 1, 2, or 30;
f) a nucleic acid sequence that encodes an amino acid sequence, wherein the amino acid sequence is different from the amino acid sequence shown in SEQ ID NO: 1, 2, or 30 by no more than 10, 9, 8, 7, 6, 5, 4, 3, 2 or no more than 1 amino acid; and/or
g) a nucleic acid sequence that encodes an amino acid sequence, wherein the amino acid sequence comprises a substitution, a deletion and/or insertion of one or more amino acids to the amino acid sequence shown in SEQ ID NO: 1, 2, or 30.

The present disclosure further relates to a IL2RG genomic DNA sequence of a humanized mouse. The DNA sequence is obtained by reverse transcription of the MRNA obtained by transcription thereof is consistent with or complementary to the DNA sequence homologous to the sequence shown in SEQ ID NO: 5, 26, or 29.

### IL2RB

Interleukin-2 receptor subunit beta (CD122), also known as IL2RB, IL2Rβ, IL15RB, or P70-75, is a protein that in humans is encoded by the IL2RB gene. The IL2 receptor, which is involved in T cell-mediated immune responses, is present in 3 forms with respect to ability to bind interleukin 2. The low affinity form is a monomer of the alpha subunit (also called CD25) and is not involved in signal transduction. The intermediate affinity form consists of a gamma/beta subunit heterodimer, while the high affinity form consists of an alpha/beta/gamma subunit heterotrimer. Both the intermediate and high affinity forms of the receptor are involved in receptor-mediated endocytosis and transduction of mitogenic signals from IL2. The protein encoded by this gene represents the beta subunit and is a type I membrane protein. Activation of the receptor increases proliferation of CD8+ effector T cells.

Typically, IL-2 is first captured by IL-2Rα through a large hydrophobic binding surface surrounded by a polar periphery that results in a relatively weak interaction with rapid on-off binding kinetics. The IL-2Rα-IL-2 binary complex leads to a very small conformational change in IL-2 that promotes association with IL-2Rβ through a distinct polar interaction between IL-2 and IL-2Rβ. Notably, the extracellular domain IL-2Rα does not interact with IL-2Rβ, but rather, the binary complex of IL-2Rα-IL-2 appears to present in cis IL-2 to IL-2Rβ. The ternary IL-2Rα-IL-2Rβ-IL-2 complex then recruits γ_{c} through a weak interaction with IL-2 and a stronger interaction with IL-2Rβ to produce a stable quaternary high-affinity IL-2R.

A detailed description of IL2RB and its function can be found, e.g., in Lin, J. et al. “The common cytokine receptor γ chain family of cytokines.” Cold Spring Harbor perspectives in biology 10.9 (2018): a028449; and Malek, T., et al. “Interleukin-2 receptor signaling: at the interface between tolerance and immunity.” Immunity 33.2 (2010): 153-165; each of which is incorporated by reference in its entirety.

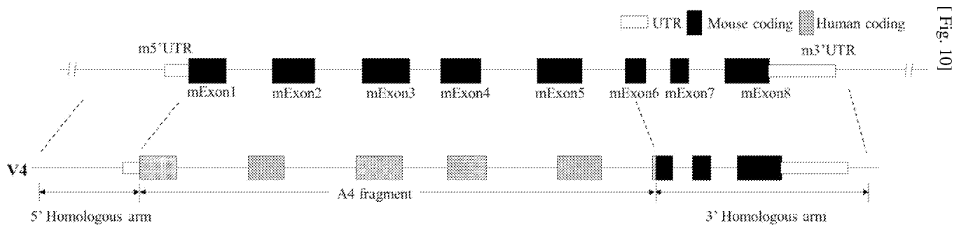

In human genomes, IL2RB gene (Gene ID: 3560) locus has ten exons, exon 1, exon 2, exon 3, exon 4, exon 5, exon 6, exon 7, exon 8, exon 9, and exon 10 (FIG. 11). The human IL2RB protein also has, from N-terminus to C-terminus, a signal peptide, an extracellular region, a transmembrane region, and a cytoplasmic region. The nucleotide sequence for human IL2RB mRNA is NM_000878.5, and the amino acid sequence for human IL2RB is NP_000869.1 (SEQ ID NO: 34). The location for each exon and each region in human IL2RB nucleotide sequence and amino acid sequence is listed below:

TABLE 3

| | | NP_000869.1 |
|---|---|---|
| Human IL2 RB | NM_000878.5 | 551 aa |
| (approximate location) | 4034 bp | SEQ ID NO: 34 |
| Exon 1 | 1 . . . 92 | Non-coding |
| Exon 2 | 93 . . . 213 | 1-29 |
| Exon 3 | 214 . . . 328 | 30-68 |
| Exon 4 | 329 . . . 407 | 69-94 |
| Exon 5 | 408 . . . 513 | 95-129 |
| Exon 6 | 514 . . . 662 | 130-179 |
| Exon 7 | 663 . . . 828 | 180-234 |
| Exon 8 | 829 . . . 943 | 235-273 |
| Exon 9 | 944 . . . 1028 | 274-301 |
| Exon 10 | 1029 . . . 4034 | 302-551 |
| Signal peptide | 126 . . . 203 | 1-26 |
| Extracellular | 204-845 | 27-240 |
| Transmembrane | 846-920 | 241-265 |
| Cytoplasmic | 921-1778 | 266-551 |
| Donor region in Example 3 | 210-836 | 29-237 |

The human IL2RB gene (Gene ID: 3560) is located in Chromosome 22 of the human genome, which is located from 37125838 to 37175118 of NC_000022.11 (GRCh38. p14 (GCF_000001405.40)). The 5′ UTR is from 37,149,825 to 37,149,916, Exon 1 is from 37,149,916 to 37,149,825, the first intron is from 37,149,824 to 37,144,206, Exon 2 is from 37,144,205 to 37,144,085, the second intron is from 37,144,084 to 37,143,636, Exon 3 is from 37,143,635 to 37,143,521, the third intron is from 37,143,520 to 37,142,513, Exon 4 is from 37,142,512 to 37,142,434, the fourth intron is from 37,142,433 to 37,139,223, Exon 5 is from 37,139,222 to 37,139,117 the fifth intron is from 37,139,116 to 37,137,736, Exon 6 is from 37,137,735 to 37,137,587, the sixth intron is from 37,137,586 to 37,136,394, Exon 7 is from 37,136,393 to 37,136,228, the seventh intron is from 37,136,227 to 37,135,443, Exon 8 is from 37,135,442 to 37,135,328, the eighth intron is from 37,135,327 to 37,132,469, Exon 9 is from 37,132,468 to 37,132,384, the ninth intron is from 37,132,383 to 37,128,849, Exon 10 is from 37,128,848 to 37,125,843, and the 3′UTR is from 37,125,843 to 37,128,095, based on transcript NM_000878.5. All relevant information for human IL2RB locus can be found in the NCBI website with Gene ID: 3560, which is incorporated by reference herein in its entirety.

In mice, IL2RB gene locus has ten exons, exon 1, exon 2, exon 3, exon 4, exon 5, exon 6, exon 7, exon 8, exon 9, and exon 10 (FIG. 11). The mouse IL2RB protein also has, from N-terminus to C-terminus, a signal peptide, an extracellular region, a transmembrane region, and a cytoplasmic region. The nucleotide sequence for mouse IL2RB mRNA is NM_008368.4, the amino acid sequence for mouse IL2RB is NP_032394.1 (SEQ ID NO: 33). The location for each exon and each region in the mouse IL2RB nucleotide sequence and amino acid sequence is listed below:

TABLE 4

| | | NP_032394.1 |
|---|---|---|
| Mouse IL2RB | NM_008368.4 | 539 aa |
| (approximate location) | 2712 bp | SEQ ID NO: 33 |
| Exon 1 | 1-119 | Non-coding |
| Exon 2 | 120-237 | 1-29 |
| Exon 3 | 238-352 | 30-68 |
| Exon 4 | 353-434 | 69-95 |
| Exon 5 | 435-540 | 96-130 |
| Exon 6 | 541-689 | 131-180 |
| Exon 7 | 690-855 | 181-235 |
| Exon 8 | 856-976 | 236-276 |
| Exon 9 | 977-1061 | 277-304 |
| Exon 10 | 1062-2697 | 305-539 |
| Signal peptide | 150-227 | 1-26 |
| Extracellular | 228-869 | 27-240 |
| Transmembrane | 870-953 | 241-268 |
| Cytoplasmic | 954-1766 | 269-539 |
| Replaced region in Example 3 | 234-863 | 29-238 |

The mouse IL2RB gene (Gene ID: 16185) is located in Chromosome 15 of the mouse genome, which is located from 78479256 to 78511621 of NC_000081.6 (GRCm38. p6 (GCF_000001635.26)). The 5′-UTR is from 78,495,271 to 78,494,948 and 78,491,879 to 78,491,850, exon 1 is from 78,495,271 to 78,494,948, the first intron is from 78,494,947 to 78,491,880, exon 2 is from 78,491,879 to 78,491,762, the second intron is from 78,491,761 to 78,490,980, exon 3 is from 78,490,979 to 78,490,865, the third intron is from 78,490,864 to 78,490,264, exon 4 is from 78,490,263 to 78,490,182, the fourth intron is from 78,490,181 to 78,488,231, exon 5 is from 78,488,230 to 78,488,125, the fifth intron is from 78,488,124 to 78,486,523, exon 6 is from 78,486,522 to 78,486,374, the sixth intron is from 78,486,373 to 78,485,898, exon 7 is from 78,485,897 to 78,485,732, the seventh intron is from 78,485,731 to 78,485,044, exon 8 is from 78,485,043 to 78,484,923, the eighth intron is from 78,484,922 to 78,484,021, exon 9 is from 78,484,020 to 78,483,936, the ninth intron is from 78,483,935 to 78,482,184, exon 10 is from 78,482,183 to 78,479,256, and the 3′-UTR is from 78,481,475 to 78,479,256, based on transcript NM_008368.4. All relevant information for mouse IL2RB locus can be found in the NCBI website with Gene ID: 16185, which is incorporated by reference herein in its entirety.

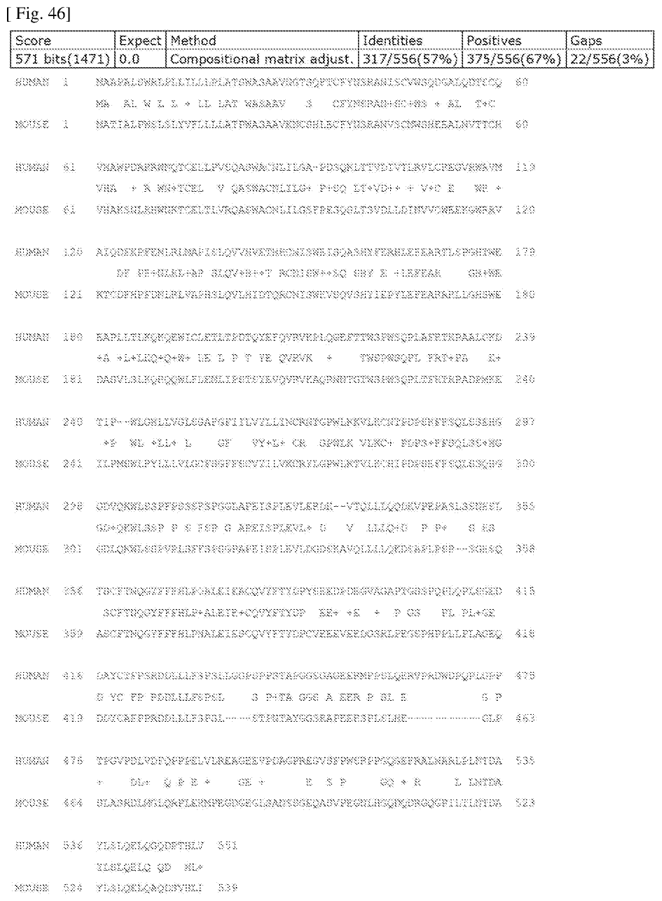

FIG. 46 shows the alignment between human IL2RB amino acid sequence (NP_000869.1; SEQ ID NO: 34) and mouse IL2RB amino acid sequence (NP_032394.1; SEQ ID NO: 33). Thus, the corresponding amino acid residue or region between human and mouse IL2RB can be found in FIG. 46.

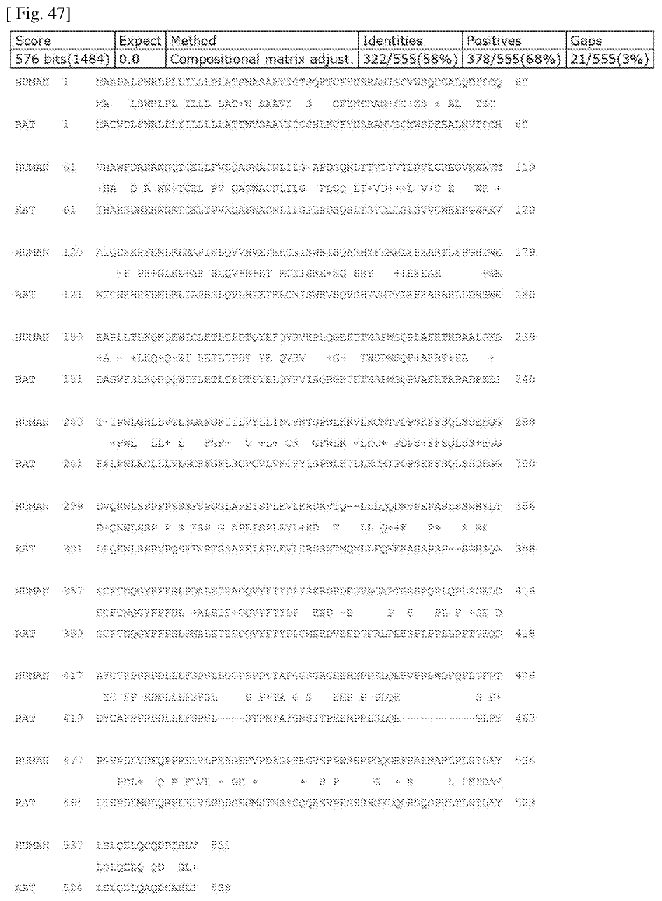

IL2RB genes, proteins, and locus of the other species are also known in the art. For example, the gene ID for IL2RB in Rattus norvegicus (rat) is 25746, the gene ID for IL2RB in Danio rerio (zebrafish) is 793920, the gene ID for IL2RB in Canis lupus familiaris (dog) is 403439, and the gene ID for IL2RB in Pan troglodytes (chimpanzee) is 470203. The relevant information for these genes (e.g., intron sequences, exon sequences, amino acid residues of these proteins) can be found, e.g., in NCBI database, which is incorporated by reference herein in its entirety. FIG. 47 shows the alignment between human IL2RB amino acid sequence (NP_000869.1; SEQ ID NO: 34) and rat IL2RB amino acid sequence (NP_037327.2; SEQ ID NO: 86. Thus, the corresponding amino acid residue or region between human and rodent IL2RB can be found in FIG. 47.

The present disclosure provides human or chimeric (e.g., humanized) IL2RB nucleotide sequence and/or amino acid sequences. In some embodiments, the entire sequence of mouse exon 1, exon 2, exon 3, exon 4, exon 5, exon 6, exon 7, exon 8, exon 9, exon 10, signal peptide, extracellular region, transmembrane region, and/or cytoplasmic region are replaced by the corresponding human sequence. In some embodiments, a “region” or “portion” of mouse exon 1, exon 2, exon 3, exon 4, exon 5, exon 6, exon 7, exon 8, exon 9, exon 10, signal peptide, extracellular region, transmembrane region, and/or cytoplasmic region are replaced by the corresponding human sequence. The term “region” or “portion” can refer to at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 250, 300, 350, 400, 450, 500, 550, 600, 610, 620, 625, 626, 627, 628, 629, 630, 650, 700, 750, 800, 850, 900, 950, 1000, 1500, 2000, 2500, 2700, 2712, 3000, 3500, 4000, or 4034 nucleotides, or at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 205, 206, 207, 208, 209, 210, 220, 230, 240, 250, 300, 350, 400, 450, 500, 530, 535, 539, 540, 550, or 551 amino acid residues. In some embodiments, the “region” or “portion” can be at least 50%, 55%, 60%, 65%, 70%, 75%, 80%, 85%, 90%, 95%, or 99% identical to exon 1, exon 2, exon 3, exon 4, exon 5, exon 6, exon 7, exon 8, exon 9, exon 10, signal peptide, extracellular region, transmembrane region, or cytoplasmic region. In some embodiments, a region, a portion, or the entire sequence of mouse exon 1, exon 2, exon 3, exon 4, exon 5, exon 6, exon 7, exon 8, exon 9, and/or exon 10 (e.g., a portion of exon 2, exons 3-7, and a portion of exon 8) are replaced by a region, a portion, or the entire sequence of the human exon 1, exon 2, exon 3, exon 4, exon 5, exon 6, exon 7, exon 8, exon 9, and/or exon 10 (e.g., a portion of exon 2, exons 3-7, and a portion of exon 8).

In some embodiments, a “region” or “portion” of the signal peptide, extracellular region, transmembrane region, cytoplasmic region, exon 1, exon 2, exon 3, exon 4, exon 5, exon 6, exon 7, exon 8, exon 9, and/or exon 10 is deleted.

In some embodiments, the present disclosure is related to a genetically-modified, non-human animal whose genome comprises a chimeric (e.g., humanized) IL2RB nucleotide sequence. In some embodiments, the chimeric (e.g., humanized) IL2RB nucleotide sequence encodes a IL2RB protein comprising a signal peptide, an extracellular region, a transmembrane region, and a cytoplasmic region. In some embodiments, the signal peptide comprises a sequence that is at least 80%, 85%, 90%, 95%, or 100% identical to amino acids 1-26 of SEQ ID NO: 33. In some embodiments, the signal peptide comprises all or part of endogenous IL2RB signal peptide. In some embodiments, the extracellular region comprises a sequence that is at least 80%, 85%, 90%, 95%, or 100% identical to amino acids 29-237 of SEQ ID NO: 34. In some embodiments, the extracellular region comprises all or part of human IL2RB extracellular region. In some embodiments, the extracellular region comprises at least 1 or 2 amino acids at the N-terminus of endogenous IL2RB extracellular region, and/or at least 1 or 2 amino acids at the C-terminus of endogenous IL2RB extracellular region. In some embodiments, the transmembrane region comprises a sequence that is at least 80%, 85%, 90%, 95%, or 100% identical to amino acids 241-268 of SEQ ID NO: 33. In some embodiments, the transmembrane region comprises all or part of endogenous IL2RB transmembrane region. In some embodiments, the cytoplasmic region comprises a sequence that is at least 80%, 85%, 90%, 95%, or 100% identical to amino acids 269-539 of SEQ ID NO: 33. In some embodiments, the cytoplasmic region comprises all or part of endogenous IL2RB cytoplasmic region. In some embodiments, the genome of the animal comprises a sequence that is at least 80%, 85%, 90%, 95%, or 100% identical to SEQ ID NO: 35, 36, 37, 38, 40, or 41.

In some embodiments, the genetically-modified non-human animal described herein comprises a sequence encoding a human or humanized IL2RB protein. In some embodiments, the IL2RB protein comprises, from N-terminus to C-terminus, a signal peptide, an extracellular region, a transmembrane region, and a cytoplasmic region. In some embodiments, the humanized IL2RB protein comprises a human or humanized signal peptide. In some embodiments, the humanized IL2RB protein comprises an endogenous signal peptide. In some embodiments, the humanized IL2RB protein comprises a human or humanized extracellular region. In some embodiments, the humanized IL2RB protein comprises an endogenous extracellular region. In some embodiments, the humanized IL2RB protein comprises a human or humanized transmembrane region. In some embodiments, the humanized IL2RB protein comprises an endogenous transmembrane region. In some embodiments, the humanized IL2RB protein comprises a human or humanized cytoplasmic region. In some embodiments, the humanized IL2RB protein comprises an endogenous cytoplasmic region. In some embodiments, the humanized IL2RB protein comprises an endogenous signal peptide, a human or humanized extracellular region, an endogenous transmembrane region, and an endogenous cytoplasmic region. In some embodiments, the humanized IL2RB protein comprises an endogenous sequence that corresponds to amino acids 1-28 and 239-539 of SEQ ID NO: 33.

In some embodiments, the genetically-modified non-human animal described herein comprises a human or humanized IL2RB gene. In some embodiments, the humanized IL2RB gene comprises 10 exons. In some embodiments, the humanized IL2RB gene comprises endogenous exon 1, humanized exon 2, human exon 3, human exon 4, human exon 5, human exon 6, human exon 7, humanized exon 8, endogenous exon 9, and/or endogenous exon 10. In some embodiments, the humanized IL2RB gene comprises 9 introns. In some embodiments, the humanized IL2RB gene comprises endogenous intron 1, human intron 2, human intron 3, human intron 4, human intron 5, human intron 6, human intron 7, endogenous intron 8, and endogenous intron 9. In some embodiments, the humanized IL2RB gene comprises human or humanized 5′ UTR. In some embodiments, the humanized IL2RB gene comprises human or humanized 3′ UTR. In some embodiments, the humanized IL2RB gene comprises endogenous 5′ UTR. In some embodiments, the humanized IL2RB gene comprises endogenous 3′ UTR.

Thus, in some embodiments, the present disclosure also provides a chimeric (e.g., humanized) IL2RB nucleotide sequence and/or amino acid sequences, wherein in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from mouse IL2RB mRNA sequence (e.g., NM_008368.4), mouse IL2RB amino acid sequence (e.g., SEQ ID NO: 33), or a portion thereof (e.g., exon 1, a portion of exon 2, a portion of exon 8, and exons 9-10); and in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from human IL2RB mRNA sequence (e.g., NM_000878.5), human IL2RB amino acid sequence (e.g., SEQ ID NO: 34), or a portion thereof (e.g., a portion of exon 2, exons 3-7, and a portion of exon 8).

In some embodiments, the sequence encoding amino acids 29-238 of mouse IL2RB (SEQ ID NO: 33) is replaced. In some embodiments, the sequence is replaced by a sequence encoding a corresponding region of human IL2RB (e.g., amino acids 29-237 of human IL2RB (SEQ ID NO: 34)).

In some embodiments, the sequence encoding amino acids 27-238, 29-240, or 27-240 of mouse IL2RB (SEQ ID NO: 33) is replaced. In some embodiments, the sequence is replaced by a sequence encoding a corresponding region of human IL2RB (e.g., amino acids 27-237, 29-240, or 27-240 of human IL2RB (SEQ ID NO: 34)).

In some embodiments, the nucleic acids as described herein are operably linked to a promotor or regulatory element, e.g., an endogenous mouse IL2RB promotor, an inducible promoter, an enhancer, and/or mouse or human regulatory elements.

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, 100, 150, 200, 250, 300, 350, 400, 450, 500, 550, 600, 620, 630, 640, 650, 700, 750, 800, 850, 900, 950, 1000, 1500, 2000, 2500, 2700, or 2712 nucleotides, e.g., contiguous or non-contiguous nucleotides) that are different from part of or the entire mouse IL2RB nucleotide sequence (e.g., a portion of exon 2, exons 3-7, and a portion of exon 8 of NM_008368.4).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, 100, 150, 200, 230, 231, 232, 233, 250, 300, 350, 400, 450, 500, 550, 600, 650, 700, 750, 800, 850, 900, 950, 1000, 1500, 1600, 1700, 1800, 1840, 1849, 1900, 2000, 2500, 2700, or 2712 nucleotides, e.g., contiguous or non-contiguous nucleotides) that is the same as part of or the entire mouse IL2RB nucleotide sequence (e.g., exon 1, a portion of exon 2, a portion of exon 8, and exons 9-10 of NM_008368.4).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, 100, 150, 200, 205, 206, 207, 208, 209, 210, 250, 300, 350, 400, 450, 500, 550, 600, 650, 700, 750, 800, 850, 900, 950, 1000, 1500, 2000, 2500, 3000, 3100, 3150, 3198, 3200, 3500, 4000, or 4034 nucleotides, e.g., contiguous or non-contiguous nucleotides) that is different from part of or the entire human IL2RB nucleotide sequence (e.g., exon 1, a portion of exon 2, a portion of exon 8, and exons 9-10 of NM_000878.5).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, 100, 150, 200, 250, 300, 350, 400, 450, 500, 550, 600, 620, 625, 626, 627, 630, 650, 700, 750, 800, 850, 900, 950, 1000, 1500, 2000, 2500, 3000, 3500, 4000, or 4034 nucleotides, e.g., contiguous or non-contiguous nucleotides) that is the same as part of or the entire human IL2RB nucleotide sequence (e.g., a portion of exon 2, exons 3-7, and a portion of exon 8 of NM_000878.5).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, 100, 110, 120, 130, 140, 150, 160, 170, 180, 190, 200, 210, 220, 230, 240, 250, 300, 350, 400, 450, 500, or 539 amino acid residues, e.g., contiguous or non-contiguous amino acid residues) that is different as part of or the entire mouse IL2RB amino acid sequence (e.g., amino acids 29-238 of NP_032394.1 (SEQ ID NO: 33)).

In some embodiments, the amino acid sequence has at least a portion (e.g., at least 1, 2, 3, 4, 5, 6, 7, 8, 9, 10, 11, 12, 13, 14, 15, 20, 28, 29, 30, 40, 50, 60, 70, 80, 90, 100, 150, 200, 250, 300, 301, 302, 303, 304, 305, 310, 350, 400, 450, 500, or 539 amino acid residues, e.g., contiguous or non-contiguous amino acid residues) that is the same from part of or the entire mouse IL2RB amino acid sequence (e.g., amino acids 1-28 and 239-539 of NP_032394.1 (SEQ ID NO: 33)).

In some embodiments, the amino acid sequence has at least a portion (e.g., at least 1, 2, 3, 4, 5, 6, 7, 8, 9, 10, 11, 12, 13, 14, 15, 20, 25, 26, 27, 28, 29, 30, 40, 50, 60, 70, 80, 90, 100, 150, 200, 250, 300, 310, 311, 312, 313, 314, 315, 320, 350, 400, 450, 500, 550, or 551 amino acid residues, e.g., contiguous or non-contiguous amino acid residues) that is different from part of or the entire human IL2RB amino acid sequence (e.g., amino acids 1-28 and 238-551 of NP_000869.1 (SEQ ID NO: 34)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, 100, 110, 120, 130, 140, 150, 160, 170, 180, 190, 200, 205, 206, 207, 208, 209, 210, 220, 230, 240, 250, 300, 350, 400, 450, 500, 550, or 551 amino acid residues, e.g., contiguous or non-contiguous amino acid residues) that is the same as part of or the entire human IL2RB amino acid sequence (e.g., amino acids 29-237 of NP_000869.1 (SEQ ID NO: 34)).

The present disclosure also provides a humanized IL2RB mouse amino acid sequence, wherein the amino acid sequence is selected from the group consisting of:
a) an amino acid sequence shown in SEQ ID NO: 33, 34, or 39;
b) an amino acid sequence having a homology of at least 90% with or at least 90% identical to the amino acid sequence shown in SEQ ID NO: 33, 34, or 39;
c) an amino acid sequence encoded by a nucleic acid sequence, wherein the nucleic acid sequence is able to hybridize to a nucleotide sequence encoding the amino acid shown in SEQ ID NO: 33, 34, or 39 under a low stringency condition or a strict stringency condition;
d) an amino acid sequence having a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the amino acid sequence shown in SEQ ID NO: 33, 34, or 39;
e) an amino acid sequence that is different from the amino acid sequence shown in SEQ ID NO: 33, 34, or 39 by no more than 10, 9, 8, 7, 6, 5, 4, 3, 2 or no more than 1 amino acid; or
f) an amino acid sequence that comprises a substitution, a deletion and/or insertion of one or more amino acids to the amino acid sequence shown in SEQ ID NO: 33, 34, or 39.

The present disclosure also provides a humanized IL2RB amino acid sequence, wherein the amino acid sequence is selected from the group consisting of:
a) all or part of amino acids 29-237 of SEQ ID NO: 34;
b) an amino acid sequence have a homology of at least 90%, 91%, 92%, 93%, 94%, 95%, 96%, 97%, 98%, or 99% to amino acids 29-237 of SEQ ID NO: 34;
c) an amino acid sequence that is different from amino acids 29-237 of SEQ ID NO: 34 by no more than 10, 9, 8, 7, 6, 5, 4, 3, 2 or no more than 1 amino acid; and
d) an amino acid sequence that comprises a substitution, a deletion and/or insertion of one or more amino acids to amino acids 29-237 of SEQ ID NO: 34.

The present disclosure also provides a humanized IL2RB amino acid sequence, wherein the amino acid sequence is selected from the group consisting of:
a) all or part of amino acids 1-28 and/or 239-539 of SEQ ID NO: 33;
b) an amino acid sequence have a homology of at least 90%, 91%, 92%, 93%, 94%, 95%, 96%, 97%, 98%, or 99% to amino acids 1-28 and/or 239-539 of SEQ ID NO: 33;
c) an amino acid sequence that is different from amino acids 1-28 and/or 239-539 of SEQ ID NO: 33 by no more than 10, 9, 8, 7, 6, 5, 4, 3, 2 or no more than 1 amino acid; and
d) an amino acid sequence that comprises a substitution, a deletion and/or insertion of one or more amino acids to amino acids 1-28 and/or 239-539 of SEQ ID NO: 33.

The present disclosure also relates to a IL2RB nucleic acid (e.g., DNA or RNA) sequence, wherein the nucleic acid sequence can be selected from the group consisting of:
a) a nucleic acid sequence as shown in SEQ ID NO: 35, 36, 37, 38, 40, or 41, or a nucleic acid sequence encoding a homologous IL2RB amino acid sequence of a humanized mouse IL2RB;
b) a nucleic acid sequence that is able to hybridize to the nucleotide sequence as shown in SEQ ID NO: 35, 36, 37, 38, 40, or 41 under a low stringency condition or a strict stringency condition;
c) a nucleic acid sequence that has a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the nucleotide sequence as shown in SEQ ID NO: 35, 36, 37, 38, 40, or 41;
d) a nucleic acid sequence that encodes an amino acid sequence, wherein the amino acid sequence has a homology of at least 90% with or at least 90% identical to the amino acid sequence shown in SEQ ID NO: 33, 34, or 39;
e) a nucleic acid sequence that encodes an amino acid sequence, wherein the amino acid sequence has a homology of at least 90%, 91%, 92%, 93%, 94%, 95%, 96%, 97%, 98%, or 99% with, or at least 90%, 91%, 92%, 93%, 94%, 95%, 96%, 97%, 98%, or 99% identical to the amino acid sequence shown in SEQ ID NO: 33, 34, or 39;
f) a nucleic acid sequence that encodes an amino acid sequence, wherein the amino acid sequence is different from the amino acid sequence shown in SEQ ID NO: 33, 34, or 39 by no more than 10, 9, 8, 7, 6, 5, 4, 3, 2 or no more than 1 amino acid; and/or
g) a nucleic acid sequence that encodes an amino acid sequence, wherein the amino acid sequence comprises a substitution, a deletion and/or insertion of one or more amino acids to the amino acid sequence shown in SEQ ID NO: 33, 34, or 39.

The present disclosure further relates to a IL2RB genomic DNA sequence of a humanized mouse. The DNA sequence is obtained by reverse transcription of the MRNA obtained by transcription thereof is consistent with or complementary to the DNA sequence homologous to the sequence shown in SEQ ID NO: 37 or 38.

### IL15RA

Interleukin 15 receptor, alpha subunit (CD215), also known as IL15RA or IL15Ra, is a subunit of the interleukin 15 receptor that in humans is encoded by the IL15RA gene. The IL-15 receptor is composed of three subunits: IL-15R alpha, CD122, and CD132. Two of these subunits, CD122 and CD132, are shared with the receptor for IL-2, but IL-2 receptor has an additional subunit (CD25). The shared subunits contain the cytoplasmic motifs required for signal transduction, and this forms the basis of many overlapping biological activities of IL15 and IL2, although in vivo the two cytokines have separate biological effects. IL-15R alpha specifically binds IL15 with very high affinity, and is capable of binding IL-15 independently of other subunits. It is suggested that this property allows IL-15 to be produced by one cell, endocytosed by another cell, and then presented to a third party cell. This receptor is reported to enhance cell proliferation and expression of apoptosis inhibitor BCL2L1/BCL2-XL and BCL2. Multiple alternatively spliced transcript variants of this gene have been reported.

IL-15Rα can be expressed on the surface of T or NK cells, forming an IL-15Rα/IL-2Rβ/γc trimeric receptor. However, IL-15Rα appears to be mainly expressed by antigen-presenting cells. It binds IL-15 with a high affinity, allowing a producing cell to present IL-15 in trans via IL-15Rα to a neighboring cell that expresses the IL-2Rβ/γc complex. This original mechanism of action is called IL-15 trans-presentation. Thus, IL-15 can act both in cis, like IL-2, but also in trans. In addition, a soluble (s) form of IL-15Rα (sIL-15Rα) can act either as an antagonist of IL-15 action, competing with membrane-bound IL-15Rα for the binding of IL-15 or, as an agonist, forming an IL-15Rα/IL-15 complex activating the IL-2Rβ/γc dimeric receptor more efficiently than IL-15 alone. Thus, numerous laboratories turned the latter observation into therapeutic applications to mimic trans-presentation of soluble IL-15Rα/IL-15 (sIL-15Rα/IL-15, also referred to as ‘trans-signaling’ of IL-15Rα/IL-15).

A detailed description of IL15RA and its function can be found, e.g., in Mishra, A., et al. “Molecular pathways: interleukin-15 signaling in health and in cancer.” Clinical Cancer Research 20.8 (2014): 2044-2050; and Quéméner, A., et al. “IL-15Rα membrane anchorage in either cis or trans is required for stabilization of IL-15 and optimal signaling.” Journal of Cell Science 133.5 (2020): jcs236802; each of which is incorporated by reference in its entirety.

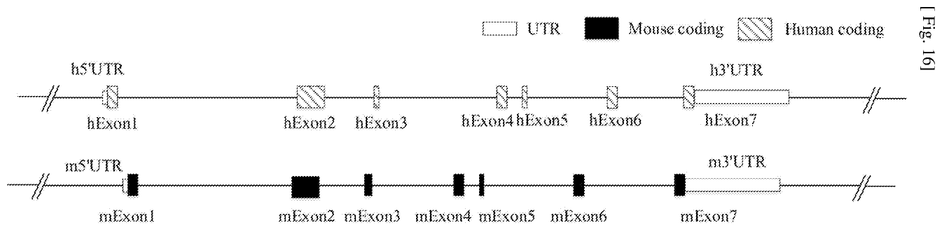

In human genomes, IL15RA gene (Gene ID: 3601) locus has seven exons, exon 1, exon 2, exon 3, exon 4, exon 5, exon 6, and exon 7 (FIG. 16). The human IL15RA protein also has, from N-terminus to C-terminus, a signal peptide, an extracellular region, a transmembrane region, and a cytoplasmic region. The nucleotide sequence for human IL15RA mRNA is NM_002189.4, and the amino acid sequence for human IL15RA is NP_002180.1 (SEQ ID NO: 43). The location for each exon and each region in human IL15RA nucleotide sequence and amino acid sequence is listed below:

TABLE 5

| | | NP_002180.1 |
|---|---|---|
| Human IL15RA | NM_002189.4 | 267 aa |
| (approximate location) | 1566 bp | SEQ ID NO: 43 |
| Exon 1 | 1-139 | 1-29 |
| Exon 2 | 140-334 | 30-94 |
| Exon 3 | 335-433 | 95-127 |
| Exon 4 | 434-634 | 128-194 |
| Exon 5 | 635-667 | 195-205 |
| Exon 6 | 668-743 | 206-231 |
| Exon 7 | 744-1566 | 232-267 |
| Signal peptide | 1-141 | 1-30 |
| Extracellular | 142-666 | 31-205 |
| Transmembrane | 667-735 | 206-228 |
| Cytoplasmic | 736-852 | 229-267 |
| Donor region in Example 5 | 160-684 | 37-211 |

The human IL15RA gene (Gene ID: 3601) is located in Chromosome 10 of the human genome, which is located from 5948897 to 5978741 of NC_000010.11 (GRCh38. p14 (GCF_000001405.40)). The 5′ UTR is from 5,977,495 to 5,977,543, Exon 1 is from 5,977,543 to 5,977,405, the first intron is from 5,977,404 to 5,966,340, Exon 2 is from 5,966,339 to 5,966,145, the second intron is from 5,966,144 to 5,963,842, Exon 3 is from 5,963,841 to 5,963,743, the third intron is from 5,963,742 to 5,960,568, Exon 4 is from 5,960,567 to 5,960,367, the fourth intron is from 5,960,366 to 5,959,787, Exon 5 is from 5,959,786 to 5,959,754, the fifth intron is from 5,959,753 to 5,956,455, Exon 6 is from 5,956,454 to 5,956,379, the sixth intron is from 5,956,378 to 5,953,207, Exon 7 is from 5,953,206 to 5,952,384, and the 3′UTR is from 5,952,384 to 5,953,094, based on transcript NM_002189.4. All relevant information for human IL15RA locus can be found in the NCBI website with Gene ID: 3601, which is incorporated by reference herein in its entirety.

In mice, IL15RA gene locus has seven exons, exon 1, exon 2, exon 3, exon 4, exon 5, exon 6, and exon 7 (FIG. 16). The mouse IL15RA protein also has, from N-terminus to C-terminus, a signal peptide, an extracellular region, a transmembrane region, and a cytoplasmic region. The nucleotide sequence for mouse IL15RA mRNA is NM_008358.2, the amino acid sequence for mouse IL15RA is NP_032384.1 (SEQ ID NO: 42). The location for each exon and each region in the mouse IL15RA nucleotide sequence and amino acid sequence is listed below:

TABLE 6

| | | NP_032384.1 |
|---|---|---|
| Mouse IL15RA | NM_008358.2 | 263 aa |
| (approximate location) | 1664 bp | SEQ ID NO: 42 |
| Exon 1 | 1-233 | 1-32 |
| Exon 2 | 234-428 | 33-97 |
| Exon 3 | 429-521 | 98-128 |
| Exon 4 | 522-719 | 129-194 |
| Exon 5 | 720-752 | 195-205 |
| Exon 6 | 753-828 | 206-231 |
| Exon 7 | 829-1664 | 232-263 |
| Signal peptide | 1-233 | 1-32 |
| Extracellular | 234-751 | 33-205 |
| Transmembrane | 752-814 | 206-226 |
| Cytoplasmic | 815-925 | 227-263 |
| Replaced region in Example 5 | 254-769 | 40-211 |

The mouse IL15RA gene (Gene ID: 16169) is located in Chromosome 2 of the mouse genome, which is located from 11709992 to 11738796 of NC_000068.8 (GRCm39 (GCF_000001635.27)). The 5′ UTR is from 11,710,588 to 11,710,658, Exon 1 is from 11,710,588 to 11,710,755, the first intron is from 11,710,756 to 11,723,074, Exon 2 is from 11,723,075 to 11,723,269, the second intron is from 11,723,270 to 11,724,823, Exon 3 is from 11,724,824 to 11,724,916, the third intron is from 11,724,917 to 11,728,223, Exon 4 is from 11,728,224 to 11,728,421, the fourth intron is from 11,728,422 to 11,728,906, Exon 5 is from 11,728,907 to 11,728,939 the fifth intron is from 11,728,940 to 11,735,381, Exon 6 is from 11,735,382 to 11,735,457, the sixth intron is from 11,735,458 to 11,737,960, Exon 7 is from 11,737,961 to 11,738,797, and the 3′UTR is from 11,738,061 to 11,738,797, based on transcript NM_008358.2. All relevant information for mouse IL15RA locus can be found in the NCBI website with Gene ID: 16169, which is incorporated by reference herein in its entirety.

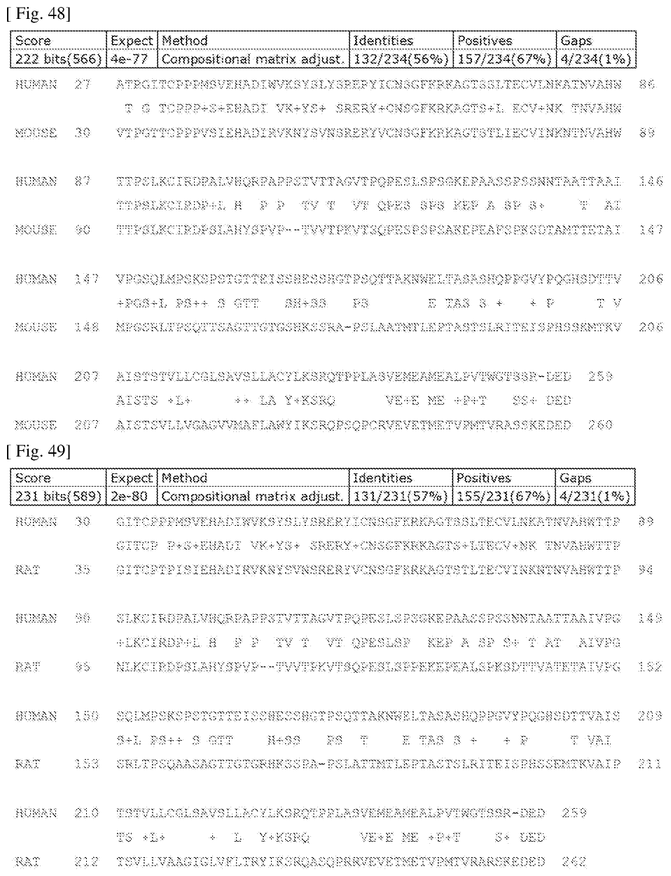

FIG. 48 shows the alignment between human IL15RA amino acid sequence (NP_002180.1; SEQ ID NO: 43) and mouse IL15RA amino acid sequence (NP_032384.1; SEQ ID NO: 42). Thus, the corresponding amino acid residue or region between human and mouse IL15RA can be found in FIG. 48.

IL15RA genes, proteins, and locus of the other species are also known in the art. For example, the gene ID for IL15RA in Rattus norvegicus (rat) is 690369, the gene ID for IL15RA in Macaca mulatta (Rhesus monkey) is 712788, the gene ID for IL15RA in Canis lupus familiaris (dog) is 487141, and the gene ID for IL15RA in Sus scrofa (pig) is 733692. The relevant information for these genes (e.g., intron sequences, exon sequences, amino acid residues of these proteins) can be found, e.g., in NCBI database, which is incorporated by reference herein in its entirety. FIG. 49 shows the alignment between human IL15RA amino acid sequence (NP_002180.1; SEQ ID NO: 43) and rat IL15RA amino acid sequence (XP_002728555.2; SEQ ID NO: 87. Thus, the corresponding amino acid residue or region between human and rodent IL15RA can be found in FIG. 49.

The present disclosure provides human or chimeric (e.g., humanized) IL15RA nucleotide sequence and/or amino acid sequences. In some embodiments, the entire sequence of mouse exon 1, exon 2, exon 3, exon 4, exon 5, exon 6, exon 7, signal peptide, extracellular region, transmembrane region, and/or cytoplasmic region are replaced by the corresponding human sequence. In some embodiments, a “region” or “portion” of mouse exon 1, exon 2, exon 3, exon 4, exon 5, exon 6, exon 7, signal peptide, extracellular region, transmembrane region, and/or cytoplasmic region are replaced by the corresponding human sequence. The term “region” or “portion” can refer to at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 250, 300, 350, 400, 450, 500, 510, 515, 516, 517, 518, 519, 520, 521, 522, 523, 524, 525, 530, 540, 550, 600, 650, 700, 750, 800, 850, 900, 950, 1000, 1100, 1200, 1300, 1400, 1500, 1550, 1566, 1600, 1650, or 1664 nucleotides, or at least 1, 2, 3, 4, 5, 6, 7, 8, 9, 10, 20, 30, 40, 50, 60, 70, 80, 90, 100, 110, 120, 130, 140, 150, 160, 170, 171, 172, 173, 174, 175, 176, 177, 178, 179, 180, 190, 200, 210, 220, 230, 240, 250, 260, 261, 262, 263, 264, 265, 266, or 267 amino acid residues. In some embodiments, the “region” or “portion” can be at least 50%, 55%, 60%, 65%, 70%, 75%, 80%, 85%, 90%, 95%, or 99% identical to exon 1, exon 2, exon 3, exon 4, exon 5, exon 6, exon 7, signal peptide, extracellular region, transmembrane region, or cytoplasmic region. In some embodiments, a region, a portion, or the entire sequence of mouse exon 1, exon 2, exon 3, exon 4, exon 5, exon 6, and/or exon 7 (e.g., a portion of exon 2, exons 3-5, and a portion of exon 6) are replaced by a region, a portion, or the entire sequence of the human exon 1, exon 2, exon 3, exon 4, exon 5, exon 6, and/or exon 7 (e.g., a portion of exon 2, exons 3-5, and a portion of exon 6).

In some embodiments, a “region” or “portion” of the signal peptide, extracellular region, transmembrane region, cytoplasmic region, exon 1, exon 2, exon 3, exon 4, exon 5, exon 6, and/or exon 7 is deleted.

In some embodiments, the present disclosure is related to a genetically-modified, non-human animal whose genome comprises a chimeric (e.g., humanized) IL15RA nucleotide sequence. In some embodiments, the chimeric (e.g., humanized) IL15RA nucleotide sequence encodes a IL15RA protein comprising a signal peptide, an extracellular region, a transmembrane region, and a cytoplasmic region. In some embodiments, the signal peptide comprises a sequence that is at least 80%, 85%, 90%, 95%, or 100% identical to amino acids 1-32 of SEQ ID NO: 42. In some embodiments, the signal peptide comprises all or part of endogenous IL15RA signal peptide. In some embodiments, the extracellular region comprises a sequence that is at least 80%, 85%, 90%, 95%, or 100% identical to amino acids 37-205 of SEQ ID NO: 43. In some embodiments, the extracellular region comprises all or part of human IL15RA extracellular region. In some embodiments, the extracellular region comprises at least 1, 2, 3, 4, 5, 6, or 7 amino acids at the N-terminus of endogenous IL15RA extracellular region. In some embodiments, the transmembrane region comprises a sequence that is at least 80%, 85%, 90%, 95%, or 100% identical to amino acids 212-226 of SEQ ID NO: 42. In some embodiments, the transmembrane region comprises all or part of endogenous IL15RA transmembrane region. In some embodiments, the transmembrane region comprises at least 1, 2, 3, 4, 5 or 6 amino acids at the N-terminus of human IL15RA transmembrane region. In some embodiments, the transmembrane region comprises a sequence that is at least 80%, 85%, 90%, 95%, or 100% identical to amino acids 206-211 of SEQ ID NO: 43. In some embodiments, the cytoplasmic region comprises a sequence that is at least 80%, 85%, 90%, 95%, or 100% identical to amino acids 227-263 of SEQ ID NO: 42. In some embodiments, the cytoplasmic region comprises all or part of endogenous IL15RA cytoplasmic region. In some embodiments, the genome of the animal comprises a sequence that is at least 80%, 85%, 90%, 95%, or 100% identical to SEQ ID NO: 44, 45, 46, 47, 48, or 49.

In some embodiments, the genetically-modified non-human animal described herein comprises a sequence encoding a human or humanized IL15RA protein. In some embodiments, the IL15RA protein comprises, from N-terminus to C-terminus, a signal peptide, an extracellular region, a transmembrane region, and a cytoplasmic region. In some embodiments, the humanized IL15RA protein comprises a human or humanized signal peptide. In some embodiments, the humanized IL15RA protein comprises an endogenous signal peptide. In some embodiments, the humanized IL15RA protein comprises a human or humanized extracellular region. In some embodiments, the humanized IL15RA protein comprises an endogenous extracellular region. In some embodiments, the humanized IL15RA protein comprises a human or humanized transmembrane region. In some embodiments, the humanized IL15RA protein comprises an endogenous transmembrane region. In some embodiments, the humanized IL15RA protein comprises a human or humanized cytoplasmic region. In some embodiments, the humanized IL15RA protein comprises an endogenous cytoplasmic region. In some embodiments, the humanized IL15RA protein comprises an endogenous signal peptide, a human or humanized extracellular region, a human or humanized transmembrane region, and an endogenous cytoplasmic region. In some embodiments, the humanized IL15RA protein comprises an endogenous sequence that corresponds to amino acids 1-39 and 212-263 of SEQ ID NO: 42.

In some embodiments, the genetically-modified non-human animal described herein comprises a human or humanized IL15RA gene. In some embodiments, the humanized IL15RA gene comprises 7 exons. In some embodiments, the humanized IL15RA gene comprises endogenous exon 1, humanized exon 2, human exon 3, human exon 4, human exon 5, humanized exon 6, and/or endogenous exon 7. In some embodiments, the humanized IL15RA gene comprises 6 introns. In some embodiments, the humanized IL15RA gene comprises endogenous intron 1, human intron 2, human intron 3, human intron 4, human intron 5, and endogenous intron 6 (e.g., optionally inserted with sequences of Neo cassette). In some embodiments, the humanized IL15RA gene comprises human or humanized 5′ UTR. In some embodiments, the humanized IL15RA gene comprises human or humanized 3′ UTR. In some embodiments, the humanized IL15RA gene comprises endogenous 5′ UTR. In some embodiments, the humanized IL15RA gene comprises endogenous 3′ UTR.

Thus, in some embodiments, the present disclosure also provides a chimeric (e.g., humanized) IL15RA nucleotide sequence and/or amino acid sequences, wherein in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from mouse IL15RA mRNA sequence (e.g., NM_008358.2), mouse IL15RA amino acid sequence (e.g., SEQ ID NO: 42), or a portion thereof (e.g., exon 1, a portion of exon 2, a portion of exon 6, and exon 7); and in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from human IL15RA mRNA sequence (e.g., NM_002189.4), human IL15RA amino acid sequence (e.g., SEQ ID NO: 43), or a portion thereof (e.g., a portion of exon 2, exons 3-5, and a portion of exon 6).

In some embodiments, the sequence encoding amino acids 40-211 of mouse IL15RA (SEQ ID NO: 42) is replaced. In some embodiments, the sequence is replaced by a sequence encoding a corresponding region of human IL15RA (e.g., amino acids 37-211 of human IL15RA (SEQ ID NO: 43)).

In some embodiments, the sequence encoding amino acids 33-205 of mouse IL15RA (SEQ ID NO: 42) is replaced. In some embodiments, the sequence is replaced by a sequence encoding a corresponding region of human IL15RA (e.g., amino acids 31-205 of human IL15RA (SEQ ID NO: 43)).

In some embodiments, the nucleic acids as described herein are operably linked to a promotor or regulatory element, e.g., an endogenous mouse IL15RA promotor, an inducible promoter, an enhancer, and/or mouse or human regulatory elements.

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, 100, 150, 200, 250, 300, 350, 400, 450, 500, 510, 514, 515, 516, 517, 518, 519, 520, 530, 550, 600, 650, 700, 750, 800, 850, 900, 950, 1000, 1100, 1200, 1300, 1400, 1500, 1600, 1650, or 1664 nucleotides, e.g., contiguous or non-contiguous nucleotides) that are different from part of or the entire mouse IL15RA nucleotide sequence (e.g., a portion of exon 2, exons 3-5, and a portion of exon 6 of NM_008358.2).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, 100, 150, 200, 250, 251, 252, 253, 254, 255, 256, 260, 300, 350, 400, 450, 500, 550, 600, 650, 700, 750, 800, 850, 880, 890, 891, 892, 893, 894, 895, 900, 950, 1000, 1100, 1200, 1300, 1400, 1500, 1600, 1650, or 1664 nucleotides, e.g., contiguous or non-contiguous nucleotides) that is the same as part of or the entire mouse IL15RA nucleotide sequence (e.g., exon 1, a portion of exon 2, a portion of exon 6, and exon 7 of NM_008358.2).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, 100, 150, 155, 156, 157, 158, 159, 160, 200, 250, 300, 350, 400, 450, 500, 550, 600, 650, 700, 750, 800, 850, 880, 881, 882, 883, 884, 885, 900, 950, 1000, 1100, 1200, 1300, 1400, 1500, 1550, or 1566 nucleotides, e.g., contiguous or non-contiguous nucleotides) that is different from part of or the entire human IL15RA nucleotide sequence (e.g., exon 1, a portion of exon 2, a portion of exon 6, and exon 7 of NM_002189.4).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, 100, 150, 200, 250, 300, 350, 400, 450, 500, 510, 520, 521, 522, 523, 524, 525, 530, 550, 600, 650, 700, 750, 800, 850, 900, 950, 1000, 1100, 1200, 1300, 1400, 1500, 1550, or 1566 nucleotides, e.g., contiguous or non-contiguous nucleotides) that is the same as part of or the entire human IL15RA nucleotide sequence (e.g., a portion of exon 2, exons 3-5, and a portion of exon 6 of NM_002189.4).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, 100, 110, 120, 130, 140, 150, 160, 170, 171, 172, 173, 174, 175, 180, 190, 200, 210, 220, 230, 240, 250, 260, 261, 262, or 263 amino acid residues, e.g., contiguous or non-contiguous amino acid residues) that is different as part of or the entire mouse IL15RA amino acid sequence (e.g., amino acids 40-211 of NP_032384.1 (SEQ ID NO: 42)).

In some embodiments, the amino acid sequence has at least a portion (e.g., at least 1, 2, 3, 4, 5, 6, 7, 8, 9, 10, 11, 12, 13, 14, 15, 20, 28, 29, 30, 35, 36, 37, 38, 39, 40, 50, 51, 52, 60, 70, 80, 90, 100, 150, 160, 170, 180, 190, 200, 210, 220, 230, 240, 250, 260, 261, 262, or 263 amino acid residues, e.g., contiguous or non-contiguous amino acid residues) that is the same from part of or the entire mouse IL15RA amino acid sequence (e.g., amino acids 1-39 and 212-263 of NP_032384.1 (SEQ ID NO: 42)).

In some embodiments, the amino acid sequence has at least a portion (e.g., at least 1, 2, 3, 4, 5, 6, 7, 8, 9, 10, 11, 12, 13, 14, 15, 20, 25, 26, 27, 28, 29, 30, 35, 36, 37, 38, 39, 40, 50, 55, 56, 57, 58, 59, 60, 70, 80, 90, 100, 160, 170, 180, 190, 200, 210, 220, 230, 240, 250, 260, 261, 262, 263, 264, 265, 266, or 267 amino acid residues, e.g., contiguous or non-contiguous amino acid residues) that is different from part of or the entire human IL15RA amino acid sequence (e.g., amino acids 1-36 and 212-267 of NP_002180.1 (SEQ ID NO: 43)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, 100, 110, 120, 130, 140, 150, 160, 170, 171, 172, 173, 174, 175, 180, 190, 200, 210, 220, 230, 240, 250, 260, 261, 262, 263, 264, 265, 266, or 267 amino acid residues, e.g., contiguous or non-contiguous amino acid residues) that is the same as part of or the entire human IL15RA amino acid sequence (e.g., amino acids 37-211 of NP_002180.1 (SEQ ID NO: 43)).

The present disclosure also provides a humanized IL15RA mouse amino acid sequence, wherein the amino acid sequence is selected from the group consisting of:
a) an amino acid sequence shown in SEQ ID NO: 42, 43, or 50;
b) an amino acid sequence having a homology of at least 90% with or at least 90% identical to the amino acid sequence shown in SEQ ID NO: 42, 43, or 50;
c) an amino acid sequence encoded by a nucleic acid sequence, wherein the nucleic acid sequence is able to hybridize to a nucleotide sequence encoding the amino acid shown in SEQ ID NO: 42, 43, or 50 under a low stringency condition or a strict stringency condition;
d) an amino acid sequence having a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the amino acid sequence shown in SEQ ID NO: 42, 43, or 50;
e) an amino acid sequence that is different from the amino acid sequence shown in SEQ ID NO: 42, 43, or 50 by no more than 10, 9, 8, 7, 6, 5, 4, 3, 2 or no more than 1 amino acid; or
f) an amino acid sequence that comprises a substitution, a deletion and/or insertion of one or more amino acids to the amino acid sequence shown in SEQ ID NO: 42, 43, or 50.

The present disclosure also provides a humanized IL15RA amino acid sequence, wherein the amino acid sequence is selected from the group consisting of:
a) all or part of amino acids 37-211 of SEQ ID NO: 43;
b) an amino acid sequence have a homology of at least 90%, 91%, 92%, 93%, 94%, 95%, 96%, 97%, 98%, or 99% to amino acids 37-211 of SEQ ID NO: 43;
c) an amino acid sequence that is different from amino acids 37-211 of SEQ ID NO: 43 by no more than 10, 9, 8, 7, 6, 5, 4, 3, 2 or no more than 1 amino acid; and
d) an amino acid sequence that comprises a substitution, a deletion and/or insertion of one or more amino acids to amino acids 37-211 of SEQ ID NO: 43.

The present disclosure also provides a humanized IL15RA amino acid sequence, wherein the amino acid sequence is selected from the group consisting of:
a) all or part of amino acids 1-39 and/or 212-263 of SEQ ID NO: 42;
b) an amino acid sequence have a homology of at least 90%, 91%, 92%, 93%, 94%, 95%, 96%, 97%, 98%, or 99% to amino acids 1-39 and/or 212-263 of SEQ ID NO: 42;
c) an amino acid sequence that is different from amino acids 1-39 and/or 212-263 of SEQ ID NO: 42 by no more than 10, 9, 8, 7, 6, 5, 4, 3, 2 or no more than 1 amino acid; and
d) an amino acid sequence that comprises a substitution, a deletion and/or insertion of one or more amino acids to amino acids 1-39 and/or 212-263 of SEQ ID NO: 42.

The present disclosure also relates to a IL15RA nucleic acid (e.g., DNA or RNA) sequence, wherein the nucleic acid sequence can be selected from the group consisting of:
a) a nucleic acid sequence as shown in SEQ ID NO: 44, 45, 46, 47, 48, or 49, or a nucleic acid sequence encoding a homologous IL15RA amino acid sequence of a humanized mouse IL15RA;
b) a nucleic acid sequence that is able to hybridize to the nucleotide sequence as shown in SEQ ID NO: 44, 45, 46, 47, 48, or 49 under a low stringency condition or a strict stringency condition;
c) a nucleic acid sequence that has a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the nucleotide sequence as shown in SEQ ID NO: 44, 45, 46, 47, 48, or 49;
d) a nucleic acid sequence that encodes an amino acid sequence, wherein the amino acid sequence has a homology of at least 90% with or at least 90% identical to the amino acid sequence shown in SEQ ID NO: 42, 43, or 50;
e) a nucleic acid sequence that encodes an amino acid sequence, wherein the amino acid sequence has a homology of at least 90%, 91%, 92%, 93%, 94%, 95%, 96%, 97%, 98%, or 99% with, or at least 90%, 91%, 92%, 93%, 94%, 95%, 96%, 97%, 98%, or 99% identical to the amino acid sequence shown in SEQ ID NO: 42, 43, or 50;
f) a nucleic acid sequence that encodes an amino acid sequence, wherein the amino acid sequence is different from the amino acid sequence shown in SEQ ID NO: 42, 43, or 50 by no more than 10, 9, 8, 7, 6, 5, 4, 3, 2 or no more than 1 amino acid; and/or
g) a nucleic acid sequence that encodes an amino acid sequence, wherein the amino acid sequence comprises a substitution, a deletion and/or insertion of one or more amino acids to the amino acid sequence shown in SEQ ID NO: 42, 43, or 50.

The present disclosure further relates to a IL15RA genomic DNA sequence of a humanized mouse. The DNA sequence is obtained by reverse transcription of the MRNA obtained by transcription thereof is consistent with or complementary to the DNA sequence homologous to the sequence shown in SEQ ID NO: 46 or 49.

### IL15

Interleukin-15 (IL-15 or IL15) is 14-15 kDa glycoprotein encoded by the 34 kb region of chromosome 4q31 in humans, and at the central region of chromosome 8 in mice. Although IL-15 mRNA can be found in many cells and tissues including mast cells, cancer cells or fibroblasts, this cytokine is produced as a mature protein mainly by dendritic cells, monocytes and macrophages.

IL15 is a 4-a-helix bundle cytokine playing a pivotal role in stimulation of both innate and adaptive immune cells. IL15 induces the activation, the proliferation, and the survival of T cells and contributes to generation and maintenance of high-avidity, antigen-specific CD8+ memory T cells in the long term. In addition, IL15 is involved in the development, the persistence, and the activation of NK and NKT as well as γ/δ T cells.

The IL15 receptor (IL15R) is composed of three different molecules, better known as the α (CD215; unique to the IL15R), the β (CD122), and the γ (CD132) chains. In particular, CD122 is also a component of the IL2R, whereas CD132, also known as the common γ chain (γ_{c}), is shared with different cytokines, including IL2, IL4, IL7, IL9, and IL21. While the IL15Rβγ complex is present on target cells, IL15Rα can be expressed as a membrane-bound complex with IL15 on the surface of many cell types, including activated monocytes, dendritic cells (DC), and endothelial cells. Such a heterodimer is presented in trans to neighboring α/β, γ/δ T or NK cells. Alternatively, it can be shed and released as a soluble factor. It was indicated that virtually all circulating IL15 in human and mouse serum is complexed with IL15Rα. Triggering of the receptor activates downstream signaling pathways that include JAK1 and JAK3 as well as STAT3 and STAT5, followed by the recruitment of the PI3K/AKT/mTOR and RAS/RAF/MAPK-ERK cascades. By inducing FOS/JUN, MYC, NF-κB, and BCL2 genes expression and by decreasing the expression of BIM and PUMA, IL15 has a stimulating effect on T-cell proliferation and survival.

Because sharing the β and γ components of the receptor, IL2 and IL15 exert similar functions on T cells. Indeed, both stimulate the proliferation of T cells, facilitate the differentiation of cytotoxic T lymphocytes (CTL), and induce the generation and maintenance of NK cells. Nevertheless, mice deficient in IL2 or IL15 have different phenotypes, and administration of IL2 and IL15 to mice, primates, or humans leads to distinct effects on cells of the immune system. As regards to antigen-activated effector cells, while IL2 promotes terminal differentiation and, eventually, their elimination by activation-induced cell death (AICD), IL15 inhibits AICD and promotes the generation of long-lived memory T cells as well as their maintenance by homeostatic proliferation.

IL15 and its IL15Rα chain are coexpressed by monocytes/macrophages and dendritic cells and subsequently displayed as a cell surface IL15: IL15Rα complex, which is trans-presented to neighboring immune cells expressing IL2Rβγ_{c}. Therefore, IL15 does not support maintenance of Tregs. Rather than inducing apoptosis of activated CD8+ T cells, IL15 provides anti-apoptotic signals. IL15 also has non-redundant roles in the development, proliferation, and activation of NK cells. IL15 does not induce significant capillary leak syndrome in mice or nonhuman primates (NHP), suggesting that IL15-based therapies may provide the immunostimulatory benefits of IL2 with fewer adverse effects.

A detailed description of IL15 and its function can be found, e.g., in Pilipow K., et al. “IL15 and T-cell Stemness in T-cell-Based Cancer Immunotherapy.” Cancer research 75.24 (2015): 5187-5193; and Rhode P. R., et al. “Comparison of the superagonist complex, ALT-803, to IL15 as cancer immunotherapeutics in animal models.” Cancer immunology research 4.1 (2016): 49-60; each of which is incorporated by reference in its entirety.

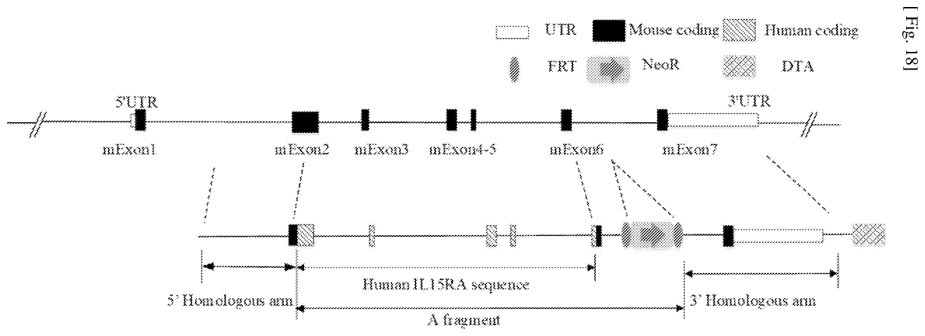

In human genomes, IL15 gene (Gene ID: 3600) locus has eight exons, exon 1, exon 2, exon 3, exon 4, exon 5, exon 6, exon 7, and exon 8 (FIG. 19). The human IL15 protein also has an N-terminal signal peptide. The nucleotide sequence for human IL15 mRNA is NM_000585.5, and the amino acid sequence for human IL15 is NP_000576.1 (SEQ ID NO: 52). The location for each exon and each region in human IL15 nucleotide sequence and amino acid sequence is listed below:

TABLE 7

| | | NP_000576.1 |
|---|---|---|
| human IL15 | NM_000585.5 | 162 aa |
| (approximate location) | 2012 bp | SEQ ID NO: 52 |
| Exon 1 | 1-166 | non-coding |
| Exon 2 | 167-288 | non-coding |
| Exon 3 | 289-399 | 1-4 |
| Exon 4 | 400-497 | 5-37 |
| Exon 5 | 498-582 | 38-65 |
| Exon 6 | 583-627 | 66-80 |
| Exon 7 | 628-765 | 81-126 |
| Exon 8 | 766-2015 | 127-162 |
| Signal peptide | 388-474 | 1-29 |
| Donor region in Example 6 | 388-876 | 1-162 |

The human IL15 gene (Gene ID: 3600) is located in Chromosome 4 of the human genome, which is located from 141636583 to 141733987 of NC_00004.12 (GRCh38. p14 (GCF_000001405.40)). The 5′ UTR is from 141,636,583 to 141,719,464, Exon 1 is from 141,636,583 to 141,636,748, the first intron is from 141,636,749 to 141,656,185, Exon 2 is from 141,656,186 to 141,656,307, the second intron is from 141,656,308 to 141,719,365, Exon 3 is from 141,719,366 to 141,719,476, the third intron is from 141,719,477 to 141,720,468, Exon 4 is from 141,720,469 to 141,720,566, the fourth intron is from 141,720,567 to 141,721,923, Exon 5 is from 141,721,924 to 141,722,008 the fifth intron is from 141,722,009 to 141,727,939, Exon 6 is from 141,727,940 to 141,727,984, the sixth intron is from 141,727,985 to 141,729,846, Exon 7 is from 141,729,847 to 141,729,984, the seventh intron is from 141,729,985 to 141,732,737, Exon 8 is from 141,732,738 to 141,733,987, and the 3′UTR is from 141,732849 to 141,733,987, based on transcript NM_000585.5. All relevant information for human IL15 locus can be found in the NCBI website with Gene ID: 3600, which is incorporated by reference herein in its entirety.

In mice, IL15 gene locus has eight exons, exon 1, exon 2, exon 3, exon 4, exon 5, exon 6, exon 7, and exon 8 (FIG. 19). The mouse IL15 protein also has an N-terminal signal peptide. The nucleotide sequence for mouse IL15 mRNA is NM_001254747.1, the amino acid sequence for mouse IL15 is NP_001241676.1 (SEQ ID NO: 51). The location for each exon and each region in the mouse IL15 nucleotide sequence and amino acid sequence is listed below:

TABLE 8

| | | NP_001241676.1 |
|---|---|---|
| Mouse IL15 | NM_001254747.1 | 162 aa |
| (approximate location) | 1287 bp | SEQ ID NO: 51 |
| Exon 1 | 1-287 | non-coding |
| Exon 2 | 288-409 | non-coding |
| Exon 3 | 410-508 | 1-4 |
| Exon 4 | 509-606 | 5-37 |
| Exon 5 | 607-691 | 38-65 |
| Exon6 | 692-736 | 66-80 |
| Exon 7 | 737-874 | 81-126 |
| Exon 8 | 875-1275 | 127-162 |
| Signal peptide | 497-583 | 1-29 |
| Replaced region in Example 6 | 497-985 | 1-162 |

The mouse IL15 gene (Gene ID: 16168) is located in Chromosome 8 of the mouse genome, which is located from 83058253 to 83129883 of NC_000074.7 (GRCm39 (GCF_000001635.27)). The 5′ UTR is from 83,129,199 to 83,072,217, Exon 1 is from 83,129,199 to 83,128,929, the first intron is from 83,128,928 to 83,106,245, Exon 2 is from 83,106,244 to 83,106,123, the second intron is from 83,106,122 to 83,072,328, Exon 3 is from 83,072,327 to 83,072,229, the third intron is from 83,072,228 to 83,071,102, Exon 4 is from 83,071,101 to 83,071,004, the fourth intron is from 83,071,003 to 83,069,953, Exon 5 is from 83,069,952 to 83,069,868, the fifth intron is from 83,069,867 to 83,064,240, Exon 6 is from 83,064,239 to 83,064,195, the sixth intron is from 83,064,194 to 83,061,236, Exon 7 is from 83,061,235 to 83,061,098, the seventh intron is from 83,061,097 to 83,058,654, Exon 8 is from 83,058,653 to 83,058,261, and the 3′UTR is from 83,058,261 to 83,058,542, based on transcript NM_001254747.1. All relevant information for mouse IL15 locus can be found in the NCBI website with Gene ID: 16168, which is incorporated by reference herein in its entirety.

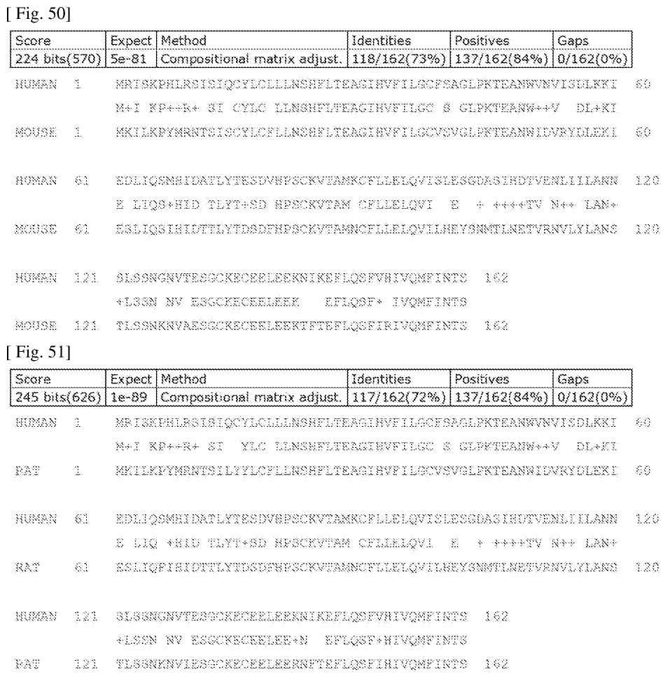

FIG. 50 shows the alignment between human IL15 amino acid sequence (NP_000576.1; SEQ ID NO: 52) and mouse IL15 amino acid sequence (NP_001241676.1; SEQ ID NO: 51). Thus, the corresponding amino acid residue or region between human and mouse IL15 can be found in FIG. 50.

IL15 genes, proteins, and locus of the other species are also known in the art. For example, the gene ID for IL15 in Rattus norvegicus (rat) is 25670, the gene ID for IL15 in Macaca mulatta (Rhesus monkey) is 699616, the gene ID for IL15 in Danio rerio (zebrafish) is 654826, and the gene ID for IL15 in Sus scrofa (pig) is 397683. The relevant information for these genes (e.g., intron sequences, exon sequences, amino acid residues of these proteins) can be found, e.g., in NCBI database, which is incorporated by reference herein in its entirety. FIG. 51 shows the alignment between human IL15 amino acid sequence (NP_000576.1; SEQ ID NO: 52) and rat IL15 amino acid sequence (NP_001388064.1; SEQ ID NO: 88. Thus, the corresponding amino acid residue or region between human and rodent IL15 can be found in FIG. 51.

The present disclosure provides human or chimeric (e.g., humanized) IL15 nucleotide sequence and/or amino acid sequences. In some embodiments, the entire sequence of mouse exon 1, exon 2, exon 3, exon 4, exon 5, exon 6, exon 7, exon 8, and/or signal peptide, are replaced by the corresponding human sequence. In some embodiments, a “region” or “portion” of mouse exon 1, exon 2, exon 3, exon 4, exon 5, exon 6, exon 7, exon 8, and/or signal peptide, are replaced by the corresponding human sequence. The term “region” or “portion” can refer to at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 250, 300, 350, 400, 450, 480, 485, 486, 487, 488, 489, 490, 500, 550, 600, 650, 700, 750, 800, 850, 900, 950, 1000, 1100, 1200, 1287, 1300, 1400, 1500, 1600, 1700, 1800, 1900, 2000, or 2012 nucleotides, or at least 1, 2, 3, 4, 5, 6, 7, 8, 9, 10, 20, 30, 40, 50, 60, 70, 80, 90, 100, 110, 120, 130, 140, 150, 160, 161, or 162 amino acid residues. In some embodiments, the “region” or “portion” can be at least 50%, 55%, 60%, 65%, 70%, 75%, 80%, 85%, 90%, 95%, or 99% identical to exon 1, exon 2, exon 3, exon 4, exon 5, exon 6, exon 7, exon 8, or signal peptide. In some embodiments, a region, a portion, or the entire sequence of mouse exon 1, exon 2, exon 3, exon 4, exon 5, exon 6, exon 7, and/or exon 8 (e.g., a portion of exon 3, exons 4-7, and a portion of exon 8) are replaced by a region, a portion, or the entire sequence of the human exon 1, exon 2, exon 3, exon 4, exon 5, exon 6, exon 7, and/or exon 8 (e.g., a portion of exon 3, exons 4-7, and a portion of exon 8).

In some embodiments, a “region” or “portion” of the signal peptide, exon 1, exon 2, exon 3, exon 4, exon 5, exon 6, exon 7, and/or exon 8 is deleted.

In some embodiments, the present disclosure is related to a genetically-modified, non-human animal whose genome comprises a chimeric (e.g., humanized) IL15 nucleotide sequence. In some embodiments, the chimeric (e.g., humanized) IL15 nucleotide sequence encodes a IL15 protein comprising an N-terminal signal peptide. In some embodiments, the signal peptide comprises a sequence that is at least 80%, 85%, 90%, 95%, or 100% identical to amino acids 1-29 of SEQ ID NO: 51 or 52. In some embodiments, the signal peptide comprises all or part of human or endogenous IL15 signal peptide. In some embodiments, the genome of the animal comprises a sequence that is at least 80%, 85%, 90%, 95%, or 100% identical to SEQ ID NO: 53, 54, 55, 56, 57, or 58.

In some embodiments, the genetically-modified non-human animal described herein comprises a sequence encoding a human or humanized IL15 protein. In some embodiments, the IL15 protein comprises an N-terminal signal peptide. In some embodiments, the humanized IL15 protein comprises a human or humanized signal peptide. In some embodiments, the humanized IL15 protein comprises an endogenous signal peptide.

In some embodiments, the genetically-modified non-human animal described herein comprises a human or humanized IL15 gene. In some embodiments, the humanized IL15 gene comprises 8 exons. In some embodiments, the humanized IL15 gene comprises endogenous exon 1, endogenous exon 2, humanized exon 3, human exon 4, human exon 5, human exon 6, human exon 7, and/or humanized exon 8. In some embodiments, the humanized IL15 gene comprises 7 introns. In some embodiments, the humanized IL15 gene comprises endogenous intron 1, endogenous intron 2, human intron 3, human intron 4, human intron 5, human intron 6, and human intron 7. In some embodiments, the humanized IL15 gene comprises human or humanized 5′ UTR. In some embodiments, the humanized IL15 gene comprises human or humanized 3′ UTR. In some embodiments, the humanized IL15 gene comprises endogenous 5′ UTR. In some embodiments, the humanized IL15 gene comprises endogenous 3′ UTR.

Thus, in some embodiments, the present disclosure also provides a chimeric (e.g., humanized) IL15 nucleotide sequence and/or amino acid sequences, wherein in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from mouse IL15 mRNA sequence (e.g., NM_001254747.1), mouse IL15 amino acid sequence (e.g., SEQ ID NO: 51), or a portion thereof (e.g., exons 1-2, a portion of exon 3, and a portion of exon 8); and in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from human IL15 mRNA sequence (e.g., NM_000585.5), human IL15 amino acid sequence (e.g., SEQ ID NO: 52), or a portion thereof (e.g., a portion of exon 3, exons 4-7, and a portion of exon 8).

In some embodiments, the sequence encoding amino acids 1-162 of mouse IL15 (SEQ ID NO: 51) is replaced. In some embodiments, the sequence is replaced by a sequence encoding a corresponding region of human IL15 (e.g., amino acids 1-162 of human IL15 (SEQ ID NO: 52)).

In some embodiments, the sequence encoding amino acids 30-162 of mouse IL15 (SEQ ID NO: 51) is replaced. In some embodiments, the sequence is replaced by a sequence encoding a corresponding region of human IL15 (e.g., amino acids 30-162 of human IL15 (SEQ ID NO: 52)).

In some embodiments, the nucleic acids as described herein are operably linked to a promotor or regulatory element, e.g., an endogenous mouse IL15 promotor, an inducible promoter, an enhancer, and/or mouse or human regulatory elements.

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, 100, 150, 200, 250, 300, 350, 400, 450, 480, 485, 486, 487, 488, 489, 490, 500, 550, 600, 650, 700, 750, 800, 850, 900, 950, 1000, 1100, 1200, 1250, or 1287 nucleotides, e.g., contiguous or non-contiguous nucleotides) that are different from part of or the entire mouse IL15 nucleotide sequence (e.g., a portion of exon 3, exons 4-7, and a portion of exon 8 of NM_001254747.1).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, 100, 150, 200, 250, 300, 301, 302, 303, 304, 305, 350, 400, 450, 480, 490, 491, 492, 493, 494, 495, 496, 497, 498, 499, 500, 550, 600, 650, 700, 750, 800, 850, 900, 950, 1000, 1100, 1200, 1250, or 1287 nucleotides, e.g., contiguous or non-contiguous nucleotides) that is the same as part of or the entire mouse IL15 nucleotide sequence (e.g., exons 1-2, a portion of exon 3, and a portion of exon 8 of NM_001254747.1).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, 100, 150, 155, 156, 157, 158, 159, 160, 200, 250, 300, 350, 360, 370, 380, 385, 386, 387, 388, 389, 390, 400, 450, 500, 550, 600, 650, 700, 750, 800, 850, 900, 950, 1000, 1100, 1130, 1135, 1136, 1200, 1300, 1400, 1500, 1600, 1700, 1800, 1900, 2000, or 2012 nucleotides, e.g., contiguous or non-contiguous nucleotides) that is different from part of or the entire human IL15 nucleotide sequence (e.g., exons 1-2, a portion of exon 3, and a portion of exon 8 of NM_000585.5).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, 100, 150, 200, 250, 300, 350, 400, 450, 480, 485, 486, 487, 488, 489, 490, 500, 550, 600, 650, 700, 750, 800, 850, 900, 950, 1000, 1100, 1200, 1300, 1400, 1500, 1600, 1700, 1800, 1900, 2000, or 2012 nucleotides, e.g., contiguous or non-contiguous nucleotides) that is the same as part of or the entire human IL15 nucleotide sequence (e.g., a portion of exon 3, exons 4-7, and a portion of exon 8 of NM_000585.5).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, 100, 110, 120, 130, 140, 150, 160, 161, or 162 amino acid residues, e.g., contiguous or non-contiguous amino acid residues) that is different as part of or the entire mouse IL15 amino acid sequence (e.g., amino acids 1-162 of NP_001241676.1 (SEQ ID NO: 51)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, 100, 110, 120, 130, 140, 150, 160, 161, or 162 amino acid residues, e.g., contiguous or non-contiguous amino acid residues) that is the same from part of or the entire mouse IL15 amino acid sequence. (e.g., amino acids 1-162 of NP_001241676.1 (SEQ ID NO: 51)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, 100, 110, 120, 130, 140, 150, 160, 161, or 162 amino acid residues, e.g., contiguous or non-contiguous amino acid residues) that is different from part of or the entire human IL15 amino acid sequence (e.g., amino acids 1-162 of NP_000576.1 (SEQ ID NO: 52)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, 100, 110, 120, 130, 140, 150, 160, 161, or 162 amino acid residues, e.g., contiguous or non-contiguous amino acid residues) that is the same as part of or the entire human IL15 amino acid sequence (e.g., amino acids 37-211 of NP_000576.1 (SEQ ID NO: 52)).

The present disclosure also provides a humanized IL15 mouse amino acid sequence, wherein the amino acid sequence is selected from the group consisting of:
a) an amino acid sequence shown in SEQ ID NO: 51 or 52;
b) an amino acid sequence having a homology of at least 90% with or at least 90% identical to the amino acid sequence shown in SEQ ID NO: 51 or 52;
c) an amino acid sequence encoded by a nucleic acid sequence, wherein the nucleic acid sequence is able to hybridize to a nucleotide sequence encoding the amino acid shown in SEQ ID NO: 51 or 52 under a low stringency condition or a strict stringency condition;
d) an amino acid sequence having a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the amino acid sequence shown in SEQ ID NO: 51 or 52;
e) an amino acid sequence that is different from the amino acid sequence shown in SEQ ID NO: 51 or 52 by no more than 10, 9, 8, 7, 6, 5, 4, 3, 2 or no more than 1 amino acid; or
f) an amino acid sequence that comprises a substitution, a deletion and/or insertion of one or more amino acids to the amino acid sequence shown in SEQ ID NO: 51 or 52.

The present disclosure also provides a humanized IL15 amino acid sequence, wherein the amino acid sequence is selected from the group consisting of:
a) all or part of amino acids 1-162 or 30-162 of SEQ ID NO: 52;
b) an amino acid sequence have a homology of at least 90%, 91%, 92%, 93%, 94%, 95%, 96%, 97%, 98%, or 99% to amino acids 1-162 or 30-162 of SEQ ID NO: 52;
c) an amino acid sequence that is different from amino acids 1-162 or 30-162 of SEQ ID NO: 52 by no more than 10, 9, 8, 7, 6, 5, 4, 3, 2 or no more than 1 amino acid; and
d) an amino acid sequence that comprises a substitution, a deletion and/or insertion of one or more amino acids to amino acids 1-162 or 30-162 of SEQ ID NO: 52.

The present disclosure also relates to a IL15 nucleic acid (e.g., DNA or RNA) sequence, wherein the nucleic acid sequence can be selected from the group consisting of:
a) a nucleic acid sequence as shown in SEQ ID NO: 53, 54, 55, 56, 57, or 58, or a nucleic acid sequence encoding a homologous IL15 amino acid sequence of a humanized mouse IL15;
b) a nucleic acid sequence that is able to hybridize to the nucleotide sequence as shown in SEQ ID NO: 53, 54, 55, 56, 57, or 58 under a low stringency condition or a strict stringency condition;
c) a nucleic acid sequence that has a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the nucleotide sequence as shown in SEQ ID NO: 53, 54, 55, 56, 57, or 58;
d) a nucleic acid sequence that encodes an amino acid sequence, wherein the amino acid sequence has a homology of at least 90% with or at least 90% identical to the amino acid sequence shown in SEQ ID NO: 51 or 52;
e) a nucleic acid sequence that encodes an amino acid sequence, wherein the amino acid sequence has a homology of at least 90%, 91%, 92%, 93%, 94%, 95%, 96%, 97%, 98%, or 99% with, or at least 90%, 91%, 92%, 93%, 94%, 95%, 96%, 97%, 98%, or 99% identical to the amino acid sequence shown in SEQ ID NO: 51 or 52;
f) a nucleic acid sequence that encodes an amino acid sequence, wherein the amino acid sequence is different from the amino acid sequence shown in SEQ ID NO: 51 or 52 by no more than 10, 9, 8, 7, 6, 5, 4, 3, 2 or no more than 1 amino acid; and/or
g) a nucleic acid sequence that encodes an amino acid sequence, wherein the amino acid sequence comprises a substitution, a deletion and/or insertion of one or more amino acids to the amino acid sequence shown in SEQ ID NO: 51 or 52.

The present disclosure further relates to a IL15 genomic DNA sequence of a humanized mouse. The DNA sequence is obtained by reverse transcription of the MRNA obtained by transcription thereof is consistent with or complementary to the DNA sequence homologous to the sequence shown in SEQ ID NO: 55 or 58.

The disclosure also provides an amino acid sequence that has a homology of at least 90% with, or at least 90% identical to the sequence shown in SEQ ID NO: 1, 2, 30, 33, 34, 39, 42, 43, 50, 51, or 52, and has protein activity. In some embodiments, the homology with the sequence shown in SEQ ID NO: 1, 2, 30, 33, 34, 39, 42, 43, 50, 51, or 52 is at least about 90%, 91%, 92%, 93%, 94%, 95%, 96%, 97%, 98%, or at least 99%. In some embodiments, the foregoing homology is at least about 60%, 61%, 62%, 63%, 64%, 65%, 66%, 67%, 68%, 69%, 70%, 71%, 72%, 73%, 74%, 75%, 80%, or 85%.

In some embodiments, the percentage identity with the sequence shown in SEQ ID NO: 1, 2, 30, 33, 34, 39, 42, 43, 50, 51, or 52 is at least about 90%, 91%, 92%, 93%, 94%, 95%, 96%, 97%, 98%, or at least 99%. In some embodiments, the foregoing percentage identity is at least about 60%, 61%, 62%, 63%, 64%, 65%, 66%, 67%, 68%, 69%, 70%, 71%, 72%, 73%, 74%, 75%, 80%, or 85%.

The disclosure also provides a nucleotide sequence that has a homology of at least 90%, or at least 90% identical to the sequence shown in SEQ ID NO: 5, 26, 29, 37, 38, 46, 49, 55, or 58, and encodes a polypeptide that has protein activity. In some embodiments, the homology with the sequence shown in SEQ ID NO: 5, 26, 29, 37, 38, 46, 49, 55, or 58 is at least about 90%, 91%, 92%, 93%, 94%, 95%, 96%, 97%, 98%, or at least 99%. In some embodiments, the foregoing homology is at least about 50%, 55%, 60%, 65%, 66%, 67%, 68%, 69%, 70%, 71%, 72%, 73%, 74%, 75%, 80%, or 85%.

In some embodiments, the percentage identity with the sequence shown in SEQ ID NO: 3, 4, 5, 6, 7, 8, 9, 24, 25, 26, 27, 28, 29, 31, 32, 35, 36, 37, 38, 40, 41, 44, 45, 46, 47, 48, 49, 53, 54, 55, 56, 57, or 58 is at least about 90%, 91%, 92%, 93%, 94%, 95%, 96%, 97%, 98%, or at least 99%. In some embodiments, the foregoing percentage identity is at least about 50%, 55%, 60%, 65%, 66%, 67%, 68%, 69%, 70%, 71%, 72%, 73%, 74%, 75%, 80%, or 85%.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein. In some embodiments, the disclosure relates to nucleotide sequences encoding any peptides that are described herein, or any amino acid sequences that are encoded by any nucleotide sequences as described herein. In some embodiments, the nucleic acid sequence is less than 10, 20, 30, 40, 50, 60, 70, 80, 90, 100, 110, 120, 130, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2500, 3000, 3500, 4000, 4500, or 5000 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, 200, 250, 300, 350, 400, 450, or 500 amino acid residues.

In some embodiments, the amino acid sequence (i) comprises an amino acid sequence; or (ii) consists of an amino acid sequence, wherein the amino acid sequence is any one of the sequences as described herein.

In some embodiments, the nucleic acid sequence (i) comprises a nucleic acid sequence; or (ii) consists of a nucleic acid sequence, wherein the nucleic acid sequence is any one of the sequences as described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For example, the comparison of sequences and determination of percent identity between two sequences can be accomplished using a Blossum 62 scoring matrix with a gap penalty of 12, a gap extend penalty of 4, and a frameshift gap penalty of 5.

The percentage of residues conserved with similar physicochemical properties (percent homology), e.g. leucine and isoleucine, can also be used to measure sequence similarity. Families of amino acid residues having similar physicochemical properties have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The homology percentage, in many cases, is higher than the identity percentage.

Cells, tissues, and animals (e.g., mouse) are also provided that comprise the nucleotide sequences as described herein, as well as cells, tissues, and animals (e.g., mouse) that express human or chimeric (e.g., humanized) IL2RG from an endogenous non-human IL2RG locus, human or chimeric (e.g., humanized) IL2RB from an endogenous non-human IL2RB locus, human or chimeric (e.g., humanized) IL15RA from an endogenous non-human IL15RA locus, and/or human or chimeric (e.g., humanized) IL15 from an endogenous non-human IL15 locus.

### Genetically Modified Animals

As used herein, the term “genetically-modified non-human animal” refers to a non-human animal having exogenous DNA in at least one chromosome of the animal's genome. In some embodiments, at least one or more cells, e.g., at least 1%, 2%, 3%, 4%, 5%, 10%, 20%, 30%, 40%, 50% of cells of the genetically-modified non-human animal have the exogenous DNA in its genome. The cell having exogenous DNA can be various kinds of cells, e.g., an endogenous cell, a somatic cell, an immune cell, a T cell, a B cell, an antigen presenting cell, a macrophage, a dendritic cell, a germ cell, a blastocyst, or an endogenous tumor cell. In some embodiments, genetically-modified non-human animals are provided that comprise modified endogenous IL2RG, IL2RB, IL15RA, and/or IL15 loci that comprise an exogenous sequence (e.g., a human sequence), e.g., a replacement of one or more non-human sequences with one or more human sequences. The animals are generally able to pass the modification to progeny, i.e., through germline transmission.

As used herein, the term “chimeric gene” or “chimeric nucleic acid” refers to a gene or a nucleic acid, wherein two or more portions of the gene or the nucleic acid are from different species, or at least one of the sequences of the gene or the nucleic acid does not correspond to the wild-type nucleic acid in the animal. In some embodiments, the chimeric gene or chimeric nucleic acid has at least one portion of the sequence that is derived from two or more different sources, e.g., sequences encoding different proteins or sequences encoding the same (or homologous) protein of two or more different species. In some embodiments, the chimeric gene or the chimeric nucleic acid is a humanized gene or humanized nucleic acid.

As used herein, the term “chimeric protein” or “chimeric polypeptide” refers to a protein or a polypeptide, wherein two or more portions of the protein or the polypeptide are from different species, or at least one of the sequences of the protein or the polypeptide does not correspond to wild-type amino acid sequence in the animal. In some embodiments, the chimeric protein or the chimeric polypeptide has at least one portion of the sequence that is derived from two or more different sources, e.g., same (or homologous) proteins of different species. In some embodiments, the chimeric protein or the chimeric polypeptide is a humanized protein or a humanized polypeptide.

As used herein, the term “humanized protein” or “humanized polypeptide” refers to a protein or a polypeptide, wherein at least a portion of the protein or the polypeptide is from the human protein or human polypeptide. In some embodiments, the humanized protein or polypeptide is a human protein or polypeptide.

As used herein, the term “humanized nucleic acid” refers to a nucleic acid, wherein at least a portion of the nucleic acid is from the human. In some embodiments, the entire nucleic acid of the humanized nucleic acid is from human. In some embodiments, the humanized nucleic acid is a humanized exon. A humanized exon can be, e.g., a human exon or a chimeric exon.

### Animals Having Humanized IL2RG Gene Locus

In some embodiments, the chimeric gene or the chimeric nucleic acid is a humanized IL2RG gene or a humanized IL2RG nucleic acid. In some embodiments, at least one or more portions of the gene or the nucleic acid is from the human IL2RG gene, at least one or more portions of the gene or the nucleic acid is from a non-human IL2RG gene. In some embodiments, the gene or the nucleic acid comprises a sequence that encodes an IL2RG protein. The encoded IL2RG protein is functional or has at least one activity of the human IL2RG protein or the non-human IL2RG protein.

In some embodiments, the chimeric protein or the chimeric polypeptide is a humanized IL2RG protein or a humanized IL2RG polypeptide. In some embodiments, at least one or more portions of the amino acid sequence of the protein or the polypeptide is from a human IL2RG protein, and at least one or more portions of the amino acid sequence of the protein or the polypeptide is from a non-human IL2RG protein. The humanized IL2RG protein or the humanized IL2RG polypeptide is functional or has at least one activity of the human IL2RG protein or the non-human IL2RG protein.

In some embodiments, the humanized IL2RG protein includes a polypeptide sequence of 5-369 amino acids (contiguous or non-contiguous) that is identical to human IL2RG protein. In some embodiments, the polypeptide sequence is 5-369, 10-256, or 10-369 amino acids in length. In some embodiments, the humanized IL2RG gene includes a nucleotide sequence of 20-4225 bp (contiguous or non-contiguous) that is identical to human IL2RG gene. In some embodiments, the nucleotide sequence is 20-4200 bp, 20-2855 bp, 20-1560 bp, 20-1442 bp, or 20-768 bp.

In some embodiments, the IL2RG extracellular region is human or humanized. In some embodiments, the IL2RG signal peptide is human or humanized. In some embodiments, the IL2RG cytoplasmic region is human or humanized. In some embodiments, the IL2RG transmembrane region is human or humanized. In some embodiments, both the IL2RG extracellular region and signal peptide are human or humanized. In some embodiments, both the IL2RG transmembrane and cytoplasmic regions are endogenous.

Genetically modified non-human animals can comprise a modification at an endogenous non-human IL2RG locus. In some embodiments, the modification can comprise a human nucleic acid sequence encoding at least a portion of a mature IL2RG protein (e.g., at least 10%, 20%, 30%, 40%, 50%, 60%, 70%, 80%, 90%, 95%, 96%, 97%, 98%, or 99% identical to the mature IL2RG protein sequence). Although genetically modified cells are also provided that can comprise the modifications described herein (e.g., ES cells, somatic cells), in many embodiments, the genetically modified non-human animals comprise the modification of the endogenous IL2RG locus in the germline of the animal.

Genetically modified animals can express a human IL2RG and/or a chimeric (e.g., humanized) IL2RG from endogenous mouse loci, wherein the endogenous mouse IL2RG gene has been replaced with a human IL2RG gene and/or a nucleotide sequence that encodes a region of human IL2RG sequence or an amino acid sequence that is at least 10%, 20%, 30%, 40%, 50%, 60%, 70%, 80%, 90%, 95%, 96%, 97%, 98%, or 99% identical to the human IL2RG sequence. In various embodiments, an endogenous non-human IL2RG locus is modified in whole or in part to comprise human nucleic acid sequence encoding at least one protein-coding sequence of a mature IL2RG protein.

In some embodiments, the genetically modified mice can express the human IL2RG and/or chimeric IL2RG (e.g., humanized IL2RG) from endogenous loci that are under control of mouse promoters and/or mouse regulatory elements. The replacement(s) at the endogenous mouse loci provide non-human animals that express human IL2RG or chimeric IL2RG (e.g., humanized IL2RG) in appropriate cell types and in a manner that does not result in the potential pathologies observed in some other transgenic mice known in the art. The human IL2RG or the chimeric IL2RG (e.g., humanized IL2RG) expressed in animal can maintain one or more functions of the wild-type mouse or human IL2RG in the animal. Furthermore, in some embodiments, the animal does not express endogenous IL2RG. In some embodiments, the animal expresses a decreased level of endogenous IL2RG as compared to IL2RG expression level in a wild-type animal. As used herein, the term “endogenous IL2RG” refers to IL2RG protein that is expressed from an endogenous IL2RG nucleotide sequence of the non-human animal (e.g., mouse) before any genetic modification.

The genome of the animal can comprise a sequence encoding an amino acid sequence that is at least 70%, 75%, 80%, 85%, 90%, 95%, 99%, or 100% identical to human IL2RG (NP_000197.1; SEQ ID NO: 2). In some embodiments, the genome comprises a sequence encoding an amino acid sequence that is at least 70%, 75%, 80%, 85%, 90%, 95%, 99%, or 100% identical to SEQ ID NO: 1, 2, or 30.

The genome of the genetically modified animal can comprise a replacement at an endogenous IL2RG gene locus of a sequence encoding a region of endogenous IL2RG with a sequence encoding a corresponding region of human IL2RG. In some embodiments, the sequence that is replaced is any sequence within the endogenous IL2RG gene locus, e.g., exon 1, exon 2, exon 3, exon 4, exon 5, exon 6, exon 7, exon 8, 5′-UTR, 3′-UTR, intron 1, intron 2, intron 3, intron 4, intron 5, intron 6, intron 7, or any combination thereof. In some embodiments, the sequence that is replaced is within the regulatory region of the endogenous IL2RG gene. In some embodiments, the sequence that is replaced is a portion of exon 1, exons 2-7, and a portion of exon 8, of an endogenous mouse IL2RG gene locus. In some embodiments, the sequence that is replaced is a portion of exon 1, exons 2-5, and a portion of exon 6, of an endogenous mouse IL2RG gene locus.

The genetically modified animal can have one or more cells expressing a human or chimeric IL2RG (e.g., humanized IL2RG) having, from N-terminus to C-terminus, a signal peptide, an extracellular region, a transmembrane region, and a cytoplasmic region. In some embodiments, the signal peptide comprises a sequence that is at least 50%, 60%, 70%, 80%, 90%, 95%, 99% identical to the signal peptide of human IL2RG. In some embodiments, the signal peptide of the humanized IL2RG has a sequence that has at least 1, 2, 3, 4, 5, 6, 7, 8, 9, 10, 11, 12, 13, 14, 15, 16, 17, 18, 19, 20, 21, or 22 amino acids (e.g., contiguously or non-contiguously) that are identical to the signal peptide of human IL2RG. In some embodiments, the extracellular region comprises a sequence that is at least 50%, 60%, 70%, 80%, 90%, 95%, 99% identical to the extracellular region of human IL2RG. In some embodiments, the extracellular region of the humanized IL2RG has a sequence that has at least 10, 20, 30, 40, 50, 60, 70, 80, 90, 100, 110, 120, 130, 140, 150, 160, 170, 180, 190, 200, 210, 220, 230, 231, 232, 233, 234, 235, 236, 237, 238, 239, or 240 amino acids (e.g., contiguously or non-contiguously) that are identical to the extracellular region of human IL2RG. In some embodiments, the extracellular region of the humanized IL2RG has a sequence that has at least 1, 2, 3, 4, 5, or 6 amino acids (contiguously or non-contiguously) that are identical to the C-terminal 1-6 amino acids in the extracellular region of endogenous IL2RG (e.g., mouse IL2RG). In some embodiments, the extracellular region described herein includes the signal peptide. In some embodiments, the extracellular region described herein does not include the signal peptide. Because human IL2RG and non-human IL2RG (e.g., mouse IL2RG) sequences, in many cases, are different, antibodies that bind to human IL2RG will not necessarily have the same binding affinity with non-human IL2RG or have the same effects to non-human IL2RG. Therefore, the genetically modified animal having a human or a humanized extracellular region can be used to better evaluate the effects of anti-human IL2RG antibodies in an animal model.

In some embodiments, the transmembrane region comprises a sequence that is at least 50%, 60%, 70%, 80%, 90%, 95%, 99% identical to the transmembrane region of human IL2RG (e.g., amino acids 263-283 of SEQ ID NO: 2). In some embodiments, the transmembrane region of the humanized IL2RG has a sequence that has at least 1, 2, 3, 4, 5, 6, 7, 8, 9, 10, 11, 12, 13, 14, 15, 16, 17, 18, 19, 20, or 21 amino acids (contiguously or non-contiguously) that are identical to the transmembrane region of human IL2RG. In some embodiments, the cytoplasmic comprises a sequence that is at least 50%, 60%, 70%, 80%, 90%, 95%, 99% identical to the cytoplasmic of human IL2RG (e.g., amino acids 284-369 of SEQ ID NO: 2). In some embodiments, the cytoplasmic region of the humanized IL2RG has a sequence that has at least 10, 15, 20, 25, 30, 35, 40, 45, 50, 55, 60, 65, 70, 75, 80, 81, 82, 83, 84, 85, or 86 amino acids (contiguously or non-contiguously) that are identical to the cytoplasmic region of human IL2RG.

In some embodiments, the transmembrane region comprises a sequence that is at least 50%, 60%, 70%, 80%, 90%, 95%, 99% identical to the transmembrane region of endogenous IL2RG (e.g., amino acids 264-284 of SEQ ID NO: 1). In some embodiments, the transmembrane region of the humanized IL2RG has a sequence that has at least 1, 2, 3, 4, 5, 6, 7, 8, 9, 10, 11, 12, 13, 14, 15, 16, 17, 18, 19, 20, or 21 amino acids (contiguously or non-contiguously) that are identical to the transmembrane region of endogenous IL2RG (e.g., mouse IL2RG). In some embodiments, the cytoplasmic comprises a sequence that is at least 50%, 60%, 70%, 80%, 90%, 95%, 99% identical to the cytoplasmic of endogenous IL2RG (e.g., amino acids 285-369 of SEQ ID NO: 1). In some embodiments, the cytoplasmic region of the humanized IL2RG has a sequence that has at least 10, 15, 20, 25, 30, 35, 40, 45, 50, 55, 60, 65, 70, 75, 80, 81, 82, 83, 84, or 85 amino acids (contiguously or non-contiguously) that are identical to the cytoplasmic region of endogenous IL2RG (e.g., mouse IL2RG).

In some embodiments, the entire transmembrane region and the entire cytoplasmic region of the humanized IL2RG described herein are derived from endogenous sequence.

In some embodiments, the genome of the genetically modified animal comprises a sequence encoding an amino acid sequence that corresponds to a portion or the entire sequence of exon 1, exon 2, exon 3, exon 4, exon 5, exon 6, exon 7, and/or exon 8 of human IL2RG; a portion or the entire sequence of the signal peptide, and/or a portion or the entire sequence of the extracellular region of human IL2RG; or a portion or the entire sequence of amino acids 1-256, 23-256, 1-262, or 23-262 of SEQ ID NO: 2.

In some embodiments, the genome of the genetically modified animal comprises a portion of exon 1, exons 2-7, and a portion of exon 8 of human IL2RG gene. In some embodiments, the portion of exon 1 includes at least 5, 10, 15, 20, 25, 28, 29, 30, 31, 32, 33, 34, 35, 40, 45, 50, 55, 60, 65, 70, 75, 80, 85, 90, 95, 100, 105, 110, 111, 112, 113, 114, 115, 120, 150, 200, or 207 nucleotides (e.g., 5-207 or 10-115 nucleotides). In some embodiments, the portion of exon 1 includes 115 nucleotides. In some embodiments, the portion of exon 1 includes a nucleotide of at least 50 bp. In some embodiments, the portion of exon 8 includes at least 1, 2, 3, 4, 5, 6, 7, 8, 9, 10, 11, 12, 13, 14, 15, 20, 25, 26, 27, 28, 29, 30, 40, 50, 60, 70, 80, 90, 100, 150, 200, 250, 300, 350, 400, 450, 500, 510, 515, 516, 517, or 518 nucleotides. In some embodiments, the portion of exon 8 includes 518 nucleotides. In some embodiments, the portion of exon 8 includes a nucleotide of at least 100 bp.

In some embodiments, the genome of the genetically modified animal comprises a portion of exon 1, exons 2-5, and a portion of exon 6 of human IL2RG gene. In some embodiments, the portion of exon 1 includes at least 5, 10, 15, 20, 25, 28, 29, 30, 31, 32, 33, 34, 35, 40, 45, 50, 55, 60, 65, 70, 75, 80, 85, 90, 95, 100, 105, 110, 111, 112, 113, 114, 115, 120, 150, 200, or 207 nucleotides. In some embodiments, the portion of exon 1 includes 115 nucleotides. In some embodiments, the portion of exon 1 includes a nucleotide of at least 50 bp. In some embodiments, the portion of exon 6 includes at least 1, 2, 3, 4, 5, 6, 7, 8, 9, 10, 11, 12, 13, 14, 15, 20, 25, 26, 27, 28, 29, 30, 40, 50, 60, 70, 80, 90, 95, 96, or 97 nucleotides. In some embodiments, the portion of exon 6 includes 11 nucleotides. In some embodiments, the portion of exon 6 starts from the first nucleotide in exon 6 and ends at a nucleotide encoding the C-terminal 1-10 (e.g., 1, 2, 3, 4, 5, 6, 7, 8, 9, or 10) amino acids in the extracellular region of human IL2RG. In some embodiments, the portion of exon 6 includes a nucleotide of at least 5 bp.

In some embodiments, the non-human animal can have, at an endogenous IL2RG gene locus, a nucleotide sequence encoding a chimeric human/non-human IL2RG polypeptide, wherein a human portion of the chimeric human/non-human IL2RG polypeptide comprises the entire human IL2RG signal peptide and all or a portion of the human IL2RG extracellular region, and wherein the animal expresses a functional IL2RG on a surface of a cell of the animal. The human portion of the chimeric human/non-human IL2RG polypeptide can comprise an amino acid sequence encoded by a portion of exon 1, exons 2-5, and/or a portion of exon 6 of human IL2RG gene. In some embodiments, the human portion of the chimeric human/non-human IL2RG polypeptide can comprise a sequence that is at least 80%, 85%, 90%, 95%, or 99% identical to amino acids 1-256 of SEQ ID NO: 2. In some embodiments, the transmembrane region includes a sequence corresponding to the entire or part of amino acids 264-284 of SEQ ID NO: 1. In some embodiments, the cytoplasmic region includes a sequence corresponding to the entire or part of amino acids 285-369 of SEQ ID NO: 1. In some embodiments, the chimeric human/non-human IL2RG polypeptide comprises a signal peptide, which includes a sequence corresponding to the entire or part of amino acids 1-22 of SEQ ID NO: 2.

In some embodiments, the non-human portion of the chimeric human/non-human IL2RG polypeptide comprises the entire transmembrane region and/or the entire cytoplasmic region of an endogenous non-human IL2RG polypeptide.

Furthermore, the genetically modified animal can be heterozygous with respect to the replacement at the endogenous IL2RG locus, or homozygous with respect to the replacement at the endogenous IL2RG locus.

In some embodiments, the humanized IL2RG locus lacks a human IL2RG gene 5′-UTR. In some embodiment, the humanized IL2RG locus comprises an endogenous (e.g., mouse) 5′-UTR. In some embodiments, the humanized IL2RG locus comprises an endogenous (e.g., mouse) 3′-UTR. In some embodiments, the humanized IL2RG locus comprises human 3′-UTR. In appropriate cases, it may be reasonable to presume that the mouse and human IL2RG genes appear to be similarly regulated based on the similarity of their 5′-flanking sequence. As shown in the present disclosure, humanized IL2RG mice that comprise a replacement at an endogenous mouse IL2RG locus, which retain mouse regulatory elements but comprise a humanization of IL2RG encoding sequence, do not exhibit pathologies. Both genetically modified mice that are heterozygous or homozygous for humanized IL2RG are grossly normal.

In another aspect, the disclosure also provides a genetically-modified, non-human animal whose genome comprise a disruption in the animal's endogenous IL2RG gene, wherein the disruption of the endogenous IL2RG gene comprises deletion of exon 1, exon 2, exon 3, exon 4, exon 5, exon 6, exon 7, and/or exon 8, or part thereof of the endogenous IL2RG gene.

In some embodiments, the disruption of the endogenous IL2RG gene comprises deletion of one or more exons or part of exons selected from the group consisting of exon 1, exon 2, exon 3, exon 4, exon 5, exon 6, exon 7, and exon 8 of the endogenous IL2RG gene.

In some embodiments, the disruption of the endogenous IL2RG gene further comprises deletion of one or more introns or part of introns selected from the group consisting of intron 1, intron 2, intron 3, intron 4, intron 5, intron 6, and intron 7 of the endogenous IL2RG gene.

In some embodiments, wherein the deletion can comprise deleting at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 300, 400, 500, 600, 700, 800, 900, 1000, 1100, 1200, 1300, 1400, 1500, 1600, 1700, 1800, 1900, 2000, 2500, 3000, 3500, 3600, 3700, 3800, 3871, or more nucleotides.

In some embodiments, the disruption of the endogenous IL2RG gene comprises the deletion of at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 300, 400, 500, 600, 700, 750, 560, 770, 771, 780, 800, 900, 1000, 1500, or 1527 nucleotides of exon 1, exon 2, exon 3, exon 4, exon 5, exon 6, exon 7, and/or exon 8 (e.g., deletion of at least 50 nucleotides from exon 1, exons 2-7, and at least 100 nucleotides from exon 8; alternatively, deletion of at least 50 nucleotides from exon 1, exons 2-5, and at least 5 nucleotides from exon 6).

### Animals Having Humanized IL2RB Gene Locus

In some embodiments, the chimeric gene or the chimeric nucleic acid is a humanized IL2RB gene or a humanized IL2RB nucleic acid. In some embodiments, at least one or more portions of the gene or the nucleic acid is from the human IL2RB gene, at least one or more portions of the gene or the nucleic acid is from a non-human IL2RB gene. In some embodiments, the gene or the nucleic acid comprises a sequence that encodes an IL2RB protein. The encoded IL2RB protein is functional or has at least one activity of the human IL2RB protein or the non-human IL2RB protein.

In some embodiments, the chimeric protein or the chimeric polypeptide is a humanized IL2RB protein or a humanized IL2RB polypeptide. In some embodiments, at least one or more portions of the amino acid sequence of the protein or the polypeptide is from a human IL2RB protein, and at least one or more portions of the amino acid sequence of the protein or the polypeptide is from a non-human IL2RB protein. The humanized IL2RB protein or the humanized IL2RB polypeptide is functional or has at least one activity of the human IL2RB protein or the non-human IL2RB protein.

In some embodiments, the IL2RB extracellular region is human or humanized. In some embodiments, the IL2RB signal peptide is human or humanized. In some embodiments, the IL2RB cytoplasmic region is human or humanized. In some embodiments, the IL2RB transmembrane region is human or humanized. In some embodiments, only the IL2RB extracellular region is human or humanized. In some embodiments, all of the IL2RB signal peptide, transmembrane region, and cytoplasmic regions are endogenous.

Genetically modified non-human animals can comprise a modification at an endogenous non-human IL2RB locus. In some embodiments, the modification can comprise a human nucleic acid sequence encoding at least a portion of a mature IL2RB protein (e.g., at least 10%, 20%, 30%, 40%, 50%, 60%, 70%, 80%, 90%, 95%, 96%, 97%, 98%, or 99% identical to the mature IL2RB protein sequence). Although genetically modified cells are also provided that can comprise the modifications described herein (e.g., ES cells, somatic cells), in many embodiments, the genetically modified non-human animals comprise the modification of the endogenous IL2RB locus in the germline of the animal.

Genetically modified animals can express a human IL2RB and/or a chimeric (e.g., humanized) IL2RB from endogenous mouse loci, wherein the endogenous mouse IL2RB gene has been replaced with a human IL2RB gene and/or a nucleotide sequence that encodes a region of human IL2RB sequence or an amino acid sequence that is at least 10%, 20%, 30%, 40%, 50%, 60%, 70%, 80%, 90%, 95%, 96%, 97%, 98%, or 99% identical to the human IL2RB sequence. In various embodiments, an endogenous non-human IL2RB locus is modified in whole or in part to comprise human nucleic acid sequence encoding at least one protein-coding sequence of a mature IL2RB protein.

In some embodiments, the genetically modified mice can express the human IL2RB and/or chimeric IL2RB (e.g., humanized IL2RB) from endogenous loci that are under control of mouse promoters and/or mouse regulatory elements. The replacement(s) at the endogenous mouse loci provide non-human animals that express human IL2RB or chimeric IL2RB (e.g., humanized IL2RB) in appropriate cell types and in a manner that does not result in the potential pathologies observed in some other transgenic mice known in the art. The human IL2RB or the chimeric IL2RB (e.g., humanized IL2RB) expressed in animal can maintain one or more functions of the wild-type mouse or human IL2RB in the animal. Furthermore, in some embodiments, the animal does not express endogenous IL2RB. In some embodiments, the animal expresses a decreased level of endogenous IL2RB as compared to IL2RB expression level in a wild-type animal. As used herein, the term “endogenous IL2RB” refers to IL2RB protein that is expressed from an endogenous IL2RB nucleotide sequence of the non-human animal (e.g., mouse) before any genetic modification.

The genome of the animal can comprise a sequence encoding an amino acid sequence that is at least 70%, 75%, 80%, 85%, 90%, 95%, 99%, or 100% identical to human IL2RB (NP_000869.1; SEQ ID NO: 34). In some embodiments, the genome comprises a sequence encoding an amino acid sequence that is at least 70%, 75%, 80%, 85%, 90%, 95%, 99%, or 100% identical to SEQ ID NO: 33, 34, or 39.

The genome of the genetically modified animal can comprise a replacement at an endogenous IL2RB gene locus of a sequence encoding a region of endogenous IL2RB with a sequence encoding a corresponding region of human IL2RB. In some embodiments, the sequence that is replaced is any sequence within the endogenous IL2RB gene locus, e.g., exon 1, exon 2, exon 3, exon 4, exon 5, exon 6, exon 7, exon 8, exon 9, exon 10, 5′-UTR, 3′-UTR, intron 1, intron 2, intron 3, intron 4, intron 5, intron 6, intron 7, intron 8, intron 9, or any combination thereof. In some embodiments, the sequence that is replaced is within the regulatory region of the endogenous IL2RB gene. In some embodiments, the sequence that is replaced is a portion of exon 2, exons 3-7, and a portion of exon 8, of an endogenous mouse IL2RB gene locus.

The genetically modified animal can have one or more cells expressing a human or chimeric IL2RB (e.g., humanized IL2RB) having, from N-terminus to C-terminus, a signal peptide, an extracellular region, a transmembrane region, and a cytoplasmic region. In some embodiments, the signal peptide comprises a sequence that is at least 50%, 60%, 70%, 80%, 90%, 95%, 99% identical to the signal peptide of endogenous IL2RB. In some embodiments, the signal peptide of the humanized IL2RB has a sequence that has at least 1, 2, 3, 4, 5, 6, 7, 8, 9, 10, 11, 12, 13, 14, 15, 16, 17, 18, 19, 20, 21, 22, 23, 24, 25, or 26 amino acids (e.g., contiguously or non-contiguously) that are identical to the signal peptide of endogenous IL2RB (e.g., mouse IL2RB). In some embodiments, the extracellular region comprises a sequence that is at least 50%, 60%, 70%, 80%, 90%, 95%, 99% identical to the extracellular region of human IL2RB. In some embodiments, the extracellular region of the humanized IL2RB has a sequence that has at least 10, 20, 30, 40, 50, 60, 70, 80, 90, 100, 110, 120, 130, 140, 150, 160, 170, 180, 190, 200, 205, 206, 207, 208, 209, 210, 211, 212, 213, or 214 amino acids (e.g., contiguously or non-contiguously) that are identical to the extracellular region of human IL2RB. In some embodiments, the extracellular region of the humanized IL2RB has a sequence that has at least 1, 2, 3, or 4 amino acids (contiguously or non-contiguously) that are identical to the N-terminal 1-2 amino acids and/or the C-terminal 1-2 amino acids in the extracellular region of endogenous IL2RB (e.g., mouse IL2RB). In some embodiments, the extracellular region described herein includes the signal peptide. In some embodiments, the extracellular region described herein does not include the signal peptide. Because human IL2RB and non-human IL2RB (e.g., mouse IL2RB) sequences, in many cases, are different, antibodies that bind to human IL2RB will not necessarily have the same binding affinity with non-human IL2RB or have the same effects to non-human IL2RB. Therefore, the genetically modified animal having a human or a humanized extracellular region can be used to better evaluate the effects of anti-human IL2RB antibodies in an animal model.

In some embodiments, the transmembrane region comprises a sequence that is at least 50%, 60%, 70%, 80%, 90%, 95%, 99% identical to the transmembrane region of endogenous IL2RB (e.g., amino acids 241-268 of SEQ ID NO: 33). In some embodiments, the transmembrane region of the humanized IL2RB has a sequence that has at least 1, 2, 3, 4, 5, 6, 7, 8, 9, 10, 11, 12, 13, 14, 15, 16, 17, 18, 19, 20, 21, 22, 23, 24, 25, 26, 27, or 28 amino acids (contiguously or non-contiguously) that are identical to the transmembrane region of endogenous IL2RB (e.g., mouse IL2RB). In some embodiments, the cytoplasmic comprises a sequence that is at least 50%, 60%, 70%, 80%, 90%, 95%, 99% identical to the cytoplasmic of endogenous IL2RB (e.g., amino acids 269-539 of SEQ ID NO: 33). In some embodiments, the cytoplasmic region of the humanized IL2RB has a sequence that has at least 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, or 271 amino acids (contiguously or non-contiguously) that are identical to the cytoplasmic region of endogenous IL2RB (e.g., mouse IL2RB).

In some embodiments, the entire signal peptide, the entire transmembrane region, and the entire cytoplasmic region of the humanized IL2RB described herein are derived from endogenous sequence.

In some embodiments, the genome of the genetically modified animal comprises a sequence encoding an amino acid sequence that corresponds to a portion or the entire sequence of exon 1, exon 2, exon 3, exon 4, exon 5, exon 6, exon 7, exon 8, exon 9, and/or exon 10 of human IL2RB; and/or a portion or the entire sequence of the extracellular region of human IL2RB; or a portion or the entire sequence of amino acids 29-237, 29-240, 27-237, or 27-240 of SEQ ID NO: 34.

In some embodiments, the genome of the genetically modified animal comprises a portion of exon 2, exons 3-7, and a portion of exon 8 of human IL2RB gene. In some embodiments, the portion of exon 2 includes at least 1, 2, 3, 4, 5, 6, 7, 8, 9, 10, 15, 20, 30, 40, 50, 60, 70, 80, 90, 100, 110, 120, or 121 nucleotides. In some embodiments, the portion of exon 2 includes 4 nucleotides. In some embodiments, the portion of exon 2 includes a nucleotide of at least 1 bp. In some embodiments, the portion of exon 8 includes at least 1, 2, 3, 4, 5, 6, 7, 8, 9, 10, 15, 20, 30, 40, 50, 60, 70, 80, 90, 100, 110, 111, 112, 113, 114, or 115 nucleotides. In some embodiments, the portion of exon 8 includes 8 nucleotides. In some embodiments, the portion of exon 8 includes a nucleotide of at least 2 bp.

In some embodiments, the non-human animal can have, at an endogenous IL2RB gene locus, a nucleotide sequence encoding a chimeric human/non-human IL2RB polypeptide, wherein a human portion of the chimeric human/non-human IL2RB polypeptide comprises all or a portion of the human IL2RB extracellular region, and wherein the animal expresses a functional IL2RB on a surface of a cell of the animal. The human portion of the chimeric human/non-human IL2RB polypeptide can comprise an amino acid sequence encoded by a portion of exon 2, exons 3-7, and/or a portion of exon 8 of human IL2RB gene. In some embodiments, the human portion of the chimeric human/non-human IL2RB polypeptide can comprise a sequence that is at least 80%, 85%, 90%, 95%, or 99% identical to amino acids 29-237 of SEQ ID NO: 34. In some embodiments, the transmembrane region includes a sequence corresponding to the entire or part of amino acids 241-268 of SEQ ID NO: 33. In some embodiments, the cytoplasmic region includes a sequence corresponding to the entire or part of amino acids 269-539 of SEQ ID NO: 33. In some embodiments, the chimeric human/non-human IL2RB polypeptide comprises a signal peptide, which includes a sequence corresponding to the entire or part of amino acids 1-26 of SEQ ID NO: 33.

In some embodiments, the non-human portion of the chimeric human/non-human IL2RB polypeptide comprises the entire signal peptide, the entire transmembrane region, and/or the entire cytoplasmic region of an endogenous non-human IL2RB polypeptide.

Furthermore, the genetically modified animal can be heterozygous with respect to the replacement at the endogenous IL2RB locus, or homozygous with respect to the replacement at the endogenous IL2RB locus.

In some embodiments, the humanized IL2RB locus lacks a human IL2RB gene 5′-UTR. In some embodiment, the humanized IL2RB locus comprises an endogenous (e.g., mouse) 5′-UTR. In some embodiments, the humanized IL2RB locus comprises an endogenous (e.g., mouse) 3′-UTR. In some embodiments, the humanized IL2RB locus comprises human 3′-UTR. In appropriate cases, it may be reasonable to presume that the mouse and human IL2RB genes appear to be similarly regulated based on the similarity of their 5′-flanking sequence. As shown in the present disclosure, humanized IL2RB mice that comprise a replacement at an endogenous mouse IL2RB locus, which retain mouse regulatory elements but comprise a humanization of IL2RB encoding sequence, do not exhibit pathologies. Both genetically modified mice that are heterozygous or homozygous for humanized IL2RB are grossly normal.

In another aspect, the disclosure also provides a genetically-modified, non-human animal whose genome comprise a disruption in the animal's endogenous IL2RB gene, wherein the disruption of the endogenous IL2RB gene comprises deletion of exon 1, exon 2, exon 3, exon 4, exon 5, exon 6, exon 7, exon 8, exon 9, and/or exon 10, or part thereof of the endogenous IL2RB gene.

In some embodiments, the disruption of the endogenous IL2RB gene comprises deletion of one or more exons or part of exons selected from the group consisting of exon 1, exon 2, exon 3, exon 4, exon 5, exon 6, exon 7, exon 8, exon 9, and exon 10 of the endogenous IL2RB gene.

In some embodiments, the disruption of the endogenous IL2RB gene further comprises deletion of one or more introns or part of introns selected from the group consisting of intron 1, intron 2, intron 3, intron 4, intron 5, intron 6, intron 7, intron 8, and intron 9 of the endogenous IL2RB gene.

In some embodiments, wherein the deletion can comprise deleting at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 300, 400, 500, 600, 700, 800, 900, 1000, 1100, 1200, 1300, 1400, 1500, 1600, 1700, 1800, 1900, 2000, 2500, 3000, 5000, 6000, 7000, 8000, 9000, 10000, 15000, 20000, 25000, 30000, 32000, 32366, or more nucleotides.

In some embodiments, the disruption of the endogenous IL2RB gene comprises the deletion of at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 300, 400, 500, 600, 610, 620, or 630 nucleotides of exon 1, exon 2, exon 3, exon 4, exon 5, exon 6, exon 7, exon 8, exon 9, and/or exon 10 (e.g., deletion of at least 1 nucleotides from exon 2, exons 3-7, and at least 2 nucleotides from exon 8).

### Animals Having Humanized IL15RA Gene Locus

In some embodiments, the chimeric gene or the chimeric nucleic acid is a humanized IL15RA gene or a humanized IL15RA nucleic acid. In some embodiments, at least one or more portions of the gene or the nucleic acid is from the human IL15RA gene, at least one or more portions of the gene or the nucleic acid is from a non-human IL15RA gene. In some embodiments, the gene or the nucleic acid comprises a sequence that encodes an IL15RA protein. The encoded IL15RA protein is functional or has at least one activity of the human IL15RA protein or the non-human IL15RA protein.

In some embodiments, the chimeric protein or the chimeric polypeptide is a humanized IL15RA protein or a humanized IL15RA polypeptide. In some embodiments, at least one or more portions of the amino acid sequence of the protein or the polypeptide is from a human IL15RA protein, and at least one or more portions of the amino acid sequence of the protein or the polypeptide is from a non-human IL15RA protein. The humanized IL15RA protein or the humanized IL15RA polypeptide is functional or has at least one activity of the human IL15RA protein or the non-human IL15RA protein.

In some embodiments, the IL15RA extracellular region is human or humanized. In some embodiments, the IL15RA signal peptide is human or humanized. In some embodiments, the IL15RA cytoplasmic region is human or humanized. In some embodiments, the IL15RA transmembrane region is human or humanized. In some embodiments, both the IL15RA extracellular region and transmembrane region are human or humanized. In some embodiments, both the IL15RA signal peptide and cytoplasmic regions are endogenous.

Genetically modified non-human animals can comprise a modification at an endogenous non-human IL15RA locus. In some embodiments, the modification can comprise a human nucleic acid sequence encoding at least a portion of a mature IL15RA protein (e.g., at least 10%, 20%, 30%, 40%, 50%, 60%, 70%, 80%, 90%, 95%, 96%, 97%, 98%, or 99% identical to the mature IL15RA protein sequence). Although genetically modified cells are also provided that can comprise the modifications described herein (e.g., ES cells, somatic cells), in many embodiments, the genetically modified non-human animals comprise the modification of the endogenous IL15RA locus in the germline of the animal.

Genetically modified animals can express a human IL15RA and/or a chimeric (e.g., humanized) IL15RA from endogenous mouse loci, wherein the endogenous mouse IL15RA gene has been replaced with a human IL15RA gene and/or a nucleotide sequence that encodes a region of human IL15RA sequence or an amino acid sequence that is at least 10%, 20%, 30%, 40%, 50%, 60%, 70%, 80%, 90%, 95%, 96%, 97%, 98%, or 99% identical to the human IL15RA sequence. In various embodiments, an endogenous non-human IL15RA locus is modified in whole or in part to comprise human nucleic acid sequence encoding at least one protein-coding sequence of a mature IL15RA protein.

In some embodiments, the genetically modified mice can express the human IL15RA and/or chimeric IL15RA (e.g., humanized IL15RA) from endogenous loci that are under control of mouse promoters and/or mouse regulatory elements. The replacement(s) at the endogenous mouse loci provide non-human animals that express human IL15RA or chimeric IL15RA (e.g., humanized IL15RA) in appropriate cell types and in a manner that does not result in the potential pathologies observed in some other transgenic mice known in the art. The human IL15RA or the chimeric IL15RA (e.g., humanized IL15RA) expressed in animal can maintain one or more functions of the wild-type mouse or human IL15RA in the animal. Furthermore, in some embodiments, the animal does not express endogenous IL15RA. In some embodiments, the animal expresses a decreased level of endogenous IL15RA as compared to IL15RA expression level in a wild-type animal. As used herein, the term “endogenous IL15RA” refers to IL15RA protein that is expressed from an endogenous IL15RA nucleotide sequence of the non-human animal (e.g., mouse) before any genetic modification.

The genome of the animal can comprise a sequence encoding an amino acid sequence that is at least 70%, 75%, 80%, 85%, 90%, 95%, 99%, or 100% identical to human IL15RA (NP_002180.1; SEQ ID NO: 43). In some embodiments, the genome comprises a sequence encoding an amino acid sequence that is at least 70%, 75%, 80%, 85%, 90%, 95%, 99%, or 100% identical to SEQ ID NO: 42, 43, or 50.

The genome of the genetically modified animal can comprise a replacement at an endogenous IL15RA gene locus of a sequence encoding a region of endogenous IL15RA with a sequence encoding a corresponding region of human IL15RA. In some embodiments, the sequence that is replaced is any sequence within the endogenous IL15RA gene locus, e.g., exon 1, exon 2, exon 3, exon 4, exon 5, exon 6, exon 7, 5′-UTR, 3′-UTR, intron 1, intron 2, intron 3, intron 4, intron 5, intron 6, or any combination thereof. In some embodiments, the sequence that is replaced is within the regulatory region of the endogenous IL15RA gene. In some embodiments, the sequence that is replaced is a portion of exon 2, exons 3-5, and a portion of exon 6, of an endogenous mouse IL15RA gene locus.

The genetically modified animal can have one or more cells expressing a human or chimeric IL15RA (e.g., humanized IL15RA) having, from N-terminus to C-terminus, a signal peptide, an extracellular region, a transmembrane region, and a cytoplasmic region. In some embodiments, the signal peptide comprises a sequence that is at least 50%, 60%, 70%, 80%, 90%, 95%, 99% identical to the signal peptide of endogenous IL15RA. In some embodiments, the signal peptide of the humanized IL15RA has a sequence that has at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 amino acids (e.g., contiguously or non-contiguously) that are identical to the signal peptide of endogenous IL15RA (e.g., mouse IL15RA). In some embodiments, the extracellular region comprises a sequence that is at least 50%, 60%, 70%, 80%, 90%, 95%, 99% identical to the extracellular region of human IL15RA. In some embodiments, the extracellular region of the humanized IL15RA has a sequence that has at least 10, 20, 30, 40, 50, 60, 70, 80, 90, 100, 110, 120, 130, 140, 150, 160, 165, 166, 167, 168, 169, 170, 171, 172, 173, 174, or 175 amino acids (e.g., contiguously or non-contiguously) that are identical to the extracellular region of human IL15RA. In some embodiments, the extracellular region of the humanized IL15RA has a sequence that has at least 1, 2, 3, 4, 5, 6, or 7 amino acids (contiguously or non-contiguously) that are identical to the N-terminal 1-7 amino acids in the extracellular region of endogenous IL15RA (e.g., mouse IL15RA). In some embodiments, the extracellular region described herein includes the signal peptide. In some embodiments, the extracellular region described herein does not include the signal peptide. Because human IL15RA and non-human IL15RA (e.g., mouse IL15RA) sequences, in many cases, are different, antibodies that bind to human IL15RA will not necessarily have the same binding affinity with non-human IL15RA or have the same effects to non-human IL15RA. Therefore, the genetically modified animal having a human or a humanized extracellular region can be used to better evaluate the effects of anti-human IL15RA antibodies in an animal model.

In some embodiments, the transmembrane region comprises a sequence that is at least 50%, 60%, 70%, 80%, 90%, 95%, 99% identical to the transmembrane region of endogenous IL15RA (e.g., amino acids 212-226 of SEQ ID NO: 42). In some embodiments, the transmembrane region of the humanized IL15RA has a sequence that has at least 1, 2, 3, 4, 5, 6, 7, 8, 9, 10, 11, 12, 13, 14, 15, 16, 17, 18, 19, 20, or 21 amino acids (contiguously or non-contiguously) that are identical to the transmembrane region of endogenous IL15RA (e.g., mouse IL15RA). In some embodiments, the transmembrane region of the humanized IL15RA has a sequence that has at least 1, 2, 3, 4, 5, or 6 amino acids (contiguously or non-contiguously) that are identical to the N-terminal 1-6 amino acids in the extracellular region of human IL15RA. In some embodiments, the cytoplasmic comprises a sequence that is at least 50%, 60%, 70%, 80%, 90%, 95%, 99% identical to the cytoplasmic of endogenous IL15RA (e.g., amino acids 227-263 of SEQ ID NO: 42). In some embodiments, the cytoplasmic region of the humanized IL15RA has a sequence that has at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 37 amino acids (contiguously or non-contiguously) that are identical to the cytoplasmic region of endogenous IL15RA (e.g., mouse IL15RA).

In some embodiments, the entire signal peptide and the entire cytoplasmic region of the humanized IL15RA described herein are derived from endogenous sequence.

In some embodiments, the genome of the genetically modified animal comprises a sequence encoding an amino acid sequence that corresponds to a portion or the entire sequence of exon 1, exon 2, exon 3, exon 4, exon 5, exon 6, and/or exon 7 of human IL15RA; and/or a portion or the entire sequence of the extracellular region and the transmembrane region of human IL15RA; or a portion or the entire sequence of amino acids 37-211 or 31-205 of SEQ ID NO: 43.

In some embodiments, the genome of the genetically modified animal comprises a portion of exon 2, exons 3-5, and a portion of exon 6 of human IL15RA gene. In some embodiments, the portion of exon 2 includes at least 1, 2, 3, 4, 5, 6, 7, 8, 9, 10, 15, 20, 30, 40, 50, 60, 70, 80, 90, 100, 110, 120, 130, 140, 150, 160, 170, 173, 174, 175, 180, 190, or 195 nucleotides. In some embodiments, the portion of exon 2 includes 175 nucleotides. In some embodiments, the portion of exon 2 includes a nucleotide of at least 50 bp. In some embodiments, the portion of exon 6 includes at least 1, 2, 3, 4, 5, 6, 7, 8, 9, 10, 11, 12, 13, 14, 15, 16, 17, 18, 19, 20, 30, 40, 50, 60, 70, 71, 72, 73, 74, 75, or 76 nucleotides. In some embodiments, the portion of exon 6 includes 17 nucleotides. In some embodiments, the portion of exon 6 includes a nucleotide of at least 5 bp.

In some embodiments, the non-human animal can have, at an endogenous IL15RA gene locus, a nucleotide sequence encoding a chimeric human/non-human IL15RA polypeptide, wherein a human portion of the chimeric human/non-human IL15RA polypeptide comprises all or a portion of the human IL15RA extracellular region, and wherein the animal expresses a functional IL15RA on a surface of a cell of the animal. The human portion of the chimeric human/non-human IL15RA polypeptide can comprise an amino acid sequence encoded by a portion of exon 2, exons 3-5, and/or a portion of exon 6 of human IL15RA gene. In some embodiments, the human portion of the chimeric human/non-human IL15RA polypeptide can comprise a sequence that is at least 80%, 85%, 90%, 95%, or 99% identical to amino acids 37-211 of SEQ ID NO: 43. In some embodiments, the transmembrane region includes a sequence corresponding to the entire or part of amino acids 212-226 of SEQ ID NO: 42. In some embodiments, the cytoplasmic region includes a sequence corresponding to the entire or part of amino acids 227-263 of SEQ ID NO: 42. In some embodiments, the chimeric human/non-human IL15RA polypeptide comprises a signal peptide, which includes a sequence corresponding to the entire or part of amino acids 1-32 of SEQ ID NO: 42.

In some embodiments, the non-human portion of the chimeric human/non-human IL15RA polypeptide comprises the entire signal peptide, and/or the entire cytoplasmic region of an endogenous non-human IL15RA polypeptide.

Furthermore, the genetically modified animal can be heterozygous with respect to the replacement at the endogenous IL15RA locus, or homozygous with respect to the replacement at the endogenous IL15RA locus.

In some embodiments, the humanized IL15RA locus lacks a human IL15RA gene 5′-UTR. In some embodiment, the humanized IL15RA locus comprises an endogenous (e.g., mouse) 5′-UTR. In some embodiments, the humanized IL15RA locus comprises an endogenous (e.g., mouse) 3′-UTR. In some embodiments, the humanized IL15RA locus comprises human 3′-UTR. In appropriate cases, it may be reasonable to presume that the mouse and human IL15RA genes appear to be similarly regulated based on the similarity of their 5′-flanking sequence. As shown in the present disclosure, humanized IL15RA mice that comprise a replacement at an endogenous mouse IL15RA locus, which retain mouse regulatory elements but comprise a humanization of IL15RA encoding sequence, do not exhibit pathologies. Both genetically modified mice that are heterozygous or homozygous for humanized IL15RA are grossly normal.

In another aspect, the disclosure also provides a genetically-modified, non-human animal whose genome comprise a disruption in the animal's endogenous IL15RA gene, wherein the disruption of the endogenous IL15RA gene comprises deletion of exon 1, exon 2, exon 3, exon 4, exon 5, exon 6, and/or exon 7, or part thereof of the endogenous IL15RA gene.

In some embodiments, the disruption of the endogenous IL15RA gene comprises deletion of one or more exons or part of exons selected from the group consisting of exon 1, exon 2, exon 3, exon 4, exon 5, exon 6, and exon 7 of the endogenous IL15RA gene.

In some embodiments, the disruption of the endogenous IL15RA gene further comprises deletion of one or more introns or part of introns selected from the group consisting of intron 1, intron 2, intron 3, intron 4, intron 5, and intron 6 of the endogenous IL15RA gene.

In some embodiments, wherein the deletion can comprise deleting at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 300, 400, 500, 600, 700, 800, 900, 1000, 1100, 1200, 1300, 1400, 1500, 1600, 1700, 1800, 1900, 2000, 2500, 3000, 5000, 6000, 7000, 8000, 9000, 10000, 15000, 20000, 25000, 26000, 27000, 28000, 28805, or more nucleotides.

In some embodiments, the disruption of the endogenous IL15RA gene comprises the deletion of at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 300, 400, 500, 510, 515, or 516 nucleotides of exon 1, exon 2, exon 3, exon 4, exon 5, exon 6, and/or exon 7 (e.g., deletion of at least 50 nucleotides from exon 2, exons 3-5, and at least 5 nucleotides from exon 6).

### Animals Having Humanized IL15 Gene Locus

In some embodiments, the chimeric gene or the chimeric nucleic acid is a humanized IL15 gene or a humanized IL15 nucleic acid. In some embodiments, at least one or more portions of the gene or the nucleic acid is from the human IL15 gene, at least one or more portions of the gene or the nucleic acid is from a non-human IL15 gene. In some embodiments, the gene or the nucleic acid comprises a sequence that encodes an IL15 protein. The encoded IL15 protein is functional or has at least one activity of the human IL15 protein or the non-human IL15 protein.

In some embodiments, the chimeric protein or the chimeric polypeptide is a humanized IL15 protein or a humanized IL15 polypeptide. In some embodiments, at least one or more portions of the amino acid sequence of the protein or the polypeptide is from a human IL15 protein, and at least one or more portions of the amino acid sequence of the protein or the polypeptide is from a non-human IL15 protein. The humanized IL15 protein or the humanized IL15 polypeptide is functional or has at least one activity of the human IL15 protein or the non-human IL15 protein.

In some embodiments, the humanized IL15 protein includes a polypeptide sequence of 5-162 amino acids (contiguous or non-contiguous) that is identical to human IL15 protein. In some embodiments, the polypeptide sequence is 10-162 amino acids in length. In some embodiments, the humanized IL15 gene includes a nucleotide sequence of 20-97405 bp (contiguous or non-contiguous) that is identical to human IL15 gene. In some embodiments, the nucleotide sequence is 20-13384 bp, 20-2012 bp, or 20-489 bp.

Genetically modified non-human animals can comprise a modification at an endogenous non-human IL15 locus. In some embodiments, the modification can comprise a human nucleic acid sequence encoding at least a portion of a mature IL15 protein (e.g., at least 10%, 20%, 30%, 40%, 50%, 60%, 70%, 80%, 90%, 95%, 96%, 97%, 98%, or 99% identical to the mature IL15 protein sequence). Although genetically modified cells are also provided that can comprise the modifications described herein (e.g., ES cells, somatic cells), in many embodiments, the genetically modified non-human animals comprise the modification of the endogenous IL15 locus in the germline of the animal.

Genetically modified animals can express a human IL15 and/or a chimeric (e.g., humanized) IL15 from endogenous mouse loci, wherein the endogenous mouse IL15 gene has been replaced with a human IL15 gene and/or a nucleotide sequence that encodes a region of human IL15 sequence or an amino acid sequence that is at least 10%, 20%, 30%, 40%, 50%, 60%, 70%, 80%, 90%, 95%, 96%, 97%, 98%, or 99% identical to the human IL15 sequence. In various embodiments, an endogenous non-human IL15 locus is modified in whole or in part to comprise human nucleic acid sequence encoding at least one protein-coding sequence of a mature IL15 protein.

In some embodiments, the genetically modified mice can express the human IL15 and/or chimeric IL15 (e.g., humanized IL15) from endogenous loci that are under control of mouse promoters and/or mouse regulatory elements. The replacement(s) at the endogenous mouse loci provide non-human animals that express human IL15 or chimeric IL15 (e.g., humanized IL15) in appropriate cell types and in a manner that does not result in the potential pathologies observed in some other transgenic mice known in the art. The human IL15 or the chimeric IL15 (e.g., humanized IL15) expressed in animal can maintain one or more functions of the wild-type mouse or human IL15 in the animal. Furthermore, in some embodiments, the animal does not express endogenous IL15. In some embodiments, the animal expresses a decreased level of endogenous IL15 as compared to IL15 expression level in a wild-type animal. As used herein, the term “endogenous IL15” refers to IL15 protein that is expressed from an endogenous IL15 nucleotide sequence of the non-human animal (e.g., mouse) before any genetic modification.

The genome of the animal can comprise a sequence encoding an amino acid sequence that is at least 70%, 75%, 80%, 85%, 90%, 95%, 99%, or 100% identical to human IL15 (NP_000576.1; SEQ ID NO: 52). In some embodiments, the genome comprises a sequence encoding an amino acid sequence that is at least 70%, 75%, 80%, 85%, 90%, 95%, 99%, or 100% identical to SEQ ID NO: 51 or 52.

The genome of the genetically modified animal can comprise a replacement at an endogenous IL15 gene locus of a sequence encoding a region of endogenous IL15 with a sequence encoding a corresponding region of human IL15. In some embodiments, the sequence that is replaced is any sequence within the endogenous IL15 gene locus, e.g., exon 1, exon 2, exon 3, exon 4, exon 5, exon 6, exon 7, exon 8, 5′-UTR, 3′-UTR, intron 1, intron 2, intron 3, intron 4, intron 5, intron 6, intron 7, or any combination thereof. In some embodiments, the sequence that is replaced is within the regulatory region of the endogenous IL15 gene. In some embodiments, the sequence that is replaced is a portion of exon 3, exons 4-7, and a portion of exon 8, of an endogenous mouse IL15 gene locus.

The genetically modified animal can have one or more cells expressing a human or chimeric IL15 (e.g., humanized IL15) having, an N-terminal signal peptide. In some embodiments, the signal peptide comprises a sequence that is at least 50%, 60%, 70%, 80%, 90%, 95%, 99% identical to the signal peptide of endogenous IL15. In some embodiments, the signal peptide of the humanized IL15 has a sequence that has at least 1, 2, 3, 4, 5, 6, 7, 8, 9, 10, 11, 12, 13, 14, 15, 16, 17, 18, 19, 20, 21, 22, 23, 24, 25, 26, 27, 28, or 29 amino acids (e.g., contiguously or non-contiguously) that are identical to the signal peptide of endogenous IL15 (e.g., mouse IL15). In some embodiments, the signal peptide comprises a sequence that is at least 50%, 60%, 70%, 80%, 90%, 95%, 99% identical to the signal peptide of human IL15. In some embodiments, the signal peptide of the humanized IL15 has a sequence that has at least 1, 2, 3, 4, 5, 6, 7, 8, 9, 10, 11, 12, 13, 14, 15, 16, 17, 18, 19, 20, 21, 22, 23, 24, 25, 26, 27, 28, or 29 amino acids (e.g., contiguously or non-contiguously) that are identical to the signal peptide of human IL15.

In some embodiments, the genome of the genetically modified animal comprises a sequence encoding an amino acid sequence that corresponds to a portion or the entire sequence of exon 1, exon 2, exon 3, exon 4, exon 5, exon 6, exon 7, and/or exon 8 of human IL15; and/or the entire sequence of human IL15; or a portion or the entire sequence of amino acids 1-162 or 30-162 of SEQ ID NO: 52.

In some embodiments, the genome of the genetically modified animal comprises a portion of exon 3, exons 4-7, and a portion of exon 8 of human IL15 gene. In some embodiments, the portion of exon 3 includes at least 1, 2, 3, 4, 5, 6, 7, 8, 9, 10, 11, 12, 13, 14, 15, 20, 30, 40, 50, 60, 70, 80, 90, 100, 105, 110, or 111 nucleotides. In some embodiments, the portion of exon 3 includes 12 nucleotides. In some embodiments, the portion of exon 3 includes a nucleotide of at least 5 bp. In some embodiments, the portion of exon 8 includes at least 1, 2, 3, 4, 5, 6, 7, 8, 9, 10, 11, 12, 13, 14, 15, 16, 17, 18, 19, 20, 30, 40, 50, 60, 70, 80, 90, 100, 110, 111, 120, 130, 140, 150, 200, 300, 400, 500, 600, 700, 800, 900, 1000, 1100, 1200, or 1250 nucleotides. In some embodiments, the portion of exon 8 includes 111 nucleotides. In some embodiments, the portion of exon 8 includes a nucleotide of at least 50 bp.

Furthermore, the genetically modified animal can be heterozygous with respect to the replacement at the endogenous IL15 locus, or homozygous with respect to the replacement at the endogenous IL15 locus.

In some embodiments, the humanized IL15 locus lacks a human IL15 gene 5′-UTR. In some embodiment, the humanized IL15 locus comprises an endogenous (e.g., mouse) 5′-UTR. In some embodiments, the humanized IL15 locus comprises an endogenous (e.g., mouse) 3′-UTR. In some embodiments, the humanized IL15 locus comprises human 3′-UTR. In appropriate cases, it may be reasonable to presume that the mouse and human IL15 genes appear to be similarly regulated based on the similarity of their 5′-flanking sequence. As shown in the present disclosure, humanized IL15 mice that comprise a replacement at an endogenous mouse IL15 locus, which retain mouse regulatory elements but comprise a humanization of IL15 encoding sequence, do not exhibit pathologies. Both genetically modified mice that are heterozygous or homozygous for humanized IL15 are grossly normal.

In another aspect, the disclosure also provides a genetically-modified, non-human animal whose genome comprise a disruption in the animal's endogenous IL15 gene, wherein the disruption of the endogenous IL15 gene comprises deletion of exon 1, exon 2, exon 3, exon 4, exon 5, exon 6, exon 7, and/or exon 8, or part thereof of the endogenous IL15 gene.

In some embodiments, the disruption of the endogenous IL15 gene comprises deletion of one or more exons or part of exons selected from the group consisting of exon 1, exon 2, exon 3, exon 4, exon 5, exon 6, exon 7, and exon 8 of the endogenous IL15 gene.

In some embodiments, the disruption of the endogenous IL15 gene further comprises deletion of one or more introns or part of introns selected from the group consisting of intron 1, intron 2, intron 3, intron 4, intron 5, intron 6, and intron 7 of the endogenous IL15 gene.

In some embodiments, wherein the deletion can comprise deleting at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 300, 400, 500, 600, 700, 800, 900, 1000, 1100, 1200, 1300, 1400, 1500, 1600, 1700, 1800, 1900, 2000, 2500, 3000, 5000, 6000, 7000, 8000, 9000, 10000, 15000, 20000, 25000, 30000, 35000, 40000, 45000, 50000, 55000, 60000, 65000, 70000, 71631, or more nucleotides.

In some embodiments, the disruption of the endogenous IL15 gene comprises the deletion of at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 300, 400, 450, 460, 470, 480, 485, or 489 nucleotides of exon 1, exon 2, exon 3, exon 4, exon 5, exon 6, exon 7, and/or exon 8 (e.g., deletion of at least 5 nucleotides from exon 3, exons 4-7, and at least 50 nucleotides from exon 8).

The genetically modified non-human animal can be various animals, e.g., a mouse, rat, rabbit, pig, bovine (e.g., cow, bull, buffalo), deer, sheep, goat, chicken, cat, dog, ferret, primate (e.g., marmoset, rhesus monkey). For the non-human animals where suitable genetically modifiable embryonic stem (ES) cells are not readily available, other methods are employed to make a non-human animal comprising the genetic modification. Such methods include, e.g., modifying a non-ES cell genome (e.g., a fibroblast or an induced pluripotent cell) and employing nuclear transfer to transfer the modified genome to a suitable cell, e.g., an oocyte, and gestating the modified cell (e.g., the modified oocyte) in a non-human animal under suitable conditions to form an embryo. These methods are known in the art, and are described, e.g., in A. Nagy, et al., “Manipulating the Mouse Embryo: A Laboratory Manual (Third Edition),” Cold Spring Harbor Laboratory Press, 2006, which is incorporated by reference herein in its entirety.

In one aspect, the animal is a mammal, e.g., of the superfamily Dipodoidea or Muroidea. In some embodiments, the genetically modified animal is a rodent. The rodent can be selected from a mouse, a rat, and a hamster. In some embodiments, the genetically modified animal is from a family selected from Calomyscidae (e.g., mouse-like hamsters), Cricetidae (e.g., hamster, New World rats and mice, voles), Muridae (true mice and rats, gerbils, spiny mice, crested rats), Nesomyidae (climbing mice, rock mice, with-tailed rats, Malagasy rats and mice), Platacanthomyidae (e.g., spiny dormice), and Spalacidae (e.g., mole rates, bamboo rats, and zokors). In some embodiments, the genetically modified rodent is selected from a true mouse or rat (family Muridae), a gerbil, a spiny mouse, and a crested rat. In some embodiments, the non-human animal is a mouse.

In some embodiments, the animal is a mouse of a C57BL strain selected from C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL/10Cr, and C57BL/Ola. In some embodiments, the mouse is a 129 strain selected from the group consisting of a strain that is 129P1, 129P2, 129P3, 129X1, 129S1 (e.g., 129S1/SV, 129S1/SvIm), 129S2, 129S4, 129S5, 129S9/SvEvH, 129S6 (129/SvEvTac), 129S7, 129S8, 129T1, 129T2. These mice are described, e.g., in Festing et al., Revised nomenclature for strain 129 mice, Mammalian Genome 10:836 (1999); Auerbach et al., Establishment and Chimera Analysis of 129/SvEv- and C57BL/6-Derived Mouse Embryonic Stem Cell Lines (2000), both of which are incorporated herein by reference in the entirety. In some embodiments, the genetically modified mouse is a mix of the 129 strain and the C57BL/6 strain. In some embodiments, the mouse is a mix of the 129 strains, or a mix of the BL/6 strains. In some embodiments, the mouse is a BALB strain, e.g., BALB/c strain. In some embodiments, the mouse is a mix of a BALB strain and another strain. In some embodiments, the mouse is from a hybrid line (e.g., 50% BALB/c-50%12954/Sv; or 50% C57BL/6-50%129). In some embodiments, the non-human animal is a rodent. In some embodiments, the non-human animal is a mouse having a BALB/c, A, A/He, A/J, A/WySN, AKR, AKR/A, AKR/J, AKR/N, TA1, TA2, RF, SWR, C3H, C57BR, SJL, C57L, DBA/2, KM, NIH, ICR, CFW, FACA, C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL (C57BL/10Cr and C57BL/Ola), C58, CBA/Br, CBA/Ca, CBA/J, CBA/st, or CBA/H background.

In one aspect, the non-human animal is a mammal. In one aspect, the non-human animal is a small mammal, e.g., a jerboa. In one embodiment, the genetically humanized non-human animal is a rodent. In one embodiment, the rodent is selected from the group consisting of mice, rats and hamsters. In one embodiment, the rodent is selected from the murine family. In one embodiment, the genetically modified animal is selected from a group consisting of hamsteridae (e.g., mouse-like hamsters), hamsteridae (e.g., hamsters, New World rats and mice, voles), murine superfamily (e.g., true mouse and rats, gerbils, spiny rats, and crested rats), Falkomuridae (e.g., climbing mice, rock mice, tailed rats, Madagascar rats and mice), Dormocidae (e.g., spiny dormouse) and Molcidae (e.g., mole rats, bamboo rats, and zokors) families. In a specific embodiment, the genetically modified rodent is selected from the group consisting of true mice or rats (Muridae), gerbils, spiny rats and crested rats. In one embodiment, the genetically modified mouse is from a member of the family Muridae. In one embodiment, the animal is a rodent. In a specific embodiment, the rodent is selected from mice and rats. In one embodiment, the non-human animal is a mouse.

In some embodiments, the animal is a rat. The rat can be selected from a Wistar rat, an LEA strain, a Sprague Dawley strain, a Fischer strain, F344, F6, and Dark Agouti. In some embodiments, the rat strain is a mix of two or more strains selected from the group consisting of Wistar, LEA, Sprague Dawley, Fischer, F344, F6, and Dark Agouti.

The animal can have one or more other genetic modifications, and/or other modifications, that are suitable for the particular purpose for which the humanized animal is made. For example, suitable mice for maintaining a xenograft (e.g., a human cancer or tumor), can have one or more modifications that compromise, inactivate, or destroy the immune system of the non-human animal in whole or in part. Compromise, inactivation, or destruction of the immune system of the non-human animal can include, for example, destruction of hematopoietic cells and/or immune cells by chemical means (e.g., administering a toxin), physical means (e.g., irradiating the animal), and/or genetic modification (e.g., knocking out one or more genes). Non-limiting examples of such mice include, e.g., NOD mice, SCID mice, NOD/SCID mice, IL2Rγknockout mice, NOD/SCID/γc^{null} mice (Ito, M. et al., NOD/SCID/γc^{null} mouse: an excellent recipient mouse model for engraftment of human cells, Blood 100 (9): 3175-3182, 2002), nude mice, and Rag1 and/or Rag2 knockout mice. These mice can optionally be irradiated, or otherwise treated to destroy one or more immune cell type. Thus, in various embodiments, a genetically modified mouse is provided that can include a humanization of at least a portion of endogenous non-human IL2RG, IL2RB, IL15RA, and/or IL15 loci, and further comprises a modification that compromises, inactivates, or destroys the immune system (or one or more cell types of the immune system) of the non-human animal in whole or in part. In some embodiments, modification is, e.g., selected from the group consisting of a modification that results in NOD mice, SCID mice, NOD/SCID mice, IL-2RY knockout mice, NOD/SCID/γc^{null} mice, nude mice, Rag1 and/or Rag2 knockout mice, NOD-Prkdc^{scid} IL-2rγ^{null} mice, NOD-Rag 1^{-/-}-IL2rg^{-/-} (NRG) mice, Rag 2^{-/-}-IL2rg^{-/-} (RG) mice, and a combination thereof. These genetically modified animals are described, e.g., in U.S. Pat. No. 10,820,580B2, which is incorporated herein by reference in its entirety. In some embodiments, the mouse can include one or more replacements of all or part of mature IL2RG, IL2RB, IL15RA, and/or IL15 coding sequences with human mature IL2RG, IL2RB, IL15RA, and/or IL15 coding sequences, respectively.

The present disclosure further relates to a non-human mammal generated through the method mentioned above. In some embodiments, the genome thereof contains human gene(s).

In some embodiments, the non-human mammal is a rodent, and preferably, the non-human mammal is a mouse.

In some embodiments, the non-human mammal expresses a protein encoded by humanized IL2RG, IL2RB, IL15RA, and/or IL15 genes.

In addition, the present disclosure also relates to a tumor bearing non-human mammal model, characterized in that the non-human mammal model is obtained through the methods as described herein. In some embodiments, the non-human mammal is a rodent (e.g., a mouse).

The present disclosure further relates to a cell or cell line, or a primary cell culture thereof derived from the non-human mammal or an offspring thereof, or the tumor bearing non-human mammal; the tissue, organ or a culture thereof derived from the non-human mammal or an offspring thereof, or the tumor bearing non-human mammal; and the tumor tissue derived from the non-human mammal or an offspring thereof when it bears a tumor, or the tumor bearing non-human mammal.

The present disclosure also provides non-human mammals produced by any of the methods described herein. In some embodiments, a non-human mammal is provided; and the genetically modified animal contains the DNA encoding human or humanized IL2RG, IL2RB, IL15RA, and/or IL15 in the genome of the animal.

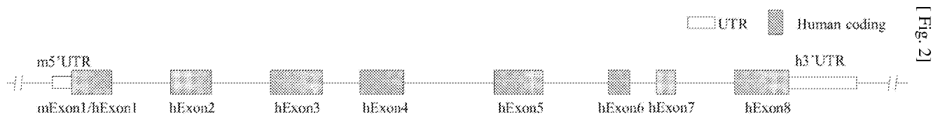

In some embodiments, the non-human mammal comprises the genetic construct as described herein (e.g., gene construct as shown in FIGS. 2, 3, 4, 8, 9, 10, 12, 17, 18, 20, 21, and 23). In some embodiments, a non-human mammal expressing human or humanized IL2RG, IL2RB, IL15RA, and/or IL15 is provided. In some embodiments, the tissue-specific expression of human or humanized IL2RG, IL2RB, IL15RA, and/or IL15 proteins is provided.

In some embodiments, the expression of human or humanized IL2RG, IL2RB, IL15RA, and/or IL15 in a genetically modified animal is controllable, as by the addition of a specific inducer or repressor substance. In some embodiments, the specific inducer is selected from Tet-Off System/Tet-On System, or Tamoxifen System.

Non-human mammals can be any non-human animal known in the art and which can be used in the methods as described herein. Preferred non-human mammals are mammals, (e.g., rodents). In some embodiments, the non-human mammal is a mouse.

Genetic, molecular and behavioral analyses for the non-human mammals described above can be performed. The present disclosure also relates to the progeny produced by the non-human mammal provided by the present disclosure mated with the same or other genotypes.

The present disclosure also provides a cell line or primary cell culture derived from the non-human mammal or a progeny thereof. A model based on cell culture can be prepared, for example, by the following methods. Cell cultures can be obtained by way of isolation from a non-human mammal, alternatively cells can be obtained from the cell culture established using the same constructs and the standard cell transfection techniques. The integration of genetic constructs containing DNA sequences encoding human IL2RG, IL2RB, IL15RA, and/or IL15 proteins can be detected by a variety of methods.

There are many analytical methods that can be used to detect exogenous DNA, including methods at the level of nucleic acid (including the mRNA quantification approaches using reverse transcriptase polymerase chain reaction (RT-PCR) or Southern blotting, and in situ hybridization) and methods at the protein level (including histochemistry, immunoblot analysis and in vitro binding studies). In addition, the expression level of the gene of interest can be quantified by ELISA techniques well known to those skilled in the art. Many standard analysis methods can be used to complete quantitative measurements. For example, transcription levels can be measured using RT-PCR and hybridization methods including RNase protection, Southern blot analysis, RNA dot analysis (RNAdot) analysis. Immunohistochemical staining, flow cytometry, Western blot analysis can also be used to assess the presence of human or humanized IL2RG, IL2RB, IL15RA, and/or IL15 proteins.

### Vectors

The present disclosure relates to a targeting vector, comprising: a) a DNA fragment homologous to the 5′ end of a region to be altered (5′ arm), which is selected from the IL2RG gene genomic DNAs in the length of 100 to 10,000 nucleotides; b) a desired/donor DNA sequence encoding a donor region; and c) a second DNA fragment homologous to the 3′ end of the region to be altered (3′ arm), which is selected from the IL2RG gene genomic DNAs in the length of 100 to 10,000 nucleotides.

In some embodiments, a) the DNA fragment homologous to the 5′ end of a conversion region to be altered (5′ arm) is selected from the nucleotide sequences that have at least 90% homology to the NCBI accession number NC_000086.8; c) the DNA fragment homologous to the 3′ end of the region to be altered (3′ arm) is selected from the nucleotide sequences that have at least 90% homology to the NCBI accession number NC_000086.8.

In some embodiments, a) the DNA fragment homologous to the 5′ end of a region to be altered (5′ arm) is selected from the nucleotides from the position 100311776 to the position 100315845 of the NCBI accession number NC_000086.8; c) the DNA fragment homologous to the 3′ end of the region to be altered (3′ arm) is selected from the nucleotides from the position 100304102 to the position 100308040 of the NCBI accession number NC_000086.8.

In some embodiments, a) the DNA fragment homologous to the 5′ end of a region to be altered (5′ arm) is selected from the nucleotides from the position 100311776 to the position 100313491 of the NCBI accession number NC_000086.8; c) the DNA fragment homologous to the 3′ end of the region to be altered (3′ arm) is selected from the nucleotides from the position 100306445 to the position 100308040 of the NCBI accession number NC_000086.8.

In some embodiments, a) the DNA fragment homologous to the 5′ end of a region to be altered (5′ arm) is selected from the nucleotides from the position 100311776 to the position 100317900 of the NCBI accession number NC_000086.8; c) the DNA fragment homologous to the 3′ end of the region to be altered (3′ arm) is selected from the nucleotides from the position 100305674 to the position 100309321 of the NCBI accession number NC_000086.8.

In some embodiments, a) the DNA fragment homologous to the 5′ end of a region to be altered (5′ arm) is selected from the nucleotides from the position 100311776 to the position 100313175 of the NCBI accession number NC_000086.8; c) the DNA fragment homologous to the 3′ end of the region to be altered (3′ arm) is selected from the nucleotides from the position 100307922 to the position 100309321 of the NCBI accession number NC_000086.8.

In some embodiments, the length of the selected genomic nucleotide sequence in the targeting vector can be more than about 2 kb, about 2.5 kb, about 2.8 kb, about 3 kb, about 3.5 kb, about 4 kb, about 4.2 kb, about 4.5 kb, or about 5 kb.

In some embodiments, the region to be altered is exon 1, exon 2, exon 3, exon 4, exon 5, exon 6, exon 7, and/or exon 8 of IL2RG gene (e.g., a portion of exon 1, exons 2-7, and a portion of exon 8 of mouse IL2RG gene; alternatively, a portion of exon 1, exons 2-5, and a portion of exon 6 of mouse IL2RG gene).

In some embodiments, the sequence of the 5′ arm is shown in SEQ ID NO: 3; and the sequence of the 3′ arm is shown in SEQ ID NO: 4. In some embodiments, the sequence of the 5′ arm is shown in SEQ ID NO: 8; and the sequence of the 3′ arm is shown in SEQ ID NO: 9. In some embodiments, the sequence of the 5′ arm is shown in SEQ ID NO: 24; and the sequence of the 3′ arm is shown in SEQ ID NO: 25. In some embodiments, the sequence of the 5′ arm is shown in SEQ ID NO: 31; and the sequence of the 3′ arm is shown in SEQ ID NO: 32.

In some embodiments, the sequence is derived from human (e.g., 71107340-71111539 of NC_000023.11; or 71108685-71111539 of NC_000023.11). For example, the target region in the targeting vector is a part or entirety of the nucleotide sequence of a human IL2RG gene, preferably exon 1, exon 2, exon 3, exon 4, exon 5, exon 6, exon 7, and/or exon 8 of the human IL2RG gene. In some embodiments, the nucleotide sequence of the humanized IL2RG gene encodes the entire or the part of human IL2RG protein with the NCBI accession number NP_000197.1 (SEQ ID NO: 2).

The disclosure also provides vectors for constructing a humanized animal model or a knock-out model. In some embodiments, the vectors comprise a sgRNA sequence, wherein the sgRNA sequence targets IL2RG gene, and the sgRNA is unique on the target sequence of the gene to be altered, and meets the sequence arrangement rule of 5′-NNN (20)-NGG3′ or 5′-CCN—N(20)-3′; and in some embodiments, the targeting site of the sgRNA in the mouse IL2RG gene is located on the exon 1, exon 2, exon 3, exon 4, exon 5, exon 6, exon 7, exon 8, intron 1, intron 2, intron 3, intron 4, intron 5, intron 6, intron 7, upstream of exon 1, or downstream of exon 8 of the mouse IL2RG gene.

In some embodiments, the targeting sequences are shown as SEQ ID NOs: 10 and 11. Thus, the disclosure provides sgRNA sequences for constructing a genetic modified animal model.

The present disclosure relates to a targeting vector, comprising: a) a DNA fragment homologous to the 5′ end of a region to be altered (5′ arm), which is selected from the IL2RB gene genomic DNAs in the length of 100 to 10,000 nucleotides; b) a desired/donor DNA sequence encoding a donor region; and c) a second DNA fragment homologous to the 3′ end of the region to be altered (3′ arm), which is selected from the IL2RB gene genomic DNAs in the length of 100 to 10,000 nucleotides.

In some embodiments, a) the DNA fragment homologous to the 5′ end of a conversion region to be altered (5′ arm) is selected from the nucleotide sequences that have at least 90% homology to the NCBI accession number NC_000081.6; c) the DNA fragment homologous to the 3′ end of the region to be altered (3′ arm) is selected from the nucleotide sequences that have at least 90% homology to the NCBI accession number NC_000081.6.

In some embodiments, a) the DNA fragment homologous to the 5′ end of a region to be altered (5′ arm) is selected from the nucleotides from the position 78495191 to the position 78491766 of the NCBI accession number NC_000081.6; c) the DNA fragment homologous to the 3′ end of the region to be altered (3′ arm) is selected from the nucleotides from the position 78484605 to the position 78479760 of the NCBI accession number NC_000081.6.

In some embodiments, the length of the selected genomic nucleotide sequence in the targeting vector can be more than about 5 kb, about 5.5 kb, about 6 kb, about 6.5 kb, about 7 kb, about 7.5 kb, about 8 kb, about 8.5 kb, or about 8.6 kb.

In some embodiments, the region to be altered is exon 1, exon 2, exon 3, exon 4, exon 5, exon 6, exon 7, exon 8, exon 9, and/or exon 10 of IL2RB gene (e.g., a portion of exon 2, exons 3-7, and a portion of exon 8 of mouse IL2RB gene).

In some embodiments, the sequence of the 5′ arm is shown in SEQ ID NO: 35; and the sequence of the 3′ arm is shown in SEQ ID NO: 36.

In some embodiments, the sequence is derived from human (e.g., 37144088-37135435 of NC_000022.11). For example, the target region in the targeting vector is a part or entirety of the nucleotide sequence of a human IL2RB gene, preferably exon 1, exon 2, exon 3, exon 4, exon 5, exon 6, exon 7, exon 8, exon 9, and/or exon 10 of the human IL2RB gene. In some embodiments, the nucleotide sequence of the humanized IL2RB gene encodes the entire or the part of human IL2RB protein with the NCBI accession number NP_000869.1 (SEQ ID NO: 34).

The disclosure also provides vectors for constructing a humanized animal model or a knock-out model. In some embodiments, the vectors comprise a sgRNA sequence, wherein the sgRNA sequence targets IL2RB gene, and the sgRNA is unique on the target sequence of the gene to be altered, and meets the sequence arrangement rule of 5′-NNN (20)-NGG3′ or 5′-CCN—N(20)-3′; and in some embodiments, the targeting site of the sgRNA in the mouse IL2RB gene is located on the exon 1, exon 2, exon 3, exon 4, exon 5, exon 6, exon 7, exon 8, exon 9, exon 10, intron 1, intron 2, intron 3, intron 4, intron 5, intron 6, intron 7, intron 8, intron 9, upstream of exon 1, or downstream of exon 10 of the mouse IL2RB gene.

The present disclosure relates to a targeting vector, comprising: a) a DNA fragment homologous to the 5′ end of a region to be altered (5′ arm), which is selected from the IL15RA gene genomic DNAs in the length of 100 to 10,000 nucleotides; b) a desired/donor DNA sequence encoding a donor region; and c) a second DNA fragment homologous to the 3′ end of the region to be altered (3′ arm), which is selected from the IL15RA gene genomic DNAs in the length of 100 to 10,000 nucleotides.

In some embodiments, a) the DNA fragment homologous to the 5′ end of a conversion region to be altered (5′ arm) is selected from the nucleotide sequences that have at least 90% homology to the NCBI accession number NC_000068.8; c) the DNA fragment homologous to the 3′ end of the region to be altered (3′ arm) is selected from the nucleotide sequences that have at least 90% homology to the NCBI accession number NC_000068.8.

In some embodiments, a) the DNA fragment homologous to the 5′ end of a region to be altered (5′ arm) is selected from the nucleotides from the position 11717833 to the position 11723094 of the NCBI accession number NC_000068.8; c) the DNA fragment homologous to the 3′ end of the region to be altered (3′ arm) is selected from the nucleotides from the position 11735827 to the position 11739583 of the NCBI accession number NC_000068.8.

In some embodiments, the length of the selected genomic nucleotide sequence in the targeting vector can be more than about 6 kb, about 6.5 kb, about 7 kb, about 7.5 kb, about 8 kb, about 8.5 kb, about 9 kb, about 9.5 kb, about 9.8 kb, or about 10 kb.

In some embodiments, the region to be altered is exon 1, exon 2, exon 3, exon 4, exon 5, exon 6, and/or exon 7 of IL15RA gene (e.g., a portion of exon 2, exons 3-5, and a portion of exon 6 of mouse IL15RA gene).

In some embodiments, the sequence of the 5′ arm is shown in SEQ ID NO: 44; and the sequence of the 3′ arm is shown in SEQ ID NO: 45.

In some embodiments, the sequence is derived from human (e.g., 11735827-11739583 of NC_000068.8). For example, the target region in the targeting vector is a part or entirety of the nucleotide sequence of a human IL15RA gene, preferably exon 1, exon 2, exon 3, exon 4, exon 5, exon 6, and/or exon 7 of the human IL15RA gene. In some embodiments, the nucleotide sequence of the humanized IL15RA gene encodes the entire or the part of human IL15RA protein with the NCBI accession number NP_002180.1 (SEQ ID NO: 43).

The disclosure also provides vectors for constructing a humanized animal model or a knock-out model. In some embodiments, the vectors comprise a sgRNA sequence, wherein the sgRNA sequence targets IL15RA gene, and the sgRNA is unique on the target sequence of the gene to be altered, and meets the sequence arrangement rule of 5′-NNN (20)-NGG3′ or 5′-CCN—N(20)-3′; and in some embodiments, the targeting site of the sgRNA in the mouse IL15RA gene is located on the exon 1, exon 2, exon 3, exon 4, exon 5, exon 6, exon 7, intron 1, intron 2, intron 3, intron 4, intron 5, intron 6, upstream of exon 1, or downstream of exon 7 of the mouse IL15RA gene.

The present disclosure relates to a targeting vector, comprising: a) a DNA fragment homologous to the 5′ end of a region to be altered (5′ arm), which is selected from the IL15 gene genomic DNAs in the length of 100 to 10,000 nucleotides; b) a desired/donor DNA sequence encoding a donor region; and c) a second DNA fragment homologous to the 3′ end of the region to be altered (3′ arm), which is selected from the IL15 gene genomic DNAs in the length of 100 to 10,000 nucleotides.

In some embodiments, a) the DNA fragment homologous to the 5′ end of a conversion region to be altered (5′ arm) is selected from the nucleotide sequences that have at least 90% homology to the NCBI accession number NC_000074.7; c) the DNA fragment homologous to the 3′ end of the region to be altered (3′ arm) is selected from the nucleotide sequences that have at least 90% homology to the NCBI accession number NC_000074.7.

In some embodiments, a) the DNA fragment homologous to the 5′ end of a region to be altered (5′ arm) is selected from the nucleotides from the position 83072241 to the position 83076085 of the NCBI accession number NC_000074.7; c) the DNA fragment homologous to the 3′ end of the region to be altered (3′ arm) is selected from the nucleotides from the position 83053728 to the position 83057763 of the NCBI accession number NC_000074.7.

In some embodiments, the length of the selected genomic nucleotide sequence in the targeting vector can be more than about 10 kb, about 10.5 kb, about 11 kb, about 11.5 kb, about 12 kb, about 12.5 kb, about 13 kb, about 13.3 kb, or about 14 kb.

In some embodiments, the region to be altered is exon 1, exon 2, exon 3, exon 4, exon 5, exon 6, exon 7, and/or exon 8 of IL15 gene (e.g., a portion of exon 3, exons 4-7, and a portion of exon 8 of mouse IL15 gene).

In some embodiments, the sequence of the 5′ arm is shown in SEQ ID NO: 53; and the sequence of the 3′ arm is shown in SEQ ID NO: 54.

In some embodiments, the sequence is derived from human (e.g., 141719465-141732848 of NC_000004.12). For example, the target region in the targeting vector is a part or entirety of the nucleotide sequence of a human IL15 gene, preferably exon 1, exon 2, exon 3, exon 4, exon 5, exon 6, exon 7, and/or exon 8 of the human IL15 gene. In some embodiments, the nucleotide sequence of the humanized IL15 gene encodes the entire or the part of human IL15 protein with the NCBI accession number NP_000576.1 (SEQ ID NO: 52).

The disclosure also provides vectors for constructing a humanized animal model or a knock-out model. In some embodiments, the vectors comprise a sgRNA sequence, wherein the sgRNA sequence targets IL15 gene, and the sgRNA is unique on the target sequence of the gene to be altered, and meets the sequence arrangement rule of 5′-NNN (20)-NGG3′ or 5′-CCN—N(20)-3′; and in some embodiments, the targeting site of the sgRNA in the mouse IL15 gene is located on the exon 1, exon 2, exon 3, exon 4, exon 5, exon 6, exon 7, exon 8, intron 1, intron 2, intron 3, intron 4, intron 5, intron 6, intron 7, upstream of exon 1, or downstream of exon 8 of the mouse IL15 gene.

The targeting vector can further include one or more selectable markers, e.g., positive or negative selectable markers. In some embodiments, the positive selectable marker is a Neo gene or Neo cassette. In some embodiments, the negative selectable marker is a DTA gene.

In some embodiments, the disclosure relates to a plasmid construct (e.g., pT7-sgRNA) including the sgRNA sequence, and/or a cell including the construct.

The disclosure also relates to a cell comprising the targeting vectors as described above.

In addition, the present disclosure further relates to a non-human mammalian cell, having any one of the foregoing targeting vectors, and one or more in vitro transcripts of the construct as described herein. In some embodiments, the cell includes Cas9 mRNA or an in vitro transcript thereof.

In some embodiments, the genes in the cell are heterozygous. In some embodiments, the genes in the cell are homozygous.

In some embodiments, the non-human mammalian cell is a mouse cell. In some embodiments, the cell is a fertilized egg cell. In some embodiments, the cell is an embryonic stem cell.

### Methods of Making Genetically Modified Animals

Genetically modified animals can be made by several techniques that are known in the art, including, e.g., nonhomologous end-joining (NHEJ), homologous recombination (HR), zinc finger nucleases (ZFNs), transcription activator-like effector-based nucleases (TALEN), and the clustered regularly interspaced short palindromic repeats (CRISPR)-Cas system. In some embodiments, homologous recombination is used. In some embodiments, CRISPR-Cas9 genome editing is used to generate genetically modified animals. Many of these genome editing techniques are known in the art, and is described, e.g., in Yin et al., “Delivery technologies for genome editing,” Nature Reviews Drug Discovery 16.6 (2017): 387-399, which is incorporated by reference in its entirety. Many other methods are also provided and can be used in genome editing, e.g., micro-injecting a genetically modified nucleus into an enucleated oocyte, and fusing an enucleated oocyte with another genetically modified cell.

Thus, in some embodiments, the disclosure provides replacing in at least one cell of the animal, at an endogenous IL2RG gene locus, a sequence encoding a region of an endogenous IL2RG with a sequence encoding a corresponding region of human or chimeric IL2RG. In some embodiments, the replacement occurs in a germ cell, a somatic cell, a blastocyst, or a fibroblast, etc. The nucleus of a somatic cell or the fibroblast can be inserted into an enucleated oocyte.

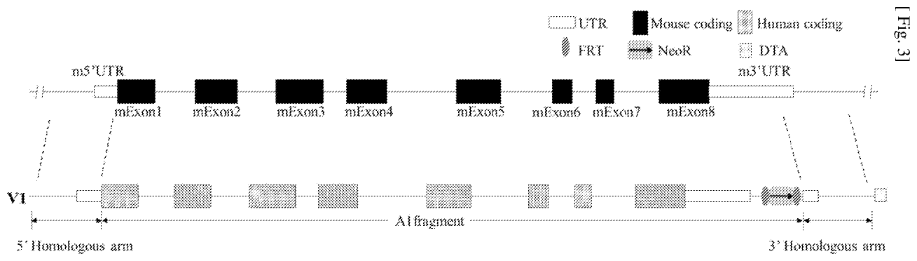

FIGS. 3, 4, 9, and 10 show humanization strategies for a mouse IL2RG locus. The targeting strategies involve a vector comprising a 5′ homologous arm, a human IL2RG gene fragment, and a 3′ homologous arm. The process can involve replacing endogenous IL2RG sequence with human sequence by homologous recombination. In some embodiments, the cleavage at the upstream and the downstream of the target site (e.g., by zinc finger nucleases, TALEN or CRISPR) can result in DNA double strands break, and the homologous recombination is used to replace endogenous IL2RG sequence with human IL2RG sequence.

Thus, in some embodiments, the methods for making a genetically modified, humanized animal, can include the step of replacing at an endogenous IL2RG locus (or site), a nucleic acid sequence encoding a region of endogenous IL2RG with a sequence encoding a corresponding region of human IL2RG. The sequence can include a region (e.g., a part or the entire region) of exon 1, exon 2, exon 3, exon 4, exon 5, exon 6, exon 7, and/or exon 8 of a human IL2RG gene. In some embodiments, the sequence includes a portion of exon 1, exons 2-7, and a portion of exon 8 of a human IL2RG gene (e.g., nucleic acids 93-1534 of NM_000206.2). In some embodiments, the sequence includes a portion of exon 1, exons 2-5, and a portion of exon 6 of a human IL2RG gene (e.g., nucleic acids 93-860 of NM_000206.2).

In some embodiments, the region includes the signal peptide of human IL2RG (e.g., amino acids 1-22 of SEQ ID NO: 2), and/or all or a portion of the extracellular region of human IL2RG (e.g., amino acids 23-256 of SEQ ID NO: 2). In some embodiments, the region includes the full-length protein of human IL2RG (e.g., amino acids 1-369 of SEQ ID NO: 2). In some embodiments, the endogenous IL2RG locus is exon 1, exon 2, exon 3, exon 4, exon 5, exon 6, exon 7, and/or exon 8 of mouse IL2RG. In some embodiments, the sequence includes a portion of exon 1 and a portion of exon 8 of mouse IL2RG gene (e.g., nucleic acids 1-86 and 1614-1663 of NM_013563.4). In some embodiments, the sequence includes a portion of exon 1, a portion of exon 6, and exons 7-8 of mouse IL2RG gene (e.g., nucleic acids 1-86 and 858-1663 of NM_013563.4).

In some embodiments, the methods of modifying a IL2RG locus of a mouse to express a chimeric human/mouse IL2RG peptide can include the steps of replacing at the endogenous mouse IL2RG locus a nucleotide sequence encoding a mouse IL2RG with a nucleotide sequence encoding a human IL2RG, thereby generating a sequence encoding a chimeric human/mouse IL2RG.

In some embodiments, the nucleotide sequence encoding the chimeric human/mouse IL2RG can include a first nucleotide sequence encoding the signal peptide and all or a portion of the extracellular region of human IL2RG; and a second nucleotide sequence encoding the transmembrane region and the cytoplasmic region of mouse IL2RG, optionally the C-terminal 1, 2, 3, 4, 5, or 6 amino acids in the extracellular region of mouse IL2RG.

The present disclosure further provides a method for establishing a IL2RG gene humanized animal model, involving the following steps:
(a) providing the cell (e.g. a fertilized egg cell) based on the methods described herein;
(b) culturing the cell in a liquid culture medium;
(c) transplanting the cultured cell to the fallopian tube or uterus of the recipient female non-human mammal, allowing the cell to develop in the uterus of the female non-human mammal;
(d) identifying the germline transmission in the offspring genetically modified humanized non-human mammal of the pregnant female in step (c).

In some embodiments, the non-human mammal in the foregoing method is a mouse (e.g., a C57BL/6 mouse).

In some embodiments, the non-human mammal in step (c) is a female with pseudopregnancy (or false pregnancy).

In some embodiments, the fertilized eggs for the methods described above are C57BL/6 fertilized eggs. Other fertilized eggs that can also be used in the methods as described herein include, but are not limited to, FVB/N fertilized eggs, BALB/c fertilized eggs, DBA/1 fertilized eggs and DBA/2 fertilized eggs.

Fertilized eggs can come from any non-human animal, e.g., any non-human animal as described herein. In some embodiments, the fertilized egg cells are derived from rodents. The genetic construct can be introduced into a fertilized egg by microinjection of DNA. For example, by way of culturing a fertilized egg after microinjection, a cultured fertilized egg can be transferred to a false pregnant non-human animal, which then gives birth of a non-human mammal, so as to generate the non-human mammal mentioned in the methods described above.

In some embodiments, methods of making the genetically modified animal comprises modifying the coding frame of the non-human animal's IL2RG gene, e.g., by inserting a nucleotide sequence (e.g., DNA or cDNA sequence) encoding human or humanized IL2RG protein, e.g., immediately after the endogenous regulatory element of the non-human animal's IL2RG gene. For example, one or more functional region sequences of the non-human animal's IL2RG gene can be knocked out, or inserted with a sequence, such that the non-human animal cannot express or expresses a decreased level of endogenous IL2RG protein. In some embodiments, the coding frame of the modified non-human animal's IL2RG gene can be all or part of the nucleotide sequence from exon 1 to exon 8 of the non-human animal's IL2RG gene.

In some embodiments, methods of making the genetically modified animal comprises inserting a nucleotide sequence encoding human or humanized IL2RG protein and/or an auxiliary sequence after the endogenous regulatory element of the non-human animal's IL2RG gene. In some embodiments, the auxiliary sequence can be a stop codon, such that the IL2RG gene humanized animal model can express human or humanized IL2RG protein in vivo, but does not express non-human animal's IL2RG protein. In some embodiments, the auxiliary sequence includes WPRE (WHP Posttranscriptional Response Element), loxP, STOP, and/or polyA.

In some embodiments, the method for making the genetically modified animal comprises:
(1) providing a plasmid comprising a human IL2RG gene fragment, flanked by a 5′ homologous arm and a 3′ homologous arm, wherein the 5′ and 3′ homologous arms target an endogenous IL2RG gene;
(2) providing one or more small guide RNAs (sgRNAs) that target the endogenous IL2RG gene;
(3) modifying genome of a fertilized egg or an embryonic stem cell by using the plasmid of step (1), the sgRNAs of step (2), and Cas9;
(4) transplanting the fertilized egg obtained in step (3) into the oviduct of a pseudopregnant female mouse or transplanting the embryonic stem cell obtained in step (3) into a blastocyst which is then transplanted into the oviduct of a pseudopregnant female mouse to produce a child mouse that functionally expresses a humanized IL2RG protein; and
(5) mating the child mouse obtained in step (2) to obtain a homozygote mouse,

In some embodiments, the fertilized egg is modified by CRISPR with sgRNAs that target a 5′-terminal targeting site and a 3′-terminal targeting site.

In some embodiments, the sequence encoding the humanized IL2RG protein is operably linked to an endogenous regulatory element at the endogenous IL2RG gene locus.

In some embodiments, the genetically-modified animal does not express an endogenous IL2RG protein.

In some embodiments, the method for making the genetically modified animal comprises:
(1) providing a plasmid comprising a human or chimeric IL2RG gene fragment, flanked by a 5′ homologous arm and a 3′ homologous arm, wherein the 5′ and 3′ homologous arms target an endogenous IL2RG gene;
(2) providing one or more small guide RNAs (sgRNAs) that target the endogenous IL2RG gene; and
(3) modifying genome of a fertilized egg or an embryonic stem cell by inserting the human or chimeric IL2RG gene fragment into the genome.

In some embodiments, the disclosure provides replacing in at least one cell of the animal, at an endogenous IL2RB gene locus, a sequence encoding a region of an endogenous IL2RB with a sequence encoding a corresponding region of human or chimeric IL2RB. In some embodiments, the replacement occurs in a germ cell, a somatic cell, a blastocyst, or a fibroblast, etc. The nucleus of a somatic cell or the fibroblast can be inserted into an enucleated oocyte.

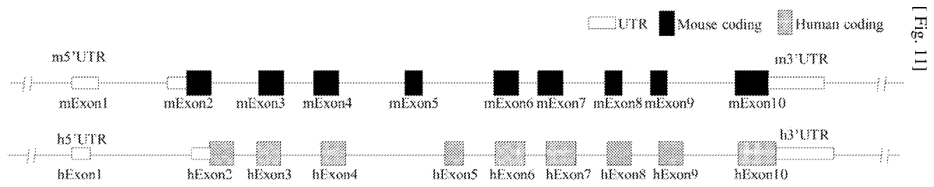

FIG. 12 shows a humanization strategy for a mouse IL2RB locus. The targeting strategies involve a vector comprising a 5′ homologous arm, a human IL2RB gene fragment, and a 3′ homologous arm. The process can involve replacing endogenous IL2RB sequence with human sequence by homologous recombination. In some embodiments, the cleavage at the upstream and the downstream of the target site (e.g., by zinc finger nucleases, TALEN or CRISPR) can result in DNA double strands break, and the homologous recombination is used to replace endogenous IL2RB sequence with human IL2RB sequence.

Thus, in some embodiments, the methods for making a genetically modified, humanized animal, can include the step of replacing at an endogenous IL2RB locus (or site), a nucleic acid sequence encoding a region of endogenous IL2RB with a sequence encoding a corresponding region of human IL2RB. The sequence can include a region (e.g., a part or the entire region) of exon 1, exon 2, exon 3, exon 4, exon 5, exon 6, exon 7, exon 8, exon 9, and/or exon 10 of a human IL2RB gene. In some embodiments, the sequence includes a portion of exon 2, exons 3-7, and a portion of exon 8 of a human IL2RB gene (e.g., nucleic acids 210-836 of NM_000878.5).

In some embodiments, the region includes the all or a portion of the extracellular region of human IL2RB (e.g., amino acids 29-237 of SEQ ID NO: 34). In some embodiments, the endogenous IL2RB locus is exon 1, exon 2, exon 3, exon 4, exon 5, exon 6, exon 7, exon 8, exon 9, and/or exon 10 of mouse IL2RB. In some embodiments, the sequence includes exon 1, a portion of exon 2, a portion of exon 8, and exons 9-10 of mouse IL2RB gene (e.g., nucleic acids 1-233 and 864-2712 of NM_008368.4).

In some embodiments, the methods of modifying a IL2RB locus of a mouse to express a chimeric human/mouse IL2RB peptide can include the steps of replacing at the endogenous mouse IL2RB locus a nucleotide sequence encoding a mouse IL2RB with a nucleotide sequence encoding a human IL2RB, thereby generating a sequence encoding a chimeric human/mouse IL2RB.

In some embodiments, the nucleotide sequence encoding the chimeric human/mouse IL2RB can include a first nucleotide sequence encoding the signal peptide of mouse IL2RB, optionally the N-terminal 1 or 2 amino acids in the extracellular region of mouse IL2RB; a second nucleotide sequence encoding all or a portion of the extracellular region of human IL2RB; and a third nucleotide sequence encoding the transmembrane region and the cytoplasmic region of mouse IL2RB, optionally the C-terminal 1 or 2 amino acids in the extracellular region of mouse IL2RB.

The present disclosure further provides a method for establishing a IL2RB gene humanized animal model, involving the following steps:
(a) providing the cell (e.g. a fertilized egg cell) based on the methods described herein;
(b) culturing the cell in a liquid culture medium;
(c) transplanting the cultured cell to the fallopian tube or uterus of the recipient female non-human mammal, allowing the cell to develop in the uterus of the female non-human mammal;
(d) identifying the germline transmission in the offspring genetically modified humanized non-human mammal of the pregnant female in step (c).

In some embodiments, the non-human mammal in the foregoing method is a mouse (e.g., a C57BL/6 mouse).

In some embodiments, the non-human mammal in step (c) is a female with pseudopregnancy (or false pregnancy).

In some embodiments, the fertilized eggs for the methods described above are C57BL/6 fertilized eggs. Other fertilized eggs that can also be used in the methods as described herein include, but are not limited to, FVB/N fertilized eggs, BALB/c fertilized eggs, DBA/1 fertilized eggs and DBA/2 fertilized eggs.

Fertilized eggs can come from any non-human animal, e.g., any non-human animal as described herein. In some embodiments, the fertilized egg cells are derived from rodents. The genetic construct can be introduced into a fertilized egg by microinjection of DNA. For example, by way of culturing a fertilized egg after microinjection, a cultured fertilized egg can be transferred to a false pregnant non-human animal, which then gives birth of a non-human mammal, so as to generate the non-human mammal mentioned in the methods described above.

In some embodiments, methods of making the genetically modified animal comprises modifying the coding frame of the non-human animal's IL2RB gene, e.g., by inserting a nucleotide sequence (e.g., DNA or cDNA sequence) encoding human or humanized IL2RB protein, e.g., immediately after the endogenous regulatory element of the non-human animal's IL2RB gene. For example, one or more functional region sequences of the non-human animal's IL2RB gene can be knocked out, or inserted with a sequence, such that the non-human animal cannot express or expresses a decreased level of endogenous IL2RB protein. In some embodiments, the coding frame of the modified non-human animal's IL2RB gene can be all or part of the nucleotide sequence from exon 1 to exon 10 of the non-human animal's IL2RB gene.

In some embodiments, methods of making the genetically modified animal comprises inserting a nucleotide sequence encoding human or humanized IL2RB protein and/or an auxiliary sequence after the endogenous regulatory element of the non-human animal's IL2RB gene. In some embodiments, the auxiliary sequence can be a stop codon, such that the IL2RB gene humanized animal model can express human or humanized IL2RB protein in vivo, but does not express non-human animal's IL2RB protein. In some embodiments, the auxiliary sequence includes WPRE (WHP Posttranscriptional Response Element), loxP, STOP, and/or polyA.

In some embodiments, the method for making the genetically modified animal comprises:
(1) providing a plasmid comprising a human IL2RB gene fragment, flanked by a 5′ homologous arm and a 3′ homologous arm, wherein the 5′ and 3′ homologous arms target an endogenous IL2RB gene;
(2) providing one or more small guide RNAs (sgRNAs) that target the endogenous IL2RB gene;
(3) modifying genome of a fertilized egg or an embryonic stem cell by using the plasmid of step (1), the sgRNAs of step (2), and Cas9;
(4) transplanting the fertilized egg obtained in step (3) into the oviduct of a pseudopregnant female mouse or transplanting the embryonic stem cell obtained in step (3) into a blastocyst which is then transplanted into the oviduct of a pseudopregnant female mouse to produce a child mouse that functionally expresses a humanized IL2RB protein; and
(5) mating the child mouse obtained in step (2) to obtain a homozygote mouse,

In some embodiments, the fertilized egg is modified by CRISPR with sgRNAs that target a 5′-terminal targeting site and a 3′-terminal targeting site.

In some embodiments, the sequence encoding the humanized IL2RB protein is operably linked to an endogenous regulatory element at the endogenous IL2RB gene locus.

In some embodiments, the genetically-modified animal does not express an endogenous IL2RB protein.

In some embodiments, the method for making the genetically modified animal comprises:
(1) providing a plasmid comprising a human or chimeric IL2RB gene fragment, flanked by a 5′ homologous arm and a 3′ homologous arm, wherein the 5′ and 3′ homologous arms target an endogenous IL2RB gene;
(2) providing one or more small guide RNAs (sgRNAs) that target the endogenous IL2RB gene; and
(3) modifying genome of a fertilized egg or an embryonic stem cell by inserting the human or chimeric IL2RB gene fragment into the genome.

In some embodiments, the disclosure provides replacing in at least one cell of the animal, at an endogenous IL15RA gene locus, a sequence encoding a region of an endogenous IL15RA with a sequence encoding a corresponding region of human or chimeric IL15RA. In some embodiments, the replacement occurs in a germ cell, a somatic cell, a blastocyst, or a fibroblast, etc. The nucleus of a somatic cell or the fibroblast can be inserted into an enucleated oocyte.

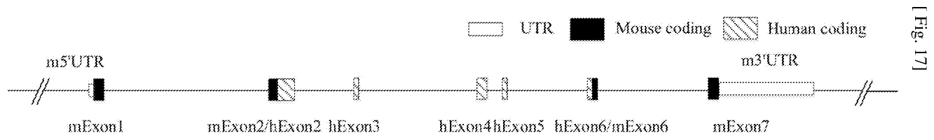

FIG. 18 shows a humanization strategy for a mouse IL15RA locus. The targeting strategies involve a vector comprising a 5′ homologous arm, a human IL15RA gene fragment, and a 3′ homologous arm. The process can involve replacing endogenous IL15RA sequence with human sequence by homologous recombination. In some embodiments, the cleavage at the upstream and the downstream of the target site (e.g., by zinc finger nucleases, TALEN or CRISPR) can result in DNA double strands break, and the homologous recombination is used to replace endogenous IL15RA sequence with human IL15RA sequence.

Thus, in some embodiments, the methods for making a genetically modified, humanized animal, can include the step of replacing at an endogenous IL15RA locus (or site), a nucleic acid sequence encoding a region of endogenous IL15RA with a sequence encoding a corresponding region of human IL15RA. The sequence can include a region (e.g., a part or the entire region) of exon 1, exon 2, exon 3, exon 4, exon 5, exon 6, and/or exon 7 of a human IL15RA gene. In some embodiments, the sequence includes a portion of exon 2, exons 3-5, and a portion of exon 6 of a human IL15RA gene (e.g., nucleic acids 160-684 of NM_002189.4).

In some embodiments, the region includes the all or a portion of the extracellular region and/or all or a portion of the transmembrane region of human IL15RA (e.g., amino acids 37-211 of SEQ ID NO: 43). In some embodiments, the endogenous IL15RA locus is exon 1, exon 2, exon 3, exon 4, exon 5, exon 6, and/or exon 7 of mouse IL15RA. In some embodiments, the sequence includes exon 1, a portion of exon 2, a portion of exon 6, and exon 7 of mouse IL15RA gene (e.g., nucleic acids 1-253 and 770-1664 of NM_008358.2).

In some embodiments, the methods of modifying a IL15RA locus of a mouse to express a chimeric human/mouse IL15RA peptide can include the steps of replacing at the endogenous mouse IL15RA locus a nucleotide sequence encoding a mouse IL15RA with a nucleotide sequence encoding a human IL15RA, thereby generating a sequence encoding a chimeric human/mouse IL15RA.

In some embodiments, the nucleotide sequence encoding the chimeric human/mouse IL15RA can include a first nucleotide sequence encoding the signal peptide of mouse IL15RA, optionally the N-terminal 1, 2, 3, 4, 5, 6, or 7 amino acids in the extracellular region of mouse IL15RA; a second nucleotide sequence encoding all or a portion of the extracellular region of human IL15RA, optionally the N-terminal 1, 2, 3, 4, 5, or 6 amino acids in the transmembrane region of human IL15RA; and a third nucleotide sequence encoding all or a portion of the transmembrane region (e.g., the C-terminal 1, 2, 3, 4, 5, 6, 7, 8, 9, 10, 11, 12, 13, 14, or 15 amino acids of the transmembrane region) and the cytoplasmic region of mouse IL15RA.

The present disclosure further provides a method for establishing a IL15RA gene humanized animal model, involving the following steps:
(a) providing the cell (e.g. a fertilized egg cell) based on the methods described herein;
(b) culturing the cell in a liquid culture medium;
(c) transplanting the cultured cell to the fallopian tube or uterus of the recipient female non-human mammal, allowing the cell to develop in the uterus of the female non-human mammal;
(d) identifying the germline transmission in the offspring genetically modified humanized non-human mammal of the pregnant female in step (c).

In some embodiments, the non-human mammal in the foregoing method is a mouse (e.g., a C57BL/6 mouse).

In some embodiments, the non-human mammal in step (c) is a female with pseudopregnancy (or false pregnancy).

In some embodiments, the fertilized eggs for the methods described above are C57BL/6 fertilized eggs. Other fertilized eggs that can also be used in the methods as described herein include, but are not limited to, FVB/N fertilized eggs, BALB/c fertilized eggs, DBA/1 fertilized eggs and DBA/2 fertilized eggs.

Fertilized eggs can come from any non-human animal, e.g., any non-human animal as described herein. In some embodiments, the fertilized egg cells are derived from rodents. The genetic construct can be introduced into a fertilized egg by microinjection of DNA. For example, by way of culturing a fertilized egg after microinjection, a cultured fertilized egg can be transferred to a false pregnant non-human animal, which then gives birth of a non-human mammal, so as to generate the non-human mammal mentioned in the methods described above.

In some embodiments, methods of making the genetically modified animal comprises modifying the coding frame of the non-human animal's IL15RA gene, e.g., by inserting a nucleotide sequence (e.g., DNA or cDNA sequence) encoding human or humanized IL15RA protein, e.g., immediately after the endogenous regulatory element of the non-human animal's IL15RA gene. For example, one or more functional region sequences of the non-human animal's IL15RA gene can be knocked out, or inserted with a sequence, such that the non-human animal cannot express or expresses a decreased level of endogenous IL15RA protein. In some embodiments, the coding frame of the modified non-human animal's IL15RA gene can be all or part of the nucleotide sequence from exon 1 to exon 7 of the non-human animal's IL15RA gene.

In some embodiments, methods of making the genetically modified animal comprises inserting a nucleotide sequence encoding human or humanized IL15RA protein and/or an auxiliary sequence after the endogenous regulatory element of the non-human animal's IL15RA gene. In some embodiments, the auxiliary sequence can be a stop codon, such that the IL15RA gene humanized animal model can express human or humanized IL15RA protein in vivo, but does not express non-human animal's IL15RA protein. In some embodiments, the auxiliary sequence includes WPRE (WHP Posttranscriptional Response Element), loxP, STOP, and/or polyA.

In some embodiments, the method for making the genetically modified animal comprises:
(1) providing a plasmid comprising a human IL15RA gene fragment, flanked by a 5′ homologous arm and a 3′ homologous arm, wherein the 5′ and 3′ homologous arms target an endogenous IL15RA gene;
(2) providing one or more small guide RNAs (sgRNAs) that target the endogenous IL15RA gene;
(3) modifying genome of a fertilized egg or an embryonic stem cell by using the plasmid of step (1), the sgRNAs of step (2), and Cas9;
(4) transplanting the fertilized egg obtained in step (3) into the oviduct of a pseudopregnant female mouse or transplanting the embryonic stem cell obtained in step (3) into a blastocyst which is then transplanted into the oviduct of a pseudopregnant female mouse to produce a child mouse that functionally expresses a humanized IL15RA protein; and
(5) mating the child mouse obtained in step (2) to obtain a homozygote mouse,

In some embodiments, the fertilized egg is modified by CRISPR with sgRNAs that target a 5′-terminal targeting site and a 3′-terminal targeting site.

In some embodiments, the sequence encoding the humanized IL15RA protein is operably linked to an endogenous regulatory element at the endogenous IL15RA gene locus.

In some embodiments, the genetically-modified animal does not express an endogenous IL15RA protein.

In some embodiments, the method for making the genetically modified animal comprises:
(1) providing a plasmid comprising a human or chimeric IL15RA gene fragment, flanked by a 5′ homologous arm and a 3′ homologous arm, wherein the 5′ and 3′ homologous arms target an endogenous IL15RA gene;
(2) providing one or more small guide RNAs (sgRNAs) that target the endogenous IL15RA gene; and
(3) modifying genome of a fertilized egg or an embryonic stem cell by inserting the human or chimeric IL15RA gene fragment into the genome.

In some embodiments, the disclosure provides replacing in at least one cell of the animal, at an endogenous IL15 gene locus, a sequence encoding a region of an endogenous IL15 with a sequence encoding a corresponding region of human or chimeric IL15. In some embodiments, the replacement occurs in a germ cell, a somatic cell, a blastocyst, or a fibroblast, etc. The nucleus of a somatic cell or the fibroblast can be inserted into an enucleated oocyte.

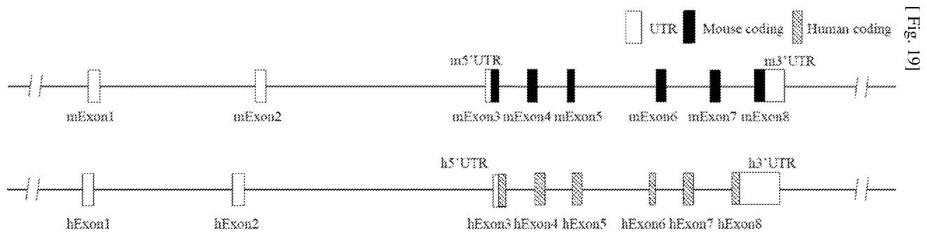

FIG. 21 shows a humanization strategy for a mouse IL15 locus. The targeting strategies involve a vector comprising a 5′ homologous arm, a human IL15 gene fragment, and a 3′ homologous arm. The process can involve replacing endogenous IL15 sequence with human sequence by homologous recombination. In some embodiments, the cleavage at the upstream and the downstream of the target site (e.g., by zinc finger nucleases, TALEN or CRISPR) can result in DNA double strands break, and the homologous recombination is used to replace endogenous IL15 sequence with human IL15 sequence.

Thus, in some embodiments, the methods for making a genetically modified, humanized animal, can include the step of replacing at an endogenous IL15 locus (or site), a nucleic acid sequence encoding a region of endogenous IL15 with a sequence encoding a corresponding region of human IL15. The sequence can include a region (e.g., a part or the entire region) of exon 1, exon 2, exon 3, exon 4, exon 5, exon 6, exon 7, and/or exon 8 of a human IL15 gene. In some embodiments, the sequence includes a portion of exon 3, exons 4-7, and a portion of exon 8 of a human IL15 gene (e.g., nucleic acids 388-876 of NM_000585.5).

In some embodiments, the region includes the entire coding region of human IL15 (e.g., amino acids 1-162 of SEQ ID NO: 52). In some embodiments, the endogenous IL15 locus is exon 1, exon 2, exon 3, exon 4, exon 5, exon 6, exon 7, and/or exon 8 of mouse IL15. In some embodiments, the sequence includes exons 1-2, a portion of exon 3, and a portion of exon 8, of mouse IL15 gene (e.g., nucleic acids 1-496 and 986-1287 of NM_001254747.1).

The present disclosure further provides a method for establishing a IL15 gene humanized animal model, involving the following steps:
(a) providing the cell (e.g. a fertilized egg cell) based on the methods described herein;
(b) culturing the cell in a liquid culture medium;
(c) transplanting the cultured cell to the fallopian tube or uterus of the recipient female non-human mammal, allowing the cell to develop in the uterus of the female non-human mammal;
(d) identifying the germline transmission in the offspring genetically modified humanized non-human mammal of the pregnant female in step (c).

In some embodiments, the non-human mammal in the foregoing method is a mouse (e.g., a C57BL/6 mouse).

In some embodiments, the non-human mammal in step (c) is a female with pseudopregnancy (or false pregnancy).

In some embodiments, the fertilized eggs for the methods described above are C57BL/6 fertilized eggs. Other fertilized eggs that can also be used in the methods as described herein include, but are not limited to, FVB/N fertilized eggs, BALB/c fertilized eggs, DBA/1 fertilized eggs and DBA/2 fertilized eggs.

Fertilized eggs can come from any non-human animal, e.g., any non-human animal as described herein. In some embodiments, the fertilized egg cells are derived from rodents. The genetic construct can be introduced into a fertilized egg by microinjection of DNA. For example, by way of culturing a fertilized egg after microinjection, a cultured fertilized egg can be transferred to a false pregnant non-human animal, which then gives birth of a non-human mammal, so as to generate the non-human mammal mentioned in the methods described above.

In some embodiments, methods of making the genetically modified animal comprises modifying the coding frame of the non-human animal's IL15 gene, e.g., by inserting a nucleotide sequence (e.g., DNA or cDNA sequence) encoding human or humanized IL15 protein, e.g., immediately after the endogenous regulatory element of the non-human animal's IL15 gene. For example, one or more functional region sequences of the non-human animal's IL15 gene can be knocked out, or inserted with a sequence, such that the non-human animal cannot express or expresses a decreased level of endogenous IL15 protein. In some embodiments, the coding frame of the modified non-human animal's IL15 gene can be all or part of the nucleotide sequence from exon 1 to exon 8 of the non-human animal's IL15 gene.

In some embodiments, methods of making the genetically modified animal comprises inserting a nucleotide sequence encoding human or humanized IL15 protein and/or an auxiliary sequence after the endogenous regulatory element of the non-human animal's IL15 gene. In some embodiments, the auxiliary sequence can be a stop codon, such that the IL15 gene humanized animal model can express human or humanized IL15 protein in vivo, but does not express non-human animal's IL15 protein. In some embodiments, the auxiliary sequence includes WPRE (WHP Posttranscriptional Response Element), loxP, STOP, and/or polyA.

In some embodiments, the method for making the genetically modified animal comprises:
(1) providing a plasmid comprising a human IL15 gene fragment, flanked by a 5′ homologous arm and a 3′ homologous arm, wherein the 5′ and 3′ homologous arms target an endogenous IL15 gene;
(2) providing one or more small guide RNAs (sgRNAs) that target the endogenous IL15 gene;
(3) modifying genome of a fertilized egg or an embryonic stem cell by using the plasmid of step (1), the sgRNAs of step (2), and Cas9;
(4) transplanting the fertilized egg obtained in step (3) into the oviduct of a pseudopregnant female mouse or transplanting the embryonic stem cell obtained in step (3) into a blastocyst which is then transplanted into the oviduct of a pseudopregnant female mouse to produce a child mouse that functionally expresses a humanized IL15 protein; and
(5) mating the child mouse obtained in step (2) to obtain a homozygote mouse,

In some embodiments, the fertilized egg is modified by CRISPR with sgRNAs that target a 5′-terminal targeting site and a 3′-terminal targeting site.

In some embodiments, the sequence encoding the humanized IL15 protein is operably linked to an endogenous regulatory element at the endogenous IL15 gene locus.

In some embodiments, the genetically-modified animal does not express an endogenous IL15 protein.

In some embodiments, the method for making the genetically modified animal comprises:
(1) providing a plasmid comprising a human or chimeric IL15 gene fragment, flanked by a 5′ homologous arm and a 3′ homologous arm, wherein the 5′ and 3′ homologous arms target an endogenous IL15 gene;
(2) providing one or more small guide RNAs (sgRNAs) that target the endogenous IL15 gene; and
(3) modifying genome of a fertilized egg or an embryonic stem cell by inserting the human or chimeric IL15 gene fragment into the genome.

In some embodiments, the nucleotide sequences as described herein do not overlap with each other (e.g., the first nucleotide sequence, the second nucleotide sequence, and/or the third nucleotide sequence do not overlap). In some embodiments, the amino acid sequences as described herein do not overlap with each other.

### Methods of Using Genetically Modified Animals

Replacement of non-human genes in a non-human animal with homologous or orthologous human genes or human sequences, at the endogenous non-human locus and under control of endogenous promoters and/or regulatory elements, can result in a non-human animal with qualities and characteristics that may be substantially different from a typical knockout-plus-transgene animal. In the typical knockout-plus-transgene animal, an endogenous locus is removed or damaged and a fully human transgene is inserted into the animal's genome and presumably integrates at random into the genome. Typically, the location of the integrated transgene is unknown; expression of the human protein is measured by transcription of the human gene and/or protein assay and/or functional assay. Inclusion in the human transgene of upstream and/or downstream human sequences are apparently presumed to be sufficient to provide suitable support for expression and/or regulation of the transgene.

In some cases, the transgene with human regulatory elements expresses in a manner that is unphysiological or otherwise unsatisfactory, and can be actually detrimental to the animal. The disclosure demonstrates that a replacement with human sequence at an endogenous locus under control of endogenous regulatory elements provides a physiologically appropriate expression pattern and level that results in a useful humanized animal whose physiology with respect to the replaced gene are meaningful and appropriate in the context of the humanized animal's physiology.

Genetically modified animals that express human or humanized IL2RG, IL2RB, IL15RA, and/or IL15 proteins, e.g., in a physiologically appropriate manner, provide a variety of uses that include, but are not limited to, developing therapeutics for human diseases and disorders, and assessing the toxicity and/or the efficacy of these human therapeutics in the animal models.

In various aspects, genetically modified animals are provided that express human or humanized IL2RG, IL2RB, IL15RA, and/or IL15, which are useful for testing therapeutic agents that can decrease or block the interaction between the interaction between IL2 and IL2 receptor complex (or the interaction between IL15 and IL15 receptor complex), testing whether antibodies targeting IL2RG, IL2RB, IL15RA, and/or IL15 can bind to their target antigens, testing whether an therapeutic agent can increase or decrease the immune response, and/or determining whether an agent is an IL2RG, IL2RB, IL15RA, and/or IL15 agonist or antagonist. The genetically modified animals can be, e.g., an animal model of a human disease, e.g., the disease is induced genetically (a knock-in or knockout). In various embodiments, the genetically modified non-human animals further comprise an impaired immune system, e.g., a non-human animal genetically modified to sustain or maintain a human xenograft, e.g., a human solid tumor (e.g., lung cancer) or a blood cell tumor (e.g., a lymphocyte tumor, a B or T cell tumor).

In some embodiments, the genetically modified animals can be used for determining effectiveness of a therapeutic agent (e.g., an antibody targeting IL2RG, IL2RB, IL15RA, and/or IL15; or a drug targeting the IL2/IL15 signaling pathways) for the treatment of cancer. In some embodiments, the methods involve administering the therapeutic agent to the animal as described herein, wherein the animal has a cancer or tumor; and determining inhibitory effects of the therapeutic agent to the cancer or tumor. The inhibitory effects that can be determined include, e.g., a decrease of tumor size or tumor volume, a decrease of tumor growth, a reduction of the increase rate of tumor volume in a subject (e.g., as compared to the rate of increase in tumor volume in the same subject prior to treatment or in another subject without such treatment), a decrease in the risk of developing a metastasis or the risk of developing one or more additional metastasis, an increase of survival rate, and an increase of life expectancy, etc. The tumor volume in a subject can be determined by various methods, e.g., as determined by direct measurement, MRI or CT. In some embodiments, the antibody can directly target cells expressing IL2RG, IL2RB, IL15RA, and/or IL15.

In some embodiments, the tumor comprises one or more cancer cells (e.g., human or mouse cancer cells) that are injected into the animal. In some embodiments, the antibody activates IL2 and/or IL15 signaling pathways. In some embodiments, the antibody does not activate IL2 and/or IL15 signaling pathways.

In some embodiments, the genetically modified animals can be used for determining whether an antibody is a IL2RG, IL2RB, IL15RA, and/or IL15 agonist or antagonist. In some embodiments, the methods as described herein are also designed to determine the effects of the therapeutic agent (e.g., an antibody targeting IL2RG, IL2RB, IL15RA, and/or IL15; or a drug targeting the IL2/IL15 signaling pathways) on IL2RG, IL2RB, IL15RA, and/or IL15, e.g., whether the agent can upregulate the immune response or downregulate immune response, and/or whether the agent can induce complement mediated cytotoxicity (CMC) or antibody dependent cellular cytotoxicity (ADCC). In some embodiments, the genetically modified animals can be used for determining the effective dosage of a therapeutic agent for treating a disease in the subject, e.g., cancer.

The inhibitory effects on tumors can also be determined by methods known in the art, e.g., measuring the tumor volume in the animal, and/or determining tumor (volume) inhibition rate (TGI_{TV}). The tumor growth inhibition rate can be calculated using the formula TGI_{TV} (%)=(1−TVt/TVc)×100, where TVt and TVc are the mean tumor volume (or weight) of treated and control groups.

In some embodiments, the therapeutic agent (e.g., an antibody targeting IL2RG, IL2RB, IL15RA, and/or IL15; or a drug targeting the IL2/IL15 signaling pathways) is designed for treating various cancers. As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “tumor” as used herein refers to cancerous cells, e.g., a mass of cancerous cells. Cancers that can be treated or diagnosed using the methods described herein include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. In some embodiments, the agents described herein are designed for treating or diagnosing a carcinoma in a subject. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the cancer is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

In some embodiments, the cancer described herein is lymphoma, non-small cell lung cancer, cervical cancer, leukemia, ovarian cancer, nasopharyngeal cancer, breast cancer, endometrial cancer, colon cancer, rectal cancer, gastric cancer, bladder cancer, glioma, lung cancer, bronchial cancer, bone cancer, prostate cancer, pancreatic cancer, liver and bile duct cancer, esophageal cancer, kidney cancer, thyroid cancer, head and neck cancer, testicular cancer, glioblastoma, astrocytoma, melanoma, myeloproliferation abnormal syndromes, and sarcomas. In some embodiments, the leukemia is selected from acute lymphocytic (lymphoblastic) leukemia, acute myeloid leukemia, myeloid leukemia, chronic lymphocytic leukemia, multiple myeloma, plasma cell leukemia, and chronic myelogenous leukemia. In some embodiments, the lymphoma is selected from Hodgkin's lymphoma and non-Hodgkin's lymphoma, including B-cell lymphoma, diffuse large B-cell lymphoma, follicular lymphoma, mantle cell lymphoma, marginal zone B-cell lymphoma, T-cell lymphoma, and Waldenstrom macroglobulinemia. In some embodiments, the sarcoma is selected from the group consisting of osteosarcoma, Ewing sarcoma, leiomyosarcoma, synovial sarcoma, soft tissue sarcoma, angiosarcoma, liposarcoma, fibrosarcoma, rhabdomyosarcoma, and chondrosarcoma. In a specific embodiment, the tumor is breast cancer, ovarian cancer, endometrial cancer, melanoma, kidney cancer, lung cancer, or liver cancer.

In some embodiments, the therapeutic agent (e.g., an antibody targeting IL2RG, IL2RB, IL15RA, and/or IL15; or a drug targeting the IL2/IL15 signaling pathways) is designed for treating various autoimmune diseases, including rheumatoid arthritis, Crohn's disease, systemic lupus erythematosus, ankylosing spondylitis, inflammatory bowel diseases (IBD), ulcerative colitis, or scleroderma. In some embodiments, the anti-IL2RG antibody is designed for treating various immune disorders, including allergy, asthma, and/or atopic dermatitis. Thus, the methods as described herein can be used to determine the effectiveness of an therapeutic agent (e.g., an antibody targeting IL2RG, IL2RB, IL15RA, and/or IL15; or a drug targeting the IL2/IL15 signaling pathways) in inhibiting immune response. In some embodiments, the immune disorders described herein is graft versus host disease (GVHD), psoriasis, allergy, asthma, myocarditis, nephritis, hepatitis, systemic lupus erythematosus, rheumatoid arthritis, scleroderma, hyperthyroidism, idiopathic thrombocytopenia purpura, autoimmune hemolytic anemia, ulcerative colitis, autoimmune liver disease, diabetes, pain or neurological disorders, etc. In some embodiments, the therapeutic agent (e.g., an antibody targeting IL2RG, IL2RB, IL15RA, and/or IL15; or a drug targeting the IL2/IL15 signaling pathways) is designed for treating various inflammations, e.g., viral inflammation. In some embodiments, the inflammation described herein includes both acute inflammation and chronic inflammation. Specifically, the inflammation includes but not limited to degenerative inflammation, exudative inflammation (e.g., serous inflammation, fibrinous inflammation, suppurative inflammation, hemorrhagic inflammation, necrotic inflammation, catarrhal inflammation), proliferative inflammation, specific inflammation (e.g., tuberculosis, syphilis, leprosy, or lymphogranuloma). In some embodiments, the inflammation described herein includes infection, and the infection refers to the local tissue and systemic inflammatory response caused by bacteria, viruses, fungi, parasites and/or other pathogens invading the human body.

The present disclosure also provides methods of determining toxicity of an antibody (e.g., an antibody targeting IL2RG, IL2RB, IL15RA, and/or IL15). The methods involve administering the antibody to the animal as described herein. The animal is then evaluated for its weight change, red blood cell count, hematocrit, and/or hemoglobin. In some embodiments, the antibody can decrease the red blood cells (RBC), hematocrit, or hemoglobin by more than 20%, 30%, 40%, or 50%. In some embodiments, the animals can have a weight that is at least 5%, 10%, 20%, 30%, or 40% smaller than the weight of the control group (e.g., average weight of the animals that are not treated with the antibody).

The present disclosure also relates to the use of the animal model generated through the methods as described herein in the development of a product related to an immunization processes of human cells, the manufacturing of a human antibody, or the model system for a research in pharmacology, immunology, microbiology and medicine.

In some embodiments, the disclosure provides the use of the animal model generated through the methods as described herein in the production and utilization of an animal experimental disease model of an immunization processes involving human cells, the study on a pathogen, or the development of a new diagnostic strategy and/or a therapeutic strategy.

The disclosure also relates to the use of the animal model generated through the methods as described herein in the screening, verifying, evaluating or studying the IL2RG, IL2RB, IL15RA, and/or IL15 gene functions, human IL2RG, IL2RB, IL15RA, and/or IL15 antibodies, drugs for human IL2RG, IL2RB, IL15RA, and/or IL15 targeting sites, the drugs or efficacies for human IL2RG, IL2RB, IL15RA, and/or IL15 targeting sites, the drugs for immune-related diseases and antitumor drugs.

In some embodiments, the disclosure provides a method to verify in vivo efficacy of TCR-T, CAR-T, and/or other immunotherapies (e.g., T-cell adoptive transfer therapies). For example, the methods include transplanting human tumor cells into the animal described herein, and applying human CAR-T to the animal with human tumor cells. Effectiveness of the CAR-T therapy can be determined and evaluated. In some embodiments, the animal is selected from the IL2RG, IL2RB, IL15RA, and/or IL15 gene humanized non-human animal prepared by the methods described herein, the IL2RG, IL2RB, IL15RA, and/or IL15 gene humanized non-human animal described herein, the double- or multi-humanized non-human animal generated by the methods described herein (or progeny thereof), a non-human animal expressing the human or humanized IL2RG, IL2RB, IL15RA, and/or IL15 protein, or the tumor-bearing or inflammatory animal models described herein. In some embodiments, the TCR-T, CAR-T, and/or other immunotherapies can treat the IL2RG, IL2RB, IL15RA, and/or IL15-associated diseases described herein. In some embodiments, the TCA-T, CAR-T, and/or other immunotherapies provides an evaluation method for treating the IL2RG, IL2RB, IL15RA, and/or IL15-associated diseases described herein.

Genetically Modified Animal Model with Two or More Human or Chimeric Genes

The present disclosure further relates to methods for generating genetically modified animal model with two or more human or chimeric genes. The animal can comprise human or chimeric IL2RG, IL2RB, IL15RA, and/or IL15 genes and a sequence encoding an additional human or chimeric protein. In some embodiments, the animal comprises human or humanized IL2RG and IL2RB genes. In some embodiments, the animal comprises human or humanized IL15RA and IL15 genes. In some embodiments, the animal comprises human or humanized IL2RG/IL2RB/IL15RA/IL15 genes.

In some embodiments, the additional human or chimeric protein can be Interleukin 2 (IL-2), Interleukin 2 Receptor Subunit Alpha (IL2RA), programmed cell death protein 1 (PD-1), programmed death-ligand 1 (PD-L1), Interleukin 10 Receptor Subunit Alpha (IL10RA), and/or cytotoxic T-lymphocyte-associated protein 4 (CTLA4).

The methods of generating genetically modified animal model with two or more human or chimeric genes (e.g., humanized genes) can include the following steps:
(a) using the methods of introducing human or chimeric IL2RG, IL2RB, IL15RA, and/or IL15 genes as described herein to obtain a genetically modified non-human animal;
(b) mating the genetically modified non-human animal with another genetically modified non-human animal, and then screening the progeny to obtain a genetically modified non-human animal with two or more human or chimeric genes.

In some embodiments, in step (b) of the method, the genetically modified animal can be mated with a genetically modified non-human animal with human or chimeric IL2, IL2RA, PD-1, PD-L1, IL10RA and/or CTLA4. Some of these genetically modified non-human animal are described, e.g., in PCT/CN2020/107886, PCT/CN2018/110069, PCT/CN2017/099574, PCT/CN2020/128201, and PCT/CN2017/099577; each of which is incorporated herein by reference in its entirety.

In some embodiments, the humanization is directly performed on a genetically modified animal having human or chimeric IL2RG, IL2RB, IL15RA, IL15, IL2, IL2RA, PD-1, PD-L1, IL10RA, and/or CTLA4 genes.

As these proteins may involve different mechanisms, a combination therapy that targets two or more of these proteins thereof may be a more effective treatment. In fact, many related clinical trials are in progress and have shown a good effect. The genetically modified animal model with two or more human or humanized genes can be used for determining effectiveness of a combination therapy that targets two or more of these proteins, e.g., an anti-IL2RG antibody (alternatively, an anti-IL2RB antibody, an anti-IL15RA antibody, or an anti-IL15 antibody) and an additional therapeutic agent for the treatment of an immune disorder (e.g., psoriasis). The methods include administering the anti-IL2RG antibody (alternatively, an anti-IL2RB antibody, an anti-IL15RA antibody, or an anti-IL15 antibody) and the additional therapeutic agent to the animal, wherein the animal has a tumor; and determining the inhibitory effects of the combined treatment to the tumor. In some embodiments, the additional therapeutic agent is an antibody that specifically binds to IL2, IL2RA, PD-1, PD-L1, IL10RA or CTLA4. In some embodiments, the additional therapeutic agent is an anti-CTLA4 antibody (e.g., ipilimumab), an anti-PD-1 antibody (e.g., nivolumab), or an anti-PD-L1 antibody.

In some embodiments, the animal further comprises a sequence encoding a human or humanized PD-1, a sequence encoding a human or humanized PD-L1, or a sequence encoding a human or humanized CTLA-4. In some embodiments, the additional therapeutic agent is an anti-PD-1 antibody (e.g., nivolumab, pembrolizumab), an anti-PD-L1 antibody, or an anti-CTLA-4 antibody. In some embodiments, the tumor comprises one or more tumor cells that express CD80, CD86, PD-L1, and/or PD-L2.

In some embodiments, the combination treatment is designed for treating various cancers as described herein, e.g., breast cancer, ovarian cancer, endometrial cancer, melanoma, kidney cancer, lung cancer, or liver cancer. In some embodiments, the combination treatment is designed for treating immune disorders as described herein, e.g., psoriasis.

In some embodiments, the methods described herein can be used to evaluate the combination treatment with some other methods. The methods of treating a cancer that can be used alone or in combination with methods described herein, include, e.g., treating the subject with chemotherapy, e.g., campothecin, doxorubicin, cisplatin, carboplatin, procarbazine, mechlorethamine, cyclophosphamide, adriamycin, ifosfamide, melphalan, chlorambucil, bisulfan, nitrosurea, dactinomycin, daunorubicin, bleomycin, plicomycin, mitomycin, etoposide, verampil, podophyllotoxin, tamoxifen, taxol, transplatinum, 5-flurouracil, vincristin, vinblastin, and/or methotrexate. Alternatively or in addition, the methods can include performing surgery on the subject to remove at least a portion of the cancer, e.g., to remove a portion of or all of a tumor(s), from the patient.

### EXAMPLES

The invention is further described in the following examples, which do not limit the scope of the invention described in the claims.

### Materials and Methods

The following materials were used in the following examples.

BspHI, BglII, MfeI, XbaI, and ScaI restriction enzymes were purchased from NEB (Cat #: R0517S, R0144S, R0589S, R0145S, and R3122S, respectively).

C57BL/6 mice and Flp transgenic mice were purchased from the China Food and Drugs Research Institute National Rodent Experimental Animal Center.

TRIzol™ reagent was purchased from Invitrogen (Cat #: 15596018).

Attune™ Nxt Acoustic Focusing Cytometer was purchased from Thermo Fisher (Model number: Attune™ Nxt).

BioTek Epoch Microplate Reader was purchased from BioTeK (Model number: EROCH).

FITC anti-Mouse CD19 was purchased from BioLegend (Cat #: 115506).

PerCP/Cy5.5 anti-mouse TCR β chain was purchased from BioLegend (Cat #: 109228).

PE/Cy™ 7 Mouse anti-mouse NK1.1 was purchased from BD Pharmingen (Cat #: 552878).

PE anti-human CD132 (common γ chain) was purchased from BioLegend (Cat #: 338605).

APC anti-mouse CD132 (common γ chain) was purchased from BioLegend (Cat #: 132307).

APC anti-mouse CD122 (IL-2Rβ) Antibody was purchased from BioLegend (Cat #: 105911).

PE anti-human CD122 (IL-2Rβ) Antibody (hIL2RB) was purchased from BioLegend (Cat #: 339005).

Mouse IL-15 ELISA Kit was purchased from Abcam (Cat #: ab100701).

Human IL-15 Quantikine ELISA Kit was purchased from R&D Systems (Cat #: D1500).

Brilliant Violet 510™ anti-mouse CD45 Antibody was purchased from BioLegend (Cat #: 103138).

BD Pharmingen™ PE Rat Anti-Mouse CD215 (IL-15Rα) was purchased from BD Biosciences (Cat #: 568235).

PE anti-human CD215 (IL-15Rα) Antibody was purchased from BioLegend (Cat #: 330207).

Brilliant Violet 711™ anti-mouse CD11c Antibody was purchased from BioLegend (Cat #: 117349).

APC Rat IgG2a, κ Isotype Ctrl Antibody was purchased from BioLegend (Cat #: 400512).

PE Mouse IgG2b, κ Isotype Ctrl Antibody (Fc) was purchased from BioLegend (Cat #: 402204).

Zombie NIR™ Fixable Viability Kit was purchased from BioLegend (Cat #: 423106).

Purified anti-mouse CD16/32 Antibody was purchased from BioLegend (Cat #: 101302).

PerCP anti-mouse CD45 Antibody was purchased from BioLegend (Cat #: 103130).

Brilliant Violet 510™ anti-mouse CD4 Antibody was purchased from BioLegend (Cat #: 100559).

PE anti-mouse CD8a Antibody was purchased from BioLegend (Cat #: 100708).

FOXP3 Monoclonal Antibody (FJK-16s), PerCP-eFluor™ 710, eBioscience™ was purchased from Thermo Fisher (Cat #: 46-5773-82).

Brilliant Violet 421™ anti-mouse NK-1.1 Antibody was purchased from BioLegend (Cat #: 108732).

Brilliant Violet 785™ anti-mouse/human CD11b Antibody was purchased from BioLegend (Cat #: 101243).

APC anti-mouse F4/80 Antibody was purchased from BioLegend (Cat #: 123116).

Brilliant Violet 650™ anti-mouse Ly-6G Antibody was purchased from BioLegend (Cat #: 127641).

PE/Cyanine7 anti-mouse CD25 Antibody was purchased from BioLegend (Cat #: 101916).

Alexa Fluor® 700 anti-mouse CD3 Antibody was purchased from BioLegend (Cat #: 100216).

FITC anti-mouse TCR β chain Antibody was purchased from BioLegend (Cat #: 109205).

Brilliant Violet 421™ anti-mouse CD4 Antibody was purchased from BioLegend (Cat #: 100438).

Brilliant Violet 711™ anti-mouse CD8a Antibody was purchased from BioLegend (Cat #: 100759).

PE Mouse IgG1, κ Isotype Ctrl Antibody was purchased from BioLegend (Cat #: 400112).

PE Rat IgG2b, κ Isotype Ctrl Antibody was purchased from BioLegend (Cat #: 400608).

APC Mouse IgG2b, κ Isotype Ctrl Antibody was purchased from BioLegend (Cat #: 402206).

IL-15/IL-15R Complex Mouse ELISA Kit was purchased from Invitrogen (Cat #: BMS6023).

Mouse IL-15 ELISA Kit was purchased from Abcam (Cat #: ab275898).

Brilliant Violet 605™ anti-mouse CD11c Antibody was purchased from BioLegend (Cat #: 117334).

PE Rat IgG1, κ Isotype Control was purchased from BD Biosciences (Cat #: 551979).

PE Mouse IgG2b, κ Isotype Ctrl Antibody (Fc, ICFC) was purchased from BioLegend (Cat #: 400314).

eBioscience™ Fixable Viability Dye eFluor™ 780 was purchased from Thermo Fisher (Cat #: 65-0865-14).

Brilliant Violet 711™ anti-mouse TCR β chain Antibody was purchased from BioLegend (Cat #: 109243).

Phospho-Stat5 (Tyr694) (D47E7) XP® Rabbit mAb was purchased from Cell Signaling Technology (Cat #: 4322S).

APC anti-mouse CD4 Antibody was purchased from BioLegend (Cat #: 100412).

Alexa Fluor® 700 anti-mouse CD8a Antibody was purchased from BioLegend (Cat #: 100730).

PE anti-STAT5 Phospho (Tyr694) Antibody was purchased from BioLegend (Cat #: 936904).

Anti-rabbit IgG (H+L), F(ab′)₂ Fragment (Alexa Fluor® 488 Conjugate) was purchased from Cell Signaling Technology (Cat #: 4412).

Human IL-2 Protein, Tag Free was purchased from ACROBiosystems (Cat #: IL2-H4113).

Biotinylated Mouse IL-2 Protein was purchased from Kactus Biosystems (Cat #: IL2-MM401B).

Human IL-15 Protein, premium grade was purchased from ACROBiosystems (Cat #: IL5-H4117).

Human IL-15 R alpha/CD215 Protein, Fc Tag was purchased from ACROBiosystems (Cat #: ILA-H5253).

Recombinant Mouse IL-15 Protein was purchased from R&D Systems (Cat #: 447-ML-010).

Recombinant Mouse IL-15R alpha Fc Chimera Protein, CF was purchased from R&D Systems (Cat #: 551-MR-100).

### Example 1: Mice with Humanized IL2RG Gene (Modification 1)

In this example, a non-human animal (e.g., a mouse) was modified to include a nucleotide sequence encoding human IL2RG protein, and the obtained genetically-modified non-human animal can express a human or humanized IL2RG protein in vivo. The mouse IL2RG gene (NCBI Gene ID: 16186, Primary source: MGI: 96551, UniProt ID: P34902) is located at 100307991 to 100311861 of chromosome X (NC_000086.8), and the human IL2RG gene (NCBI Gene ID: 3561, Primary source: HGNC: 6010, UniProt ID: P31785) is located at 71107404 to 71111631 of chromosome X (NC_000023.11). The mouse IL2RG transcript is NM_013563.4, and the corresponding protein sequence NP_038591.1 is set forth in SEQ ID NO: 1. The human IL2RG transcript is NM_000206.2, and the corresponding protein sequence NP_000197.1 is set forth in SEQ ID NO: 2. Mouse and human IL2RG gene loci are shown in FIG. 1.

All or part of nucleotide sequences encoding human IL2RG protein can be introduced into the mouse endogenous IL2RG locus, so that the mouse expresses human or humanized IL2RG protein. Specifically, using gene-editing techniques, under control of mouse IL2RG gene regulatory elements, a nucleotide sequence encoding human IL2RG protein can be introduced into the mouse endogenous IL2RG locus. For example, a sequence starting from within exon 1 and ending within exon 8 of mouse IL2RG gene was replaced with a corresponding sequence starting from within exon 1 and ending within exon 8 of human IL2RG gene, to obtain a humanized IL2RG gene locus as shown in FIG. 2, thereby humanizing mouse IL2RG gene.

As shown in the schematic diagram of the targeting strategy in FIG. 3, the targeting vector V1 contains homologous arm sequences upstream and downstream of the mouse IL2RG gene, and an “A1 Fragment” containing DNA sequences of human IL2RG gene. Specifically, sequence of the upstream homologous arm (5′ homologous arm, SEQ ID NO: 3) is identical to nucleotide sequence of 100311776-100315845 of NCBI accession number NC_000086.8, and sequence of the downstream homologous arm (3′ homologous arm, SEQ ID NO: 4) is identical to nucleotide sequence of 100304102-100308040 of NCBI accession number NC_000086.8. The genomic DNA sequence from human IL2RG gene (SEQ ID NO: 5) in the A1 Fragment is identical to nucleotide sequence of 71107340-71111539 of NCBI accession number NC_000023.11.

The targeting vector also includes an antibiotic resistance gene for positive clone screening (neomycin phosphotransferase gene, or Neo), and two Frt recombination sites flanking the antibiotic resistance gene, that formed a Neo cassette. The connection between the 5′ end of the Neo cassette and the human sequence was designed as:

(SEQ ID NO: 6)
5′-GAAGCCTGGGATGCAATGAGGGTGGGAGGGGAAGAAAAAGCTTGAT
ATCGAATTCCGAAGTTCCTATTCTCTA-3′,

wherein the last “A” in sequence “GAAA” is the last nucleotide of the human sequence, and the first “A” in sequence “AAGC” is the first nucleotide of the Neo cassette. The connection between the 3′ end of the Neo cassette and the mouse sequence was designed as:

(SEQ ID NO: 7)
5′-TTCATCAGTCAGGTACATAATGGTGGATCCGATATCATTCAACTG
TTTCCAAATCAACAAGAAA-3′,

wherein the “C” in sequence “TATC” is the last nucleotide of the Neo cassette, and the “A” in sequence “ATTC” is the first nucleotide of the mouse sequence. The mRNA sequence of the engineered mouse IL2RG after humanization and its encoded protein sequence are shown in SEQ ID NO: 84 and SEQ ID NO: 2, respectively.

In addition, the CRISPR/Cas system can also be used for gene editing, and the targeting strategy shown in FIG. 4 was designed. The targeting vector V2 contains homologous arm sequences upstream and downstream of the mouse IL2RG gene, and an “A2 Fragment” containing DNA sequences of human IL2RG gene. Specifically, sequence of the upstream homologous arm (5′ homologous arm, SEQ ID NO: 8) is identical to nucleotide sequence of 100311776-100313491 of NCBI accession number NC_000086.8, and sequence of the downstream homologous arm (3′ homologous arm, SEQ ID NO: 9) is identical to nucleotide sequence of 100306445-100308040 of NCBI accession number NC_000086.8. The genomic DNA sequence from human IL2RG gene (SEQ ID NO: 5) in the A2 Fragment is identical to nucleotide sequence of 71107340-71111539 of NCBI accession number NC_000023.11. The protein sequence expressed by the genetically modified mice having a humanized IL2RG gene locus is shown in SEQ ID NO: 2.

The targeting vector was constructed, e.g., by restriction enzyme digestion, ligation, or direct synthesis. The constructed targeting vector sequences were preliminarily confirmed by restriction enzyme digestion, and then verified by sequencing. Targeting vectors with verified sequences were used for subsequent experiments.

Specific sgRNA sequences were designed and synthesized that recognize the targeting site. The targeting site sequences of the sgRNAs on the IL2RG gene locus are as follows: sgRNA1 targeting site (SEQ ID NO: 10): 5′-GTTGTTGAGAGGAAGGCTATGGG-3′; sgRNA2 targeting site (SEQ ID NO: 11): 5′-TTTGGAAACAGTTGAATCATAGG-3′.

UCA kit was used to detect the activity of the sgRNAs. After confirming that the sgRNAs can induce efficient Cas9 cleavage, restriction enzyme cleavage sites were added to its 5′ end and a complementary strand to obtain a forward oligonucleotide and a reverse oligonucleotide. After annealing, the products were ligated to the pT7-sgRNA plasmid (the plasmid was first linearized with BbsI), to obtain expression vector pT7-IL2RG-1 and pT7-IL2RG-2. The pT7-sgRNA vector was synthesized, which included a DNA fragment containing the T7 promoter and sgRNA scaffold (SEQ ID NO: 12), and was ligated to the backbone vector (Takara, Catalog number: 3299) after restriction enzyme digestion (EcoRI and BamHI). The resulting plasmid was confirmed by sequencing.

The pre-mixed Cas9 mRNA, the targeting vector, and in vitro transcription products of the pT7-IL2RG-1 and pT7-IL2RG-2 plasmids (using Ambion™ in vitro transcription kit to carry out the transcription according to the method provided in the product instruction) were injected into the cytoplasm or nucleus of fertilized eggs of C57BL/6 or BALB/c mice with a microinjection instrument. The embryo microinjection was carried out according to the method described, e.g., in A. Nagy, et al., “Manipulating the Mouse Embryo: A Laboratory Manual (Third Edition),” Cold Spring Harbor Laboratory Press, 2006. The injected fertilized eggs were then transferred to a culture medium to culture for a short time and then was transplanted into the oviduct of the recipient mouse to produce the genetically modified mice (F0 generation). The mouse population was further expanded by cross-breeding and self-breeding to establish stable homozygous mouse lines having a humanized IL2RG gene locus.

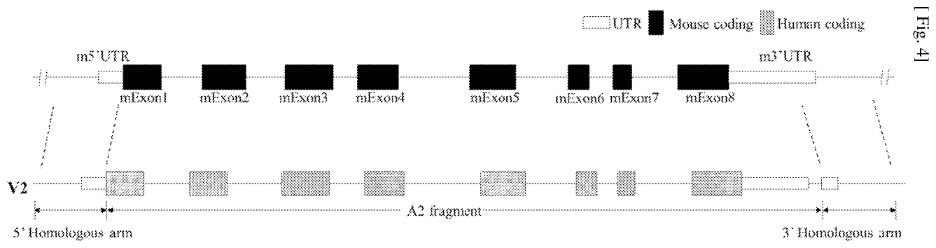

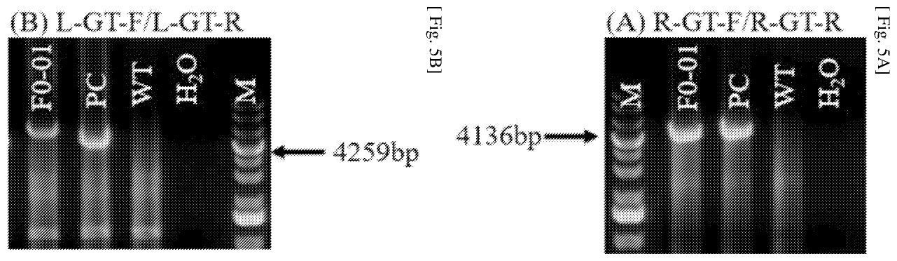

The genotype of the somatic cells of the F0 generation mice can be identified by PCR analysis. The identification results of some F0 generation mice are shown in FIGS. 5A-5B. Combined with the PCR detection results and further verification by sequencing, F0-01 was identified as a positive mouse. The PCR primers are shown in the table below.

TABLE 9
PCR primer sequences and recombinant fragment sizes (F0 generation
genotype)

| | | | Recombinant |
|---|---|---|---|
| Primer | SEQ ID NO | Primer sequence (5′-3′) | fragment size |
| L-GT-F | SEQ ID NO: 13 | CAAGCTTGCCATGCCAGTGTTTAGG | Mut: 4259 bp |
| L-GT-R | SEQ ID NO: 14 | CATTCACTCCAATGCTGAGCACTTCC | |
| R-GT-F | SEQ ID NO: 15 | GAAGCCATTAAGGGGATCCAGAGGG | Mut: 4136 bp |
| R-GT-R | SEQ ID NO: 16 | GAGCTGGTACACATTTTTCTTCCCC | |

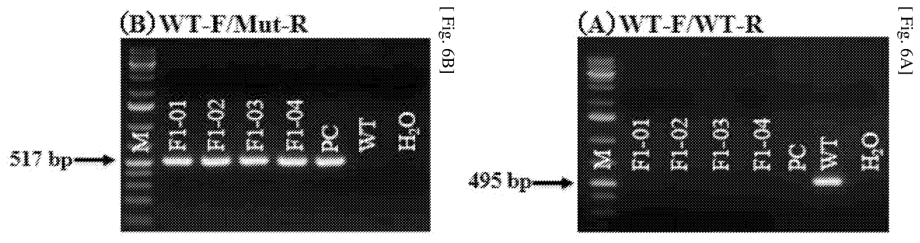

The F0 generation mice identified as positive were bred with wild-type mice to generate F1 generation mice. The F1 generation mice can be genotyped using the same PCR method described above. The identification results are shown in FIGS. 6A-6B, and mice numbered F1-01, F1-02, F1-03, and F1-04 were identified as positive mice. The PCR primers are shown in the table below.

TABLE 10
PCR primer sequences and recombinant fragment sizes (F1 generation
genotype)

| | | | Recombinant |
|---|---|---|---|
| Primer | SEQ ID NO | Primer sequence (5′-3′) | fragment size |
| WT-F | SEQ ID NO: 17 | GGACCTAGGGTCCTGAAGGGTCTTG | WT: 495 bp |
| WT-R | SEQ ID NO: 18 | CCCCCTCTGCACACTTCGTTTTAGT | |
| WT-F | SEQ ID NO: 17 | GGACCTAGGGTCCTGAAGGGTCTTG | Mut: 517 bp |
| Mut-R | SEQ ID NO: 19 | CCCACTCCACTTTTCAATTCTGCCC | |

The F1 generation mice identified as positive by PCR were further verified by Southern Blot to confirm whether there was random insertion. Specifically, genomic DNA from the mouse tail was extracted, which was digested with BspHI or BglII restriction enzyme. The digested genomic DNA was then transferred to a membrane and hybridized with respective probes. The restriction enzymes, probes, and the size of target fragment are shown in the table below.

TABLE 11
Enzymes and probes used in Southern Blot

| Restriction | | Wild-type | Recombinant |
|---|---|---|---|
| ezyme | Probe | fragment size | fragment size |
| BspHI | 3′Probe | 7.5 kb | 6.2 kb |
| BglII | LR Probe | 6.4 kb | 4.0 kb |

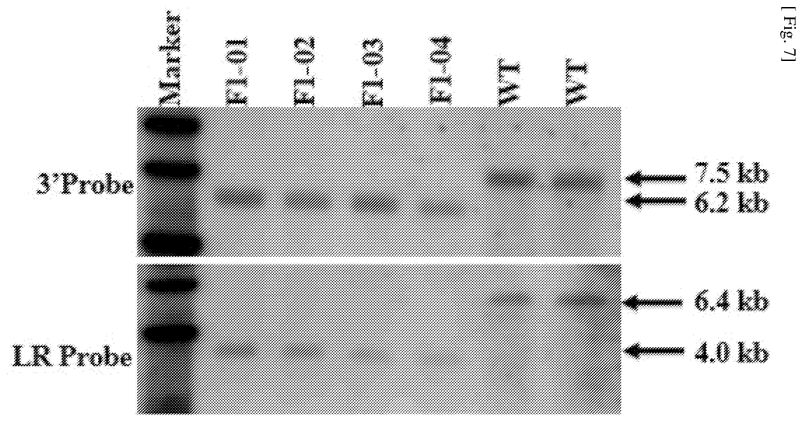

The Southern Blot detection results are shown in FIG. 7. The results showed that mice numbered F1-01, F1-02, F1-03, and F1-04 were verified to have no random insertions. The results indicate that the IL2RG gene humanized mice constructed using the methods described herein can be stably passaged without random insertions.

The following primers were used for probe synthesis in Southern Blot assays:

3′Probe:
3′Probe-F:
(SEQ ID NO: 20)
5′-GAATGTAGGGGTGGGGCTAGCATAG-3′,
3′Probe-R:
(SEQ ID NO: 21)
5′-CTAGTAGTCTGCTGGGCTCCACCAC-3′;
LR Probe:
LR Probe-F:
(SEQ ID NO: 22)
5′-GTTGGCTGGATAAACAATTTCAGTAAA-3′,
LR Probe-R:
(SEQ ID NO: 23)
5′-CTAACTAGTACTAACTTCAGACTTCC-3′.

The expression of human or humanized IL2RG protein in positive mice can also be confirmed, e.g., by flow cytometry. Specifically, one 7-week-old female C57BL/6 wild-type mouse and one IL2RG gene humanized heterozygous mouse were selected. Spleen tissues were collected after euthanasia by cervical dislocation. Cells were stained with: PerCP/Cyanine5.5 anti-mouse TCRβ chain Antibody (mTCRβ; a mouse T cell marker antibody), FITC anti-Mouse CD19 (mCD19; a B cell marker antibody), PE-Cy™ 7 Mouse anti-mouse NK1.1 (mNK1.1; an NK cell marker antibody), APC anti-mouse CD132 (common γ chain) (mIL2RG; an anti-mouse CD132 antibody), and/or PE anti-human CD132 (common γ chain) (hIL2RG; an anti-human CD132 antibody), and then subjected to flow cytometry analysis.

The results showed that in wild-type C57BL/6 mouse NK cells (characterized by mCD45+mTCRβ-mNK1.1+), the proportion of mouse IL2RG positive cells (characterized by mCD45+mTCRβ-mNK1.1+mIL2RG+) was 43.0%. In IL2RG gene humanized heterozygous mouse NK cells, the proportion of mouse IL2RG positive cells was 2.37%, and the proportion of human IL2RG positive cells was 42.8%. The results indicate that the IL2RG gene humanized mice prepared by the method described herein can successfully express human IL2RG protein in vivo.

### Example 2: Mice with Humanized IL2RG Gene (Modification 2)

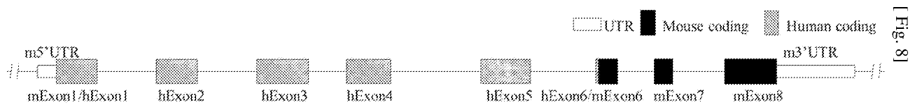

A sequence starting from within exon 1 and ending within exon 6 of mouse IL2RG gene was replaced with a corresponding sequence starting from within exon 1 and ending within exon 6 of human IL2RG gene, to obtain a humanized IL2RG gene locus as shown in FIG. 8, thereby humanizing mouse IL2RG gene.

As shown in the schematic diagram of the targeting strategy in FIG. 9, the targeting vector V3 contains homologous arm sequences upstream and downstream of the mouse IL2RG gene, and an “A3 Fragment” containing DNA sequences of human IL2RG gene. Specifically, sequence of the upstream homologous arm (5′ homologous arm, SEQ ID NO: 24) is identical to nucleotide sequence of 100311776-100317900 of NCBI accession number NC_000086.8, and sequence of the downstream homologous arm (3′ homologous arm, SEQ ID NO: 25) is identical to nucleotide sequence of 100305674-100309321 of NCBI accession number NC_000086.8. The genomic DNA sequence from human IL2RG gene (SEQ ID NO: 26) in the A3 Fragment is identical to nucleotide sequence of 71108685-71111539 of NCBI accession number NC_000023.11.

The targeting vector also includes an antibiotic resistance gene for positive clone screening (neomycin phosphotransferase gene, or Neo), and two Frt recombination sites flanking the antibiotic resistance gene, that formed a Neo cassette. The connection between the 5′ end of the Neo cassette and the human sequence was designed as:

(SEQ ID NO: 27)
5′-AATGTATAGGATTTCCCTGAAGCAT
TCCTAGAGAGCAAGCTTGATATCGAATTCCGAAGTTCCTATTCTCT-3′,

7), wherein the “C” in sequence “GAGC” is the last nucleotide of the human sequence, and the first “A” in sequence “AAGC” is the first nucleotide of the Neo cassette. The connection between the 3′ end of the Neo cassette and the human sequence was designed as:

(SEQ ID NO: 28)
5′-TAGGAACTTCATCAGTCAGGTACATAATGGTGGATCCGATATC
CTGCAAGGTGAAGATGGCTTTGGAACCAGCTG-3′,

wherein the “C” in sequence “TATC” is the last nucleotide of the Neo cassette, and the first “C” in sequence “CTGC” is the first nucleotide of the human sequence. The mRNA sequence of the engineered mouse IL2RG after humanization and its encoded protein sequence are shown in SEQ ID NO: 29 and SEQ ID NO: 30, respectively.

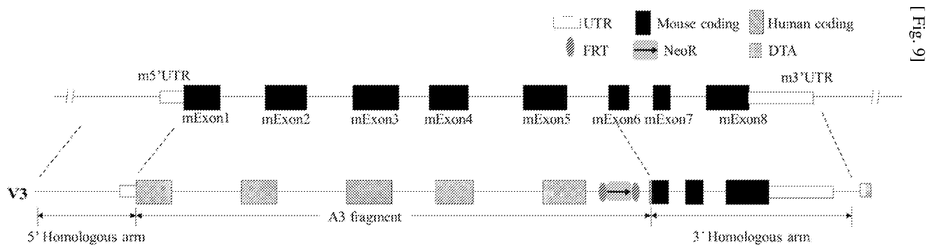

In addition, the CRISPR/Cas system can also be used for gene editing, and the targeting strategy shown in FIG. 10 was designed. The targeting vector V4 contains homologous arm sequences upstream and downstream of the mouse IL2RG gene, and an “A4 Fragment” containing DNA sequences of human IL2RG gene. Specifically, sequence of the upstream homologous arm (5′ homologous arm, SEQ ID NO: 31) is identical to nucleotide sequence of 100311776-100313175 of NCBI accession number NC_000086.8, and sequence of the downstream homologous arm (3′ homologous arm, SEQ ID NO: 32) is identical to nucleotide sequence of 100307922-100309321 of NCBI accession number NC_000086.8. The genomic DNA sequence from human IL2RG gene (SEQ ID NO: 26) in the A4 Fragment is identical to nucleotide sequence of 71108685-71111539 of NCBI accession number NC_000023.11. The protein sequence expressed by the genetically modified mice having a humanized IL2RG gene locus is shown in SEQ ID NO: 30.

### Example 3: Mice with Humanized IL2RB Gene

In this example, a non-human animal (e.g., a mouse) was modified to include a nucleotide sequence encoding human IL2RB protein, and the obtained genetically-modified non-human animal can express a human or humanized IL2RB protein in vivo. The mouse IL2RB gene (NCBI Gene ID: 16185, Primary source: MGI: 96550, UniProt ID: P16297) is located at 78479256 to 78511621 of chromosome 15 (NC_000081.6), and the human IL2RB gene (NCBI Gene ID: 3560, Primary source: HGNC: 6006, UniProt ID: P14784) is located at 37125838 to 37175118 of chromosome 22 (NC_000022.11). The mouse IL2RB transcript is NM_008368.4, and the corresponding protein sequence NP_032394.1 is set forth in SEQ ID NO: 33. The human IL2RB transcript is NM_000878.5, and the corresponding protein sequence NP_000869.1 is set forth in SEQ ID NO: 34. Mouse and human IL2RB gene loci are shown in FIG. 11.

All or part of nucleotide sequences encoding human IL2RB protein can be introduced into the mouse endogenous IL2RB locus, so that the mouse expresses human or humanized IL2RB protein. For example, a sequence (e.g., a DNA or cDNA sequence) containing human IL2RB gene sequences can be directly inserted at the mouse endogenous IL2RB locus. The inserted sequence can also include auxiliary sequences (e.g., a stop codon), or other methods can be used (e.g., inverted sequences or knockout sequences), such that the mouse endogenous IL2RB genomic sequence after the insertion site cannot be expressed normally. Strategies such as in situ replacement can also be used. For example, a sequence at the mouse endogenous IL2RB locus can be directly replaced with a human IL2RB gene sequence (e.g., a DNA or cDNA sequence). This example illustrates how to humanize the mouse IL2RB gene by in situ replacement of DNA sequence.

In this example, mouse cells were modified by gene editing technology, and a mouse IL2RB gene sequence was replaced with a corresponding human IL2RB gene sequence at the mouse endogenous IL2RB gene locus. Specifically, at the mouse endogenous IL2RB gene locus, a sequence starting from within exon 2 and ending within exon 8 of mouse IL2RB gene was replaced with a corresponding sequence starting from within exon 2 and ending within exon 8 of human IL2RB gene, to obtain a humanized IL2RB gene locus as shown in FIG. 12, thereby humanizing mouse IL2RB gene.

The targeting vector contains homologous arm sequences upstream and downstream of the mouse IL2RB gene, and a fragment containing DNA sequences of human IL2RB gene. Specifically, sequence of the upstream homologous arm (5′ homologous arm, SEQ ID NO: 35) is identical to nucleotide sequence of 78495191-78491766 of NCBI accession number NC_000081.6, and sequence of the downstream homologous arm (3′ homologous arm, SEQ ID NO: 36) is identical to nucleotide sequence of 78484605-78479760 of NCBI accession number NC_000081.6. The genomic DNA sequence from human IL2RB gene (SEQ ID NO: 37) is identical to nucleotide sequence of 37144088-37135435 of NCBI accession number NC_000022.11.

The mRNA sequence of the engineered mouse IL2RB after humanization and its encoded protein sequence are shown in SEQ ID NO: 38 and SEQ ID NO: 39, respectively.

The targeting vector also includes an antibiotic resistance gene for positive clone screening (neomycin phosphotransferase gene, or Neo), and two Frt recombination sites flanking the antibiotic resistance gene, that formed a Neo cassette. The connection between the 5′ end of the Neo cassette and the mouse sequence was designed as:

(SEQ ID NO: 40)
5′-ACTCTTGTCTAACCCTCCTTAGAGATTC
CCCCATATGCCGAAGTTCCTATTCTCTAGAAAGTATAGGAACTTC-3′,

0), wherein the last “C” in sequence “TCCCC” is the last nucleotide of the mouse sequence, and the “C” in sequence “CATA” is the first nucleotide of the Neo cassette. The connection between the 3′ end of the Neo cassette and the mouse sequence was designed as:

(SEQ ID NO: 41)
5′-GAAGTTCCTATTC
TCTAGAAAGTATAGGAACTTCATCAGTCAGGTACATAATGGTGGATCC
ACCACACCAGTCATACATCCTGACCCTTGAGGG-3′,

(SEQ ID NO: 41), wherein the last “C” in sequence “GATCC” is the last nucleotide of the Neo cassette, and the first “A” in sequence “ACCAC” is the first nucleotide of the mouse sequence.

The expression of humanized IL2RB protein in positive mice can also be confirmed, e.g., by flow cytometry. Specifically, one 6-week-old C57BL/6 wild-type mouse (+/+) and one IL2RB gene humanized heterozygous mouse (H/+) were selected. 7.5 μg/200 μL of Anti-mCD3 (Alexa Fluor® 700 anti-mouse CD3 Antibody; purchased from BioLegend (Cat #: 100216)) was injected intraperitoneally, and spleen cells were collected for flow cytometric detection 24 hours later. Cells were first stained with: Brilliant Violet 510™ anti-mouse CD45 (mCD45; an anti-mouse CD45 antibody), PerCP/Cyanine5.5 anti-mouse TCR β chain Antibody (mTCRβ), Brilliant Violet 605™ anti-mouse CD4 Antibody (mCD4), Brilliant Violet 711™ anti-mouse CD8a Antibody (mCD8; a T cell marker antibody), and/or PE-Cy™ 7 Mouse anti-mouse NK1.1 (mNK1.1; an NK cell marker antibody), followed by: APC anti-mouse CD122 (IL-2Rβ) Antibody (mIL2RB; an anti-mouse IL2RB antibody) or PE anti-human CD122 (IL-2Rβ) Antibody (hIL2RB; an anti-human IL2RB antibody), and then subjected to flow cytometry analysis.

T cells are characterized by mCD45+mTCRβ+, among which mouse IL2RB-positive (mIL2RB+) T cells are characterized by CD45+mTCRβ+mIL2RB+, and human IL2RB-positive (hIL2RB+) T cells are characterized by mCD45+mTCRβ+hIL2RB+. In addition, NK cells are characterized by mCD45+mTCRβ-mNK1.1+, in which mouse IL2RB-positive NK cells are characterized by mCD45+mTCRβ-mNK1.1+mIL2RB+, and human IL2RB-positive NK cells are characterized by mCD45+mTCRβ-mNK1.1+hIL2RB+.

The flow cytometry analysis results are shown in the table below. The results show that humanized IL2RB protein was detected in IL2RB gene humanized heterozygous mouse, but not in the wild-type C57BL/6 mouse.

TABLE 12
Flow cytometry detection results in IL2RB
gene humanized heterozygous mouse

| | |
|---|---|
| T cell | NK cell |

| Group | mIL2RB+ | hIL2RB+ | mIL2RB+ | hIL2RB+ |
|---|---|---|---|---|
| +/+ | 9.91% | 0.27% | 1.41% | 0.15% |
| H/+ | 8.62% | 6.50% | 2.79% | 2.79% |

### Example 4: Generation of IL2RB/IL2RG Double-Gene Humanized Mice

Because the mouse IL2RB and IL2RG genes are located on chromosome 15 and chromosome X, respectively, the IL2RG gene humanized mice prepared in Example 1 were selected to breed with the IL2RB gene humanized mice prepared in Example 3, and the positive progeny mice were screened to obtain IL2RB/IL2RG double-gene humanized mice.

The expression of IL2RB protein and IL2RG protein in IL2RB/IL2RG double-gene humanized mice were detected by flow cytometry. Specifically, one 9-week-old female wild-type C57BL/6 mouse (+/+) and one 9-week-old female IL2RB/IL2RG double-gene humanized homozygous mouse (H/H; H/H) prepared in this example were selected. 7.5 μg/200 μL of Anti-mCD3 was injected intraperitoneally, and spleen cells were collected for flow cytometric detection 24 hours later. Cells were stained with: Brilliant Violet 510™ anti-mouse CD45 (mCD45; an anti-mouse CD45 antibody), PerCP/Cyanine5.5 anti-mouse TCR β chain Antibody (mTCRβ), APC anti-mouse CD122 (IL-2Rβ) Antibody (mIL2RB), PE anti-human CD122 (IL-2Rβ) Antibody (hIL2RB), PE anti-human CD132 (common γ chain) antibody (mIL2RG), and/or APC anti-mouse CD132 (common γ chain) antibody (hIL2RG), and then subjected to flow cytometry analysis. The results are shown in the table below.

Mouse IL2RB-positive (mIL2RB+) and mouse IL2RG-positive (mIL2RG+) T cells are characterized by mCD45+mTCRβ+mIL2RB+ and mCD45+mTCRβ+mIL2RG+, whereas human IL2RB-positive (hIL2RB+) and human IL2RG-positive (hIL2RG+) T cells are characterized by mCD45+mTCRβ+hIL2RB+ and mCD45+mTCRβ+hIL2RG+, respectively.

TABLE 13
Flow cytometry detection results in IL2RB/IL2RG
double-gene humanized mouse

| Group | mIL2RB+ | hIL2RB+ | mIL2RG+ | hIL2RG+ |
|---|---|---|---|---|
| +/+ | 10.6% | 0.65% | 14.4% | 1.12% |
| H/H; H/H | 0.12% | 8.43% | 0.68% | 6.89% |

The results showed that the IL2RB/IL2RG double-gene humanized mouse prepared in this example successfully expressed human IL2RB and IL2RG proteins in vivo.

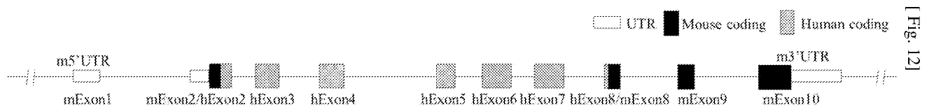

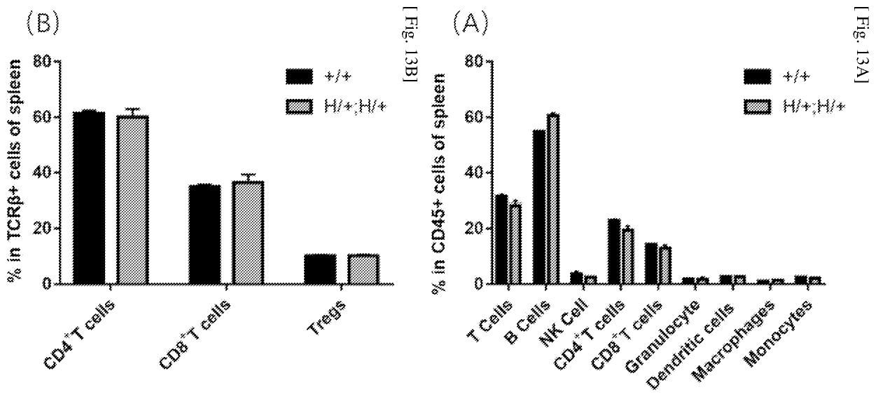

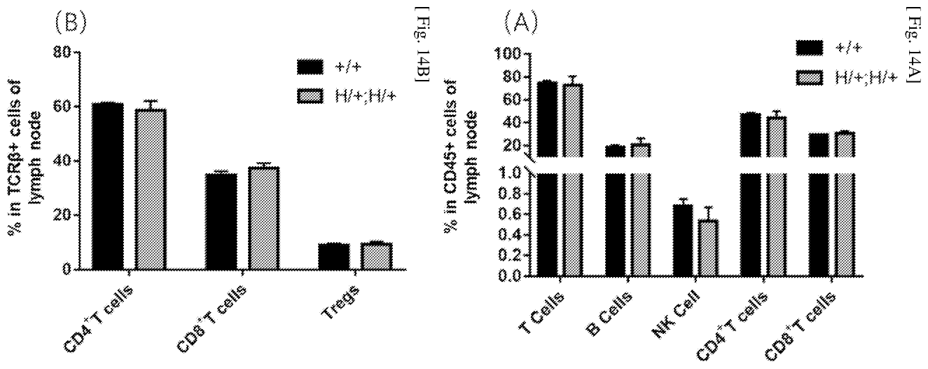

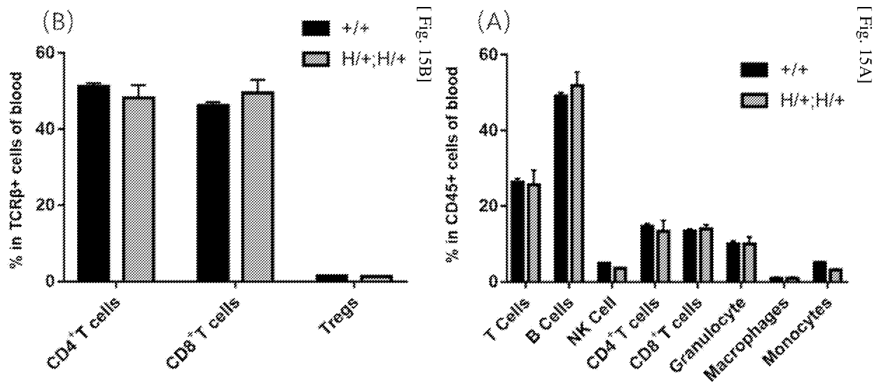

Further, the spleen, lymph nodes, and peripheral blood from C57BL/6 wild-type mice (+/+) and IL2RB/IL2RG double-gene humanized heterozygous mice (H/+; H/+) were collected for immuno-phenotyping detection by flow cytometry. The detection results of leukocyte subtypes (e.g., T cells, B cells, NK cells, granulocytes, macrophages, and monocytes) and T cell subtypes (e.g., CD4+ T cells, CD8+ T cells and Tregs cells) in the spleen, lymph nodes, and peripheral blood are shown in FIGS. 13A-13B, FIGS. 14A-14B, and FIGS. 15A-15B, respectively. The results showed that the percentages of leukocyte subtypes and T cell subtypes in the spleen, lymph nodes, and peripheral blood of IL2RB/IL2RG double-gene humanized mice were basically the same as those in C57BL/6 wild-type mice. The results indicate that the humanization of IL2RB and IL2RG genes did not affect the differentiation, development and distribution of leukocytes and T cells in the spleen, lymph nodes and peripheral blood of the IL2RB/IL2RG double-gene humanized mice.

The IL2 and IL15 signaling pathways in IL2RB/IL2RG double-gene humanized mice were detected by flow cytometry. Specifically, three 6-week old female C57BL/6 wild-type mice (+/+), three IL2RB/IL2RG double-gene humanized heterozygous mice (H/+; H/+), and three IL2RB/IL2RG double-gene humanized homozygous mice (H/H; H/H) were selected. Spleen tissues were collected and stimulated by 200 U human IL2 (Human IL-2 Protein, Tag Free), 10 ng/ml human IL15/IL15RA complex (Human IL-15 Protein, premium grade and Human IL-15 R alpha/CD215 Protein, Fc Tag mixed at a volume ratio of 1:1), or 10 ng/ml mouse IL15/IL15RA complex (Recombinant Mouse IL-15 Protein and Recombinant Mouse IL-15R alpha Fc Chimera Protein, CF mixed at a volume ratio of 1:1) for 30 minutes. eBioscience™ Fixable Viability Dye eFluor™ 780, Brilliant Violet 711™ anti-mouse TCR β chain Antibody, Phospho-Stat5 (Tyr694) (D47E7) XP® Rabbit mAb (used as primary antibodies) and Anti-rabbit IgG (H+L), F(ab′)₂ Fragment (Alexa Fluor® 488 Conjugate) (used as a secondary antibody) were used to fix and stain the cells, followed by flow cytometric detection. No primary antibody was added to the corresponding secondary antibody control groups.

The results are shown in the table below. The results show that after stimulation by human IL2, human IL15/IL15RA complex, or mouse IL15/IL15RA complex, phosphorylation of STAT5 can be detected in the spleen cells of wild-type C57BL/6 mice (+/+), IL2RB/IL2RG double-gene humanized heterozygous mice (H/+; H/+), and IL2RB/IL2RG double-gene humanized homozygous mice (H/H; H/H). The induced expression of STAT5 phosphorylation under the stimulation of human IL2, or the stimulation of human and mouse IL15/IL15RA complex indicates that the IL2 and IL15 signaling pathways in the IL2RB/IL2RG double-gene humanized mice function normally.

TABLE 14
Flow cytometry detection results of T cell pSTAT5 expression

| | | | | Human | | Mouse: |
|---|---|---|---|---|---|---|
| | | | | IL15/ | | IL15/ |
| | | | Human | IL15RA | Mouse | IL15RA |
| | | Human | IL15/ | com- | IL15/ | com- |
| | | IL2 | IL15RA | plex | IL15RA | plex |
| | Human | (2^{nd} Ab | com- | (2^{nd} Ab | com- | (2^{nd} Ab |
| Group | IL2 | control) | plex | control) | plex | control) |
| +/+ | 26.6% | 0.089% | 56.7% | 0.21% | 65.0% | 0.083% |
| H/+; H/+ | 35.7% | 0.12% | 55.5% | 0.21% | 52.3% | 0.14% |
| H/H; H/H | 33.7% | 0 | 47.6% | 0.30% | 21.3% | 0.27% |

### Example 5: Mice with Humanized IL15RA Gene

In this example, a non-human animal (e.g., a mouse) was modified to include a nucleotide sequence encoding human IL15RA protein, and the obtained genetically-modified non-human animal can express a human or humanized IL15RA protein in vivo. The mouse IL15RA gene (NCBI Gene ID: 16169, Primary source: MGI: 104644, UniProt ID: Q60819) is located at 11709992 to 11738796 of chromosome 2 (NC_000068.8), and the human IL15RA gene (NCBI Gene ID: 3601, Primary source: HGNC: 5978, UniProt ID: Q13261) is located at 5948897 to 5978741 of chromosome 10 (NC_000010.11). The mouse IL15RA transcript is NM_008358.2, and the corresponding protein sequence NP_032384.1 is set forth in SEQ ID NO: 42. The human IL15RA transcript is NM_002189.4, and the corresponding protein sequence NP_002180.1 is set forth in SEQ ID NO: 43. Mouse and human IL15RA gene loci are shown in FIG. 16.

All or part of nucleotide sequences encoding human IL15RA protein can be introduced into the mouse endogenous IL15RA locus, so that the mouse expresses human or humanized IL15RA protein. Specifically, a nucleotide sequence (e.g., a DNA or cDNA sequence) of the human IL15RA gene can be used to replace the corresponding mouse sequence at the endogenous IL15RA gene locus of the mouse by gene editing technology. For example, at the mouse endogenous IL15RA gene locus, a sequence starting from within exon 2 and ending within exon 6 of mouse IL15RA gene was replaced with a corresponding sequence starting from within exon 2 and ending within exon 6 of human IL15RA gene, to obtain a humanized IL15RA gene locus as shown in FIG. 17, thereby humanizing mouse IL15RA gene.

As shown in the schematic diagram of the targeting strategy in FIG. 18, the targeting vector contains homologous arm sequences upstream and downstream of the mouse IL15RA gene, and an “A Fragment” containing DNA sequences of human IL15RA gene. Specifically, sequence of the upstream homologous arm (5′ homologous arm, SEQ ID NO: 44) is identical to nucleotide sequence of 11717833-11723094 of NCBI accession number NC_000068.8, and sequence of the downstream homologous arm (3′ homologous arm, SEQ ID NO: 45) is identical to nucleotide sequence of 11735827-11739583 of NCBI accession number NC_000068.8. The genomic DNA sequence from human IL15RA gene (SEQ ID NO: 46) in the A Fragment is identical to nucleotide sequence of 5956438-5966319 of NCBI accession number NC_000010.11.

The targeting vector also includes an antibiotic resistance gene for positive clone screening (neomycin phosphotransferase gene, or Neo), and two Frt recombination sites flanking the antibiotic resistance gene, that formed a Neo cassette. The connection between the 5′ end of the Neo cassette and the mouse sequence was designed as:

(SEQ ID NO: 47)
5′-CATGTCAGCCTTGATTCTGTATTTCTAATAGCAGAACATATGGAAT
TCCGAAGTTCCTATTCTCTAGAAAGTATAGGAACTTCAGGT-3′,

wherein the last “A” in sequence “GCAGAA” is the last nucleotide of the mouse sequence, and the “C” in sequence “CATATG” is the first nucleotide of the Neo cassette. The connection between the 3′ end of the Neo cassette and the mouse sequence was designed as:

(SEQ ID NO: 48)
5′-TCTCTAGAAAGTATAGGAACTTCATCAGTCAGGTACATAATGGTGG
ATCCATGACACAGCGCAGCTCACTCACCAGTTCCCTCCACTTCCTGACA
TTTCAGT-3′,

wherein the last “C” in sequence “GGATCC” is the last nucleotide of the Neo cassette, and the first “A” in sequence “ATGACA” is the first nucleotide of the mouse sequence. In addition, a coding gene with a negative selectable marker (a gene encoding diphtheria toxin A subunit (DTA)) was also constructed downstream of the 3′ homologous arm of the targeting vector. The mRNA sequence of the engineered mouse IL15RA after humanization and its encoded protein sequence are shown in SEQ ID NO: 49 and SEQ ID NO: 50, respectively.

### Example 6: Mice with Humanized IL15 Gene

In this example, a non-human animal (e.g., a mouse) was modified to include a nucleotide sequence encoding human IL15 protein, and the obtained genetically-modified non-human animal can express a human or humanized IL15 protein in vivo. The mouse IL15 gene (NCBI Gene ID: 16168, Primary source: MGI: 103014, UniProt ID: P48346) is located at 83058253 to 83129883 of chromosome 8 (NC_000074.7), and the human IL15 gene (NCBI Gene ID: 3600, Primary source: HGNC: 5977, UniProt ID: P40933-1) is located at 141636583 to 141733987 of chromosome 4 (NC_000004.12). The mouse IL15 transcript is NM_001254747.1, and the corresponding protein sequence NP_001241676.1 is set forth in SEQ ID NO: 51. The human IL15 transcript is NM_000585.5, and the corresponding protein sequence NP_000576.1 is set forth in SEQ ID NO: 52. Mouse and human IL15 gene loci are shown in FIG. 19.

All or part of nucleotide sequences encoding human IL15 protein can be introduced into the mouse endogenous IL15 locus, so that the mouse expresses human or humanized IL15 protein. Specifically, using gene-editing technologies, a human IL15 gene sequence (e.g., a DNA or cDNA sequence) can be used to replace a corresponding mouse sequence at the mouse endogenous IL15 gene locus, such as replacing the sequence from the start codon to the stop codon of the mouse IL15 gene with the corresponding human DNA sequence, to obtain a humanized IL15 gene locus as shown in FIG. 20, thereby humanizing mouse IL15 gene.

As shown in the schematic diagram of the targeting strategy in FIG. 21, the targeting vector contains homologous arm sequences upstream and downstream of the mouse IL15 gene, and an “A Fragment” containing DNA sequences of human IL15 gene. Specifically, sequence of the upstream homologous arm (5′ homologous arm, SEQ ID NO: 53) is identical to nucleotide sequence of 83072241-83076085 of NCBI accession number NC_000074.7, and sequence of the downstream homologous arm (3′ homologous arm, SEQ ID NO: 54) is identical to nucleotide sequence of 83053728-83057763 of NCBI accession number NC_000074.7. The genomic DNA sequence from human IL15 gene (SEQ ID NO: 55) in the A Fragment is identical to nucleotide sequence of 141719465-141732848 of NCBI accession number NC_000004.12.

The targeting vector also includes an antibiotic resistance gene for positive clone screening (neomycin phosphotransferase gene, or Neo), and two Frt recombination sites flanking the antibiotic resistance gene, that formed a Neo cassette. The connection between the 5′ end of the Neo cassette and the mouse sequence was designed as:

(SEQ ID NO: 56)
5′-GTGATAGTCCTTCACGGAAAGTACAAGAATACACAGAAGTCGACGG
TATCGATAAGCTTGATATCGAATTCCGAAGTTCCTATTCTC-3′,

wherein the last “A” in sequence “CAGAA” is the last nucleotide of the mouse sequence, and the first “G” in sequence “GTCGA” is the first nucleotide of the Neo cassette. The connection between the 3′ end of the Neo cassette and the mouse sequence was designed as:

5′-CTCTAGAAAGTATAGGAACTTCATCAGTCAGGTACATAATGGTGGA
TCCAACTGCTGTTTACATTAGTCTTTCAC

the last “C” in sequence “GATCC” is the last nucleotide of the Neo cassette, and the first “A” in sequence “AACTG” is the first nucleotide of the mouse sequence. In addition, a coding gene with a negative selectable marker (a gene encoding diphtheria toxin A subunit (DTA)) was also constructed downstream of the 3′ homologous arm of the targeting vector. The mRNA sequence of the engineered mouse IL15 after humanization and its encoded protein sequence are shown in SEQ ID NO: 58 and SEQ ID NO: 52, respectively.

The targeting vector was constructed, e.g., by restriction enzyme digestion and ligation. The constructed targeting vector sequences were preliminarily confirmed by restriction enzyme digestion, and then verified by sequencing. Embryonic stem cells of C57BL/6 or BALB/c mice were transfected with the correct targeting vector by electroporation. The positive selectable marker genes were used to screen the cells, and the integration of exogenous genes was confirmed by PCR and Southern Blot. The clones identified as positive by PCR were then verified by Southern Blot. The restriction enzymes, probes, and the size of target fragment are shown in the table below.

TABLE 15
Enzymes and probes used in Southern Blot

| Restriction | | Wild-type | Recombinant | |
|---|---|---|---|---|
| enzyme | Probe | fragment size | fragment size | |
| MfeI | 5′ Probe | — | 14.6 | kb |
| XbaI | 3′ Probe | 11.3 kb | 6.8 | kb |
| ScaI | Neo Probe-5 (3′) | — | 8.7 | kb |

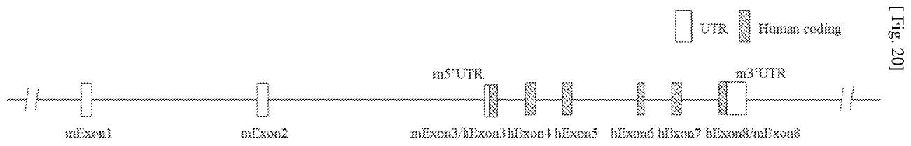

The Southern Blot detection results are shown in FIG. 22. The results showed that the mouse numbered ES-01 was verified as a positive clone.

The following primers were used for probe synthesis in Southern Blot assays:

5′Probe:
5′Probe-F:
(SEQ ID NO: 59)
5′-GGGCTTGGTATCAAGAATGAGGGGT-3′,
5′Probe-R:
(SEQ ID NO: 60)
5′-ACCCCACAGAACCTCTACTGGGA-3′;
3′Probe:
3′Probe-F:
(SEQ ID NO: 61)
5′-CCCCAAGTCATGTTTGCACCC-3′,
3′Probe-R:
(SEQ ID NO: 62)
5′-GTCCTCCCTGAATCCTGCACCTG-3′;
Neo Probe-5 (3′):
Neo Probe-F:
(SEQ ID NO: 63)
5′-GGATCGGCCATTGAACAAGAT-3′,
Neo Probe-R:
(SEQ ID NO: 64)
5′-CAGAAGAACTCGTCAAGAAGGC-3′.

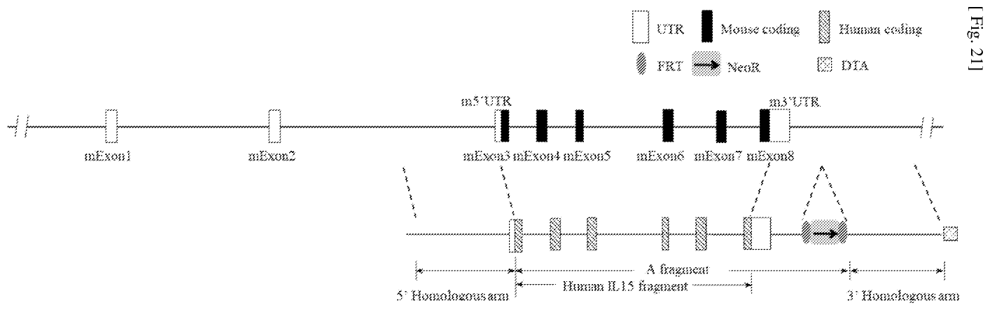

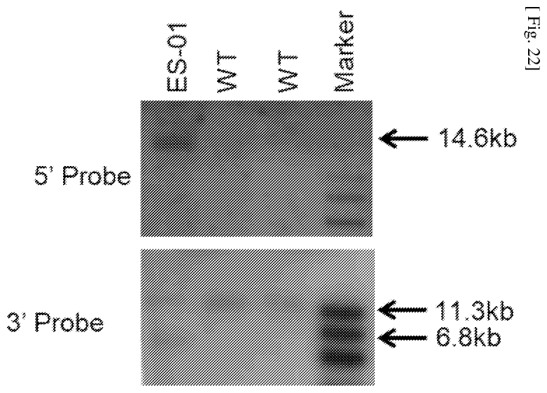

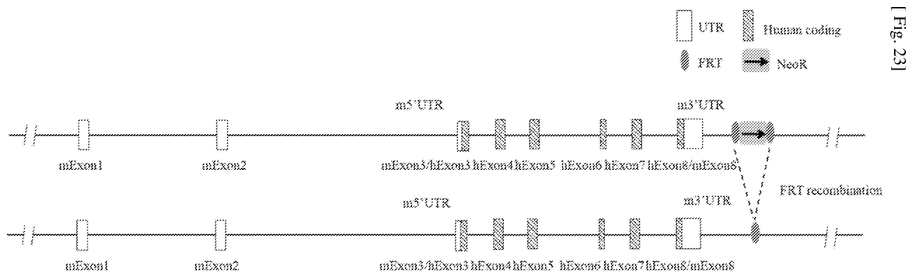

The positive clones that had been screened (black mice) were introduced into isolated blastocysts (white mice), and the resulted chimeric blastocysts were transferred to a culture medium for short-term culture and then transplanted to the fallopian tubes of the recipient mother (white mice) to produce the F0 chimeric mice (black and white). The F2 generation homozygous mice were obtained by backcrossing the F0 generation chimeric mice with wild-type mice to obtain the F1 generation mice, and then breeding the F1 generation heterozygous mice with each other. The positive mice were also bred with the Flp transgenic mice to remove the positive selectable marker genes (schematic diagram shown in FIG. 23), and then the humanized homozygous mice with a humanized IL15 gene were obtained by breeding the heterozygous mice with each other. The identification results of F1 generation mice are shown in FIGS. 24A-24D. The results showed that the mouse numbered F1-01 was verified as a positive heterozygous mouse. The PCR primers used are shown in the table below. The results indicate that the IL15 gene humanized mice constructed using the methods described herein can be stably passaged without random insertions.

TABLE 16
PCR primer sequences and recombinant fragment sizes (F1 generation
genotype)

| | | | Recombinant |
|---|---|---|---|
| | | | fragment size |
| Primer | SEQ ID NO | Primer sequence (5′-3′) | (bp) |
| WT-F | SEQ ID NO: 65 | AGTCCGGGCAGTCATGGGAT | WT: 427 |
| WT-R | SEQ ID NO: 66 | TGTCTGTCGCAGCCAGAGATGTT | |
| WT-F | SEQ ID NO: 65 | AGTCCGGGCAGTCATGGGAT | Mut: 600 |
| Mut-R | SEQ ID NO: 67 | CCTGCCCTCTAAGACTACACAATTT | |
| Frt-F1 | SEQ ID NO: 68 | GATTACAGGCACTTGCCACATCC | WT: 391 |
| Frt-R1 | SEQ ID NO: 69 | GGATCCCCATGGTTGTGCAGGAA | Mut: 486 |
| | | | (Neo cassette |
| | | | removed) |
| Frt-F2 | SEQ ID NO: 70 | GACAAGCGTTAGTAGGCACATATAC | Mut: 325 |
| Frt-R2 | SEQ ID NO: 71 | GCTCCAATTTCCCACAACATTAGT | |

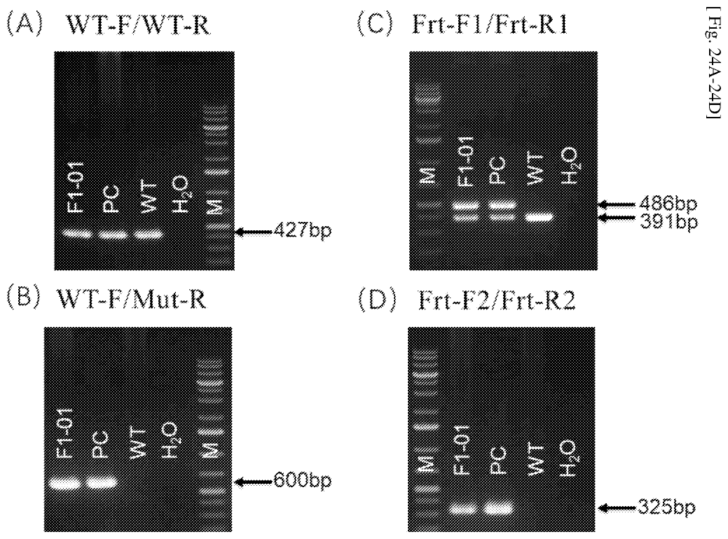

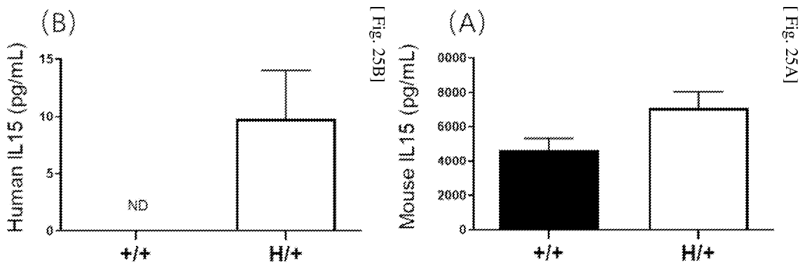

The expression of human or humanized IL15 protein in positive mice can also be confirmed, e.g., by ELISA. Specifically, two 6-week-old female C57BL/6 wild-type mice and two 14-week-old male IL15 gene humanized heterozygous mice were selected. 20 μg/200 μL of lipopolysaccharide (LPS) was injected intraperitoneally, and lung grinding fluid was collected for detection 2 hours later. The protein expression was detected using the Mouse IL-15 ELISA Kit and the Human IL-15 Quantikine ELISA Kit. As show in FIGS. 25A-25B, expression of mouse IL15 protein, but not humanized IL15 protein, was detected in the C57BL/6 mice. By contrast, expression of both mouse IL15 protein and humanized IL15 protein was detected in the IL15 gene humanized heterozygous mice. The results indicate that human or humanized IL15 protein can be normally expressed in mice after humanization.

### Example 7: Generation of IL15/IL15RA Double-Gene Humanized Mice

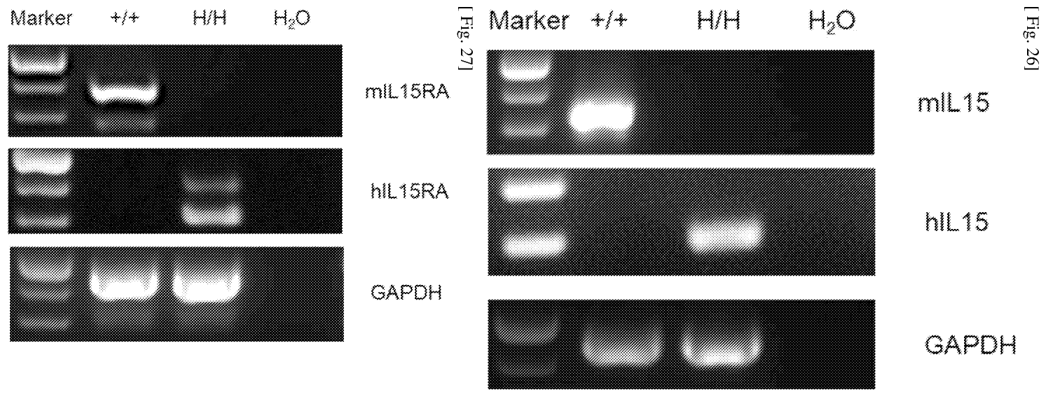

The IL15RA gene humanized mice prepared in Example 5 were bred with the IL15 gene humanized mice prepared in Example 6, and the IL15/IL15RA double-gene humanized mice were obtained by screening the positive progeny mice. RT-PCR was used for genotype identification of IL15/IL15RA double-gene humanized homozygous mice. Specifically, one 8-week old female C57BL/6 wild-type mouse (+/+) and one 8-week old female IL15/IL15RA double-gene humanized homozygous mouse (H/H) prepared herein were selected. Spleen tissues were collected after euthanasia by cervical dislocation. RT-PCR detection was performed using primers shown in the table below, and the identification results are shown in FIG. 26 and FIG. 27.

TABLE 17
RT-PCR detection primer sequences and recombinant fragment sizes

| | | | Recombinant |
|---|---|---|---|
| | | | fragment size |
| Primer | SEQ ID NO | Primer sequence (5′-3′) | (bp) |
| mIL15RA-RT-F1 | SEQ ID NO: 72 | GCTGACATCCGGGTCAAGAA | Mut: 370 |
| mIL15RA-RT-R1 | SEQ ID NO: 73 | TGCCTGTCCCTGTAGTTCCT | |
| hIL15RA-RT-F1 | SEQ ID NO: 74 | TACTCCAGGGAGGGGTACATT | Mut: 402 |
| hIL15RA-RT-R1 | SEQ ID NO: 75 | GAGTTCCCAGTTCTTGGCTGT | |
| GAPDH-F | SEQ ID NO: 76 | TCACCATCTTCCAGGAGCGAGA | WT: 479 |
| GAPDH-R | SEQ ID NO: 77 | GAAGGCCATGCCAGTGAGCTT | |
| mIL15-RT-F1 | SEQ ID NO: 78 | GGGCTGTGTCAGTGTAGGTC | Mut: 332 |
| mIL15-RT-R1 | SEQ ID NO: 79 | ACTCTGTGAAGGTTTTCTCCTCC | |
| hIL 15-RT-F1 | SEQ ID NO: 80 | CCATCCAGTGCTACTTGTGTT | Mut: 209 |
| hIL15-RT-R1 | SEQ ID NO: 81 | GGGGTGAACATCACTTTCCG | |

The results showed that only mouse IL15 and IL15RA mRNA could be detected in splenocytes of the C57BL/6 wild-type mouse, whereas only human IL15 and IL15RA mRNA could be detected in the IL15/IL15RA double-gene humanized homozygous mouse. In particular, additional bands appeared when detecting human and mouse IL15RA mRNA. It is contemplated that the additional bands were produced because of the existence of multiple transcriptional variants of human and mouse IL15RA.

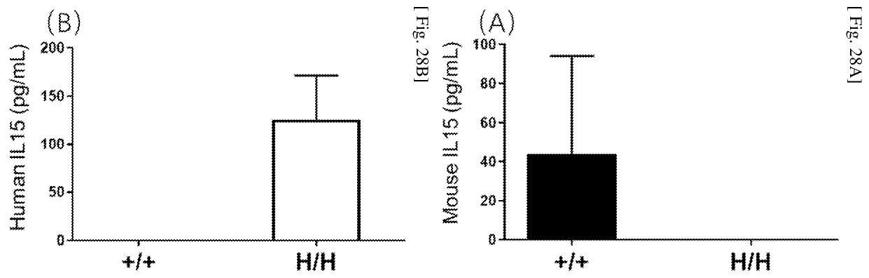

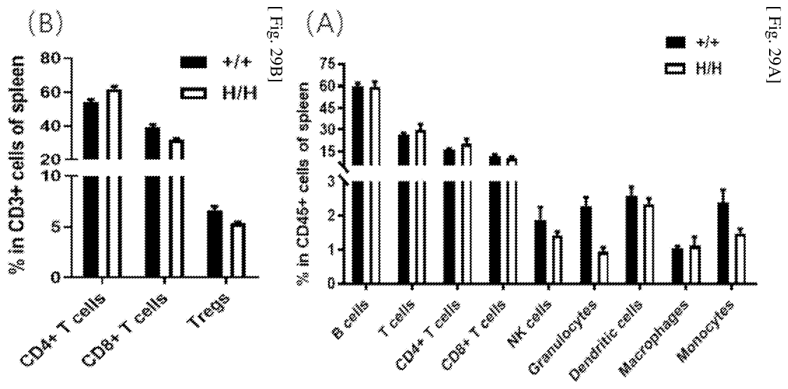

The expression of human or humanized IL15 protein in positive mice can also be confirmed, e.g., by ELISA. Specifically, three 8-week-old female C57BL/6 wild-type mice (+/+) and three 8-week-old female IL15/IL15RA double-gene humanized homozygous mice (H/H) prepared herein were selected. After stimulation of acetaminophen (350 mg/kg) injected by intraperitoneal administration for 24 hours, blood serums were collected after euthanasia by cervical dislocation. The protein expression was detected using the IL-15/IL-15R Complex Mouse ELISA Kit and the Human IL-15 Quantikine ELISA Kit. As shown in FIGS. 28A-28B, mouse IL15 protein was only detected in the wild-type C57BL/6 mice, whereas human IL15 protein was only detected in the IL15/IL15RA double-gene humanized homozygous mice. The results indicate that human IL15 protein can be normally expressed in mice after humanization.

Flow cytometry can be used to detect the expression of human or humanized IL15RA protein in IL15/IL15RA double-gene humanized homozygous mice. Specifically, one 7-week-old female C57BL/6 wild-type mice (+/+), and one 7-week-old female IL15/IL15RA double-gene humanized homozygous mouse (H/H) prepared herein were selected. Dendritic cells (DC cells) from bone marrow were collected after euthanasia by cervical dislocation. The cells were stained with: Brilliant Violet 510™ anti-mouse CD45, PE Rat Anti-Mouse CD215 (IL-15Rα), PE anti-human CD215 (IL-15Rα) Antibody, Brilliant Violet 711™ anti-mouse CD11c Antibody, APC Rat IgG2a, κ Isotype Ctrl Antibody, PE Mouse IgG2b, κ Isotype Ctrl Antibody (Fc), Zombie NIR™ Fixable Viability Kit, and/or Purified anti-mouse CD16/32, and then subjected to flow cytometry analysis. The results showed that there were 2.53% hIL15RA positive cells and 19.0% mIL15RA positive cells in DC cells from the C57BL/6 mouse. By contrast, there were 1.41% mIL15RA-positive cells and 29.7% hIL15RA-positive cells in DC cells from the IL15/IL15RA double-gene humanized homozygous mouse. In summary, the expression of mouse IL15RA, but not human or humanized IL15RA, was detected in the wild-type mouse C57BL/6 mouse, whereas the expression of humanized IL15RA, but not mouse IL15RA, was detected in the IL15/IL15RA double-gene humanized homozygous mouse.

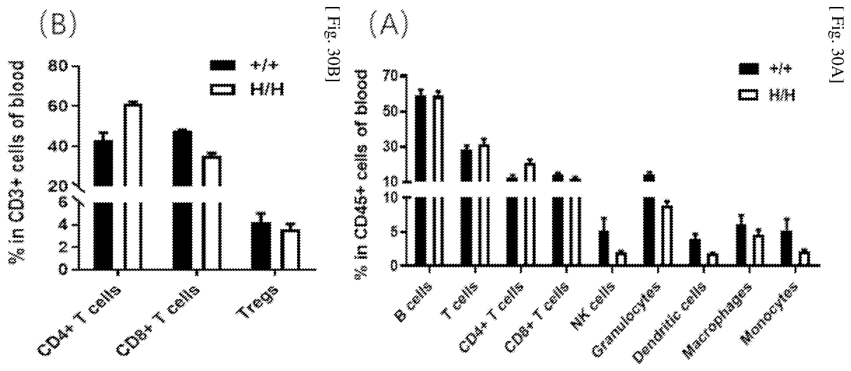

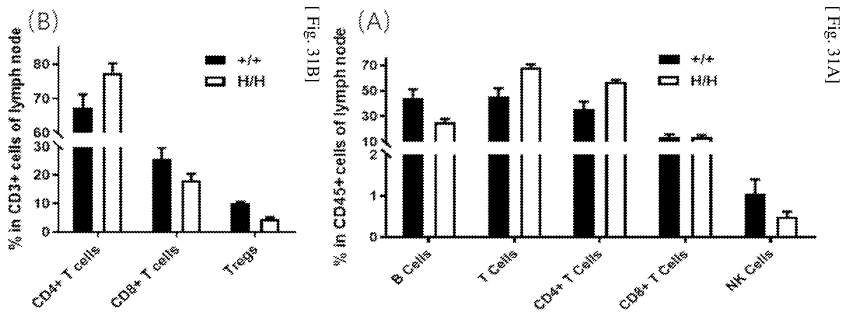

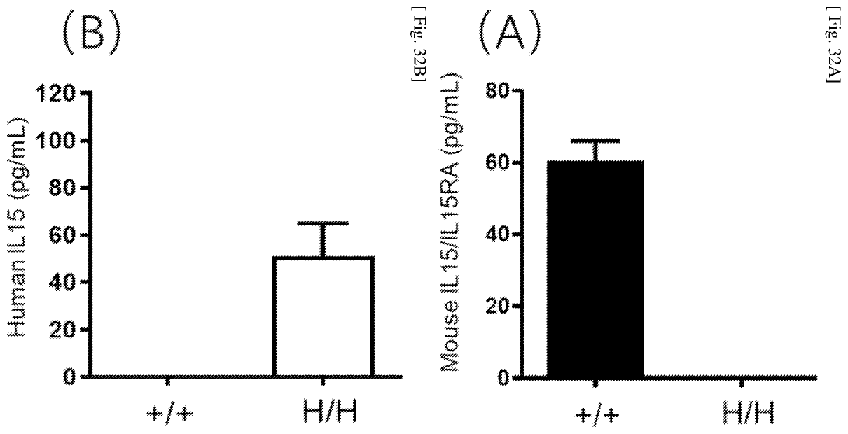

Further, the spleen, lymph nodes, and peripheral blood from C57BL/6 wild-type mice (+/+) and IL15/IL15RA double-gene humanized homozygous mice (H/H) were collected for immuno-phenotyping detection by flow cytometry. Specifically, three 8-week-old female wild-type C57BL/6 mice and three IL15/IL15RA double-gene humanized homozygous mice were selected. The spleen, lymph nodes, and peripheral blood were collected after euthanasia by cervical dislocation. Cells were stained with: Purified anti-mouse CD16/32 Antibody, Zombie NIR™ Fixable Viability Kit, PerCP anti-mouse CD45 Antibody, Brilliant Violet 510™ anti-mouse CD4, PE anti-mouse CD8a Antibody, FOXP3 Monoclonal Antibody (FJK-16s), PerCP-eFluor™ 710, eBioscience™, FITC anti-Mouse CD19, Brilliant Violet 421™ anti-mouse NK-1.1 Antibody, Brilliant Violet 785™ anti-mouse/human CD11b Antibody, Brilliant Violet 711™ anti-mouse CD11c Antibody, APC anti-mouse F4/80 Antibody, Brilliant Violet 650™ anti-mouse Ly-6G Antibody, PE/Cyanine7 anti-mouse CD25 Antibody, and/or Alexa Fluor® 700 anti-mouse CD3 Antibody, for immunophenotyping. The detection results of leukocyte subtypes and T cell subtypes in the spleen and peripheral blood are shown in FIGS. 29A-29B and FIGS. 30A-30B, respectively. The results showed that the percentages of B cells, T cells, CD4+ T cells, CD8+ T cells, NK cells, granulocytes, dendritic cells, macrophages, monocytes, and other leukocyte subtypes in the spleen and peripheral blood of IL15/IL15RA double-gene humanized homozygous mice were basically the same as those in C57BL/6 wild-type mice (FIG. 29A and FIG. 30A). In addition, the percentages of CD4+ T cells, CD8+ T cells, and Treg cells (Tregs) were basically the same as those in C57BL/6 wild-type mice (FIG. 29B and FIG. 30B).

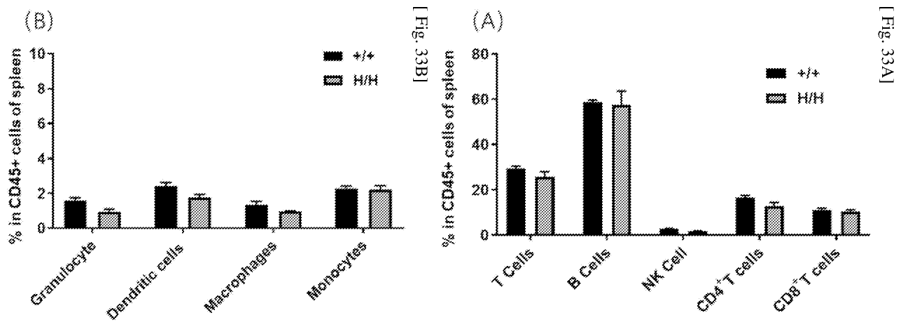

The detection results of leukocyte subtypes and T cell subtypes in lymph nodes are shown in FIG. 31A and FIG. 31B, respectively. The results showed that the leukocyte subtypes, e.g., B cells, T cells, CD4+ T cells, CD8+ T cells, NK cells, and other leukocyte subtypes in the lymph nodes of IL15/IL15RA double-gene humanized homozygous mice were basically the same as those of C57BL/6 wild-type mice (FIG. 31A). In addition, the percentages of T cell subtypes, e.g., CD4+ T cells, CD8+ T cells and Tregs cells were basically the same as those of C57BL/6 wild-type mice (FIG. 31B).

The results indicate that the humanization of both IL15 gene and IL15RA gene did not affect the differentiation, development and distribution of leukocytes and T cells in the spleen, lymph nodes and peripheral blood of mice.

### Example 8: Generation of Multi-Gene Humanized Mice

To generate IL2RB/IL2RG/IL15/IL15RA four-gene humanized mice, the IL2RB/IL2RG double-gene humanized mice prepared in Example 4 were bred with the IL15/IL15RA double-gene humanized mice prepared in Example 7. After multiple generations of screening, IL2RB/IL2RG/IL15/IL15RA four-gene humanized mice were obtained.

Flow cytometry can be used to detect the expression of human or humanized IL2RB protein in IL2RB/IL2RG/IL15/IL15RA four-gene humanized homozygous mice. Specifically, one 10-week old female wild-type C57BL/6 mouse and one IL2RB/IL2RG/IL15/IL15RA four-gene humanized homozygous mouse were selected. 7.5 μg/200 μL of Anti-mCD3e was injected intraperitoneally, and spleen cells were collected for flow cytometric detection 24 hours later. Cells were first stained with: Brilliant Violet 510™ anti-mouse CD45 (mCD45; an anti-mouse CD45 antibody), FITC anti-mouse TCR β chain Antibody (an anti-mouse TCRβ antibody), Brilliant Violet 421™ anti-mouse CD4 (a mouse T cell marker antibody), Brilliant Violet 711™ anti-mouse CD8a, PE/Cy™ 7 Mouse anti-mouse NK1.1, APC anti-mouse CD122 (IL-2Rβ) Antibody (an anti-mouse IL-2Rβ antibody), PE anti-human CD122 (IL-2Rβ) Antibody (an anti-human IL-2Rβ antibody), APC Rat IgG2a, κ Isotype Ctrl Antibody, PE Mouse IgG1, κ Isotype Ctrl Antibody, PE anti-human CD132 (common γ chain), APC anti-mouse CD132 (common γchain), PE Rat IgG2b, K Isotype Ctrl Antibody, APC Mouse IgG2b, κ Isotype Ctrl Antibody, Zombie NIR™ Fixable Viability Kit, and/or Purified anti-mouse CD16/32, and then subjected to flow cytometry analysis.

The results showed that T cells in the spleen of the C57BL/6 mouse had 0.78% hIL2RB positive cells (characterized by mCD45+mTCRβ+hIL2RB+) and 11.0% mIL2RB positive cells (characterized by mCD45+mTCRβ+mIL2RB+). There were 8.80% hIL2RB positive cells and 0.80% mIL2RB positive cells in spleen T cells of the IL2RB/IL2RG/IL15/IL15RA four-gene humanized homozygous mouse.

The results showed that CD4+ T cells in the spleen of the C57BL/6 mouse had 0.11% hIL2RB positive cells (characterized by mCD45+mTCRβ+mCD4+hIL2RB+) and 8.19% mIL2RB positive cells (characterized by mCD45+mTCRβ+mCD4+mIL2RB+). There were 4.83% hIL2RB positive cells and 0.39% mIL2RB positive cells in spleen CD4+ T cells of the IL2RB/IL2RG/IL15/IL15RA four-gene humanized homozygous mouse.

The results showed that CD8+ T cells in the spleen of the C57BL/6 mouse had 0.22% hIL2RB positive cells (characterized by mCD45+mTCRβ+mCD8+hIL2RB+) and 2.56% mIL2RB positive cells (characterized by mCD45+mTCRβ+mCD8+mIL2RB+). There were 3.24% hIL2RB positive cells and 0.26% mIL2RB positive cells in spleen CD8+ T cells of the IL2RB/IL2RG/IL15/IL15RA four-gene humanized homozygous mouse.

The results showed that NK cells in the spleen of the C57BL/6 mouse had 0.74% hIL2RB positive cells (characterized by mCD45+mTCRβ+mNK1.1+hIL2RB+) and 1.71% mIL2RB positive cells (characterized by mCD45+mTCRβ+mNK1.1+mIL2RB+). There were 1.84% hIL2RB positive cells and 0.72% mIL2RB positive cells in spleen NK cells of the IL2RB/IL2RG/IL15/IL15RA four-gene humanized homozygous mouse.

A similar method can be used to detect the expression of human or humanized IL2RG protein in IL2RB/IL2RG/IL15/IL15RA four-gene humanized homozygous mice. Specifically, one 7-week old female wild-type C57BL/6 mouse and one IL2RB/IL2RG/IL15/IL15RA four-gene humanized homozygous mouse were selected. 7.5 μg/200 μL of Anti-mCD3e was injected intraperitoneally, and spleen cells were collected for flow cytometric detection 24 hours later. The method described above was used for flow cytometry analysis.

The results showed that T cells in the spleen of the C57BL/6 mouse had 1.71% hIL2RG positive cells (characterized by mCD45+mTCRβ+hIL2RG+) and 9.36% mIL2RG positive cells (characterized by mCD45+mTCRβ+mIL2RG+). There were 1.30% hIL2RG positive cells and 0.94% mIL2RG positive cells in spleen T cells of the IL2RB/IL2RG/IL15/IL15RA four-gene humanized homozygous mouse.

The results showed that CD4+ T cells in the spleen of the C57BL/6 mouse had 0.68% hIL2RG positive cells (characterized by mCD45+mTCRβ+mCD4+hIL2RG+) and 5.43% mIL2RG positive cells (characterized by mCD45+mTCRβ+mCD4+mIL2RG+). There were 1.50% hIL2RG positive cells and 0.46% mIL2RG positive cells in spleen CD4+ T cells of the IL2RB/IL2RG/IL15/IL15RA four-gene humanized homozygous mouse.

The results showed that CD8+ T cells in the spleen of the C57BL/6 mouse had 0.94% hIL2RG positive cells (characterized by mCD45+mTCRβ+mCD8+hIL2RG+) and 3.15% mIL2RG positive cells (characterized by mCD45+mTCRβ+mCD8+mIL2RG+). There were 2.49% hIL2RG positive cells and 0.45% mIL2RG positive cells in spleen CD8+ T cells of the IL2RB/IL2RG/IL15/IL15RA four-gene humanized homozygous mouse.

The results showed that NK cells in the spleen of the C57BL/6 mouse had 0.47% hIL2RG positive cells (characterized by mCD45+mTCRβ+mNK1.1+hIL2RG+) and 2.28% mIL2RG positive cells (characterized by mCD45+mTCRβ+mNK1.1+mIL2RG+). There were 1.40% hIL2RG positive cells and 0.94% mIL2RG positive cells in spleen NK cells of the IL2RB/IL2RG/IL15/IL15RA four-gene humanized homozygous mouse.

The above results showed that only mouse IL2RB and IL2RG protein expressions can be detected in the C57BL/6 mouse, and humanized IL2RB and IL2RG protein expressions can be detected in the IL2RB/IL2RG/IL15/IL15RA four-gene humanized homozygous mouse.

Flow cytometry can be used to detect the expression of human or humanized IL15RA protein IL2RB/IL2RG/IL15/IL15RA four-gene humanized homozygous mice. Specifically, one 8-week old female C57BL/6 wild-type mouse and one IL2RB/IL2RG/IL15/IL15RA four-gene humanized homozygous mouse were selected. Bone marrow dendritic cells (BMDC cells) were collected after euthanasia by cervical dislocation. The cells were stained with: Brilliant Violet 510™ anti-mouse CD45, FITC anti-mouse TCR β chain Antibody, Brilliant Violet 605™ anti-mouse CD11c, PE Rat Anti-Mouse CD215 (IL-15Rα), PE anti-human CD215 (IL-15Rα) Antibody, PE Rat IgG1, κ Isotype Control, PE Mouse IgG2b, κ Isotype Ctrl Antibody (Fc, ICFC), Zombie NIR™ Fixable Viability Kit, and/or Purified anti-mouse CD16/32, and then subjected to flow cytometry analysis.

The results showed that BMDC cells of the C57BL/6 mouse had 0.62% hIL15RA positive cells and 52.3% mIL15RA positive cells. There were 25.8% hIL15RA positive cells and 0.26% mIL15RA positive cells in BMDC cells of the IL2RB/IL2RG/IL15/IL15RA four-gene humanized homozygous mouse. The results indicate that C57BL/6 mice can express mouse IL15RA protein, and IL2RB/IL2RG/IL15/IL15RA four-gene humanized homozygous mice can express human or humanized IL5RA protein in vivo.

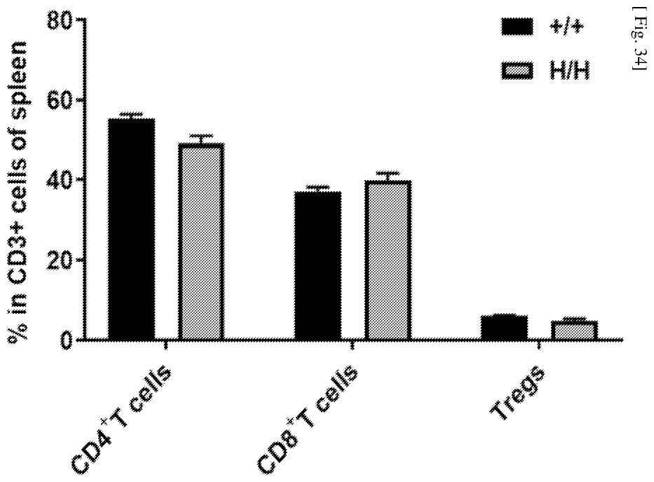

Expression of hIL15 protein in IL2RB/IL2RG/IL15/IL15RA four-gene humanized homozygous mice was also detected by ELISA. Specifically, three 8-week-old female C57BL/6 wild-type mice and three IL2RB/IL2RG/IL15/IL15RA four-gene humanized homozygous mice were selected. After stimulation of acetaminophen (350 mg/kg) injected by intraperitoneal administration for 24 hours, blood serums were collected after euthanasia by cervical dislocation. The protein expression was detected using the IL-15/IL-15R Complex Mouse ELISA Kit and the Human IL-15 Quantikine ELISA Kit. As shown in FIGS. 32A-32B, only mIL15/mIL15RA protein expression was detected in C57BL/6 wild-type mice, and only hIL15 protein expression was detected in IL2RB/IL2RG/IL15/IL15RA four-gene humanized homozygous mice. The results indicate that human IL15 protein can be normally expressed in mice after humanization.

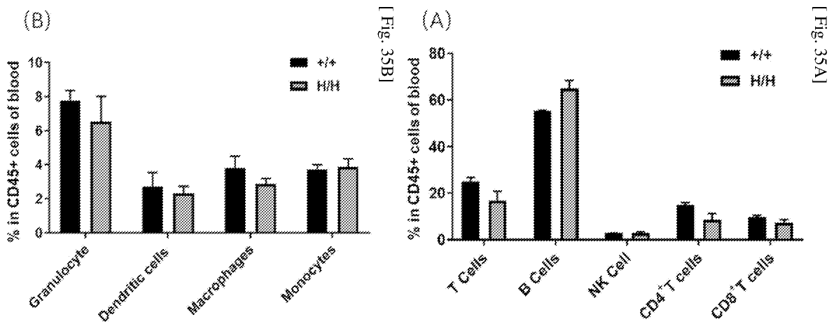

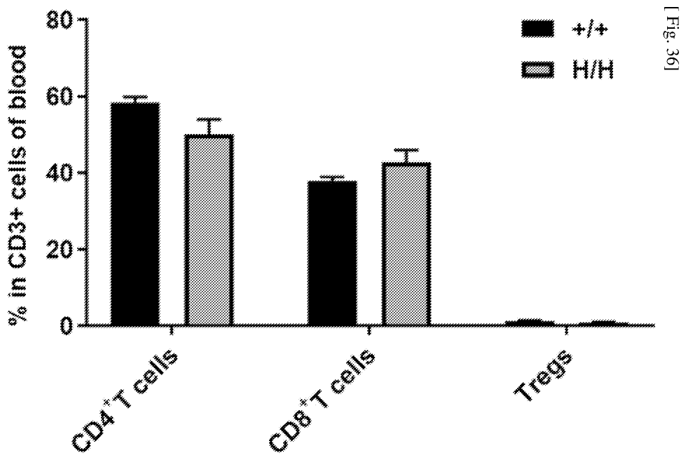

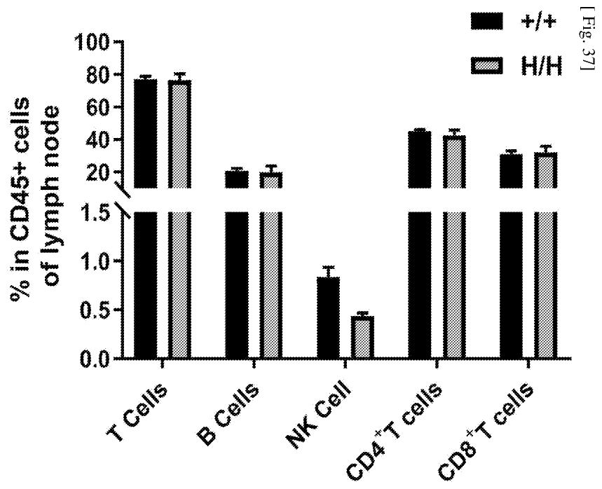

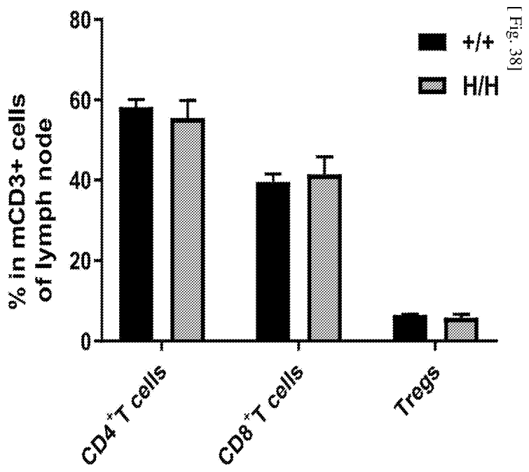

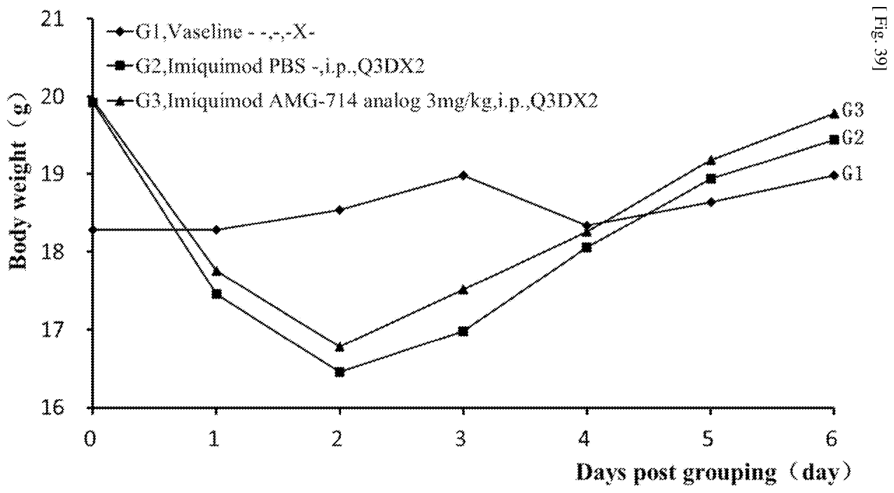

Further, the spleen, lymph nodes, and peripheral blood from C57BL/6 wild-type mice and IL2RB/IL2RG/IL15/IL15RA four-gene humanized homozygous mice were collected for immuno-phenotyping detection by flow cytometry. Specifically, three 7-week-old female wild-type C57BL/6 mice and three IL2RB/IL2RG/IL15/IL15RA four-gene humanized homozygous mice were selected. The spleen, lymph nodes, and peripheral blood were collected after euthanasia by cervical dislocation. Cells were stained with: Purified anti-mouse CD16/32 Antibody, Zombie NIR™ Fixable Viability Kit, PerCP anti-mouse CD45 Antibody, Alexa Fluor® 700 anti-mouse CD3 Antibody, PE anti-mouse CD8a Antibody, Brilliant Violet 510™ anti-mouse CD4, PE/Cyanine7 anti-mouse CD25 Antibody, FITC anti-Mouse CD19, FOXP3 Monoclonal Antibody (FJK-16s), PerCP-eFluor™ 710, eBioscience™. Brilliant Violet 421™ anti-mouse NK-1.1 Antibody, Brilliant Violet 785™ anti-mouse/human CD11b Antibody, Brilliant Violet 71111 anti-mouse CD11c Antibody, APC anti-mouse F4/80 Antibody, and/or Brilliant Violet 650™ anti-mouse Ly-6G Antibody, for immunophenotyping. The detection results of leukocyte subtypes and T cell subtypes in the spleen and peripheral blood are shown in FIGS. 33A-33B, 34 and FIGS. 35A-35B, 36, respectively. The results showed that the percentages of B cells, T cells, NK cells, CD4+ T cells, CD8+ T cells, granulocytes, dendritic cells, macrophages, monocytes, and other leukocyte subtypes in the spleen and peripheral blood of IL2RB/IL2RG/IL15/IL15RA four-gene humanized homozygous mice were basically the same as those in C57BL/6 wild-type mice (FIGS. 33A-33B and FIGS. 35A-35B). In addition, the percentages of CD4+ T cells, CD8+ T cells, and Treg cells (Tregs) were basically the same as those in C57BL/6 wild-type mice (FIG. 34 and FIG. 36).

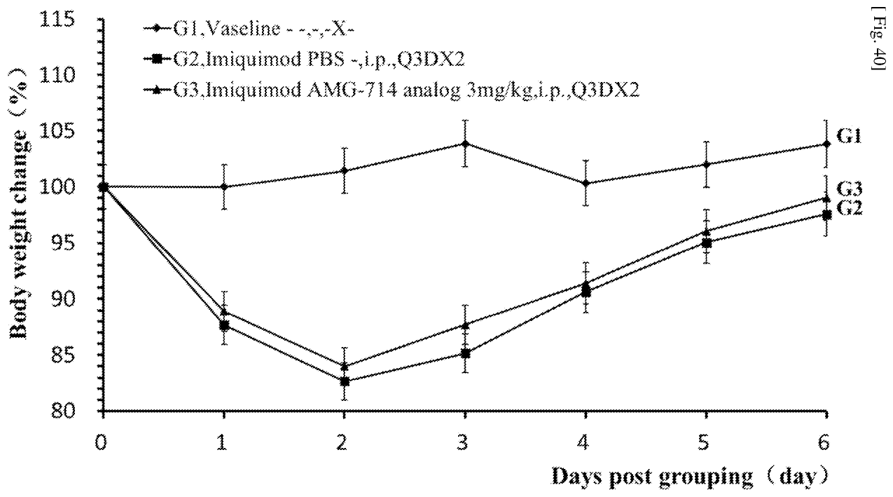

The detection results of leukocyte subtypes and T cell subtypes in lymph nodes are shown in FIG. 37 and FIG. 38, respectively. The results showed that the leukocyte subtypes, e.g., B cells, T cells, NK cells, CD4+ T cells, CD8+ T cells, and other leukocyte subtypes in the lymph nodes of IL2RB/IL2RG/IL15/IL15RA four-gene humanized homozygous mice were basically the same as those of C57BL/6 wild-type mice (FIG. 37). In addition, the percentages of T cell subtypes, e.g., CD4+ T cells, CD8+ T cells and Tregs cells were basically the same as those of C57BL/6 wild-type mice (FIG. 38).

The results indicate that the humanization of IL2RB/IL2RG/IL15/IL15RA genes did not affect the differentiation, development and distribution of leukocytes and T cells in the spleen, lymph nodes and peripheral blood of mice.

The IL2 and IL15 signaling pathways in IL2RB/IL2RG/IL15/IL15RA four-gene humanized mice were detected by flow cytometry. Similar to the IL2 and IL15 signaling pathway detection method for IL2RB/IL2RG double-gene humanized mice described herein, either 10 μg/mL human IL2 or mouse IL2 (Biotinylated Mouse IL-2 Protein) was used to stimulate mouse splenocytes. The cells were stained with: Brilliant Violet 421™ anti-mouse NK-1.1 Antibody, APC anti-mouse CD4 Antibody, and/or Alexa Fluor 700 anti-mouse CD8a Antibody, and then subjected to flow cytometry analysis. The results are shown in the table below. The results showed that after stimulation with human or mouse IL2 protein, phosphorylated expression of STAT5 was detected in the splenocytes of wild-type C57BL/6 mice (+/+) and IL2RB/IL2RG/IL15/IL15RA four-gene humanized homozygous mice (H/H).

TABLE 18
Flow cytometry detection results of pSTAT5
expression in T cells and NK cells

| | hIL2 | mIL2 | hIL2 | mIL2 | hIL2 | mIL2 |
|---|---|---|---|---|---|---|
| | (CD4+ | (CD4+ | (CD8a+ | (CD8a+ | (NK1.1+ | (NK1.1+ |
| Group | T cell) | T cell) | cell) | cell) | cell) | cell) |
| +/+ | 55.2% | 16.6% | 94.5% | 89.9% | 83.2% | 41.6% |
| H/H | 23.8% | 5.3% | 93.5% | 12.5% | 81.9% | 70.7% |

Similar to the method described above, mouse splenocytes were stimulated with 1 μg/mL human IL15/IL15RA complex, or 1 μg/mL mouse IL15/IL15RA complex, followed by flow cytometry analysis. The results are shown in the table below, which showed that after stimulation with human or mouse IL15/IL15RA complex, phosphorylated expression of STAT5 was detected in the splenocytes of wild-type C57BL/6 mice (+/+) and IL2RB/IL2RG/IL15/IL15RA four-gene humanized homozygous mice (H/H).

TABLE 19
Flow cytometry detection results of pSTAT5 expression in T cells

| | hIL15 | mIL15 | hIL15 | mIL15 | | |
|---|---|---|---|---|---|---|
| | (CD4+ | (CD4+ | (CD8+ | (CD8+ | hIL15 | mIL15 |
| Group | T cell) | T cell) | T cell) | T cell) | (T cell) | (T cell) |
| +/+ | 6.77% | 5.91% | 63.8% | 74.1% | 26.0% | 44.9% |
| H/H | 8.85% | 2.84% | 61.9% | 31.6% | 25.6% | 15.8% |

The results showed that the phosphorylation of STAT5 can be induced by stimulation with human and mouse IL2, or human and mouse IL15/IL15RA complex, indicating that IL2 and IL15 signaling pathways in IL2RB/IL2RG/IL15/IL15RA four-gene humanized mice function normally.

### Example 9: In Vivo Efficacy Verification in IL15/IL15RA Double-Gene Humanized Mice

AMG-741 is a fully human monoclonal antibody against IL-15, jointly developed by Amgen and Provention Bio, and is currently in phase II clinical trial. By blocking IL-15, AMG-714 has therapeutic potential in various inflammatory diseases, such as psoriasis, inflammatory bowel disease (IBD), lupus, multiple sclerosis (MS), etc.

Fifteen female IL15/IL15RA double-gene humanized homozygous mice (6-7 weeks old) were selected and randomly placed into a control group G1, a model group G2, and an administration group G3 (5 mice per group). Two days before the experiment, the hair on the back of the mice was removed with a shaver to expose a 2 cm×4 cm skin area. On Day 0-5 of the experiment, 5% Imiquimod (IMQ) cream (10 mg/cm²) was smeared at the back skin area of the mice in the model group (G2) and the administration group (G3) for psoriasis modeling. Vaseline (10 mg/cm²) was smeared at the back skin area of mice in the control group (G1) for 6 consecutive days. During the experiment, GI group mice received no drug treatment, G2 group mice were intraperitoneally injected with PBS, and G3 group mice were intraperitoneally injected with AMG-714 analog (heavy chain sequence set forth in SEQ ID NO: 82; light chain sequence set forth in SEQ ID NO: 83). The mice in the G2-G3 groups were administered on Day 0 and Day 3 of the experiment, with a total of 2 administrations. The entire experimental period was 9 days. The specific dosage and administration method are shown in the table below.

TABLE 20
Dosage and administration methods

| | Animal | Modeling | | | Administration | Administration |
|---|---|---|---|---|---|---|
| Group | number | reagent | Treatment | Dosage | route | frequency |
| G1 | 5 | Vaseline | — | — | — | — |
| G2 | 5 | IMQ | PBS | — | i.p. | Q3D, 2 administrations in total |
| G3 | 5 | IMQ | AMG-714 | 3 mg/kg | i.p. | Q3D, 2 administrations in total |
| | | | analog | | | |

Starting from the grouping day (Day 0), the mice were weighed every day, and photos were taken to record the mouse back skin conditions. The incidence of psoriasis was clinically scored. Scoring items included erythema and scales in mouse skin lesions. Each item was scaled into 0-4 points according to the severity, and the PASI (Psoriasis Area Severity Index) scoring standards were as follows: 0—none; 1—mild; 2—moderate; 3—severe; and 4—extremely severe. A PASI score is a tool used to measure the severity and extent of psoriasis. The average of each score and the average of the total scores of each group of mice were calculated and compared.

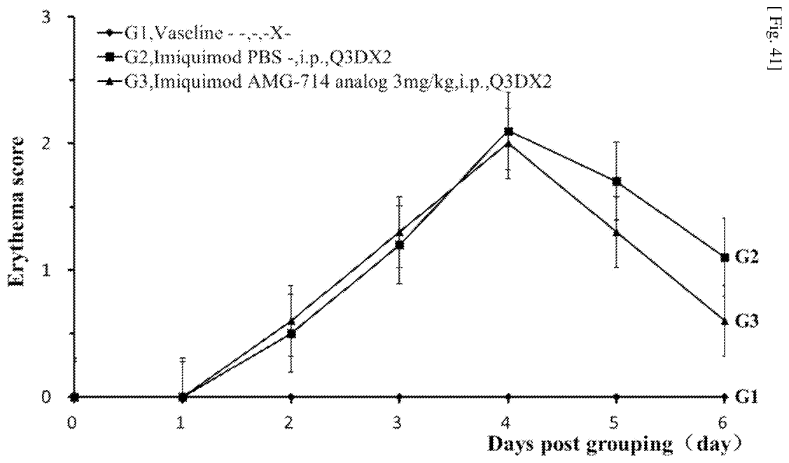

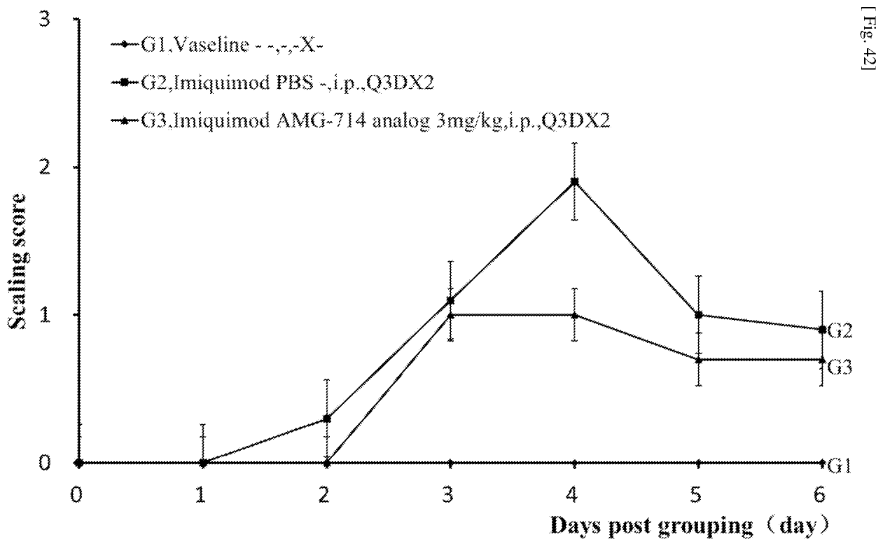

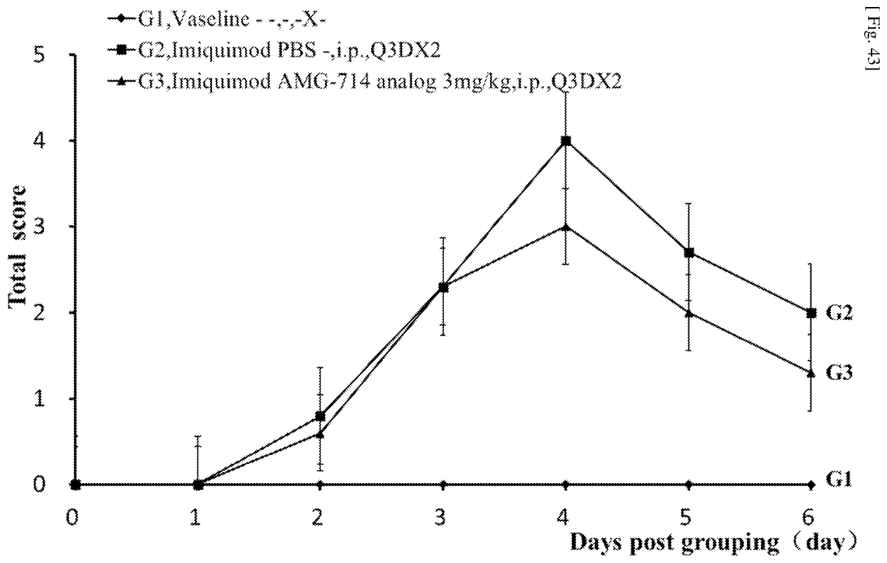

According to the body weight of the mice and its change over time (FIGS. 39-40), the weight of the control group mice (G1) was stable throughout the experimental period. The body weight of the model group mice (G2) and the administration group mice (G3) had the same changing trend over time, and they all showed a trend of falling first and then slowly rising. During the experiment, the body weight of mice from G2-G3 groups showed no observable difference. At the end of the experiment, the body weight of mice in all groups was close and there was no significant difference. The results of erythema, scaly, and comprehensive PASI scores on the back skin of the mice are shown in FIGS. 41-43. None of the mice in the control group (G1) became ill, while the model group (G2) and the administration group (G3) mice showed different degrees of disease progression. Compared with the model group, the mouse skin PASI scores of the administration group mice (G3) were lower than that of the model group mice (G2). The results indicate that administration of AMG-714 analog to psoriasis model mice exhibited a therapeutic effect on psoriasis.

The above results prove that the humanized mice as described herein can be used to establish a psoriasis model to evaluate the in vivo efficacy and dose screening of drugs targeting the human IL15/IL15RA signaling pathway.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A genetically-modified, non-human animal whose genome comprises at least one chromosome comprising a sequence encoding a human or chimeric Interleukin 2 Receptor Subunit Gamma (IL2RG).

2. The animal of claim 1, wherein the sequence encoding the human or chimeric IL2RG is operably linked to an endogenous regulatory element at the endogenous IL2RG gene locus in the at least one chromosome.

3. The animal of claim 1 or 2, wherein the sequence encoding a human or chimeric IL2RG comprises a sequence encoding an amino acid sequence that is at least 70%, 75%, 80%, 85%, 90%, 95%, 99%, or 100% identical to human IL2RG (NP_000197.1 (SEQ ID NO: 2)).

4. The animal of claim 1 or 2, wherein the sequence encoding a human or chimeric IL2RG comprises a sequence encoding an amino acid sequence that is at least 70%, 75%, 80%, 85%, 90%, 95%, 99%, or 100% identical to SEQ ID NO: 30.

5. The animal of claim 1 or 2, wherein the sequence encoding a human or chimeric IL2RG comprises a sequence encoding an amino acid sequence that is at least 70%, 75%, 80%, 85%, 90%, 95%, 99%, or 100% identical to amino acids 1-256 of SEQ ID NO: 2.

6. The animal of any one of claims 1-5, wherein the animal is a mammal, e.g., a monkey, a rodent, a mouse, or a rat.

7. The animal of any one of claims 1-6, wherein the animal does not express endogenous IL2RG or expresses a decreased level of endogenous IL2RG as compared to IL2RG expression level in a wild-type animal.

8. The animal of any one of claims 1-7, wherein the animal has one or more cells expressing human or chimeric IL2RG.

9. The animal of any one of claims 1-8, wherein the animal has one or more cells expressing human or chimeric IL2RG, and the expressed human or chimeric IL2RG can interact with human or endogenous Interleukin-2 Receptor Subunit Alpha (IL2RA) and Interleukin 2 Receptor Subunit Beta (IL2RB), forming a functional IL2 receptor complex.

10. The animal of any one of claims 1-8, wherein the animal has one or more cells expressing human or chimeric IL2RG, and the expressed human or chimeric IL2RG can interact with human or endogenous Interleukin-15 Receptor Subunit Alpha (IL15RA) and Interleukin 2 Receptor Subunit Beta (IL2RB), forming a functional IL15 receptor complex.

11. A genetically-modified, non-human animal, wherein the genome of the animal comprises a replacement of a sequence encoding a region of endogenous IL2RG with a sequence encoding a corresponding region of human IL2RG at an endogenous IL2RG gene locus.

12. The animal of claim 11, wherein the sequence encoding the corresponding region of human IL2RG is operably linked to an endogenous regulatory element at the endogenous IL2RG locus, and one or more cells of the animal expresses a human or chimeric IL2RG.

13. The animal of claim 11 or 12, wherein the animal does not express endogenous IL2RG or expresses a decreased level of endogenous IL2RG as compared to IL2RG expression level in a wild-type animal.

14. The animal of any one of claims 11-13, wherein the replaced sequence encodes the full-length IL2RG; or the signal peptide and all or a portion of the extracellular region of IL2RG.

15. The animal of any one of claims 11-14, wherein the animal has one or more cells expressing human IL2RG.

16. The animal of any one of claims 11-14, wherein the animal has one or more cells expressing a chimeric IL2RG having a signal peptide, an extracellular region, a transmembrane region, and a cytoplasmic region, wherein the extracellular region comprises a sequence that is at least 50%, 60%, 70%, 80%, 90%, 95%, or 99% identical to the extracellular region of human IL2RG (NP_000197.1 (SEQ ID NO: 2)).

17. The animal of claim 16, wherein the extracellular region of the chimeric IL2RG has a sequence that has at least 10, 20, 30, 40, 50, 60, 70, 80, 90, 100, 110, 120, 130, 140, 150, 160, 170, 180, 190, 200, 210, 220, 230, 231, 232, 233, 234, 235, 236, 237, 238, 239, or 240 contiguous amino acids that are identical to a contiguous sequence present in the extracellular region of human IL2RG (e.g., amino acids 23-256 or 23-262 of SEQ ID NO: 2).

18. The animal of claim 16 or 17, wherein the signal peptide of the chimeric IL2RG has a sequence that has at least 1, 2, 3, 4, 5, 6, 7, 8, 9, 10, 11, 12, 13, 14, 15, 16, 17, 18, 19, 20, 21, or 22 contiguous amino acids that are identical to a contiguous sequence present in the signal peptide of human IL2RG (e.g., amino acids 1-22 of SEQ ID NO: 2).

19. The animal of any one of claims 11-18, wherein the sequence encoding a region of endogenous IL2RG (e.g., mouse IL2RG) comprises exon 1, exon 2, exon 3, exon 4, exon 5, exon 6, exon 7, and/or exon 8 (e.g., exon 1, exon 2, exon 3, exon 4, exon 5, and exon 6), or a part thereof, of the endogenous IL2RG gene.

20. The animal of any one of claims 11-19, wherein the animal is heterozygous or homozygous with respect to the replacement at the endogenous IL2RG gene locus.

21. A non-human animal comprising at least one cell comprising a nucleotide sequence encoding a human or humanized IL2RG polypeptide, wherein the human or humanized IL2RG polypeptide comprises at least 50 contiguous amino acid residues that are identical to the corresponding contiguous amino acid sequence of a human IL2RG, wherein the animal expresses the human or humanized IL2RG polypeptide.

22. The animal of claim 21, wherein the human or humanized IL2RG polypeptide has at least 50, 60, 70, 80, 90, 100, 110, 120, 130, 140, 150, 160, 170, 180, 190, 200, 210, 220, 230, 231, 232, 233, 234, 235, 236, 237, 238, 239, or 240 contiguous amino acid residues that are identical to the corresponding contiguous amino acid sequence of human IL2RG extracellular region (e.g., amino acids 23-256 or 23-262 of SEQ ID NO: 2).

23. The animal of claim 21 or 22, wherein the human or humanized IL2RG polypeptide has at least 1, 2, 3, 4, 5, 6, 7, 8, 9, 10, 11, 12, 13, 14, 15, 16, 17, 18, 19, 20, 21, or 22 contiguous amino acid residues that are identical to the corresponding contiguous amino acid sequence of human IL2RG signal peptide (e.g., amino acids 1-22 of SEQ ID NO: 2).

24. The animal of any one of claims 21-23, wherein the human or humanized IL2RG polypeptide comprises a sequence that is at least 90%, 95%, or 99% identical to amino acids 1-256 or 1-262 of SEQ ID NO: 2.

25. The animal of any one of claims 21-24, wherein the nucleotide sequence is operably linked to an endogenous IL2RG regulatory element of the animal.

26. The animal of any one of claims 21-25, wherein the nucleotide sequence is integrated to an endogenous IL2RG gene locus of the animal.

27. The animal of any one of claims 21-26, wherein the nucleotide sequence encodes a humanized IL2RG polypeptide, wherein the humanized IL2RG polypeptide comprises an endogenous IL2RG transmembrane region and/or an endogenous IL2RG cytoplasmic region.

28. The animal of claim 27, wherein the humanized IL2RG polypeptide has at least one mouse IL2RG activity and/or at least one human IL2RG activity.

29. A method for making a genetically-modified, non-human animal, comprising:
replacing in at least one cell of the animal, at an endogenous IL2RG gene locus, a sequence encoding a region of endogenous IL2RG with a sequence encoding a corresponding region of human IL2RG.

30. The method of claim 29, wherein the sequence encoding the corresponding region of human IL2RG comprises exon 1, exon 2, exon 3, exon 4, exon 5, exon 6, exon 7, and/or exon 8, or a part thereof, of a human IL2RG gene.

31. The method of claim 29 or 30, wherein the sequence encoding the corresponding region of human IL2RG comprises a portion of exon 1, exon 2, exon 3, exon 4, exon 5, exon 6, exon 7, and exon 8, optionally at least 1, 2, 3, 4, 5, 6, 7, 8, 9, 10, 20, 30, 40, 50, 60, 61, 62, 63, 64, 65, 70, 80, 90, or 100 nucleotides downstream of exon 8, of a human IL2RG gene.

32. The method of claim 29 or 30, wherein the sequence encoding the corresponding region of human IL2RG comprises a portion of exon 1, exon 2, exon 3, exon 4, exon 5, and a portion of exon 6, of a human IL2RG gene.

33. The method of any one of claims 29-32, wherein the sequence encoding the corresponding region of human IL2RG encodes amino acids 1-256, 1-262, or 1-369 of SEQ ID NO: 2.

34. The method of any one of claims 29-33, wherein the region comprises the full-length IL2RG; or the signal peptide and all or a portion of the extracellular region of IL2RG.

35. The method of any one of claims 29-34, wherein the sequence encoding a region of endogenous IL2RG comprises exon 1, exon 2, exon 3, exon 4, exon 5, exon 6, exon 7, and/or exon 8, or a part thereof, of the endogenous IL2RG gene.

36. The method of any one of claims 29-35, wherein the animal is a mouse, and the sequence encoding a region of endogenous IL2RG comprises a portion of exon 1, exon 2, exon 3, exon 4, exon 5, exon 6, exon 7, and a portion of exon 8 of the endogenous IL2RG gene.

37. The method of any one of claims 29-35, wherein the animal is a mouse, and the sequence encoding a region of endogenous IL2RG comprises a portion of exon 1, exon 2, exon 3, exon 4, exon 5, and a portion of exon 6 of the endogenous IL2RG gene.

38. A method of making a genetically-modified animal cell that expresses a chimeric IL2RG, the method comprising:
replacing at an endogenous IL2RG gene locus, a nucleotide sequence encoding a region of endogenous IL2RG with a nucleotide sequence encoding a corresponding region of human IL2RG, thereby generating a genetically-modified animal cell that includes a nucleotide sequence that encodes the chimeric IL2RG, wherein the animal cell expresses the chimeric IL2RG.

39. The method of claim 38, wherein the chimeric IL2RG comprises a human or humanized IL2RG extracellular region; and a transmembrane and/or a cytoplasmic region of mouse IL2RG.

40. The method of claim 39, wherein the chimeric IL2RG further comprises a human or humanized IL2RG signal peptide.

41. A method of making a genetically-modified animal cell that expresses a human IL2RG, the method comprising:
replacing at an endogenous IL2RG gene locus, a nucleotide sequence encoding a region of endogenous IL2RG with a nucleotide sequence encoding a corresponding region of human IL2RG, thereby generating a genetically-modified animal cell that includes a nucleotide sequence that encodes the human IL2RG, wherein the animal cell expresses the human IL2RG.

42. The method of any one of claims 38-41, wherein the animal is a mouse.

43. The method of any one of 38-42, wherein the nucleotide sequence encoding the chimeric IL2RG is operably linked to an endogenous IL2RG regulatory region, e.g., promoter.

44. The animal of any one of claims 1-28, wherein the animal further comprises a sequence encoding an additional human or chimeric protein, e.g., Interleukin 2 Receptor Subunit Beta (IL2RB), Interleukin 15 (IL15), Interleukin-15 Receptor Subunit Alpha (IL15RA), Interleukin 2 (IL2), Interleukin 2 Receptor Subunit Alpha (IL2RA), programmed cell death protein 1 (PD-1), programmed death-ligand 1 (PD-L1), Interleukin 10 Receptor Subunit Alpha (IL10RA), and/or cytotoxic T-lymphocyte-associated protein 4 (CTLA4).

45. The method of any one of claims 29-43, wherein the animal or mouse further comprises a sequence encoding an additional human or chimeric protein, e.g., IL2RB, IL15, IL15RA, IL2, IL2RA, PD-1, PD-L1, IL10RA, and/or CTLA4.

46. A genetically-modified, non-human animal whose genome comprises at least one chromosome comprising a sequence encoding a human or chimeric Interleukin-15 (IL15).

47. The animal of claim 46, wherein the sequence encoding the human or chimeric IL15 is operably linked to an endogenous regulatory element at the endogenous IL15 gene locus in the at least one chromosome.

48. The animal of claim 46 or 47, wherein the sequence encoding a human or chimeric IL15 comprises a sequence encoding an amino acid sequence that is at least 70%, 75%, 80%, 85%, 90%, 95%, 99%, or 100% identical to human IL15 (NP_000576.1 (SEQ ID NO: 52)).

49. The animal of claim 46 or 47, wherein the sequence encoding a human or chimeric IL15 comprises a sequence that is at least 70%, 75%, 80%, 85%, 90%, 95%, 99%, or 100% identical to SEQ ID NO: 55.

50. The animal of any one of claims 46-49, wherein the animal is a mammal, e.g., a monkey, a rodent, a mouse, or a rat.

51. The animal of any one of claims 46-50, wherein the animal does not express endogenous IL15 or expresses a decreased level of endogenous IL15 as compared to IL15 expression level in a wild-type animal.

52. The animal of any one of claims 46-51, wherein the animal has one or more cells expressing human or chimeric IL15.

53. The animal of any one of claims 46-52, wherein the animal has one or more cells expressing human or chimeric IL15, and the expressed human or chimeric IL15 is functional that can interact with a human, chimeric, or endogenous IL15 receptor complex.

54. A genetically-modified, non-human animal, wherein the genome of the animal comprises a replacement of a sequence encoding a region of endogenous IL15 with a sequence encoding a corresponding region of human IL15 at an endogenous IL15 gene locus.

55. The animal of claim 54, wherein the sequence encoding the corresponding region of human IL15 is operably linked to an endogenous regulatory element at the endogenous IL15 locus, and one or more cells of the animal expresses a human or chimeric IL15.

56. The animal of claim 54 or 55, wherein the animal does not express endogenous IL15 or expresses a decreased level of endogenous IL15 as compared to IL15 expression level in a wild-type animal.

57. The animal of any one of claims 54-56, wherein the replaced sequence encodes the full-length IL15.

58. The animal of any one of claims 54-57, wherein the sequence encoding a region of endogenous IL15 (e.g., mouse IL15) comprises exon 1, exon 2, exon 3, exon 4, exon 5, exon 6, exon 7, and/or exon 8 (e.g., exon 3, exon 4, exon 5, exon 6, exon 7, and exon 8), or a part thereof, of the endogenous IL15 gene.

59. The animal of any one of claims 54-58, wherein the animal is heterozygous or homozygous with respect to the replacement at the endogenous IL15 gene locus.

60. A non-human animal comprising at least one cell comprising a nucleotide sequence encoding a human or humanized IL15 polypeptide, wherein the human or humanized IL15 polypeptide comprises at least 50, 60, 70, 80, 90, 100, 110, 120, 130, 140, 150, 160, 161, or 162 contiguous amino acid residues that are identical to the corresponding contiguous amino acid sequence of a human IL15, wherein the animal expresses the human or humanized IL15 polypeptide.

61. The animal of claim 60, wherein the human or humanized IL15 polypeptide comprises a sequence that is at least 90%, 95%, or 99% identical to SEQ ID NO: 52.

62. The animal of claim 60 or 61, wherein the nucleotide sequence is operably linked to an endogenous IL15 regulatory element of the animal.

63. The animal of any one of claims 60-62, wherein the nucleotide sequence is integrated to an endogenous IL15 gene locus of the animal.

64. A method for making a genetically-modified, non-human animal, comprising:
replacing in at least one cell of the animal, at an endogenous IL15 gene locus, a sequence encoding a region of endogenous IL15 with a sequence encoding a corresponding region of human IL15.

65. The method of claim 64, wherein the sequence encoding the corresponding region of human IL15 comprises exon 1, exon 2, exon 3, exon 4, exon 5, exon 6, exon 7, and/or exon 8, or a part thereof, of a human IL15 gene.

66. The method of claim 64 or 65, wherein the sequence encoding the corresponding region of human IL15 comprises a portion of exon 3, exon 4, exon 5, exon 6, exon 7, and a portion of exon 8, of a human IL15 gene.

67. The method of any one of claims 64-66, wherein the sequence encoding the corresponding region of human IL15 encodes SEQ ID NO: 52.

68. The method of any one of claims 64-67, wherein the sequence encoding a region of endogenous IL15 comprises exon 1, exon 2, exon 3, exon 4, exon 5, exon 6, exon 7, and/or exon 8, or a part thereof, of the endogenous IL15 gene.

69. The method of any one of claims 64-68, wherein the animal is a mouse, and the sequence encoding a region of endogenous IL15 comprises a portion of exon 3, exon 4, exon 5, exon 6, exon 7, and a portion of exon 8, of the endogenous IL15 gene.

70. A method of making a genetically-modified animal cell that expresses a human or humanized IL15, the method comprising:
replacing at an endogenous IL15 gene locus, a nucleotide sequence encoding a region of endogenous IL15 with a nucleotide sequence encoding a corresponding region of human IL15, thereby generating a genetically-modified animal cell that includes a nucleotide sequence that encodes the human or humanized IL15, wherein the animal cell expresses the human or humanized IL15.

71. The method of claim 70, wherein the animal is a mouse.

72. The method of claim 70 or 71, wherein the nucleotide sequence encoding the human or humanized IL15 is operably linked to an endogenous IL15 regulatory region, e.g., promoter.

73. The animal of any one of claims 46-63, wherein the animal further comprises a sequence encoding an additional human or chimeric protein, e.g., Interleukin 2 Receptor Subunit Beta (IL2RB), Interleukin 2 Receptor Subunit Gamma (IL2RG), Interleukin-15 Receptor Subunit Alpha (IL15RA), Interleukin 2 (IL2), Interleukin 2 Receptor Subunit Alpha (IL2RA), programmed cell death protein 1 (PD-1), programmed death-ligand 1 (PD-L1), Interleukin 10 Receptor Subunit Alpha (IL10RA), and/or cytotoxic T-lymphocyte-associated protein 4 (CTLA4).

74. The method of any one of claims 64-72, wherein the animal or mouse further comprises a sequence encoding an additional human or chimeric protein, e.g., IL2RB, IL2RG, IL15RA, IL2, IL2RA, PD-1, PD-L1, IL10RA, and/or CTLA4.

75. A method of determining effectiveness of a therapeutic agent for treating an allergic disorder (e.g., allergy, asthma, and/or atopic dermatitis), comprising:
a) administering the therapeutic agent to the animal of any one of claims 1-28, 44, 46-63, and 73, wherein the animal has the allergic disorder; and
b) determining effects of the therapeutic agent in treating the allergic disorder.

76. A method of determining effectiveness of a therapeutic agent for reducing an inflammation (e.g., skin inflammation or infection), comprising:
a) administering the therapeutic agent to the animal of any one of claims 1-28, 44, 46-63, and 73, wherein the animal has the inflammation; and
b) determining effects of the therapeutic agent for reducing the inflammation.

77. A method of determining effectiveness of a therapeutic agent for treating an immune disorder, comprising:
a) administering the agent to the animal of any one of claims 1-28, 44, 46-63, and 73, wherein the animal has the immune disorder; and
b) determining effects of the therapeutic agent for treating the immune disorder.

78. The method of claim 77, wherein the immune disorder is psoriasis.

79. The method of claim 77, wherein the immune disorder is an autoimmune disease, e.g., graft versus host disease (GVHD), psoriasis, allergy, asthma, myocarditis, nephritis, hepatitis, systemic lupus erythematosus, rheumatoid arthritis, scleroderma, hyperthyroidism, idiopathic thrombocytopenia purpura, autoimmune hemolytic anemia, ulcerative colitis, autoimmune liver disease, diabetes, pain or neurological disorders.

80. The method of any one of claims 75-79, wherein the therapeutic agent includes an anti-IL2RB antibody, an anti-IL2RG antibody, an anti-IL15RA antibody, and/or an anti-IL15 antibody, or a corticosteroid (e.g., dexamethasone).

81. A method of determining effectiveness of a therapeutic agent for treating a cancer, comprising:
a) administering the therapeutic agent to the animal of any one of claims 1-28, 44, 46-63, and 73, wherein the animal has the cancer; and
b) determining inhibitory effects of the therapeutic agent for treating the cancer.

82. The method of claim 81, wherein the therapeutic agent includes an anti-IL2RB antibody, an anti-IL2RG antibody, an anti-IL15RA antibody, and/or an anti-IL15 antibody.

83. The method of claim 81 or 82, wherein the cancer is a tumor, and determining the inhibitory effects of the treatment involves measuring the tumor volume in the animal.

84. The method of any one of claims 81-83, wherein the cancer comprises one or more cancer cells that are injected into the animal.

85. The method of any one of claims 81-84, wherein the cancer is breast cancer, ovarian cancer, endometrial cancer, melanoma, kidney cancer, lung cancer, or liver cancer.

86. A method of determining toxicity of an anti-IL2RB antibody, an anti-IL2RG antibody, an anti-IL15RA antibody, and/or an anti-IL15 antibody, comprising:
a) administering the anti-IL2RB antibody, the anti-IL2RG antibody, the anti-IL15RA antibody, and/or the anti-IL15 antibody to the animal of any one of claims 1-28, 44, 46-63, and 73; and
b) determining effects of the therapeutic agent to the animal.

87. The method of claim 86, wherein determining effects of the therapeutic agent to the animal involves measuring the body weight, red blood cell count, hematocrit, and/or hemoglobin of the animal.

88. A protein comprising an amino acid sequence, wherein the amino acid sequence is one of the following:
(a) an amino acid sequence set forth in SEQ ID NO: 1, 2, 30, 33, 34, 39, 42, 43, 50, 51, or 52;
(b) an amino acid sequence that is at least 90% identical to SEQ ID NO: 1, 2, 30, 33, 34, 39, 42, 43, 50, 51, or 52;
(c) an amino acid sequence that is at least 91%, 92%, 93%, 94%, 95%, 96%, 97%, 98%, or 99% identical to SEQ ID NO: 1, 2, 30, 33, 34, 39, 42, 43, 50, 51, or 52;
(d) an amino acid sequence that is different from the amino acid sequence set forth in SEQ ID NO: 1, 2, 30, 33, 34, 39, 42, 43, 50, 51, or 52 by no more than 10, 9, 8, 7, 6, 5, 4, 3, 2 or 1 amino acid; and
(e) an amino acid sequence that comprises a substitution, a deletion and/or insertion of one, two, three, four, five or more amino acids to the amino acid sequence set forth in SEQ ID NO: 1, 2, 30, 33, 34, 39, 42, 43, 50, 51, or 52.

89. A nucleic acid comprising a nucleotide sequence, wherein the nucleotide sequence is one of the following:
(a) a sequence that encodes the protein of claim 88;
(b) SEQ ID NO: 3, 4, 5, 6, 7, 8, 9, 24, 25, 26, 27, 28, 29, 31, 32, 35, 36, 37, 38, 40, 41, 44, 45, 46, 47, 48, 49, 53, 54, 55, 56, 57, or 58;
(c) a sequence that is at least 90% identical to SEQ ID NO: 3, 4, 5, 6, 7, 8, 9, 24, 25, 26, 27, 28, 29, 31, 32, 35, 36, 37, 38, 40, 41, 44, 45, 46, 47, 48, 49, 53, 54, 55, 56, 57, or 58; and
(d) a sequence that is at least 91%, 92%, 93%, 94%, 95%, 96%, 97%, 98%, or 99% identical to SEQ ID NO: 3, 4, 5, 6, 7, 8, 9, 24, 25, 26, 27, 28, 29, 31, 32, 35, 36, 37, 38, 40, 41, 44, 45, 46, 47, 48, 49, 53, 54, 55, 56, 57, or 58.

90. A cell comprising the protein of claim 88 and/or the nucleic acid of claim 89.

91. An animal comprising the protein of claim 88 and/or the nucleic acid of claim 89.
